(19) United States
(12) Patent Application Publication
(10) Pub. No.: US 2023/0116223 A1
(43) Pub. Date: Apr. 13, 2023

(54) NUCLEASE-SCAFFOLD COMPOSITION DELIVERY PLATFORM

(71) Applicant: JENTHERA THERAPEUTICS INC., Montreal (CA)

(72) Inventor: Philip ROCHE, Montreal (CA)

(21) Appl. No.: 17/795,914

(22) PCT Filed: Jan. 28, 2021
(86) PCT No.: PCT/IB2021/000073

§ 371 Date: Jul. 28, 2022

Related U.S. Application Data
(60) Provisional application No. 62/967,259, filed on Jan. 29, 2020.

(51) Int. Cl. C12N 15/90; C12N 15/10; C07K 16/28; C07K 19/00; C12N 9/22; A61P 31/18

(52) U.S. Cl. CPC C12N 15/907; C12N 15/102; C07K 16/2863; C07K 19/00; C12N 9/22; A61P 31/18; A61K 2039/505

(57) ABSTRACT

Described herein are methods, compositions, and systems for gene editing using polynucleotide modifying enzymes that do not require the use of chemical transfection agents for entry into cells.

### CROSS-REFERENCE STATEMENT

This application claims the benefit of U.S. Provisional Application 62/967,259, entitled “NUCLEASE-SCAFFOLD COMPOSITION DELIVERY PLATFORM”, filed on Jan. 29, 2020, which is incorporated by reference herein in its entirety.

### BACKGROUND OF THE INVENTION

CRISPR (clustered regularly interspaced short palindromic repeats) RNA-directed DNA nucleases are firmly established as a major gene editing methodology with potential applications in research, pharmaceutical development and therapeutics. Prior to CRISPR programmable nucleases, less versatile programmable nucleases which rely on protein engineering (such as Zn-finger Nucleases, TALENS and Meganucleases such as natural and engineered derivatives of I-Cre1 and others) or nucleases that require insertion of a targeting site (e.g. RAD52/51, CRE) had been used to achieve double stranded breaks in DNA. However, the rapid design and programmability CRISPR nucleases by guide RNA creates a readily addressable gene editing solution that truncates the experimental workflow for testing hypotheses at the genomic level. Since the only engineered component required for CRISPR genome targeting is a guide RNA which can be synthesized according to predictable rules, genomic regions can be targeted with much less unpredictable experimentation. Further, CRISPR nucleases active in mammalian cells have provided a new avenue for programmable nuclease therapeutics, allowing targeting of genomic locations difficult to target by other methodologies.

### SUMMARY OF THE INVENTION

In some aspects, the present disclosure provides for a composition for modifying a gene comprising: a cell recognition domain; an endosome escape domain; and a polynucleotide-modifying enzyme domain; wherein the endosome escape domain is covalently coupled to the cell recognition domain. In some embodiments, the composition further comprises a hapten binding-domain. In some embodiments, the cell recognition domain, endosome escape domain, polynucleotide-modify enzyme domain, and the optional hapten-binding domain are physically linked. In some embodiments, the composition further comprises a bispecific scaffold, wherein the bispecific scaffold binds non-covalently to the cell recognition domain and the polynucleotide-modifying enzyme domain. In some embodiments, the bispecific scaffold comprises a hapten and the hapten-binding domain binds to the hapten. In some embodiments, one or more of the domains are physically linked by protein ligation. In some embodiments, one or more of the domains are linked in the order according to FIG. 1. In some embodiments, one or more of the domains are linked in the order of any one of the following: (a) PNME-CRD-EE; (b) CRD-PNME-EE; (c) EE-CRD-PNME; (d) PNME-Hapten binding domain-EE; (e) PNME-Hapten binding domain-CRD-EE; (f) EE-CRD-PNME-Hapten binding domain; or (g) EE-Hapten binding domain-PNME-CRD. In some embodiments, one or more of the domains are linked in the order of any one of the following: (a) PNME-CRD-EE; or (b) PNME-Hapten binding domain-CRD-EE. In some embodiments, one or more of the domains are physically linked by one or more peptide linkers described in Table 4, or one or more chemical cross-linkers. In some embodiments, one or more of the cell recognition domain, the endosome escape domain, and the polynucleotide-modifying enzyme domain are physically linked in the form of a fusion polypeptide. In some embodiments, the fusion peptide further comprises a non-structural linker domain. In some embodiments, the fusion peptide comprises the cell recognition domain and the endosome escape domain. In some embodiments, the fusion polypeptide comprises the cell recognition domain, the endosome escape domain, and the polynucleotide-modifying enzyme domain. In some embodiments, the fusion polypeptide further comprises the hapten-binding domain. In some embodiments, the polynucleotide-modifying enzyme domain is located at the N-terminus of the fusion polypeptide. In some embodiments, the cell recognition domain is located at the N-terminus of the fusion polypeptide. In some embodiments, the endosome escape domain is located at the N-terminus of the fusion polypeptide. In some embodiments, the endosome escape domain is located at the C-terminus of the fusion polypeptide. In some embodiments, the cell recognition domain is located at the C-terminus of the fusion polypeptide. In some embodiments, the polynucleotide-modifying enzyme domain is located at the C-terminus of the fusion polypeptide. In some embodiments, the hapten-binging domain is located at the C-terminus of the fusion polypeptide. In some embodiments, the total molecular weight of the composition is between 100 kDa and 240 kDa. In some embodiments, the total molecular weight of the composition is between 100 kDa and 200 kDa. In some embodiments, the hydrodynamic radius of the composition is less than 100 nm. In some embodiments, the hydrodynamic radius of the composition is less than 90 nm, 80 nm, 70 nm or 60 nm. In some embodiments, the cell recognition domain binds to one or more epitopes on a cell-surface antigen. In some embodiments, the epitope is an epitope of a receptor displayed on the surface of a cell. In some embodiments, the epitope is a protein ligand and the ligand binds to a receptor displayed on the surface of a cell. In some embodiments, the cell internalizes the receptor by clathrin-mediated endocytosis, calveolin-mediated endocytosis, or micropinocytosis. In some embodiments, binding of the cell recognition domain to the receptor induces the cell to internalize the receptor. In some embodiments, the receptor is selectively expressed on a target cell or class of target cells, and the receptor is not expressed, or poorly expressed on a cell that is not the target cell. In some embodiments, the target cell is a diseased cell or a cancer cell. In some embodiments, the epitope is an epitope of a G-protein coupled receptor. In some embodiments, the epitope is an epitope of a protein selected from the group consisting of L-SIGN (also known as CLEC4M, C-Type Lectin Domain Family 4 Member M, CD299), ASGPR (also known as ASGR1, ASGR2, Asialoglycoprotein receptor 1 or 2), AT1 (also known as Angiotensin II Receptor Type 1, AGTR1), B2/B1 receptor (also known as Bradykinin Receptor B1 or B2, BDKRB1, BDKRB2, BKRB1, BKRB2), and Muscarinic receptors (also known as Muscarinic acetylcholine receptors, mAChRs). In some embodiments, the epitope is selected from the group consisting of L-SIGN (also known as CLEC4M, C-Type Lectin Domain Family 4 Member M, CD299), ASGPR (also known as ASGR1, ASGR2, Asialoglycoprotein receptor 1 or 2), AT1 (also known as Angiotensin II Receptor Type 1, AGTR1), B2/B1 receptor (also known as Bradykinin Receptor B1 or B2, BDKRB1, BDKRB2, BKRB1, BKRB2), Muscarinic receptors (also known as Muscarinic acetylcholine receptors, mAChRs), FGFR4 (also known as Fibroblast Growth Factor Receptor 4), FGFR3 (also known as Fibroblast Growth Factor Receptor 3), FGFR1 (also known as Fibroblast Growth Factor Receptor 1), Frizzled 4 (also known as Frizzled Class Receptor 4, FZD4), S1PR1 (also known as Sphingosine-1-Phosphate Receptor 1), TSHR (also known as Thyroid Stimulating Hormone Receptor), GPR41 (also known as Free Fatty Acid Receptor 3, G Protein-Coupled Receptor 41, FFAR3), GPR43 (also known as G Protein-Coupled Receptor 43, FFAR2, Free Fatty Acid Receptor 2), GPR109A (also known as G Protein-Coupled Receptor 109A, Niacin Receptor 1, NIACR1, Hydroxycarboxylic Acid Receptor 2, HCAR2), TFRC (also known as Transferrin Receptor, CD71, TFR1), Insulin receptor (also known as INSR, CD220), Insulin-like growth factor 2 receptor (also known as IGF2R, Cation-independent mannose-6-prosphate receptor, CI-MPR, MPRI), LRP1 (also known as LDL Receptor Related Protein 1, Apolipoprotein E Receptor, APOER, CD91), IGF1R (also known as Insulin Like Growth Factor 1 Receptor, CD221), Prolactin receptor (also known as PRLR), and Follicle stimulating hormone receptor (also known as FSHR, FSH receptor, Follitropin Receptor, LGR1). In some embodiments, the epitope is selected from the group consisting of cd44v6, CAIX (also known as Carbonic Anhydrase 9, CA9), CEA (also known as CEA Cell Adhesion Molecule 5, CEACAM5, Carcinoembryonic antigen), CD133 (also known as Prominin 1, PROM1), cMet hepatocyte growth factor receptor (also known as MET), EGFR (also known as Epidermal Growth Factor Receptor, HER1), EGFR vIII, EPCAM (also known as Epithelial Cell Adhesion Molecule), EphA2 (also known as EPH Receptor A2), Fetal acetylcholine receptor, FRalpha folate receptor (also known as FOLR1), GD2 (also known as Ganglioside G2), GPC3 (also known as Glypican 3), GUCY2C (also known as Guanylate Cyclase 2C), HER2 (also known as ERBB2), ICAM1 (also known as Intercellular Adhesion Molecule 1), IL13Ralpha2 (also known as IL13RA2), IL11 receptor alpha (also known as IL11RA), Kras, Kras G12D, L1cam (also known as L1 Cell Adhesion Molecule), MAGE (also known as melanoma-associated antigen), Mesothelin (also known as MSLN), MUC1 (also known as Mucin 1, Cell Surface Associated), MUC16 (also known as Mucin 16, Cell Surface Associated), NKG2D (also known as Killer Cell Lectin Like Receptor K1, KLRK1, NK Cell receptor D, CD314), NY-ESO1 (also known as New York Esophageal Squamous Cell Carcinoma 1, CTAG1B, Cancer/Testis Antigen 1B), PSCA (also known as Prostate Stem Cell Antigen, PRO232), WT1 (also known as WT1 Transcription Factor, Wilms Tumor Protein), PSMA (also known as prostate-specific membrane antigen, Glutamate carboxypeptidase II, GCPII, N-acetyl-L-aspartyl-L-glutamate peptidase I, NAALADase I, NAAG peptidase, FOLH1, folate hydrolase 1), 5t4 or TPBG (also known as Trophoblast Glycoprotein), Transferrin receptor (also known as TFRC, CD71, TFR1), GPNMB Breast cancer, melanoma (also known as Glycoprotein Nmb), LeY (also known as Lewis y antigen, Lewis y Tetrasaccharide), CA6 (also known as Carbonic anhydrase 6, CA-VI), Av integrin (also known as ITGAV, Integrin Subunit Alpha V), SLC44A4 (also known as Solute Carrier Family 44 Member 4), Nectin-4 (also known as NECTIN4, NECT4, PVRL4, EDSS1) Solid tumors, AGS-16 (also known as Ectonucleotide Pyrophosphatase/Phosphodiesterase 3, ENPP3), Cripto (also known as CFC1, FRL-1, Cryptic Family 1), TENB2 (also known as Transmembrane Protein With EGF Like And Two Follistatin Like Domains 2, TMEFF2, Tomoregulin-2, HPP1, TPEF), EPCAM, and CD166. In some embodiments, the cell recognition domain comprises two or more binding components, wherein the first binding component binds to a first epitope and the second binding component binds to a second epitope. In some embodiments, the cell recognition domain comprises at least three binding components, and the third binding component binds to a third epitope. In some embodiments, the cell recognition domain comprises at least four binding components, and the fourth binding component binds to a fourth epitope. In some embodiments, the first epitope and the second epitope, and, optionally, the third epitope and the fourth epitope are located on the same cell surface antigen or receptor. In some embodiments, the first epitope is located on a first cell surface antigen or receptor and the second epitope is located on a second cell surface antigen or receptor and, optionally, the third epitope is located on a third cell surface antigen or receptor and, optionally, the fourth epitope is located on a fourth cell surface antigen or receptor. In some embodiments, the first cell surface receptor is a driver receptor that is rapidly internalized by a target cell and the second cell surface receptor is a passenger receptor that is not rapidly internalized by the target cell. In some embodiments, the first cell surface receptor is EPCAM and the second cell surface receptor is ALCAM. In some embodiments, the cell recognition domain is a protein ligand. In some embodiments, the protein ligand comprises 5 to 15 amino acids in length. In some embodiments, the protein ligand has a globular or cyclical structure. In some embodiments, the protein ligand is an antibody or antigen-binding domain thereof. In some embodiments, the antigen-binding domain is a Fab, scFv, single-domain antibody (sdAb), V_{HH}, or camelid antibody domain. In some embodiments, the protein ligand is an antibody mimetic. In some embodiments, the antibody mimetic is selected from the group consisting of affibody, an affilin, an affimer, an affitin, an alphabody, an anticalin, an atrimer, an avimer, a DARPin, a fynomer, a knottin, a Kunitz domain peptide, a monobody, a nanoCLAMP, and a linear peptide comprising 6-20 amino acids in length. In some embodiments, the cell recognition domain is an oligonucleotide. In some embodiments, the oligonucleotide is a ribonucleotide or deoxyribonucleotide. In some embodiments, the oligonucleotide comprises a non-canonical nucleotide. In some embodiments, the non-canonical nucleotide is selected from the group consisting of 2′-OMe, 2′-F, or 4′-S nucleotides, 2′-FANAs, HNAs, or locked nucleic acid residues. In some embodiments, the cell recognition domain comprises a chemical ligand with a molecular weight of less than about 800 Da. In some embodiments, the endosome escape domain comprises between 3 and 9 amino acids. In some embodiments: the amino acid residue at position 1 of the endosome escape domain is a proline or cysteine; the amino acid residues at positions 2-5 of the endosome escape domain are cysteines, arginines, or lysines; and/or the amino acid residues at positions 6-9 of the endosome escape domain are cysteines, arginines, lysines, alanines or tryptophans. In some embodiments, the endosome escape domain comprises at least 3 cysteines and no more than 8 cysteines. In some embodiments, the polynucleotide-modifying enzyme domain comprises a nuclear localization sequence (NLS). In some embodiments, the NLS sequence is located in a linker domain fused to the N-terminus of the polynucleotide-modifying enzyme domain. In some embodiments, the NLS sequence is located in a linker domain fused to the C-terminus of the polynucleotide-modifying enzyme domain. In some embodiments, the NLS sequence comprises 7-25 amino acid residues. In some embodiments, the NLS is a bipartite NLS wherein amino acids within an N-terminal portion of the NLS involved in the recognition of an importin and amino acids within an a C-terminal portion of the NLS involved in the recognition of an importin are split by an amino acid sequence not involved in the recognition of an importin. In some embodiments, the polynucleotide-modifying enzyme domain further comprises a linker sequence separating the NLS from the polynucleotide-modifying enzyme. In some embodiments, the linker comprises between 6 and 20 amino acid residues. In some embodiments, the NLS comprises a sequence having at least 90% or 95% identity to a sequence selected from the group consisting of SEQ ID NOs: 1-16. In some embodiments, the polynucleotide-modifying enzyme domain comprises two or more NLSs. In some embodiments, the two or more NLSs comprise a first NLS and a second NLS, wherein the first NLS has the same sequence as the second NLS, and wherein the first NLS is separated from the second NLS by a linker sequence comprising 1-7 amino acid residues. In some embodiments, the composition further comprises a third NLS with the same sequence as the first NLS and the second NLS. In some embodiments, the two or more NLSs comprise a first NLS and a second NLS, and the first NLS has a different sequence than the second NLS. In some embodiments, the hapten binding domain can bind to a hapten that is covalently attached to a peptide, a protein, an oligonucleotide, or a polynucleotide. In some embodiments, the protein is selected from the group consisting of an adenosine deaminase, a cytosine deaminase, a transcriptional activator, and a transcriptional suppressor. In some embodiments, the oligonucleotide is a deoxyoligoribonucleotide or ribooligonucleotide. In some embodiments, the oligonucleotide is a single-stranded oligonucleotide or a double-stranded oligonucleotide. In some embodiments, the hapten is selected form the group consisting of fluorescein, biotin, and digoxin. In some embodiments, the polynucleotide-modifying enzyme domain is a nuclease, a recombinase, or an RNA editing enzyme. In some embodiments, the nuclease comprises a programmable component that directs the nuclease against either DNA or RNA in response to target nucleotide sequence. In some embodiments, the nuclease cleaves a ribonucleic acid target or a deoxyribonucleic acid target. In some embodiments, the nuclease cleaves a single-stranded polynucleotide target. In some embodiments, the nuclease cleaves a double-stranded polynucleotide target. In some embodiments, the cleaved double-stranded polynucleotide target has a blunt end, two staggered ends, or a nick in one strand and an intact second strand. In some embodiments, the polynucleotide target is a double stranded polynucleotide target and the nuclease cleaves one strand of the double-stranded polynucleotide target. In some embodiments, the polynucleotide-modifying enzyme domain comprises a programmable endonuclease. In some embodiments, the site-specific endonuclease comprises a Class II Cas enzyme, a TALEN, a meganuclease, a Zn-finger nuclease derivatives, or nuclease-deficient variants thereof. In some embodiments, the class II Cas enzyme comprises a type II, type V, or type VI Cas enzyme. In some embodiments, the class II Cas enzyme comprises a type V Cas enzyme. In some embodiments, the type V Cas enzyme comprises asCpfI or MAD7. In some embodiments, the composition further comprises a guide oligonucleotide complementary to a target gene, wherein the guide oligonucleotide is non-covalently bound to the polynucleotide-modifying enzyme domain. In some embodiments, guide oligonucleotide comprises a non-complementary region derived from a naturally occurring type II, type V, or type VI crRNA or tracrRNA. In some embodiments, the guide oligonucleotide comprises a ribonucleotide or a ribonucleotide and a deoxyribonucleotide. In some embodiments, the guide oligonucleotide comprises a non-canonical nucleotide. In some embodiments, the non-canonical nucleotide comprises a modification at the 2′ position of a sugar moiety. In some embodiments, the non-canonical nucleotide is selected from the group consisting of 2′-OMe, 2′-F, or 4′-S nucleotides, 2′-FANAs, HNAs, or locked nucleic acid residues. In some embodiments, the guide oligonucleotide comprises one or more bridged nucleotides in a seed region of the guide oligonucleotide. In some embodiments, the guide oligonucleotide comprises a sequence of n nucleotides counting from a 1^{st} nucleotide at a 5′ end to an n^{th} nucleotide at a 3′ end, wherein one or more of the nucleotides at positions 1, 2, n-1 and n are phosphorothioate modified nucleotides. In some embodiments, the nuclease-deficient polynucleotide-modifying domain can bind DNA and is fused to second enzyme that is capable of epigenetic modifications or base chemical conversion. In some embodiments, the epigenetic modification is selected from the group consisting of methylation, RNA cleavage, cytosine deamination, and adenosine deamination. In some embodiments, the base chemical conversion is selected from adenosine deamidation and cytosine deamidation. In some embodiments, the recombinase is a mammalian recombinase or a eukaryotic recombinase. In some embodiments, the recombinase is a Rad52/51 recombinase or a CRE recombinase. In some embodiments, the composition further comprises a donor DNA polynucleotide comprising a 5′ homology region and a 3′ homology region, wherein the 5′ homology region comprises a nucleotide sequence with sequence identity to a nucleotide sequence on the 5′ side of the target nucleotide sequence and the 3′ homology region comprises a nucleotide sequence with sequence identity to a nucleotide sequence on the 3′ side of the target nucleotide sequence. In some embodiments, the donor DNA polynucleotide further comprises an insert region, and the insert region lies between the 5′ homology region and the 3′ homology region. In some embodiments, the insert region comprises an exon, an intron, a transgene, a selectable marker, or a stop codon. In some embodiments, the target nucleotide sequence comprises a mutation and the insert region does not comprise a mutation. In some embodiments, the 5′ homology region and the 3′ homology region have the same length. In some embodiments, the 5′ homology region and the 3′ homology region have different lengths. In some embodiments, the donor DNA polynucleotide is a single stranded polynucleotide and the 5′ homology region comprises 50-100 nucleotides and the 3′ homology region comprises 20-60 nucleotides. In some embodiments, the 3′ end of the 5′ homology region is homologous to a sequence within 5 nucleotides of the double-stranded break and the 5′ end of the 3′ homology region is homologous to a sequence within 5 nucleotides of the double strand break. In some embodiments, the nuclease is a type II or a type V nuclease. In some embodiments, the nuclease is a type V nuclease, the target polynucleotide sequence comprises a protospacer adjacent motif (PAM) located within 30 nucleotides of the cleavage site, the cleaved double-stranded polynucleotide target has two staggered ends, and the staggered ends have 4 nucleotide 5′ or 3′ overhangs. In some embodiments, a hapten is conjugated to the donor DNA polynucleotide and the hapten binds to the hapten-binding domain. In some embodiments, a peptide of less than 20 amino acids in length is conjugated to the donor DNA polynucleotide and the peptide binds to the cell recognition domain. In some embodiments, the composition does not comprise a PEI, PEG, PAMAN, or sugar (dextran) derivative polymer comprising more than three subunits. In some embodiments, the composition comprises a protein sequence having at least 80% identity to any one of SEQ ID NOs: 16-26, 44, 46, 48, 50, 52, 54, 56, 58, 60, 61-65, 67, 69, 71, 73, 75, 77, 79, 81, 83, 85, 87, or a variant thereof. In some embodiments, the composition comprises a protein sequence having at least 80% identity to any one of SEQ ID NOs 67, 69, 71, 73, 75, 77, 79, 81, 83, 85, 87, or a variant thereof. In some embodiments, the composition comprises a protein sequence having at least 80% identity to SEQ ID NO 77, 85, 87, or a variant thereof. In some embodiments, the composition comprises a guide oligonucleotide complementary to a target gene, wherein the guide oligonucleotide comprises a nucleotide sequence having at least 80% identity to any one of SEQ ID NOs: 88-109, or a variant thereof. In some embodiments, the composition comprises a guide oligonucleotide complementary to a target gene, wherein the guide oligonucleotide comprises a nucleotide sequence having at least 80% identity to any one of SEQ ID NOs: 94, 95, 96, 97, 98 99, 100, 101, or a variant thereof.

In some aspects the present disclosure provides for a vector comprising a nucleotide sequence encoding a cell recognition domain, an endosome escape domain, and a polynucleotide-modifying enzyme domain. In some embodiments, the vector further comprises a nucleotide sequence encoding a hapten-binding domain.

In some aspects the present disclosure provides for a vector comprising a nucleotide sequence encoding the any of the compositions described herein. In some embodiments, the vector is a plasmid.

In some aspects, the present disclosure provides for a host cell comprising any of the vectors described herein. In some embodiments, the any of the fusion proteins described herein are secreted from the cell. In some embodiments, the host cell is a prokaryotic cell, a eukaryotic cell, an E. coli cell, an insect cell, or an Sf9 cell.

In some aspects, the present disclosure provides for a kit for editing a gene in a cell comprising any of the compositions described herein, a guide oligonucleotide and a donor DNA polynucleotide.

In some aspects, the present disclosure provides for a kit for editing a gene in a cell comprising any of the vectors described herein, a guide oligonucleotide and a donor DNA polynucleotide.

In some aspects, the present disclosure provides for a kit for editing a gene in a cell comprising any of the host cells described herein, a guide oligonucleotide and a donor DNA polynucleotide.

In some aspects, the present disclosure provides for a method of editing a gene by random insertion or deletion comprising contacting any of the compositions described herein to a cell.

In some aspects, the present disclosure provides for a method of editing a gene by homology directed repair comprising any of the compositions described herein to a cell. In some embodiments, the gene is modified by insertion of a label. In some embodiments, the label is selected from the list consisting of epitope tag or a fluorescent protein tag. In some embodiments, a mutation in the gene is repaired.

In some aspects, the present disclosure provides for a method of inserting a transgene into the genome of a cell by homologous recombination comprising contacting any of the compositions described herein to the cell.

In some aspects, the present disclosure provides for a method of generating a cell amenable to gene editing comprising expressing a receptor in the cell, wherein the cell recognition domain of any of the compositions described herein binds to the receptor.

In some aspects, the present disclosure provides for a method of editing a gene in a cell comprising, expressing a receptor on the surface of the cell, and contacting the cell with any of the compositions described herein.

In some aspects the present disclosure provides for a method of targeting any of the compositions described herein to the nucleus of a cell comprising contacting the cell with any of the compositions described herein, wherein the composition is detected in the nucleus.

In some aspects, the present disclosure provides for a method of generating the cell recognition domain of any of the compositions described herein comprising displaying a receptor on a solid surface. In some embodiments, the solid surface is a well of a multi-well plate or a bead. In some embodiments, the method further comprises screening a library of polypeptides displayed on a mammalian cell, a yeast cell, a bacterial cell, or a bacteriophage by ribosomal display, DNA/RNA systematic evolution of ligands by exponential enrichment (SELEX™), or DNA-encoded library approaches.

In some aspects, the present disclosure provides for a method for inducing death of cells bearing an EML4-ALK fusion gene, comprising contacting to said cell a composition comprising: a protein having at least 80% identity to SEQ ID NO 77, or a variant thereof, and a guide RNA targeting ALK4. In some embodiments, the guide RNA has at least 80% identity to any one of SEQ ID NOs: 88-105, or a variant thereof.

In some aspects, the present disclosure provides for a method for increasing cell resistance to HIV infection, comprising contacting to said cell a composition comprising: a protein having at least 80% identity to SEQ ID NO: 87, or a variant thereof, and a guide RNA targeting the CXCR4 locus. In some embodiments, the guide RNA targeting the CXCR4 locus has at least 80% identity to any one of SEQ ID NOs:108-109, or a variant thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts example nuclease compositions according to the current disclosure. Shown are domain diagrams illustrating N- to C-terminal domain organization for polypeptides or polypeptide compositions. In the figure, “PNME” denotes polynucleotide modifying enzyme, “L” denotes non-structural linker optionally with NLS/2×NLS, “CRD” denotes a cell recognition domain (which can be in the form of a linear peptide 7-15mer, a triple alpha helix scaffold, a VHH or ScFv scaffold, or a tri-bivalent form of any of the previous), “EE” denotes endosome escape domain, and “Hapten BD” denotes a Hapten binding domain.

FIG. 2 depicts an illustrative mechanism by which nuclease compositions according to the current disclosure may enter cells and be transported to the nucleus for gene editing. “PNME-CRD” refers to a composition with a polynucleotide-modifying enzyme domain and a cell recognition domain.

FIG. 3 illustrates the modular nature of nuclease compositions of the current invention. Shown is a flow chart depicting how various binding scaffold libraries can be optimized to select for binding to a particular cell receptor (left panel), which can then be combined with a programmable nuclease (center panel) to generate a cell-specific programmable nuclease platform. Receptor targets are chosen to be overexpressed or cell-specific as a requirement to be entered into the screening process.

FIG. 4 shows nuclear localization sequences that can be used with nuclease compositions according to the current disclosure. Shown are sequences from N- to C-terminus of various nuclear localization peptide sequences in one-letter amino acid code. These NLSes can be optionally utilized in linkers of PNME-CRD compositions according to the present disclosure, optionally between the PNME domain and the CRD.

FIG. 5 demonstrates delivery of nuclease compositions to the interior of cultured cells. Shown are 20× DIC-brightfield (left) and 20× epifluorescence (with 530 nm excitation/560 nm emission filter, right) photomicrographs of A549 cells treated with a TAMRA-labelled PNME-CRD composition comprising the anti-EGFR camelid nanoantibody 7D12 covalently linked to a type II Cas9 and then washed to remove non-internalized complexes. The images illustrate that PNME-CRD has been internalized within the cytosol and nucleus, which is shown by distribution throughout the body of the cells.

FIG. 6 demonstrates that nuclease composition (PNME-CRD) particles prepared as in FIG. 5 can cleave genomic DNA. Shown are the results of a T7 endonuclease INDEL agarose gel assay, where nuclease compositions directed against the EGFR receptor bearing a gRNA directed against the BRCA1 locus have been delivered to A549 cells. In this assay PCR gene amplicons generated from genomic DNA from the BRCA1 locus of edited cells are annealed to PCR amplicons from the BRCA1 locus of control cells followed by incubation with T7 endonuclease; mismatches due to indels generated by successful editing allow cleavage by T7 endonuclease to generate products of smaller size (100-300 bp) than the original PCR amplicon (500 bp). Lanes: 1 (100 bp ladder), 2 (blank), 3/7/11 (unedited control A549 treated with nuclease composition lacking gRNA), 4/5/6/8/9/10/12/13/14 (independent replicates of experiments where a nuclease composition with a BRCA1 gRNA was delivered to A549 cells).

FIG. 7 demonstrates that nuclease composition (PNME-CRD) particles have homologous-recombination mediated gene editing activity. Shown is a bar graph depicting remaining cell surface CXCR4 expression (“knockout percentage”) for 3T3 and A549 cells (n=4 biological replicates) treated with PNME-CRD compositions using Cas9 as a nuclease and 7D12 nanobody as a cell recognition domain after complexing with a guide RNA directed against CXCR4.

FIG. 8 illustrates recombinant expression (left) and activity assay (right) of a PNME-CRD molecule according to some embodiments of the disclosure. Left panel: SDS Page analysis of MDL4 purification and FLPC Elutes demonstrating IMAC (nickel NTA:agaraose) capture. Molecular weight determined by size markers of MDL4 is 168 kDa as indicated by the arrow. The gel demonstrates purification from the supernatant media of SF9 insect cell culture without cell lysis, as the protein is secreted under a cleavable IL2 secretion leader peptide. Lane order: 1) Page ruler marker, 2) FL-ON-flow through over night wash, 2) FL1-PBS-5 mM imidazole wash, 3)FL2-PBS-5 mM imidazole wash, 4)FL3-PBS-5 mM imidazole wash, 5/6) FL6 & 7-PBS-5 mM imidazole wash. Right panel: 1.5% agarose gel (TBE) illustrating an in-vitro cleavage assay using pGuide plasmid target. MDL4 PNME-CRD complexed with GFP guide was configured to garget a GFP-containing plasmid. Lanes MDL4 (1) and (2) are dye conjugated IMAC/SEC purified aliquots expressed in Sf9 cells as in left panel. 2 ul of protein was complexed with an excess of IVT synthesised gRNA (GFP) and incubated with 2 ug of pGuide plasmid target in1× nuclease buffer for 45 mins. Uncomplexed protein was incubated with plasmid as a control (no gRNA not nuclease activity), labelled as pGuide on gel. Complete cleavage of plasmid validates MDL4 activity is unchanged from IMAC purified samples, purified in test batch (4 ml SF9 culture).

FIG. 9 illustrates distinct cell populations identified by FACS in H2228 (EGFR-positive) and A549 (EGFR-negative) cells incubated with the MDL4 PNME-CRD molecule. The distinct populations indicate distinct mechanisms of uptake between the EGFR-negative and EGFR-positive cells, indicating that the MDL4 molecule containing an anti-EGFR CRD has a different mechanism of uptake in EGFR positive vs EGFR negative cells.

FIG. 10 illustrates that the distinct uptake mechanisms observed in FIG. 9 are not due to differences in general endocytosis between A549 (EGFR-positive) and H2228 (EGFR-positive cells) in FACS traces. Both A549 (EGFR-positive) and H2228 (EGFR-positive cells), when incubated with a nonspecific uptake control (BSA-TAMRA) indicate a left-shifted population (top row) that is distinct from cells incubated with MDL4-TAMRA that binds receptors on the surface of the cells (bottom two rows). This is true for increasing concentrations of MDL4-TAMRA (37.5 nM, middle row and 100 nM, bottom row).

FIG. 11 illustrates that 100 nM concentration of the MDL4 PNME-CRD has a maximal effect on cell proliferation and cell uptake of the PNME-CRD. Show in the top row are brightfield images illustrating a dose response of control (MDL4, no gRNA), 6 nM MDL4+gRNA, 37.5 nM MDL4+gRNA, and 100 nM MDL4+gRNA, showing that the biggest effect on cell confluency is observed at 100 nM. Shown in the bottom row are FACS traces of cells transfected with either 6 nM (left) or 100 nM (right) MDL4-TAMRA, demonstrating that ˜90% of the cells become positive for MDL4 in the 100 nM condition.

FIG. 12 illustrates that toxicity of MDL4 PNME-CRD is dependent on a gRNA molecule. Shown are fluorescence images showing acridine orange (viability) and propidium iodide (death) staining of H2228 cells dependent on the EML4-ALK gene transfected with either MDL4 with no gRNA (left column) or MDL4 with 12 gRNA targeting the EML4-ALK gene (right column). Cell death accumulates in the MDL4:I2 condition (right column) but not the MDL4:no gRNA condition (left column), indicating that activity of the 12 gRNA was necessary to inhibit proliferation or cause death of the H2228 cells.

FIG. 13 illustrates that toxicity of gRNA targeted against the ALK4 gene in H2228 cells is general to other gRNAs targeting the EML4-ALK gene. Shown are fluorescence images showing acridine orange (viability) and propidium iodide (death) staining of H2228 cells (EGFR-positive, columns 1 and 3) or A549 (EGFR-negative, columns 2 and 4) cells dependent on the EML4-ALK gene transfected with EML4-ALK targeting gRNAs I1, I2, I3, I4, V3A, and V3b in combination with the MDL4 molecule. All conditions with EML4-ALK targeted gRNAs indicate decreases of cell numbers in EGFR-positive cells but not EGFR-negative cells, indicating specificity of the cell-killing effect on the anti-EGFR CRD.

FIG. 14 illustrates that ALK4 editing coincides with anti-EGFR-positive activity. Shown in FIG. 14A is a time course from 24 to 72 hours of acridine orange-staining in H2228 (EGFR positive, left) or A549 cells (EGFR negative, right) transfected with MDL4 molecule plus I4 gRNA, which indicates that the I4 gRNA effectively inhibits cell growth in an EGFR-dependent manner. Shown in FIG. 14B are corresponding agarose gels of T7 endonuclease assays on amplicons from the cell conditions treated in FIG. 14A. EGFR-positive (H2) cells indicate increases in ALK4 amplicon size versus EGFR-negative (EG) samples (top panel). The same EGFR-positive (H2) cells are also selectively degraded in T7 endonuclease assays in complex with I2 guide, indicating that large fractions of the EGFR-positive cell populations undergo editing of the ALK4 amplicon (middle panel). The lack of degradation of ALK4 amplicons in EGFR-negative cells (EG) is similar to the lack of degradation of ALK4 amplicons isolated from H2228 edit-negative cells (bottom panel), confirming that the lack of degradation of ALK4 amplicon from EGFR-negative cells is due to lack of edits in the ALK4 amplicon.

FIG. 15 illustrates that gRNAs I1 and I3 have similar activity to the I2 and I4 gRNAs. Shown in the left panel is an agarose gel of T7 endonuclease assays on amplicons from the corresponding cell conditions (lane order: 1-molecular weight ladder; 2-I1 gRNA+MDL4 in H2228 cells; 3-I3 gRNA+MDL4 in H2228 cells; 4-I1 gRNA+MDL4 in A549 EGFR null cells; 5-I4 gRNA+MDL4 in A549 EGFR null cells; 5-no gRNA+MDL4 in H2228 cells; and 6-no gRNA+MDL4 in A549 EGFR null cells), indicating that the I1/I3 gRNAs combos are selective for editing in EGFR positive cells. Shown in the right panel are AO/PI stained images of either H2228 EGFR positive cells (right) or EGFR-null A549 cells (left) transfected with either I1 gRNA+MDL4 (top row) or I3 gRNA+MDL4 (bottom row), showing that the effect on viability is also selective between EGFR-positive and EGFR-null cells.

### DETAILED DESCRIPTION OF THE INVENTION

### Overview

Delivery of polynucleotide modifying enzymes (e.g. programmable nucleases, such as CRISPR nucleases) to cells for genome editing typically involves DNA-based, infectious vector-based, or mRNA transfection-based methodologies; however, each of these strategies has notable disadvantages.

Polynucleotide modifying enzymes delivered encoded on plasmids or other DNA-based material suffer from poor temporal control of nuclease expression, non-specific targeting, and limited efficiency depending on format. Because DNA-based delivery requires intracellular transcription and translation of the polynucleotide modifying enzyme (as well as any needed guide RNAs, in the case of RNA-directed programmable DNA nucleases), there is a significant time lag between delivery and maximum activity of the polynucleotide modifying enzyme; the polynucleotide modifying enzyme also persists for an indefinite amount of time as termination of expression depends on DNA dilution or degradation. Also, because DNA is poorly delivered to the cytoplasm of cells on its own, such strategies typically require use of a chemical transfection agent (e.g. cationic lipids or cationic polymers) or electroporation/nucleofection, limiting delivery to cells in vitro or in vivo with poor efficiency and nonselective targeting to tissues other than the liver (as cationic lipids and polymers are known to accumulate there).

Polynucleotide modifying enzymes delivered by infectious vectors (e.g. adeno-associated viruses, AAVs, or other retroviruses) suffer from the fact that such viruses are antigenic in humans and are associated with high production costs. As a result of antigenicity, such infectious vectors are associated with inflammatory immune responses which may result in undesirable side effects. Pre-existing antibodies against related wild-type viruses may additionally exacerbate side effects, limit the half-life of the vector in the body, or exclude the vector from the desired site of delivery. Antibodies generated as a result of an initial dose of such vectors to a subject may preclude efficacy of future doses of the polynucleotide modifying enzyme vector to the subject. Additionally, production of such infectious vectors is poorly scalable in industrial processes and is associated with variable amounts of payload-free vector, increasing production costs.

Polynucleotide modifying enzymes delivered by mRNA (e.g. via synthetic IVT mRNAs with non-natural nucleobases encoding the oligonucleotide modifying enzymes optionally in combination with related components) suffer from similar (though reduced) temporal concerns and targeting concerns as DNA-based vectors. Such a delivery strategy still requires translation of the mRNA and relies on variable cellular mechanisms to control when expression of the polynucleotide modifying enzyme ceases. Also, since delivery of such agents also typically depends on use of a chemical transfection agent (e.g. cationic lipids or cationic polymers) or electroporation/nucleofection, the efficiency/specificity of in vivo targeting is limited.

Liposomal protein-based delivery offers improvements versus the methodologies above, having tighter temporal control of activity and higher delivery to cells, as the active polynucleotide modifying enzyme (in complex with guide RNA if necessary) is transfected into cells. As activity of the polynucleotide modifying enzyme ceases once the polynucleotide modifying enzyme and/or guide RNA is degraded by endogenous proteases/nucleases in the cytoplasm, this delivery method is also potentially associated with lower off-target and re-cleavage of the target site. However, this method still typically requires use of a chemical transfection agent (e.g. cationic lipids or cationic polymers) or electroporation/nucleofection, limiting delivery to cells in vitro or in vivo with poor efficiency and nonselective tissue targeting other than the liver (as cationic lipids and polymers are known to accumulate there).

Accordingly, there is need for protein-based polynucleotide modifying enzyme transfection methodologies that do not depend on use of chemical transfection agents or electronic disruption of cellular membranes but preserve the beneficial features of polynucleotide modifying enzyme protein (or RNP) transfection. Described herein are methods, compositions, systems, and kits involving polynucleotide modifying enzyme compositions which are capable of cell entry without the use of chemical transfection agents or electric membrane disruption. In some embodiments, methods, compositions, systems, and kits herein are capable of targeted delivery of polynucleotide modifying enzyme to a particular population of cells, or to particular tissues using such compositions.

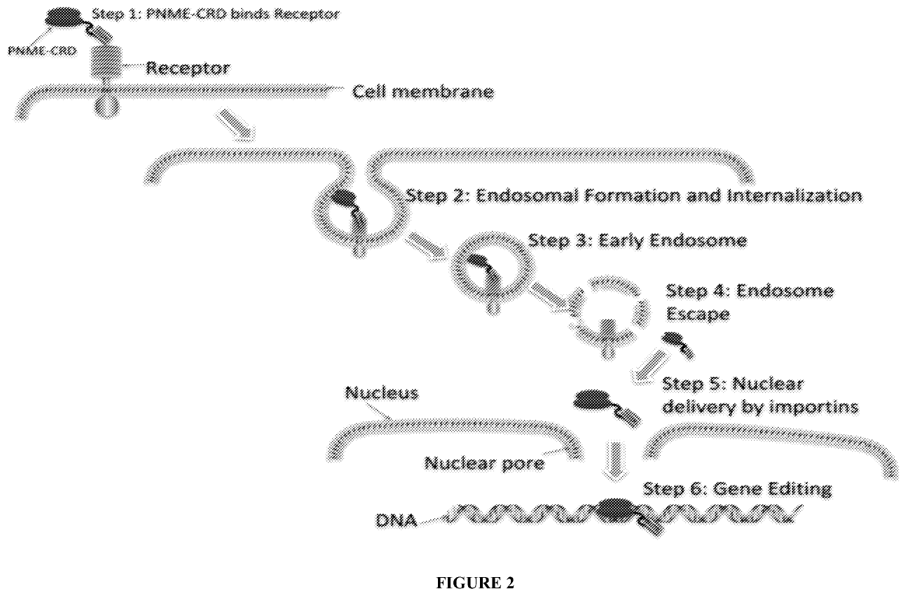

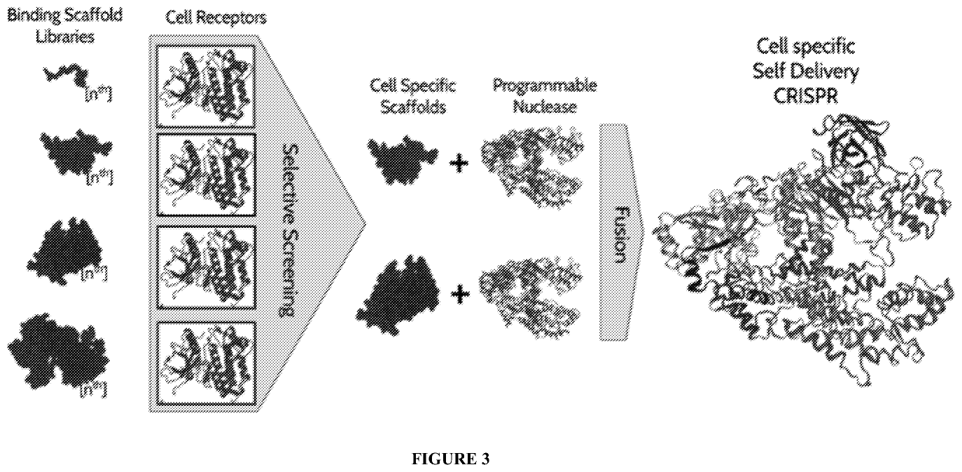

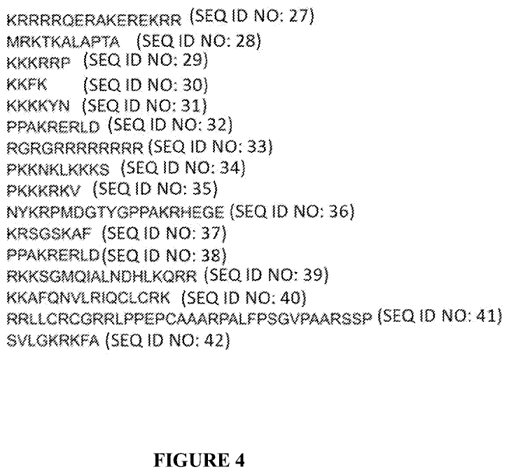

FIG. 2 illustrates a proposed mechanism by which some polynucleotide modifying enzyme compositions according to some embodiments of the current disclosure can enter cells without the aid of electric membrane disruption or chemical transfection agents. In a first embodiment, such compositions comprise a polynucleotide modifying enzyme (PNME), a cell recognition domain (CRD), and an endosome escape (EE) domain. Such compositions are envisioned as entering via the endosomal pathway; binding of the composition to a cellular antigen receptor via the cell recognition domain (“step 1) provides entry into the early endosomal pathway (“step 3”) after the receptor bound to the PNME-CRD composition is internalized via its association with the cell surface antigen or receptor, e.g. by clathrin-mediated endocytosis, calveolin-mediated endocytosis, or micropinocytosis (“step 2”). In some cases, binding of the PNME-CRD composition may stimulate endocytosis of the receptor or cell-surface antigen. After endocytosis, the endosome escape domain facilitates escape of the PNME-CRD from the endosomal pathway into the cytosol (“step 4”), after which the PNME-CRD composition can diffuse to its site of activity in the nucleus through nuclear pores or, alternatively (if a nuclear localization sequence is included in the PNME composition), via active transport into the nucleus via importins (“step 5”). Once in the nucleus, the PNME composition is then able to access DNA and perform a DNA cleavage or other DNA modifying reaction. Alternatively, if the PNME has an RNA target, the PNME composition need not be delivered to the nucleus to access nucleic acids upon which it acts (e.g. if the PNME is an RNA-modifying enzyme).

### Definitions

The practice of some methods disclosed herein employ, unless otherwise indicated, techniques of immunology, biochemistry, chemistry, molecular biology, microbiology, cell biology, genomics and recombinant DNA. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)) (which are entirely incorporated by reference herein).

As used herein, the term “cell recognition domain” (or “CRD”) refers to a natural or synthetic peptide or nucleic acid domain capable of specific non-covalent association with a cell-surface antigen or receptor.

As used herein, the term “polynucleotide modifying enzyme” (or “PNME”) refers to a peptide enzyme capable of cleaving the phosphodiester backbone of a nucleic acid (e.g. DNA or RNA) or altering the identity of one or more nitrogenous bases within a nucleic acid.

As used herein, the term “endosome escape domain” (or “EE domain”) refers to a peptide sequence which, when associated with a molecular cargo, facilitates diffusion of the cargo from the endosomal compartment to the cytosol and/or alters the steady state distribution of the cargo between the endosomal compartment and in favor of the cytosol.

As used herein, the term “hapten” refers to a small molecule, which when combined with a larger carrier such as a protein, is capable of high affinity binding to an antibody or antibody mimetic (“hapten binding domain”). In some embodiments, the molecular weight of the organic compound is less than 500 Daltons. In some embodiments, the affinity (K_{D}) of the hapten for the hapten binding domain is less than 10⁻⁶ molar. In some embodiments, the affinity (K_{D}) of the hapten for the peptide or nucleic acid aptamer is less than 10⁻⁷ molar. In some embodiments, the affinity (K_{D}) of the hapten for the peptide or nucleic acid aptamer is less than 10⁻⁸ molar. In some embodiments, the affinity (K_{D}) of the hapten for the peptide or nucleic acid aptamer is less than 10⁻⁹ molar.

As used herein, the term “linker”, “linker group” or “linker domain” means a group that can link one chemical moiety to another chemical moiety. In some embodiments, a linker is a bond. In some embodiments, the linker is an organic molecule, group, polymer, or chemical moiety. In some embodiments, the linker is a cleavable linker, e.g., the linker comprises a linkage that can be cleaved upon exposure to a cleavage activity such as UV light or a hydrolase, such as a lysosomal protease. In some embodiments, the linker may comprise one or more, two or more, three or more, four or more, five or more, six or more, seven or more, eight or more, nine or more, ten or more, 20 or more, 25 or more, 30 or more, 40 or more, 50 or more amino acids. In some embodiments, the peptide linker comprises a repeat of a tri-peptide Gly-Gly-Ser, including, for example, sequence (GGS)ₙ, wherein n is at least 1, 2, 3, 4, 5, 6, 7, 8, 9, 10 or more repeats. In some embodiments, the linker can comprise at least two polyethyleneglycol (PEG) residues. In some embodiments, a PEG linker comprises three or more, four or more, five or more, six or more, seven or more, eight or more, nine or more, or ten or more PEG residues. In some embodiments, the PNME compositions described herein comprise linkers joining two or more domains described herein, such as any combination of two or more of cell recognition domains, endosome escape domains, nuclear localization sequences, or PNME domains.

The term “tracrRNA” or “tracr sequence”, as used herein, can generally refer to a nucleic acid with at least about 5%, 10%, 20%, 30%, 40%, 50%, 60%, 70%, 80%, 90%, or 100% sequence identity and/or sequence similarity to a wild type exemplary tracrRNA sequence (e.g., a tracrRNA from S. pyogenes, S. aureus, etc). tracrRNA can refer to a nucleic acid with at most about 5%, 10%, 20%, 30%, 40%, 50%, 60%, 70%, 80%, 90%, or 100% sequence identity and/or sequence similarity to a wild type exemplary tracrRNA sequence. tracrRNA may refer to a modified form of a tracrRNA that can comprise a nucleotide change such as a deletion, insertion, or substitution, variant, mutation, or chimera. A tracrRNA may refer to a nucleic acid that can be at least about 60% identical to a wild type exemplary tracrRNA sequence over a stretch of at least 6 contiguous nucleotides. For example, a tracrRNA sequence can be at least about 60% identical, at least about 65% identical, at least about 70% identical, at least about 75% identical, at least about 80% identical, at least about 85% identical, at least about 90% identical, at least about 95% identical, at least about 98% identical, at least about 99% identical, or 100% identical to a wild type exemplary tracrRNA sequence over a stretch of at least 6 contiguous nucleotides.

As used herein, a “guide nucleic acid” can refer to a nucleic acid that may hybridize to another nucleic acid. A guide nucleic acid may be RNA. A guide nucleic acid may be DNA. The guide nucleic acid may be programmed to bind specifically to a nucleic acid with a particular sequence. The nucleic acid to be targeted, or the target nucleic acid, may comprise nucleotides. The guide nucleic acid may comprise nucleotides. A portion of the target nucleic acid may be complementary to a portion of the guide nucleic acid. The strand of a double-stranded target polynucleotide that is complementary to and hybridizes with the guide nucleic acid may be called the complementary strand. The strand of the double-stranded target polynucleotide that is complementary to the complementary strand, and therefore may not be complementary to the guide nucleic acid may be called a noncomplementary strand. A guide nucleic acid may comprise a polynucleotide chain and can be called a “single guide nucleic acid.” A guide nucleic acid may comprise two polynucleotide chains and may be called a “double guide nucleic acid.” If not otherwise specified, the term “guide nucleic acid” may be inclusive, referring to both single guide nucleic acids and double guide nucleic acids. Guide nucleic acids may comprise a nucleic acid targeting segment (e.g. a crRNA) and a protein binding sequence. Guide nucleic acids may comprise a nucleic acid targeting segment (e.g. a crRNA) a protein binding sequence, and a trans-activating RNA (e.g. a tracrRNA). In some cases, a guide RNA described herein comprises a sequence of n nucleotides counting from a 1^{st} nucleotide at a 5′ end to an n^{th} nucleotide at a 3′ end, wherein one or more of the nucleotides at positions 1, 2, n-1 and n are phosphorothioate modified nucleotides. The guide nucleic acid can comprise one or more bridged nucleotides in a seed region of the guide oligonucleotide. A guide nucleic acid that is part of a PNME-CDR composition may target the composition to a target nucleic acid

A guide nucleic acid may comprise a segment that can be referred to as a “nucleic acid-targeting segment” a “nucleic acid-targeting sequence” or a “seed sequence”. In some cases, the sequence is 19-21 nucleotides in length. In some cases, “nucleic acid-targeting segment” or a “nucleic acid-targeting sequence” comprises a crRNA. A nucleic acid-targeting segment may comprise a sub-segment that may be referred to as a “protein binding segment” or “protein binding sequence” or “Cas protein binding segment”.

A “host cell” generally includes an individual cell or cell culture which can be or has been a recipient for the subject vectors into which exogenous nucleic acid has been introduced, such as those described herein. Host cells include progeny of a single host cell. The progeny may not necessarily be completely identical (in morphology or in genomic of total DNA complement) to the original parent cell due to natural, accidental, or deliberate mutation. A host cell includes cells transfected in vivo with a vector of this invention.

### Compositions for Genomic Editing

In some aspects, the present disclosure provides for a composition for modifying a gene, comprising a cell recognition domain, an endosome escape domain, and a polynucleotide-modifying enzyme domain. In some embodiments, the endosome escape domain is covalently coupled to the cell recognition domain.

The cell recognition domain can be a natural or synthetic peptide or nucleic acid domain capable of specific non-covalent association with a cell-surface antigen or receptor. The cell recognition domain can bind to an epitope of the cell-surface antigen or receptor. In some embodiments, the cell recognition domain is an antibody or antigen-binding fragment thereof, or an antibody mimetic. Antibodies include camelid antibodies. Antigen-binding fragments include Fab fragments, Fab′ fragments, F(ab′)₂ fragments, fragments produced by Fab expression libraries, Fd fragments, Fv fragments, disulfide linked Fv (dsFv) domains, single chain antibody (e.g. scFv) domains, VHH domains, or single domain antibodies. Antibody mimetics are non-antibody derived peptides or nucleic acids that bind with similar affinity to antibodies and include affibodies, affilins, affimers, affitins, alphabodies, anticalins, atrimers, avimers, aptamers, DARPins, fynomers, knottins, Kunitz domain peptides, monobodies, nanoCLAMPs, and linear peptides of 6-20 amino acids. See, e.g., Yu et al., Annu Rev Anal Chem (Palo Alto Calif.). 2017 Jun. 12; 10(1): 293-320. Suitable antibody mimetics can be derived by mammalian cell, bacterial cell, or bacteriophage display by systematic evolution of ligands by exponential enrichment (SELEX™) or DNA encoded library approaches involving e.g. immobilization of a given antigen on a surface followed by binding selection. In some cases, the cell recognition domain is an aptamer oligonucleotide, such as a polyribonucleotide or a polydeoxyribonucleotide; design and selection of example aptamers can be found in e.g. Sun et al. Mol Ther Nucleic Acids. 2014 Aug.; 3(8): e182. Such oligonucleotide aptamers can comprise non-canonical nucleotides, such as 2′-OMe, 2′-F, or 4′-S nucleotides, 2′-FANAs, HNAs, or locked nucleic acid residues. In some embodiments, the cell recognition domain comprises a chemical ligand with a molecular weight of less than about 800 Da. Such ligands include small-molecule ligands of cell-surface small-molecule receptors such as folate (which binds to the folate receptor), piperidine carboxyamides (which bind to FSHR), phenylpyrazole or thienopyrimidine compounds (which bind to LHR), cinacalcet or analogs (which bind to CRF1) or nitro-bezoxadiazole compounds (which bind to EGFR). Such ligands also include protein ligands of cell-surface receptors such as IL2 (which binds to IL2alpha receptor), EGF (which binds to EGFR), or HFG (which binds to HFGR). In some cases, the cell recognition domain does not directly associate with a cell surface antigen but rather is capable of binding a protein ligand that is selective for a cell-surface receptor or carbohydrate. In some cases, the cell recognition domain comprises a protein ligand that is selective for a cell-surface receptor or carbohydrate. In some cases, the protein ligand that is selective for a cell-surface receptor or carbohydrate comprises 5-15 amino acids in length. In some cases, the protein ligand is a peptide growth hormone. In some cases, the protein ligand has a globular or cyclical structure.

In some embodiments, the cell recognition domain binds to one or more epitopes on a cell-surface antigen to direct the PNME composition to a cell expressing the cell surface antigen. In some cases, the cell-surface antigen can be a cell-surface glycan or protein. Cell surface glycans include glycans linked to cell-surface proteins, as well as those linked to cell membrane lipids. In some cases, the cell recognition domain drives association of the composition for modifying a gene with a specific type of cell or tissue such as a diseased cell or tissue or a cancerous cell or tissue; for this purpose, cell-surface antigens selectively expressed on a particular target cell or class of target cells and lacking expression on non-target cells can be used. For cancer-specific delivery, the cell recognition domain can bind an epitope of a G-protein coupled receptor, an epitope of a tyrosine kinase receptor, an epitope of a membrane channel or membrane transporter, an epitope of a cell surface proteoglycan, proteolipid, or glycoprotein, or an epitope of an integral membrane protein. For example, for cancer-specific delivery, the cell recognition domain can bind to an epitope of any of the antigens set forth in Table 1 below. In some cases, a particular cell surface antigen or receptor is expressed in a target cell type prior to delivery of the PNME composition to the cell.

TABLE 1
List of Cancer-associated Antigens that can be used for specific delivery
of nucleases according to some embodiments described herein

| | Example UniProt Accession ID, Chemical Name, |
|---|---|
| Target | or Literature Reference |
| cd44v6 | Tremmel et al. Blood 114: 5236-5244(2009) |
| CAIX (Carbonic Anhydrase 9, CA9) | Q16790 (CAH9_HUMAN) |
| CEA (CEA Cell Adhesion Molecule 5, | P06731 (CEAM5_HUMAN) |
| CEACAM5, Carcinoembryonic antigen) | |
| CD133 (Prominin 1, PROM1) | O43490 (PROM1_HUMAN) |
| cMet hepatocyte growth factor receptor | P08581 (MET_HUMAN) |
| (MET) | |
| EGFR (Epidermal Growth Factor | P00533 (EGFR_HUMAN) |
| Receptor, HER1) | |
| EGFR vIII | Koga et al. Neuro Oncol. 2018 September; 20(10): 1310- |
| | 1320. |
| EPCAM (Epithelial Cell Adhesion | P16422 (EPCAM_HUMAN) |
| Molecule) | |
| EphA2 (EPH Receptor A2) | P29317 (EPHA2_HUMAN) |
| Fetal acetylcholine receptor | Nayak et al. Proc Natl Acad Sci USA. 2013 Aug. |
| | 13; 110(33): 13654-9. |
| FRalpha folate receptor (FOLR1) | P15328 (FOLR1_HUMAN) |
| GD2 (Ganglioside G2) | (2R,4R,5S,6S)-2-[3-[(2S,3S,4R,6S)-6- |
| | [(2S,3R,4R,5S,6R)-5-[(2S,3R,4R,5R,6R)-3- |
| | acetamido-4,5-dihydroxy-6-(hydroxymethyl)oxan- |
| | 2-yl]oxy-2-[(2R,3S,4R,5R,6R)-4,5-dihydroxy-2- |
| | (hydroxymethyl)-6-[(E)-3-hydroxy-2- |
| | (octadecanoylamino)octadec-4-enoxy]oxan-3- |
| | yl]oxy-3-hydroxy-6-(hydroxymethyl)oxan-4- |
| | yl]oxy-3-amino-6-carboxy-4-hydroxyoxan-2-yl]- |
| | 2,3-dihydroxypropoxy]-5-amino-4-hydroxy-6- |
| | (1,2,3-trihydroxypropyl)oxane-2-carboxylic acid |
| GPC3 (Glypican 3) | P51654 (GPC3_HUMAN) |
| GUCY2C (Guanylate Cyclase 2C) | P25092 (GUC2C_HUMAN) |
| HER2 (ERBB2) | P04626 (ERBB2_HUMAN) |
| ICAM1 (Intercellular Adhesion Molecule 1) | P05362 (ICAM1_HUMAN) |
| IL13Ralpha2 (IL13RA2) | Q14627 (I13R2_HUMAN) |
| IL11 receptor alpha (IL11RA) | Q14626 (I11RA_HUMAN) |
| Kras | P01116 (RASK_HUMAN) |
| Kras G12D | P01116 (RASK_HUMAN) with G12D substitution |
| L1cam (L1 Cell Adhesion Molecule) | P32004 (L1CAM_HUMAN) |
| MAGE (melanoma-associated antigen) | P43360 (MAGA6_HUMAN) |
| | P43355 (MAGA1_HUMAN) |
| | Q9Y5V3 (MAGD1_HUMAN) |
| | P43356 (MAGA2_HUMAN) |
| | Q9UBF1 (MAGC2_HUMAN) |
| | P43364 (MAGAB_HUMAN) |
| | P43365 (MAGAC_HUMAN) |
| | Q9UNF1 (MAGD2_HUMAN) |
| | P43357 (MAGA3_HUMAN) |
| | Q9HCI5 (MAGE1_HUMAN) |
| | P43358 (MAGA4_HUMAN) |
| | P43361 (MAGA8_HUMAN) |
| | Q96JG8 (MAGD4_HUMAN) |
| | Q9HAY2 (MAGF1_HUMAN) |
| | O15481 (MAGB4_HUMAN) |
| | O15479 (MAGB2_HUMAN) |
| | P43363 (MAGAA_HUMAN) |
| | Q96M61 (MAGBI_HUMAN) |
| | P43362 (MAGA9_HUMAN) |
| | Q8TD91 (MAGC3_HUMAN) |
| | O60732 (MAGC1_HUMAN) |
| | Q9H213 (MAGH1_HUMAN) |
| | P43359 (MAGA5_HUMAN) |
| Mesothelin (MSLN) | Q13421 (MSLN_HUMAN) |
| MUC1 (Mucin 1, Cell Surface | P15941 (MUC1_HUMAN) |
| Associated) | |
| MUC16 (Mucin 16, Cell Surface | Q8WXI7 (MUC16_HUMAN) |
| Associated) | |
| NKG2D (Killer Cell Lectin Like | P26718 (NKG2D_HUMAN) |
| Receptor K1, KLRK1, NK Cell receptor | |
| D, CD314) | |
| NY-ESO1 (New York Esophageal | P78358 (CTG1B_HUMAN) |
| Squamous Cell Carcinoma 1, CTAG1B, | |
| Cancer/Testis Antigen 1B) | |
| PSCA (Prostate Stem Cell Antigen, | O43653 (PSCA_HUMAN) |
| PRO232) | |
| WT1 (WT1 Transcription Factor, Wilms | P19544 (WT1_HUMAN) |
| Tumor Protein) | |
| PSMA (prostate-specific membrane | Q04609 (FOLH1_HUMAN) |
| antigen, Glutamate carboxypeptidase II, | |
| GCPII, N-acetyl-L-aspartyl-L-glutamate | |
| peptidase I, NAALADase I, NAAG | |
| peptidase, FOLH1, folate hydrolase 1) | |
| 5t4 or TPBG (Trophoblast Glycoprotein) | Q13641 (TPBG_HUMAN) |
| Transferrin receptor (TFRC, CD71, TFR1) | P02786 (TFR1_HUMAN) |
| GPNMB Breast cancer, melanoma | Q14956 (GPNMB_HUMAN) |
| (Glycoprotein Nmb) | |
| LeY (Lewis y antigen, Lewis y | N-[(3R,4R,5S,6R)-5-[(2S,3R,4S,5R,6R)-4,5- |
| Tetrasaccharide) | dihydroxy-6-(hydroxymethyl)-3- |
| | [(2R,3R,4S,5R,6R)-3,4,5-trihydroxy-6-methyloxan- |
| | 2-yl]oxyoxan-2-yl]oxy-2-hydroxy-6- |
| | (hydroxymethyl)-4-[(2R,3R,4S,5R,6R)-3,4,5- |
| | trihydroxy-6-methyloxan-2-yl]oxyoxan-3- |
| | yl]acetamide |
| CA6 (Carbonic anhydrase 6, CA-VI) | P23280 (CAH6_HUMAN) |
| Av integrin (ITGAV, Integrin Subunit | P06756 (ITAV_HUMAN) |
| Alpha V) | |
| SLC44A4 (Solute Carrier Family 44 | Q53GD3 (CTL4_HUMAN) |
| Member 4) | |
| Nectin-4 (NECTIN4, NECT4, PVRL4, | Q96NY8 (NECT4_HUMAN) |
| EDSS1) Solid tumors | |
| AGS-16 (Ectonucleotide | O14638 (ENPP3_HUMAN) |
| Pyrophosphatase/Phosphodiesterase 3, | |
| ENPP3) | |
| Cripto (CFC1, FRL-1, Cryptic Family 1) | P0CG37 (CFC1_HUMAN) |
| ALCAM (Activated Leukocyte Cell | Q13740 (CD166_HUMAN) |
| Adhesion Molecule, CD 166, MEMD) | |
| TENB2 (Transmembrane Protein With | Q9UIK5 (TEFF2_HUMAN) |
| EGF Like And Two Follistatin Like | |
| Domains 2, TMEFF2, Tomoregulin-2, | |
| HPP1, TPEF) | |
| EPCAM (Epithelial Cell Adhesion | P16422 (EPCAM_HUMAN) |
| Molecule, Tumor-Associated Calcium | |
| Signal Transducer 1, Major Gastrointestinal | |
| Tumor-Associated Protein GA733-2, | |
| Trophoblast Cell Surface Antigen 1, | |
| TACSTD1, EGP314, CD326) | |

For tissue-specific delivery, the cell recognition domain can bind to e.g. an epitope of any of the antigens set forth in Table 2 below.

TABLE 2
Examples of receptors with high tissue expression that may be used for tissue
specific delivery according to some embodiments of the current disclosure

| | Example Gene/Protein | |
|---|---|---|
| Receptor | Symbol or Uniprot Accession | Tissue |
| L-SIGN (CLEC4M, C-Type Lectin | Q9H2X3 | liver |
| Domain Family 4 Member M, CD299) | (CLC4M_HUMAN) | |
| ASGPR (ASGR1, ASGR2, | P07306 (ASGR1_HUMAN) | liver |
| Asialoglycoprotein receptor 1 or 2) | P07307 (ASGR2_HUMAN) | |
| AT1 (Angiotensin II Receptor Type 1, | P30556 (AGTR1_HUMAN) | kidney |
| AGTR1) | | |
| B2/B1 receptor (Bradykinin Receptor | P46663 (BKRB1_HUMAN) | lung |
| B1 or B2, BDKRB1, BDKRB2, | P30411 (BKRB2_HUMAN) | |
| BKRB1, BKRB2) | | |
| Muscarinic receptors (Muscarinic | CHRM1, CHRM2, CHRM3, | lung/Bladder |
| acetylcholine receptors, mAChRs) | CHRM4, CHRM5 | |
| FGFR4 (Fibroblast Growth Factor | P22455 (FGFR4_HUMAN) | Liver, kidney lung pancreatic |
| Receptor 4) | | cells |
| FGFR3 (Fibroblast Growth Factor | P22607 (FGFR3_HUMAN) | Brain kidney testes |
| Receptor 3) | | |
| FGFR1 (Fibroblast Growth Factor | P11362 (FGFR1_HUMAN) | Epithelial, endothelial |
| Receptor 1) | | fibroblasts |
| | | mesenchymal, |
| Frizzled 4 (Frizzled Class Receptor 4, | Q9ULV1 (FZD4_HUMAN) | Ubiquitous |
| FZD4) | | |
| S1PR1 (Sphingosine-1-Phosphate | P21453 (S1PR1_HUMAN) | Endosomal |
| Receptor 1) | | vascular smooth |
| | | muscle |
| TSHR (Thyroid Stimulating Hormone | P16473 (TSHR_HUMAN) | thyroid |
| Receptor) | | |
| GPR41 (Free Fatty Acid Receptor 3, | O14843 (FFAR3_HUMAN) | colon |
| G Protein-Coupled Receptor 41, | | |
| FFAR3) | | |
| GPR43 (G Protein-Coupled Receptor | O15552 (FFAR2_HUMAN) | colon |
| 43, FFAR2, Free Fatty Acid Receptor | | |
| 2) | | |
| GPR109A (G Protein-Coupled | Q8TDS4 | colon |
| Receptor 109A, Niacin Receptor 1, | (HCAR2_HUMAN) | |
| NIACR1, Hydroxycarboxylic Acid | | |
| Receptor 2, HCAR2) | | |
| TFRC (Transferrin Receptor, CD71, | P02786 (TFR1_HUMAN) | Blood brain barrier |
| TFR1) | | |
| Insulin receptor (INSR, CD220) | P06213 (INSR_HUMAN) | Blood brain barrier |
| Insulin-like growth factor 2 receptor | P11717 (MPRI_HUMAN) | Blood brain barrier |
| (IGF2R, Cation-independent | | |
| mannose-6-prosphate receptor, CI- | | |
| MPR, MPRI) | | |
| LRP1 (LDL Receptor Related Protein | Q07954 (LRP1_HUMAN) | General cell delivery |
| 1, Apolipoprotein E Receptor, | | |
| APOER, CD91) | | |
| IGF1R (Insulin Like Growth Factor 1 | P08069 (IGF1R_HUMAN) | Prostate |
| Receptor, CD221) | | |
| Prolactin receptor (PRLR) | P16471 (PRLR_HUMAN) | Ovarian normal and cancer |
| Follicle stimulating hormone receptor | P23945 (FSHR_HUMAN) | Ovarian |
| (FSHR, FSH receptor, Follitropin | | |
| Receptor, LGR1) | | |

In some embodiments, the cell recognition domain can bind an epitope of more than one cell-surface antigen. This can be accomplished by utilizing more than one binding components (e.g. more than one antibody or antigen-binding fragment thereof, or more than one antibody mimetic) in the polynucleotide-modifying enzyme composition. In some cases, the PNME composition comprises at least two, at least three, at least four, or at least five binding components (e.g. antibodies or antigen-binding fragments thereof, or antibody mimetics). In some cases, all the binding components are the same class of binding component. In some embodiments, the binding components bind epitopes on the same cell surface antigen or receptor; such embodiments can be useful to increase the affinity of the PNME composition for a cell surface antigen or receptor. In some embodiments, the binding components bind epitopes on different cell surface receptors or antigens; such embodiments can be useful to increase specificity of the PNME composition for a particular cell type (e.g. when each cell surface antigen or receptor is cell-type specific). In cases where the PNME composition comprises more than one binding component, the function of each binding component may be different; for example, one binding component can have specificity for a cell surface receptor or antigen that is rapidly internalized by a target cell and a second binding component can have specificity for a second cell surface receptor or antigen that is not rapidly internalized by the target cell. In some embodiments, a first binding component of a PNME composition can have specificity for EPCAM and a second binding component of a PNME composition can have specificity for ALCAM.

In some embodiments, the polynucleotide modifying enzyme composition comprises an endosome escape (EE) domain or sequence. Endosome escape domains or sequences, when associated with a molecular cargo, facilitate diffusion of the cargo from the endosomal compartment to the cytosol and/or alter the steady state distribution of the cargo between the endosomal compartment and cytosol in favor of the cytosol. Endosome escape domains may comprise hydrophobic peptide sequences which result in disruption of the endosome (e.g. early or late endosome) membrane, or lysis of the endosome. In some cases, the endosome escape sequences are between 3 and 9 amino acids. In some embodiments, the polynucleotide modifying enzyme compositions comprise one or more endosome escape domain or sequence described below in Table 3.

TABLE 3
Examples of Endosome escape sequences that can be used with
polynucleotide-modifying enzyme compositions according to some
embodiments described herein

| SEQ ID NO: | Peptide Sequence (N- to C-terminus) |
|---|---|
| 16 | X₁X₂X₃X₄X₅X₆X₇X₈X₉; wherein |
| | X₁ is P or C; |
| | X₂, X₃, X₄, and X₅ are independently selected |
| | from C, R, or K; and |
| | X₆, X₇, X₈, and X₉ are independently selected |
| | from C, R, K, A, or W. |
| 17 | X₁X₂X₃X₄X₅X₆X₇X₈X₉; wherein |
| | X₁ is P or C; |
| | X₂, X₃, X₄, and X₅ are independently selected |
| | from C, R, or K; and |
| | X₆, X₇, X₈, and X₉ are independently selected |
| | from C, R, K, A, or W., and wherein at least 3 |
| | of X₁-X₉ are C and no more than 8 of X₁-X₉ are |
| | C. |
| 18 | PCRKCACCA |
| 19 | PRCCRWCCA |
| 20 | PRRCKRCKC |
| 21 | CKKCRKCCK |
| 22 | CCRCKCWCC |
| 23 | CCRKCCCCC |
| 24 | PRKCCCCCC |
| 25 | HHHHHHHHHH |
| 26 | CCCCCC |

Polynucleotide modifying enzymes included in the PNME compositions described herein include enzymes which cleave the phosphodiester backbone of the nucleic acid or alter the identity of one or more nitrogenous bases within the nucleic acid. PNMEs that cleave the phosphodiester backbone of the nucleic acid can cleave double- or single-stranded polynucleotides. PNMEs that cleave the phosphodiester backbone of double-stranded nucleic acid can result in blunt-ended or staggered cuts. PNMEs may be capable of associating with a nucleic acid (e.g. DNA or RNA).

In some cases, the PNME enzymes are programmable nucleases. Such nucleases can be engineered to target a specific DNA or RNA sequence for cleavage, and include Cas9, Cas12a (Cpf1), Cas12b, Cas12c, Cas12d, Cas12e, Cas13a, Cas13b, Cas14, other CRISPR endonucleases, Argonaute endonucleases, transcription activator-like (TAL) effector and nucleases (TALEN), or zinc finger nucleases (ZFN). In some cases, CRISPR endonucleases are class II CRISPR endonucleases. In some cases, CRISPR endonucleases are class II, type II, V, or VI endonucleases. In some cases, such nucleases comprise at least one nuclease deficient nuclease domain. In some cases, CRISPR endonucleases are CpfI or MAD7.

CRISPR endonucleases typically require the use of a guide RNA (gRNA) or guide nucleic acid complexed (e.g. non-covalently associated) with the CRISPR endonuclease (or “Cas enzyme”) to specify targeting of a specific sequence of DNA for cleavage. Accordingly, a composition for gene editing that comprises a PNME composition involving a CRISPR/Cas endonuclease can also comprise a guide RNA as described herein. Guide nucleic acids generally direct cleavage of a target sequence when the target sequence is located within about 30 nucleotides of a protospacer adjacent sequence (PAM) sequence characteristic of the CRISPR endonuclease

In some cases, PNME enzymes are RNA editing enzymes. Such enzymes can act on RNA (e.g. cytosolic mRNA) to alter base identities within an RNA sequence, thereby altering the activity of the RNA (e.g. increasing or decreasing transcription of an mRNA). RNA editing enzymes include, but are not limited to, cytidine deaminases, double-stranded RNA-specific adenosine deaminase (ADAR), IFIT2, eIF4a, eIF4e, PABP, PAIP, SLBP,BOLL, ICP27, YTHDF1, YTHDF2, YTHDF3, TOB2, ZFP36, CNOT7, RNaseA, RNaseL, RNaseP, RNase4, RNasel, RNaseU2, or HRSP12.

In some cases, PNME enzymes are recombinases. Recombinases include, but are not limited to, Rad52 recombinase, Rad51 recombinase, CRE recombinase, Flippase (Flp), lambda integrase from bacteriophage lambda, Dre, KD, B2, B3, HK022, HP1, ParA, Tn3, Gin, phiC31, Bxb1, or R4.

In some cases, PNMEs or PNME compositions described herein comprise a nuclear localization sequence (NLS). The NLS can be located at the N- or C-terminus of the PNME, or both. The NLS can be separated from the PNME peptide sequence by a linker or can be directly fused to the PNME sequence without intervening amino acids. In some cases, the NLS is within a linker domain separating two other domains of the PNME composition (e.g. PNME enzyme, CRD, EE domain). In some cases, the PNME or PNME composition comprises at least one, at least two, at least 3, at least 4, at least 5, or more NLSs. In some embodiments, NLSs comprise 7-25 amino acid residues. In some embodiments, NLSs are derived from mammalian nuclear entering proteins such as splicing factors or transcription factors. In some embodiments, an NLS interacts with an importin. In some embodiments, the NLS is a bipartite NLS wherein amino acids within an N-terminal portion of the NLS involved in the recognition of an importin and amino acids within a C-terminal portion of the NLS involved in the recognition of an importin are split by an amino acid sequence not involved in the recognition of an importin. In some embodiments, an NLS comprises at least one sequence depicted in Table 4 below or a combination of sequences from Table 4, a sequence having at least 70%, at least 75%, at least 80%, at least 85%, at least 90%, at least 95%, at least 99% sequence identity to a sequence described in Table 4, or a sequence substantially identical to any of the sequences in Table 4. When more than one NLS is included in a PNME or PNME composition, the NLSs may comprise the same sequence or comprise different sequences.

TABLE 4
Examples of Nuclear Localization Sequences (NLSs)
that can be used with polynucleotide-
modifying enzyme compositions according to some
embodiments described herein

| SEQ ID NO: | Peptide Sequence (N- to C-terminus) |
|---|---|
| 27 | KRRRRQERAKEREKRR |
| 28 | MRKTKALAPTA |
| 29 | KKKRRP |
| 30 | KKFK |
| 31 | KKKKYN |
| 32 | PPAKRERLD |
| 33 | RGRGRRRRRRRR |
| 34 | PKKNKLKKKS |
| 35 | PKKKRKV |
| 36 | NYKRPMDGTYGPPAKRHEGE |
| 37 | KRSGSKAF |
| 38 | PPAKRERLD |
| 39 | RKKSGMQIALNDHLKQRR |
| 40 | KKAFQNVLRIQCLCRK |
| 41 | RRLLCRCGRRLPPEPCAAARPALFPSGVPAARSSP |
| 42 | SVLGKRKFA |

In some embodiments, the PNME composition further comprises a hapten binding domain to link an additional protein or nucleic acid ligand to the PNME composition. A “hapten binding domain” is a peptide or oligonucleotide domain that binds a hapten. “Hapten” refers to a small molecule, which when combined with a larger carrier such as a protein, is capable of high affinity binding to an antibody or antibody mimetic (“hapten binding domain”). In some embodiments, hapten/hapten binding domain pairs are derived from natural proteins or engineered variants thereof, such as the biotin/avidin pair or amylose/MBP pair. Engineered alternatives for biotin include D-desthiobiotin. Alternatives for avidin include streptavidin, NeutrAvidin, and CaptAvidin. In some embodiments, hapten/hapten binding domain pairs are synthetically engineered pairs such as 3-methylindole/anti-3-methylindole monoclonal antibody (such as 14G8, 3F12, 4A1G, 8F2, or 8H1 monoclonal antibodies), fumonisin B1/anti-fumonisin antibody, 1,2-Naphthoquinone/anti-1,2-Naphthoquinone antibody, 15-Acetyldeoxynivalenol/anti-15-Acetyldeoxynivalenol antibody, (2-(2,4-dichlorophenyl)-3(1H-1,2,4-triazol-1-yl)propanol)/anti-(2-(2,4-dichlorophenyl)-3(1H-1,2,4-triazol-1-yl)propanol) antibody, 22-oxacalcitriol/anti-22-oxacalcitriol antibody, (24,25(OH)2D3)/anti-(24,25(OH)2D3) antibody, 2,4,5-Trichlorophenoxyacetic acid/anti-2,4,5-Trichlorophenoxyacetic acid antibody, 2,4,6-Trichlorophenol/anti-2,4,6-Trichlorophenol antibody, 2,4,6-Trinitrotoluene/anti-2,4,6-Trinitrotoluene antibody, 2,4-Dichlorophenoxyacetic acid/anti-2,4-Dichlorophenoxyacetic acid antibody, 2-hydroxybiphenyl/anti-2-hydroxybiphenyl antibody, 3,5,6-trichloro-2-pyridinol/anti-3,5,6-trichloro-2-pyridinol antibody, 3-Acetyldeoxynivalenol/anti-3-Acetyldeoxynivalenol antibody, 3-phenoxybenzoic acid/anti-3-phenoxybenzoic acid antibody, digoxin/anti-digoxin antibody, fluorescein/anti-fluorescein antibody, or hexahistidine/Ni-NTA. The hapten binding domain can be located N- or C-terminal to the PNME, or both. The hapten binding domain can be separated from another domain described herein by a linker or can be directly fused to the domain sequence without intervening amino acids. In some cases, the hapten binding domain is within a linker domain separating two other domains of the PNME composition (e.g. PNME enzyme, CRD, EE domain). In some cases, the PNME composition comprises at least one, at least two, at least 3, at least 4, at least 5, or more hapten binding domains.

When the PNME composition comprises a hapten-binding domain, the composition can further comprise a peptide, protein, oligonucleotide, or polynucleotide linked to the corresponding hapten. The oligonucleotide can comprise a deoxyribonucleotide or a ribonucleotide. The oligonucleotide can comprise a single-stranded or double-stranded oligonucleotide.

In some embodiments when the PNME composition comprises a hapten-binding domain and a programmable or site directed nuclease, the composition further comprises a nucleic acid with homology arms complementary to regions flanking the target site for the programmable or site directed nuclease (e.g. a repair template or donor DNA). By this method, a nuclease can be delivered to the cell in vicinity of the site to be cleaved. In some cases, the repair template or donor DNA is a single- or double-stranded DNA repair template or donor DNA comprising from 5′ to 3′: a first homology arm comprising a sequence of at least about 20 nucleotides 5′ to the target sequence, an insert DNA sequence or region of at least about 10 nucleotides, and a second homology arm comprising a sequence of at least about 20 nucleotides 3′ to the target sequence. In some embodiments, the first or said second homology arms comprise a sequence of at least about 20, 40, 50, 80, 120, 150, 200, 300, 500, or 1000 nucleotides. In some cases, the 5′ and 3′ homology regions have different lengths. In some cases, the 5′ and 3′ homology regions have the same length. In some cases, the repair template or donor DNA is a single stranded polynucleotide and the 5′ homology region comprises 50-100 nucleotides and the 3′ homology region comprises 20-60 nucleotides. In some embodiments, the 3′ end of the 5′ homology region is homologous to a sequence within 5 nucleotides of the double-stranded break. In some cases, the 5′ end of the 3′ homology region is homologous to a sequence within 5 nucleotides of the double strand break. The insert region can comprise an exon, an intron, a transgene, a stop codon (e.g. a stop codon in frame with the gene ORF into which it is inserted), a coding sequence of a gene comprising at least one nonsense or missense mutation, or a mutation ablating activity of a PAM site in the vicinity of a sequence targeted by a PNME CRISPR enzyme. Example transgenes include selectable markers such as BlaS, HSV-tk, puromycin N-acetyl-transferase, or Tn5 NEO gene, which can be used to select for cells that have undergone recombination with the donor DNA or repair template. Example transgenes also include detectable labels such as fluorescent enzymes, proteins sequences capable of high-affinity detection with antibodies, epitope tags, or fluorescent proteins.

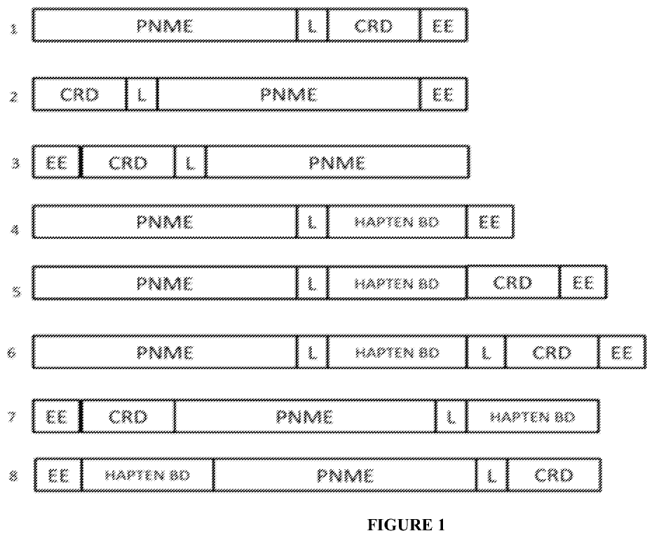

In some cases, PSME compositions described have various different orders of domains from N- to C-terminus within the PSME composition. In some embodiments, PNME compositions described herein are organized according to domain structure 1, 2, 3, 4, 5, 6, 7, or 8 depicted in FIG. 1. Example sequences for each of the domains depicted in FIG. 1 are illustrated in Table 5 and Table 6 below, alongside example combinations of domains to produce PNME composition fusion proteins.

In some embodiments, the PNME comprises one or more of the protein or nucleotide sequences in Table 5 or Table 6 below. In some embodiments, the PNME comprises a PNME having the combination and/or order of domains present in the sequences in Table 5 or Table 6 below. In some embodiments, the PNME comprises one or more of the sequences in Table 5 or Table 6 below absent one or more optional components such as an IL-2 secretion signal, a start codon, a stop codon, a His-tag, or a His-TEV tag. In some embodiments, any of the linker sequences in the PNME-CRD fusion proteins annotated in Table 6 below is replaced with one or more of the linker sequences from SEQ ID NOs: 61-65. In some embodiments, any of the endosomal escape sequences in the PNME-CRD fusion proteins annotated in Table 6 below is replaced with one or more of the endosomal escape sequences from SEQ ID NOs: 16-26.

In some embodiments, the present disclosure provides for a vector encoding any of the nucleotide sequences provided in Table 5 or Table 6 below. In some embodiments, the vector comprises one or more of the sequences in Table 5 or Table 6 below absent one or more optional components such as an IL-2 secretion signal, a start codon, a stop codon, a His-tag, a leader sequence, or a His-TEV tag. In some embodiments, the vector comprises one or more nucleotide sequences with codons optimized for expression in a particular organism encoding one or more of the protein sequences in Table 5 or Table 6 below. In some embodiments, the particular organism is mammalian, prokaryotic, E. coli, or insect.

TABLE 5
Example Protein or DNA Sequences for Domains Depicted in FIG. 1

| SEQ | | |
|---|---|---|
| ID NO: | Protein | Sequence |
| 43 | spCas9 | ATGGATAAAAAATACAGCATTGGTCTGGACATTGGCACGAATAGC |
| | (nucleotide | GTTGGTTGGGCAGTGATTACCGATGAATACAAAGTCCCGTCGAAAA |
| | sequence) | AATTCAAAGTGCTGGGTAACACCGATCGCCATAGCATTAAGAAAA |
| | | ACCTGATCGGTGCGCTGCTGTTTGATTCTGGCGAAACCGCGGAAGC |
| | | AACGCGTCTGAAACGTACCGCACGTCGCCGTTACACGCGCCGTAAA |
| | | AATCGTATTTGCTATCTGCAGGAAATCTTTAGCAACGAAATGGCGA |
| | | AAGTCGATGACTCATTTTTCCACCGCCTGGAAGAATCGTTTCTGGT |
| | | GGAAGAAGATAAAAAACATGAACGTCACCCGATTTTCGGCAATAT |
| | | CGTTGATGAAGTCGCGTACCATGAAAAATATCCGACGATTTACCAC |
| | | CTGCGTAAAAAACTGGTGGATTCTACCGACAAAGCCGATCTGCGCC |
| | | TGATTTATCTGGCACTGGCTCATATGATCAAATTTCGTGGTCACTTC |
| | | CTGATTGAAGGCGACCTGAACCCGGATAATAGTGACGTCGATAAA |
| | | CTGTTTATTCAGCTGGTGCAAACCTATAATCAGCTGTTCGAAGAAA |
| | | ACCCGATCAATGCAAGTGGTGTTGATGCGAAAGCCATTCTGTCCGC |
| | | TCGCCTGAGTAAATCCCGCCGTCTGGAAAACCTGATTGCACAGCTG |
| | | CCGGGTGAAAAGAAAAACGGTCTGTTTGGCAATCTGATCGCTCTGT |
| | | CACTGGGCCTGACGCCGAACTTTAAATCGAATTTCGACCTGGCAGA |
| | | AGATGCTAAACTGCAGCTGAGCAAAGATACCTACGATGACGATCT |
| | | GGACAACCTGCTGGCGCAAATTGGCGACCAGTATGCCGACCTGTTT |
| | | CTGGCGGCCAAAAATCTGTCAGATGCCATTCTGCTGTCGGACATCC |
| | | TGCGCGTGAACACCGAAATCACGAAAGCGCCGCTGTCAGCCTCGA |
| | | TGATTAAACGCTACGATGAACATCACCAGGACCTGACCCTGCTGAA |
| | | AGCACTGGTTCGTCAGCAACTGCCGGAAAAATACAAAGAAATTTTC |
| | | TTTGACCAAAGTAAAAATGGTTATGCAGGCTACATCGATGGCGGTG |
| | | CTTCCCAGGAAGAATTCTACAAATTCATCAAACCGATCCTGGAAAA |
| | | AATGGATGGTACGGAAGAACTGCTGGTGAAACTGAATCGTGAAGA |
| | | TCTGCTGCGTAAACAACGCACCTTTGACAACGGTAGCATTCCGCAT |
| | | CAGATCCACCTGGGCGAACTGCATGCGATTCTGCGCCGTCAGGAAG |
| | | ATTTTTATCCGTTCCTGAAAGACAACCGTGAAAAAATCGAAAAAAT |
| | | CCTGACGTTTCGCATCCCGTATTACGTTGGTCCGCTGGCACGTGGT |
| | | AATAGCCGCTTCGCATGGATGACCCGCAAATCTGAAGAAACCATTA |
| | | CGCCGTGGAACTTTGAAGAAGTGGTTGATAAAGGCGCAAGCGCTC |
| | | AGTCTTTTATCGAACGTATGACCAATTTCGATAAAAACCTGCCGAA |
| | | TGAAAAAGTGCTGCCGAAACATTCTCTGCTGTATGAATACTTTACC |
| | | GTTTAGAACGAACTGACGAAAGTGAAATATGTTACCGAGGGTATG |
| | | CGGAAACCGGCGTTTCTGAGTGGCGAACAGAAAAAAGCCATTGTG |
| | | GATCTGCTGTTCAAAACCAATCGTAAAGTTACGGTCAAACAGCTGA |
| | | AAGAAGATTACTTCAAGAAAATTGAATGTTTCGACAGCGTGGAAA |
| | | TTTCTGGTGTTGAAGATCGTTTCAACGCCTCTCTGGGCACCTATCAT |
| | | GACCTGCTGAAAATCATCAAAGACAAAGATTTTCTGGATAACGAA |
| | | GAAAACGAAGACATTCTGGAAGATATCGTGCTGACCCTGACGCTGT |
| | | TCGAAGATCGTGAAATGATTGAAGAACGCCTGAAAACGTACGCAC |
| | | ACCTGTTTGACGATAAAGTTATGAAACAGCTGAAACGCCGTCGCTA |
| | | TACCGGTTGGGGCCGTCTGAGCCGCAAACTGATTAATGGTATCCGC |
| | | GATAAACAATCAGGCAAAACGATTCTGGATTTCCTGAAATCGGAC |
| | | GGCTTTGCCAACCGTAATTTCATGCAGCTGATCCATGACGATTCCC |
| | | TGACCTTTAAAGAAGACATTCAGAAAGCACAAGTGTCAGGTCAAG |
| | | GCGATTCGCTGCATGAACACATTGCGAACCTGGCCGGTTCACCGGC |
| | | TATCAAAAAAGGCATCCTGCAGACCGTGAAAGTCGTGGATGAACT |
| | | GGTGAAAGTTATGGGTCGTCACAAACCGGAAAACATTGTTATCGA |
| | | AATGGCGCGCGAAAATCAGACCACGCAAAAAGGCCAGAAAAACTC |
| | | GCGTGAACGCATGAAACGCATTGAAGAAGGTATCAAAGAACTGGG |
| | | CAGCCAGATTCTGAAAGAACATCCGGTCGAAAACACCCAGCTGCA |
| | | AAATGAAAAACTGTACCTGTATTACCTGCAAAATGGTCGTGACATG |
| | | TATGTGGATCAGGAACTGGACATCAACCGCCTGTCTGACTATGATG |
| | | TCGACCACATTGTGCCGCAGAGCTTTCTGAAAGACGATTCTATCGA |
| | | TAACAAAGTTCTGACCCGTAGTGATAAAAACCGCGGCAAAAGCGA |
| | | CAATGTCCCGTCTGAAGAAGTTGTGAAGAAAATGAAAAACTACTG |
| | | GCGTCAACTGCTGAATGCGAAACTGATTACGCAGCGTAAATTCGAT |
| | | AACCTGACCAAAGCGGAACGCGGCGGTCTGTCCGAACTGGATAAA |
| | | GCCGGTTTTATCAAACGTCAACTGGTTGAAACCCGCCAGATTACGA |
| | | AACATGTCGCCCAGATCCTGGATTCACGCATGAACACGAAATACG |
| | | ACGAAAACGATAAACTGATCCGTGAAGTCAAAGTGATCACCCTGA |
| | | AAAGTAAACTGGTTTCCGATTTCCGTAAAGACTTTCAGTTCTACAA |
| | | AGTCCGCGAAATTAACAATTACCATCACGCACACGATGCTTATCTG |
| | | AATGCAGTGGTTGGTACCGCTCTGATCAAAAAATATCCGAAACTGG |
| | | AAAGCGAATTTGTGTATGGCGATTACAAAGTCTATGACGTGCGCAA |
| | | AATGATTGCGAAATCCGAACAGGAAATCGGCAAAGCGACCGCCAA |
| | | ATACTTTTTCTATTCAAACATCATGAACTTTTTCAAAACCGAAATTA |
| | | CGCTGGCAAATGGTGAAATTCGTAAACGCCCGCTGATCGAAACCA |
| | | ACGGTGAAACGGGCGAAATTGTGTGGGATAAAGGCCGTGACTTCG |
| | | CGACCGTTCGCAAAGTCCTGTCGATGCCGCAAGTGAATATCGTGAA |
| | | GAAAACCGAAGTGCAGACGGGCGGTTTTAGTAAAGAATCCATCCT |
| | | GCCGAAACGTAACAGCGATAAACTGATTGCGCGCAAAAAAGATTG |
| | | GGACCCGAAAAAATACGGCGGTTTTGATAGTCCGACGGTTGCATAT |
| | | TCCGTCCTGGTCGTGGCTAAAGTCGAAAAAGGTAAAAGTAAAAAA |
| | | CTGAAATCCGTGAAAGAACTGCTGGGCATTACCATCATGGAACGTA |
| | | GCTCTTTTGAGAAAAACCCGATTGACTTCCTGGAAGCCAAAGGTTA |
| | | CAAAGAAGTGAAAAAAGATCTGATCATCAAACTGCCGAAATATAG |
| | | CCTGTTCGAACTGGAAAACGGCCGTAAACGCATGCTGGCATCTGCT |
| | | GGTGAACTGCAGAAAGGCAATGAACTGGCACTGCCGAGTAAATAT |
| | | GTTAACTTTCTGTACCTGGCTAGCCATTATGAAAAACTGAAAGGTT |
| | | CTCCGGAAGATAACGAACAGAAACAACTGTTCGTCGAACAACATA |
| | | AACACTACCTGGATGAAATCATCGAACAGATCTCAGAATTCTCGAA |
| | | ACGCGTGATTCTGGCGGATGCCAATCTGGACAAAGTTCTGAGCGCG |
| | | TATAACAAACATCGTGATAAACCGATTCGCGAACAGGCCGAAAAT |
| | | ATTATCCACCTGTTTACCCTGACGAACCTGGGCGCACCGGCAGCTT |
| | | TTAAATACTTCGATACCACGATCGACCGTAAACGCTATACCTCAAC |
| | | GAAAGAAGTTCTGGATGCTACCCTGATTCATCAATCGATCACCGGT |
| | | CTGTATGAAACGCGTATTGATCTGAGTCAGCTGGGCGGTGAC |
| 44 | spCas9 | MDKKYSIGLDIGTNSVGWAVITDEYKVPSKKFKVLGNTDRHSIKKNLI |
| | (protein | GALLFDSGETAEATRLKRTARRRYTRRKNRICYLQEIFSNEMAKVDDS |
| | sequence) | FFHRLEESFLVEEDKKHERHPIFGNIVDEVAYHEKYPTIYHLRKKLVDS |
| | | TDKADLRLIYLALAHMIKFRGHFLIEGDLNPDNSDVDKLFIQLVQTYN |
| | | QLFEENPINASGVDAKAILSARLSKSRRLENLIAQLPGEKKNGLFGNLI |
| | | ALSLGLTPNFKSNFDLAEDAKLQLSKDTYDDDLDNLLAQIGDQYADL |
| | | FLAAKNLSDAILLSDILRVNTEITKAPLSASMIKRYDEHHQDLTLLKAL |
| | | VRQQLPEKYKEIFFDQSKNGYAGYIDGGASQEEFYKFIKPILEKMDGT |
| | | EELLVKLNREDLLRKQRTFDNGSIPHQIHLGELHAILRRQEDFYPFLKD |
| | | NREKIEKILTFRIPYYVGPLARGNSRFAWMTRKSEETITPWNFEEVVDK |
| | | GASAQSFIERMTNFDKNLPNEKVLPKHSLLYEYFTVYNELTKVKYVTE |
| | | GMRKPAFLSGEQKKAIVDLLFKTNRKVTVKQLKEDYFKKIECFDSVEI |
| | | SGVEDRFNASLGTYHDLLKIIKDKDFLDNEENEDILEDIVLTLTLFEDR |
| | | EMIEERLKTYAHLFDDKVMKQLKRRRYTGWGRLSRKLINGIRDKQSG |
| | | KTILDFLKSDGFANRNFMQLIHDDSLTFKEDIQKAQVSGQGDSLHEHI |
| | | ANLAGSPAIKKGILQTVKVVDELVKVMGRHKPENIVIEMARENQTTQ |
| | | KGQKNSRERMKRIEEGIKELGSQILKEHPVENTQLQNEKLYLYYLQNG |
| | | RDMYVDQELDINRLSDYDVDHIVPQSFLKDDSIDNKVLTRSDKNRGK |
| | | SDNVPSEEVVKKMKNYWRQLLNAKLITQRKFDNLTKAERGGLSELD |
| | | KAGFIKRQLVETRQITKHVAQILDSRMNTKYDENDKLIREVKVITLKS |
| | | KLVSDFRKDFQFYKVREINNYHHAHDAYLNAVVGTALIKKYPKLESE |
| | | FVYGDYKVYDVRKMIAKSEQEIGKATAKYFFYSNIMNFFKTEITLANG |
| | | EIRKRPLIETNGETGEIVWDKGRDFATVRKVLSMPQVNIVKKTEVQTG |
| | | GFSKESILPKRNSDKLIARKKDWDPKKYGGFDSPTVAYSVLVVAKVE |
| | | KGKSKKLKSVKELLGITIMERSSFEKNPIDFLEAKGYKEVKKDLIIKLP |
| | | KYSLFELENGRKRMLASAGELQKGNELALPSKYVNFLYLASHYEKLK |
| | | GSPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVLD |
| | | ATLIHQSITGLYETRIDLSQLGG |
| 45 | lbCPF1 | ATGTCAAAGCTGGAGAAATTCACCAACTGTTATAGCCTGTCTAAGA |
| | (nucleotide | CCCTGCGCTTCAAGGCAATCCCAGTGGGCAAGACACAAGAGAACA |
| | sequence) | TTGACAACAAACGGCTCCTGGTGGAGGATGAGAAGAGGGCTGAAG |
| | | ATTACAAGGGCGTTAAGAAGCTGCTGGATAGGTACTATCTGTCATT |
| | | CATCAACGATGTCCTCCACAGTATCAAGCTGAAGAATCTGAACAA |
| | | TTACATTTCTCTGTTCCGGAAGAAGACACGGACCGAGAAGGAGAA |
| | | CAAAGAGCTGGAGAATCTGGAGATCAACCTGAGGAAAGAAATAG |
| | | CTAAGGCTTTCAAAGGGAACGAGGGTTACAAGTCCCTGTTCAAGA |
| | | AAGACATTATCGAGACTATTCTGCCTGAGTTCCTGGACGATAAAGA |
| | | TGAGATCGCCCTCGTCAATTCCTTCAATGGGTTTACCACAGCCTTT |
| | | ACCGGCTTCTTCGACAATAGAGAGAATATGTTCTCTGAAGAGGCC |
| | | AAATCCACTAGCATCGCCTTTCGCTGCATAAACGAGAACCTGACTA |
| | | GGTACATCAGCAATATGGACATCTTTGAGAAAGTCGATGCCATATT |
| | | CGACAAACATGAGGTGCAGGAGATTAAGGAGAAGATCCTGAACTC |
| | | AGATTACGATGTCGAAGATTTCTTCGAGGGAGAGTTCTTCAACTTC |
| | | GTGCTCACACAAGAGGGCATTGATGTGTACAATGCAATCATTGGA |
| | | GGGTTCGTGACAGAGAGTGGCGAGAAGATAAAGGGCCTGAACGA |
| | | GTATATCAACCTCTACAACCAGAAAACCAAGCAGAAACTGCCTAA |
| | | GTTCAAGCCACTGTACAAACAAGTGCTCTCAGATAGGGAAAGCCT |
| | | GAGCTTCTACGGTGAAGGGTATACATCAGATGAAGAAGTGCTCGA |
| | | AGTGTTCCGCAACACCCTCAATAAGAACAGTGAAATCTTCTCTTCA |
| | | ATCAAGAAGCTGGAGAAACTGTTCAAGAATTTCGATGAGTACTCC |
| | | TCTGCCGGAATCTTTGTGAAGAATGGCCCTGCAATATCCACTATTA |
| | | GCAAAGACATCTTTGGCGAGTGGAACGTTATCAGGGATAAGTGGA |
| | | ATGCCGAGTACGATGATATTCATCTCAAGAAGAAAGCCGTGGTTA |
| | | CAGAGAAATACGAGGATGATAGACGCAAGAGCTTTAAGAAGATTG |
| | | GTAGCTTCTCTCTCGAACAGCTGCAGGAGTACGCCGACGCTGACCT |
| | | GTCAGTCGTGGAGAAACTCAAGGAGATCATAATCCAGAAGGTGGA |
| | | TGAAATCTACAAAGTGTATGGAAGCTCTGAGAAACTCTTCGATGC |
| | | AGACTTTGTTCTGGAGAAGAGTCTGAAGAAGAACGACGCAGTGGT |
| | | TGCTATCATGAAGGACCTGCTGGATTCTGTTAAGTCTTTCGAGAAT |
| | | TACATTAAGGCATTCTTTGGTGAAGGGAAGGAGACAAATAGGGAC |
| | | GAGAGCTTCTATGGCGACTTTGTTCTGGCCTACGACATCCTCCTCA |
| | | AGGTTGACCACATCTATGACGCTATACGGAATTACGTTACCCAGAA |
| | | GCCCTATAGCAAAGACAAGTTCAAGCTGTATTTCCAGAATCCACA |
| | | GTTTATGGGTGGGTGGGATAAAGACAAAGAAACAGATTACAGGGC |
| | | CACTATCCTGCGGTACGGCAGCAAATACTATCTGGCTATCATGGAT |
| | | AAGAAGTACGCCAAATGCCTCCAGAAGATCGACAAGGACGACGTG |
| | | AACGGTAACTACGAGAAGATCAATTACAAGCTCCTGCCAGGACCT |
| | | AACAAGATGCTGCCCAAGGTGTTCTTCTCCAAGAAATGGATGGCCT |
| | | ACTATAACCCAAGCGAGGACATTCAGAAGATATACAAGAATGGGA |
| | | CATTCAAGAAGGGCGATATGTTCAACCTCAACGACTGCCACAAGC |
| | | TGATTGATTTCTTCAAGGATAGCATTTCTCGCTATCCCAAGTGGTCT |
| | | AATGCATACGATTTCAACTTCAGCGAGACTGAGAAGTACAAAGAC |
| | | ATCGCTGGCTTCTACCGGGAGGTGGAAGAGCAAGGCTATAAGGTG |
| | | TCATTCGAATCCGCTTCTAAGAAGGAAGTGGATAAGCTCGTGGAA |
| | | GAGGGTAAGCTGTACATGTTCCAGATATACAACAAAGACTTCAGC |
| | | GATAAGAGCCACGGCACTCCAAACCTCCATACTATGTATTTCAAGC |
| | | TGCTGTTTGACGAGAACAACCACGGACAGATTAGGCTGTCAGGAG |
| | | GCGCAGAACTCTTCATGCGCAGAGCTTCACTGAAGAAGGAGGAAC |
| | | TCGTTGTCCACCCAGCCAATAGCCCTATAGCCAATAAGAATCCAGA |
| | | CAATCCTAAGAAAACCACTACTCTGTCTTACGATGTGTATAAGGAT |
| | | AAGAGATTCTCTGAAGATCAGTACGAACTGCACATACCCATTGCC |
| | | ATTAACAAGTGCCCTAAGAACATCTTCAAGATTAACACAGAGGTT |
| | | AGAGTGCTCCTGAAACACGACGATAACCCTTATGTTATAGGCATTG |
| | | ATCGCGGAGAGAGAAACCTGCTGTACATCGTCGTGGTGGACGGCA |
| | | AAGGCAACATCGTGGAACAGTACAGTCTCAATGAAATCATTAACA |
| | | ATTTCAACGGAATCCGCATTAAGACCGACTACCATTCTCTCCTCGA |
| | | CAAGAAGGAGAAAGAAAGGTTCGAAGCAAGACAGAATTGGACAA |
| | | GTATAGAGAATATCAAAGAACTGAAGGCTGGGTACATCTCTCAGG |
| | | TTGTGCACAAGATATGTGAGCTGGTGGAGAAGTACGACGCTGTTA |
| | | TCGCCCTCGAGGACCTGAATAGCGGCTTCAAGAACTCCAGGGTGA |
| | | AGGTGGAGAAGCAGGTGTATCAGAAGTTCGAGAAGATGCTGATCG |
| | | ACAAGCTCAACTATATGGTGGACAAGAAATCCAATCCTTGCGCTA |
| | | CTGGTGGAGCCCTGAAGGGCTATCAAATCACCAATAAGTTCGAAT |
| | | CTTTCAAGTCTATGAGCACCCAGAATGGCTTCATCTTCTACATACC |
| | | CGCATGGCTGACATCCAAGATTGATCCCTCTACCGGATTTGTTAAT |
| | | CTGCTCAAGACTAAGTACACCTCTATTGCTGACTCAAAGAAGTTCA |
| | | TATCATCATTTGACCGCATCATGTACGTGCCAGAAGAGGACCTGTT |
| | | CGAGTTTGCCCTGGATTACAAGAATTTCTCTCGGACTGACGCCGAC |
| | | TACATCAAGAAGTGGAAGCTCTACTCTTATGGTAATCGGATTCGCA |
| | | TATTCCGCAATCCCAAGAAGAATAACGTGTTCGATTGGGAGGAAG |
| | | TTTGCCTCACCAGCGCTTACAAGGAGCTGTTCAATAAGTATGGGAT |
| | | TAACTACCAGCAGGGCGACATAAGAGCCCTGCTGTGCGAACAATC |
| | | TGATAAGGCATTCTATTCCTCTTTCATGGCACTGATGTCACTGATG |
| | | CTGCAAATGCGCAATTCCATCACCGGAAGAACAGACGTGGACTTT |
| | | CTGATCTCTCCTGTCAAGAACTCAGATGGCATCTTCTACGATTCCC |
| | | GCAACTATGAAGCACAGGAGAATGCTATCCTGCCTAAGAATGCCG |
| | | ATGCAAATGGAGCCTATAACATCGCCAGAAAGGTCCTCTGGGCCA |
| | | TAGGACAATTCAAGAAAGCTGAAGATGAGAAGCTGGACAAGGTG |
| | | AAGATCGCCATTTCAAACAAAGAGTGGCTCGAATATGCTCAGACC |
| | | TCAGTGAAGCAT |
| 46 | lbCPF1 | MSKLEKFTNCYSLSKTLRFKAIPVGKTQENIDNKRLLVEDEKRAEDYK |
| | (protein | GVKKLLDRYYLSFINDVLHSIKLKNLNNYISLFRKKTRTEKENKELENL |
| | sequence) | EINLRKEIAKAFKGNEGYKSLFKKDIIETILPEFLDDKDEIALVNSFNGF |
| | | TTAFTGFFDNRENMFSEEAKSTSIAFRCINENLTRYISNMDIFEKVDAIF |
| | | DKHEVQEIKEKILNSDYDVEDFFEGEFFNFVLTQEGIDVYNAIIGGFVT |
| | | ESGEKIKGLNEYINLYNQKTKQKLPKFKPLYKQVLSDRESLSFYGEGY |
| | | TSDEEVLEVFRNTLNKNSEIFSSIKKLEKLFKNFDEYSSAGIFVKNGPAI |
| | | STISKDIFGEWNVIRDKWNAEYDDIHLKKKAVVTEKYEDDRRKSFKKI |
| | | GSFSLEQLQEYADADLSVVEKLKEIIIQKVDEIYKVYGSSEKLFDADFV |
| | | LEKSLKKNDAVVAIMKDLLDSVKSFENYIKAFFGEGKETNRDESFYGD |
| | | FVLAYDILLKVDHIYDAIRNYVTQKPYSKDKFKLYFQNPQFMGGWDK |
| | | DKETDYRATILRYGSKYYLAIMDKKYAKCLQKIDKDDVNGNYEKINY |
| | | KLLPGPNKMLPKVFFSKKWMAYYNPSEDIQKIYKNGTFKKGDMFNLN |
| | | DCHKLIDFFKDSISRYPKWSNAYDFNFSETEKYKDIAGFYREVEEQGY |
| | | KVSFESASKKEVDKLVEEGKLYMFQIYNKDFSDKSHGTPNLHTMYFK |
| | | LLFDENNHGQIRLSGGAELFMRRASLKKEELVVHPANSPIANKNPDNP |
| | | KKTTTLSYDVYKDKRFSEDQYELHIPIAINKCPKNIFKINTEVRVLLKH |
| | | DDNPYVIGIDRGERNLLYIVVVDGKGNIVEQYSLNEIINNFNGIRIKTDY |
| | | HSLLDKKEKERFEARQNWTSIENIKELKAGYISQVVHKICELVEKYDA |
| | | VIALEDLNSGFKNSRVKVEKQVYQKFEKMLIDKLNYMVDKKSNPCAT |
| | | GGALKGYQITNKFESFKSMSTQNGFIFYIPAWLTSKIDPSTGFVNLLKT |
| | | KYTSIADSKKFISSFDRIMYVPEEDLFEFALDYKNFSRTDADYIKKWKL |
| | | YSYGNRIRIFRNPKKNNVFDWEEVCLTSAYKELFNKYGINYQQGDIRA |
| | | LLCEQSDKAFYSSFMALMSLMLQMRNSITGRTDVDFLISPVKNSDGIF |
| | | YDSRNYEAQENAILPKNADANGAYNIARKVLWAIGQFKKAEDEKLD |
| | | KVKIAISNKEWLEYAQTSVKH |
| 47 | Mad7 | ATGAACAACGGCACAAATAATTTTCAGAACTTCATCGGGATCTCAA |
| | (nucleotide | GTTTGCAGAAAACGCTGCGCAATGCTCTGATCCCCACGGAAACCAC |
| | sequence) | GCAACAGTTCATCGTCAAGAACGGAATAATTAAAGAAGATGAGTT |
| | | ACGTGGCGAGAACCGCCAGATTCTGAAAGATATCATGGATGACTA |
| | | CTACCGCGGATTCATCTCTGAGACTCTGAGTTCTATTGATGACATA |
| | | GATTGGACTAGCCTGTTCGAAAAAATGGAAATTCAGCTGAAAAAT |
| | | GGTGATAATAAAGATACCTTAATTAAGGAACAGACAGAGTATCGG |
| | | AAAGCAATCCATAAAAAATTTGCGAACGACGATCGGTTTAAGAAC |
| | | ATGTTTAGCGCCAAACTGATTAGTGACATATTACCTGAATTTGTCA |
| | | TCCACAACAATAATTATTCGGCATCAGAGAAAGAGGAAAAAACCC |
| | | AGGTGATAAAATTGTTTTCGCGCTTTGCGACTAGCTTTAAAGATTA |
| | | CTTCAAGAACCGTGCAAATTGCTTTTCAGCGGACGATATTTCATCA |
| | | AGCAGCTGCCATCGCATCGTCAACGACAATGCAGAGATATTCTTTT |
| | | CAAATGCGCTGGTCTACCGCCGGATCGTAAAATCGCTGAGCAATGA |
| | | CGATATCAACAAAATTTCGGGCGATATGAAAGATTCATTAAAAGA |
| | | AATGAGTCTGGAAGAAATATATTCTTACGAGAAGTATGGGGAATTT |
| | | ATTACCCAGGAAGGCATTAGCTTCTATAATGATATCTGTGGGAAAG |
| | | TGAATTCTTTTATGAACCTGTATTGTCAGAAAAATAAAGAAAACAA |
| | | AAATTTATACAAACTTCAGAAACTTCACAAACAGATTCTATGCATT |
| | | GCGGACACTAGCTATGAGGTCCCGTATAAATTTGAAAGTGACGAG |
| | | GAAGTGTACCAATCAGTTAACGGCTTCCTTGATAACATTAGCAGCA |
| | | AACATATAGTCGAAAGATTACGCAAAATCGGCGATAACTATAACG |
| | | GCTACAACCTGGATAAAATTTATATCGTGTCCAAATTTTACGAGAG |
| | | CGTTAGCCAAAAAACCTACCGCGACTGGGAAACAATTAATACCGC |
| | | CCTCGAAATTCATTACAATAATATCTTGCCGGGTAACGGTAAAAGT |
| | | AAAGCCGACAAAGTAAAAAAAGCGGTTAAGAATGATTTACAGAAA |
| | | TCCATCACCGAAATAAATGAACTAGTGTCAAACTATAAGCTGTGCA |
| | | GTGACGACAACATCAAAGCGGAGACTTATATACATGAGATTAGCC |
| | | ATATCTTGAATAACTTTGAAGCACAGGAATTGAAATACAATCCGGA |
| | | AATTCACCTAGTTGAATCCGAGCTCAAAGCGAGTGAGCTTAAAAAC |
| | | GTGCTGGACGTGATCATGAATGCGTTTCATTGGTGTTCGGTTTTTAT |
| | | GACTGAGGAACTTGTTGATAAAGACAACAATTTTTATGCGGAACTG |
| | | GAGGAGATTTACGATGAAATTTATCCAGTAATTAGTCTGTACAACC |
| | | TGGTTCGTAACTACGTTACCCAGAAACCGTACAGCACGAAAAAGA |
| | | TTAAATTGAACTTTGGAATACCGACGTTAGCAGACGGTTGGTCAAA |
| | | GTCCAAAGAGTATTCTAATAACGCTATCATACTGATGCGCGACAAT |
| | | CTGTATTATCTGGGCATCTTTAATGCGAAGAATAAACCGGACAAGA |
| | | AGATTATCGAGGGTAATACGTCAGAAAATAAGGGTGACTACAAAA |
| | | AGATGATTTATAATTTGCTCCCGGGTCCCAACAAAATGATCCCGAA |
| | | AGTTTTCTTGAGCAGCAAGACGGGGGTGGAAACGTATAAACCGAG |
| | | CGCCTATATCCTAGAGGGGTATAAACAGAATAAACATATCAAGTCT |
| | | TCAAAAGACTTTGATATCACTTTCTGTCATGATCTGATCGACTACTT |
| | | CAAAAACTGTATTGCAATTCATCCCGAGTGGAAAAACTTCGGTTTT |
| | | GATTTTAGCGACACCAGTACTTATGAAGACATTTCCGGGTTTTATC |
| | | GTGAGGTAGAGTTACAAGGTTACAAGATTGATTGGACATACATTAG |
| | | CGAAAAAGACATTGATCTGCTGCAGGAAAAAGGTCAACTGTATCT |
| | | GTTCCAGATATATAACAAAGATTTTTCGAAAAAATCAACCGGGAAT |
| | | GACAACCTTCACACCATGTACCTGAAAAATCTTTTCTCAGAAGAAA |
| | | ATCTTAAGGATATCGTCCTGAAACTTAACGGCGAAGCGGAAATCTT |
| | | CTTCAGGAAGAGCAGCATAAAGAACCCAATCATTCATAAAAAAGG |
| | | CTCGATTTTAGTCAACCGTACCTACGAAGCAGAAGAAAAAGACCA |
| | | GTTTGGCAACATTCAAATTGTGCGTAAAAATATTCCGGAAAACATT |
| | | TATCAGGAGCTGTACAAATACTTCAACGATAAAAGCGACAAAGAG |
| | | CTGTCTGATGAAGCAGCCAAACTGAAGAATGTAGTGGGACACCAC |
| | | GAGGCAGCGACGAATATAGTCAAGGACTATCGCTACACGTATGAT |
| | | AAATACTTCCTTCATATGCCTATTACGATCAATTTCAAAGCCAATA |
| | | AAACGGGTTTTATTAATGATAGGATCTTACAGTATATCGCTAAAGA |
| | | AAAAGACTTACATGTGATCGGCATTGATCGGGGCGAGCGTAACCT |
| | | GATCTACGTGTCCGTGATTGATACTTGTGGTAATATAGTTGAACAG |
| | | AAAAGCTTTAACATTGTAAACGGCTACGACTATCAGATAAAACTGA |
| | | AACAACAGGAGGGCGCTAGACAGATTGCGCGGAAAGAATGGAAA |
| | | GAAATTGGTAAAATTAAAGAGATCAAAGAGGGCTACCTGAGCTTA |
| | | GTAATCCACGAGATCTCTAAAATGGTAATCAAATACAATGCAATTA |
| | | TAGCGATGGAGGATTTGTCTTATGGTTTTAAAAAAGGGCGCTTTAA |
| | | GGTCGAACGGCAAGTTTACCAGAAATTTGAAACCATGCTCATCAAT |
| | | AAACTCAACTATCTGGTATTTAAAGATATTTCGATTACCGAGAATG |
| | | GCGGTCTCCTGAAAGGTTATCAGCTGACATACATTCCTGATAAACT |
| | | TAAAAACGTGGGTCATCAGTGCGGCTGCATTTTTTATGTGCCTGCT |
| | | GCATACACGAGCAAAATTGATCCGACCACCGGCTTTGTGAATATCT |
| | | TTAAATTTAAAGACCTGACAGTGGACGCAAAACGTGAATTCATTAA |
| | | AAAATTTGACTCAATTCGTTATGACAGTGAAAAAAATCTGTTCTGC |
| | | TTTACATTTGACTACAATAACTTTATTACGCAAAACACGGTCATGA |
| | | GCAAATCATCGTGGAGTGTGTATACATACGGCGTGCGCATCAAACG |
| | | TCGCTTTGTGAACGGCCGCTTCTCAAACGAAAGTGATACCATTGAC |
| | | ATAACCAAAGATATGGAGAAAACGTTGGAAATGACGGACATTAAC |
| | | TGGCGCGATGGCCACGATCTTCGTCAAGACATTATAGATTATGAAA |
| | | TTGTTCAGCACATATTCGAAATTTTCCGTTTAACAGTGCAAATGCGT |
| | | AACTCCTTGTCTGAACTGGAGGACCGTGATTACGATCGTCTCATTT |
| | | CACCTGTACTGAACGAAAATAACATTTTTTATGACAGCGCGAAAGC |
| | | GGGGGATGCACTTCCTAAGGATGCCGATGCAAATGGTGCGTATTGT |
| | | ATTGCATTAAAAGGGTTATATGAAATTAAACAAATTACCGAAAATT |
| | | GGAAAGAAGATGGTAAATTTTCGCGCGATAAACTCAAAATCAGCA |
| | | ATAAAGATTGGTTCGACTTTATCCAGAATAAGCGCTATCTCTAA |
| 48 | Mad7 | MNNGTNNFQNFIGISSLQKTLRNALIPTETTQQFIVKNGIIKEDELRGEN |
| | (protein | RQILKDIMDDYYRGFISETLSSIDDIDWTSLFEKMEIQLKNGDNKDTLI |
| | sequence) | KEQTEYRKAIHKKFANDDRFKNMFSAKLISDILPEFVIHNNNYSASEKE |
| | | EKTQVIKLFSRFATSFKDYFKNRANCFSADDISSSSCHRIVNDNAEIFFS |
| | | NALVYRRIVKSLSNDDINKISGDMKDSLKEMSLEEIYSYEKYGEFITQE |
| | | GISFYNDICGKVNSFMNLYCQKNKENKNLYKLQKLHKQILCIADTSYE |
| | | VPYKFESDEEVYQSVNGFLDNISSKHIVERLRKIGDNYNGYNLDKIYIV |
| | | SKFYESVSQKTYRDWETINTALEIHYNNILPGNGKSKADKVKKAVKN |
| | | DLQKSITEINELVSNYKLCSDDNIKAETYIHEISHILNNFEAQELKYNPEI |
| | | HLVESELKASELKNVLDVIMNAFHWCSVFMTEELVDKDNNFYAELEE |
| | | IYDEIYPVISLYNLVRNYVTQKPYSTKKIKLNFGIPTLADGWSKSKEYS |
| | | NNAIILMRDNLYYLGIFNAKNKPDKKIIEGNTSENKGDYKKMIYNLLP |
| | | GPNKMIPKVFLSSKTGVETYKPSAYILEGYKQNKHIKSSKDFDITFCHD |
| | | LIDYFKNCIAIHPEWKNFGFDFSDTSTYEDISGFYREVELQGYKIDWTY |
| | | ISEKDIDLLQEKGQLYLFQIYNKDFSKKSTGNDNLHTMYLKNLFSEEN |
| | | LKDIVLKLNGEAEIFFRKSSIKNPIIHKKGSILVNRTYEAEEKDQFGNIQI |
| | | VRKNIPENIYQELYKYFNDKSDKELSDEAAKLKNVVGHHEAATNIVK |
| | | DYRYTYDKYFLHMPITINFKANKTGFINDRILQYIAKEKDLHVIGIDRG |
| | | ERNLIYVSVIDTCGNIVEQKSFNIVNGYDYQIKLKQQEGARQIARKEW |
| | | KEIGKIKEIKEGYLSLVIHEISKMVIKYNAIIAMEDLSYGFKKGRFKVER |
| | | QVYQKFETMLINKLNYLVFKDISITENGGLLKGYQLTYIPDKLKNVGH |
| | | QCGCIFYVPAAYTSKIDPTTGFVNIFKFKDLTVDAKREFIKKFDSIRYDS |
| | | EKNLFCFTFDYNNFITQNTVMSKSSWSVYTYGVRIKRRFVNGRFSNES |
| | | DTIDITKDMEKTLEMTDINWRDGHDLRQDIIDYEIVQHIFEIFRLTVQM |
| | | RNSLSELEDRDYDRLISPVLNENNIFYDSAKAGDALPKDADANGAYCI |
| | | ALKGLYEIKQITENWKEDGKFSRDKLKISNKDWFDFIQNKRYL |
| 49 | saCas9 | ATGAAAAGGAACTACATTCTGGGGCTGGACATCGGGATTACAAGC |
| | (nucleotide | GTGGGGTATGGGATTATTGACTATGAAACAAGGGACGTGATCGAC |
| | sequence) | GCAGGCGTCAGACTGTTCAAGGAGGCCAACGTGGAAAACAATGAG |
| | | GGACGGAGAAGCAAGAGGGGAGCCAGGCGCCTGAAACGACGGAG |
| | | AAGGCACAGAATCCAGAGGGTGAAGAAACTGCTGTTCGATTACAA |
| | | CCTGCTGACCGACCATTCTGAGCTGAGTGGAATTAATCCTTATGAA |
| | | GCCAGGGTGAAAGGCCTGAGTCAGAAGCTGTCAGAGGAAGAGTTT |
| | | TCCGCAGCTCTGCTGCACCTGGCTAAGCGCCGAGGAGTGCATAACG |
| | | TCAATGAGGTGGAAGAGGACACCGGCAACGAGCTGTCTACAAAGG |
| | | AACAG |
| | | ATCTCACGCAATAGCAAAGCTCTGGAAGAGAAGTATGTCGCAGAG |
| | | CTACAGCTGGAACGGCTGAAGAAAGATGGCGAGGTGAGAGGGTCA |
| | | ATTAATAGGTTCAAGACAAGCGACTACGTCAAAGAAGCCAAGCAG |
| | | CTGCTGAAAGTGCAGAAGGCTTACCACCAGCTGGATCAGAGCTTCA |
| | | TCGATACTTATATCGACCTGCTGGAGACTCGGAGAACCTACTATGA |
| | | GGGACCAGGAGAAGGGAGCCCCTTCGGATGGAAAGACATCAAGGA |
| | | ATGGTACGAGATGCTGATGGGACATTGCACCTATTTTCCAGAAGAG |
| | | CTGAGAAGCGTCAAGTACGCTTATAACGCAGATCTGTACAACGCCC |
| | | TGAATGACCTGAACAACCTGGTCATCACCAGGGATGAAAACGAGA |
| | | AACTGGAATACTATGAGAAGTTCCAGATCATCGAAAACGTGTTTAA |
| | | GCAGAAGAAAAAGCCTACACTGAAACAGATTGCTAAGGAGATCCT |
| | | GGTCAACGAAGAGGACATCAAGGGCTACCGGGTGACAAGCACTGG |
| | | AAAACCAGAGTTCACCAATCTGAAAGTGTATCACGATATTAAGGA |
| | | CATCACAGCACGGAAAGAAATCATTGAGAACGCCGAACTGCTGGA |
| | | TCAGATTGCTAAGATCCTGACTATCTACCAGAGTTCCGAGGACATC |
| | | CAGGAAGAGCTGACTAACCTGAACAGCGAGCTGACCCAGGAAGAG |
| | | ATCGAACAGATTAGTAATCTGAAGGGGTACACCGGAACACACAAC |
| | | CTGTCCCTGAAAGCTATCAATCTGATTCTGGATGAGCTGTGGCATA |
| | | CAAACGACAATCAGATTGCAATCTTTAACCGGCTGAAGCTGGTACC |
| | | AAAAAAGGTGGACCTGAGTCAGCAGAAAGAGATCCCAACCACACT |
| | | GGTGGACGATTTCATTCTGTCACCCGTGGTCAAGCGGAGCTTCATC |
| | | CAGAGCATCAAAGTGATCAACGCCATCATCAAGAAGTACGGCCTG |
| | | CCCAATGATATCATTATCGAGCTGGCTAGGGAGAAGAACAGCAAG |
| | | GACGCACAGAAGATGATCAATGAGATGCAGAAACGAAACCGGCAG |
| | | ACCAATGAACGCATTGAAGAGATTATCCGAACTACCGGGAAAGAG |
| | | AACGCAAAGTACCTGATTGAAAAAATCAAGCTGCACGATATGCAG |
| | | GAGGGAAAGTGTCTGTATTCTCTGGAGGCCATCCCCCTGGAGGACC |
| | | TGCTGAACAATCCATTCAACTACGAGGTCGATCATATTATCCCCAG |
| | | AAGCGTGTCCTTCGACAATTCCTTTAACAACAAGGTGCTGGTCAAG |
| | | CAGGAAGAGAACTCTAAAAAGGGCAATAGGACTCCTTTCCAGTAC |
| | | CTGTCTAGTTCAGATTCCAAGATCTCTTACGAAACCTTTAAAAAGC |
| | | ACATTCTGAATCTGGCCAAAGGAAAGGGCCGCATCAGCAAGACCA |
| | | AAAAGGAGTACCTGCTGGAAGAGCGGGACATCAACAGATTCTCCG |
| | | TCCAGAAGGATTTTATTAACCGGAATCTGGTGGACACAAGATACGC |
| | | TACTCGCGGCCTGATGAATCTGCTGCGATCCTATTTCCGGGTGAAC |
| | | AATCTGGATGTGAAAGTCAAGTCCATCAACGGCGGGTTCACATCTT |
| | | TTCTGAGGCGCAAATGGAAGTTTAAAAAGGAGCGCAACAAAGGGT |
| | | ACAAGCACCATGCCGAAGATGCTCTGATTATCGCAAATGCCGACTT |
| | | CATCTTTAAGGAGTGGAAAAAGCTGGACAAAGCCAAGAAAGTGAT |
| | | GGAGAACCAGATGTTCGAAGAGAAGCAGGCCGAATCTATGCCCGA |
| | | AATCGAGACAGAACAGGAGTACAAGGAGATTTTCATCACTCCTCA |
| | | CCAGATCAAGCATATCAAGGATTTCAAGGACTACAAGTACTCTCAC |
| | | CGGGTGGATAAAAAGCCCAACAGAGAGCTGATCAATGACACCCTG |
| | | TATAGTACAAGAAAAGACGATAAGGGGAATACCCTGATTGTGAAC |
| | | AATCTGAACGGACTGTACGACAAAGATAATGACAAGCTGAAAAAG |
| | | CTGATCAACAAAAGTCCCGAGAAGCTGCTGATGTACCACCATGATC |
| | | CTCAGACATATCAGAAACTGAAGCTGATTATGGAGCAGTACGGCG |
| | | ACGAGAAGAACCCACTGTATAAGTACTATGAAGAGACTGGGAACT |
| | | ACCTGACCAAGTATAGCAAAAAGGATAATGGCCCCGTGATCAAGA |
| | | AGATCAAGTACTATGGGAACAAGCTGAATGCCCATCTGGACATCA |
| | | CAGACGATTACCCTAACAGTCGCAACAAGGTGGTCAAGCTGTCACT |
| | | GAAGCCATACAGATTCGATGTCTATCTGGACAACGGCGTGTATAAA |
| | | TTTGTGACTGTCAAGAATCTGGATGTCATCAAAAAGGAGAACTACT |
| | | ATGAAGTGAATAGCAAGTGCTACGAAGAGGCTAAAAAGCTGAAAA |
| | | AGATTAGCAACCAGGCAGAGTTCATCGCCTCCTTTTACAACAACGA |
| | | CCTGATTAAGATCAATGGCGAACTGTATAGGGTCATCGGGGTGAAC |
| | | AATGATCTGCTGAACCGCATTGAAGTGAATATGATTGACATCACTT |
| | | ACCGAGAGTATCTGGAAAACATGAATGATAAGCGCCCCCCTCGAA |
| | | TTATCAAAACAATCGCCTCTAAGACTCAGAGTATCAAAAAGTACTC |
| | | AACCGACATTCTGGGAAACCTGTATGAGGTGAAGAGCAAAAAGCA |
| | | CCCTCAGATTATCAAAAAGGGCTAA |
| 50 | saCas9 | MKRNYILGLDIGITSVGYGIIDYETRDVIDAGVRLFKEANVENNEGRRS |
| | (protein | KRGARRLKRRRRHRIQRVKKLLFDYNLLTDHSELSGINPYEARVKGLS |
| | sequence) | QKLSEEEFSAALLHLAKRRGVHNVNEVEEDTGNELSTKEQISRNSKAL |
| | | EEKYVAELQLERLKKDGEVRGSINRFKTSDYVKEAKQLLKVQKAYHQ |
| | | LDQSFIDTYIDLLETRRTYYEGPGEGSPFGWKDIKEWYEMLMGHCTYF |
| | | PEELRSVKYAYNADLYNALNDLNNLVITRDENEKLEYYEKFQIIENVF |
| | | KQKKKPTLKQIAKEILVNEEDIKGYRVTSTGKPEFTNLKVYHDIKDITA |
| | | RKEIIENAELLDQIAKILTIYQSSEDIQEELTNLNSELTQEEIEQISNLKGY |
| | | TGTHNLSLKAINLILDELWHTNDNQIAIFNRLKLVPKKVDLSQQKEIPT |
| | | TLVDDFILSPVVKRSFIQSIKVINAIIKKYGLPNDIIIELAREKNSKDAQK |
| | | MINEMQKRNRQTNERIEEIIRTTGKENAKYLIEKIKLHDMQEGKCLYSL |
| | | EAIPLEDLLNNPFNYEVDHIIPRSVSFDNSFNNKVLVKQEENSKKGNRT |
| | | PFQYLSSSDSKISYETFKKHILNLAKGKGRISKTKKEYLLEERDINRFSV |
| | | QKDFINRNLVDTRYATRGLMNLLRSYFRVNNLDVKVKSINGGFTSFLR |
| | | RKWKFKKERNKGYKHHAEDALIIANADFIFKEWKKLDKAKKVMENQ |
| | | MFEEKQAESMPEIETEQEYKEIFITPHQIKHIKDFKDYKYSHRVDKKPN |
| | | RELINDTLYSTRKDDKGNTLIVNNLNGLYDKDNDKLKKLINKSPEKLL |
| | | MYHHDPQTYQKLKLIMEQYGDEKNPLYKYYEETGNYLTKYSKKDNG |
| | | PVIKKIKYYGNKLNAHLDITDDYPNSRNKVVKLSLKPYRFDVYLDNG |
| | | VYKFVTVKNLDVIKKENYYEVNSKCYEEAKKLKKISNQAEFIASFYNN |
| | | DLIKINGELYRVIGVNNDLLNRIEVNMIDITYREYLENMNDKRPPRIIKT |
| | | IASKTQSIKKYSTDILGNLYEVKSKKHPQIIKKG |
| 51 | asCPF1 | ATGACCCAGTTCGAGGGGTTTACCAATCTGTATCAAGTGAGCAAGA |
| | (nucleotide | CGCTGCGCTTTGAACTGATCCCACAGGGAAAAACCTTAAAACATAT |
| | sequence) | TCAAGAGCAGGGCTTTATCGAAGAAGATAAGGCCCGTAATGACCA |
| | | TTACAAAGAGTTAAAGCCGATTATTGATCGTATCTACAAGACCTAT |
| | | GCGGACCAGTGCTTACAATTGGTACAGCTTGATTGGGAGAACCTCT |
| | | CTGCCGCCATCGATTCCTATCGTAAAGAAAAAACTGAAGAAACGC |
| | | GCAACGCCCTGATTGAAGAGCAGGCCACCTATCGTAACGCGATTCA |
| | | TGACTATTTTATTGGCCGTACGGACAATCTGACGGACGCGATCAAC |
| | | AAGCGCCATGCGGAGATTTACAAAGGACTGTTTAAGGCTGAACTGT |
| | | TCAATGGTAAGGTCCTTAAACAGCTTGGGACCGTCACAACGACGG |
| | | AACATGAAAACGCGTTATTACGTAGCTTCGACAAGTTTACCACGTA |
| | | TTTCTCCGGCTTTTACGAAAATCGCAAAAACGTTTTCAGTGCCGAG |
| | | GATATTTCCACTGCTATCCCTCATCGCATTGTGCAAGACAACTTCCC |
| | | AAAATTCAAAGAAAATTGTCATATCTTCACCCGCTTAATCACCGCT |
| | | GTACCGTCCCTGCGTGAGCATTTCGAAAACGTGAAAAAGGCCATTG |
| | | GTATCTTCGTGTCTACTTCGATTGAGGAGGTATTTTCCTTTCCATTC |
| | | TATAATCAGCTGCTGACCCAGACCCAAATTGATCTGTACAACCAGC |
| | | TGCTTGGCGGTATTTCTCGTGAAGCAGGAACCGAAAAAATCAAAG |
| | | GGTTGAACGAGGTGCTTAATCTGGCAATCCAGAAAAATGATGAAA |
| | | CCGCCCACATCATTGCTTCGTTACCTCATCGTTTTATCCCGTTGTTC |
| | | AAGCAAATTTTAAGTGATCGCAATACGCTGTCGTTTATTCTGGAAG |
| | | AATTCAAAAGTGATGAAGAGGTAATTCAGTCGTTTTGCAAATATAA |
| | | AACCCTGTTACGTAACGAAAATGTCCTGGAAACAGCCGAGGCTTTG |
| | | TTTAACGAACTGAATAGCATTGACCTGACGCATATCTTTATTAGCC |
| | | ACAAAAAATTAGAGACCATCTCATCAGCTCTGTGCGATCATTGGGA |
| | | TACACTGCGCAATGCGCTGTATGAACGTCGTATTTCGGAATTGACT |
| | | GGCAAAATCACTAAAAGCGCGAAAGAGAAAGTACAGCGCTCGCTT |
| | | AAACATGAAGATATCAACCTGCAGGAGATCATCAGCGCCGCGGGT |
| | | AAAGAACTGTCGGAGGCATTTAAACAGAAGACGAGCGAGATTCTG |
| | | TCCCACGCACATGCCGCCTTAGACCAGCCGCTCCCGACCACTCTGA |
| | | AGAAACAGGAAGAGAAAGAAATCCTTAAAAGTCAACTGGACAGTT |
| | | TACTGGGTCTCTATCATCTGCTGGATTGGTTTGCGGTAGACGAAAG |
| | | CAATGAAGTGGATCCGGAGTTTAGTGCCCGTCTGACAGGAATCAA |
| | | GCTGGAAATGGAGCCTTCGCTTAGCTTCTACAACAAAGCCCGCAAT |
| | | TATGCCACGAAAAAACCCTATAGTGTCGAAAAATTTAAACTCAACT |
| | | TTCAAATGCCGACCCTTGCGTCGGGCTGGGATGTCAACAAAGAAA |
| | | AAAACAACGGAGCTATTCTGTTCGTTAAAAATGGTCTGTACTACCT |
| | | GGGCATCATGCCGAAACAGAAAGGTCGCTACAAAGCCCTTTCGTTC |
| | | GAGCCCACGGAAAAAACAAGCGAAGGCTTCGACAAAATGTACTAC |
| | | GATTACTTTCCGGATGCAGCAAAAATGATCCCGAAATGTTCCACAC |
| | | AGCTGAAAGCCGTTACAGCACATTTTCAGACGCACACCACCCCCAT |
| | | CTTACTGTCCAACAATTTTATTGAACCGCTGGAGATTACTAAAGAA |
| | | ATTTATGATTTGAACAATCCGGAAAAAGAGCCAAAAAAGTTTCAA |
| | | ACCGCCTACGCTAAAAAAACCGGGGATCAGAAAGGGTACCGCGAA |
| | | GCGTTGTGCAAGTGGATTGATTTCACCCGCGATTTTCTCAGTAAAT |
| | | ATACCAAGACTACCTCGATTGACCTGAGCTCACTGCGCCCGAGCTC |
| | | TCAATATAAGGATTTGGGTGAGTACTATGCTGAATTAAACCCTTTA |
| | | TTGTACCACATTTCTTTTCAGCGCATCGCCGAAAAGGAAATTATGG |
| | | ACGCAGTCGAAACCGGGAAACTGTACCTGTTCCAGATCTATAATAA |
| | | GGACTTCGCCAAAGGACATCATGGCAAACCGAACCTGCACACCCTT |
| | | TACTGGACCGGGCTTTTCTCTCCGGAAAATTTGGCGAAAACCTCGA |
| | | TCAAGCTTAACGGTCAAGCTGAGCTGTTTTACCGTCCAAAATCCCG |
| | | CATGAAGCGCATGGCGCATCGTTTAGGTGAAAAAATGCTGAATAA |
| | | GAAACTGAAAGATCAGAAAACCCCTATCCCGGATACCCTCTACCA |
| | | GGAACTGTATGATTACGTGAACCATCGTCTCTCGCATGACCTGTCA |
| | | GACGAAGCGCGTGCGTTACTGCCCAATGTAATCACAAAAGAAGTTT |
| | | CGCATGAAATTATTAAAGATCGTCGTTTTACATCTGATAAATTCTTT |
| | | TTTCATGTTCCGATCACCCTCAACTATCAGGCCGCAAACAGTCCAA |
| | | GTAAGTTTAACCAGCGCGTTAATGCTTACCTGAAGGAACATCCGGA |
| | | GACTCCGATTATTGGAATTGATCGCGGTGAACGTAATTTGATCTAT |
| | | ATCACTGTGATCGATAGTACCGGTAAGATTCTGGAGCAGCGCAGCT |
| | | TGAACACAATTCAACAGTTTGATTATCAGAAAAAATTAGACAACCG |
| | | CGAAAAAGAGCGCGTGGCTGCCCGTCAGGCGTGGTCTGTTGTCGGT |
| | | ACCATTAAAGATCTGAAGCAGGGCTATCTTTCTCAGGTTATTCACG |
| | | AAATTGTAGATCTGATGATCCATTATCAGGCGGTTGTTGTGTTGGA |
| | | GAATCTCAATTTCGGTTTTAAGAGTAAGCGCACAGGCATCGCTGAA |
| | | AAAGCAGTTTATCAGCAGTTTGAAAAAATGCTGATCGACAAATTGA |
| | | ACTGTTTAGTTCTCAAAGATTACCCAGCGGAAAAGGTGGGCGGAGT |
| | | GCTGAATCCGTACCAATTAACGGATCAATTCACTTCCTTCGCAAAG |
| | | ATGGGTACCCAAAGCGGCTTTCTGTTCTATGTGCCGGCCCCGTATA |
| | | CCTCGAAAATCGATCCACTGACGGGCTTCGTAGATCCGTTCGTGTG |
| | | GAAAACCATTAAAAATCATGAAAGTCGTAAACATTTTCTCGAAGGC |
| | | TTCGACTTCCTGCACTACGACGTGAAAACTGGCGATTTCATTCTGC |
| | | ATTTTAAAATGAACCGCAACCTTTCGTTTCAGCGCGGTCTGCCGGG |
| | | CTTTATGCCGGCTTGGGACATTGTTTTTGAGAAAAATGAAACCCAG |
| | | TTTGATGCTAAAGGCACTCCTTTCATCGCCGGTAAACGCATCGTAC |
| | | CTGTGATTGAAAACCATCGTTTTACAGGGCGTTACCGTGATTTATA |
| | | CCCGGCGAACGAATTGATCGCGCTGCTGGAGGAAAAGGGCATCGT |
| | | TTTCCGTGACGGCTCCAATATTCTGCCGAAATTACTGGAAAACGAC |
| | | GATTCACACGCAATTGATACCATGGTCGCACTGATTCGCTCAGTCT |
| | | TACAGATGCGTAACTCTAATGCAGCCACAGGAGAAGATTATATTAA |
| | | TTCGCCAGTCCGCGATTTGAACGGTGTTTGCTTCGACAGCCGTTTTC |
| | | AGAATCCTGAATGGCCGATGGACGCTGATGCCAACGGAGCTTATC |
| | | ATATCGCCCTGAAAGGCCAGCTCCTGCTGAACCACCTGAAGGAAA |
| | | GCAAAGATCTGAAATTGCAGAACGGCATTAGCAACCAGGACTGGT |
| | | TAGCATACATCCAGGAACTGCGTAAC |
| 52 | asCPF1 | MTQFEGFTNLYQVSKTLRFELIPQGKTLKHIQEQGFIEEDKARNDHYK |
| | (protein | ELKPIIDRIYKTYADQCLQLVQLDWENLSAAIDSYRKEKTEETRNALIE |
| | sequence) | EQATYRNAIHDYFIGRTDNLTDAINKRHAEIYKGLFKAELFNGKVLKQ |
| | | LGTVTTTEHENALLRSFDKFTTYFSGFYENRKNVFSAEDISTAIPHRIVQ |
| | | DNFPKFKENCHIFTRLITAVPSLREHFENVKKAIGIFVSTSIEEVFSFPFY |
| | | NQLLTQTQIDLYNQLLGGISREAGTEKIKGLNEVLNLAIQKNDETAHII |
| | | ASLPHRFIPLFKQILSDRNTLSFILEEFKSDEEVIQSFCKYKTLLRNENVL |
| | | ETAEALFNELNSIDLTHIFISHKKLETISSALCDHWDTLRNALYERRISE |
| | | LTGKITKSAKEKVQRSLKHEDINLQEIISAAGKELSEAFKQKTSEILSHA |
| | | HAALDQPLPTTLKKQEEKEILKSQLDSLLGLYHLLDWFAVDESNEVDP |
| | | EFSARLTGIKLEMEPSLSFYNKARNYATKKPYSVEKFKLNFQMPTLAS |
| | | GWDVNKEKNNGAILFVKNGLYYLGIMPKQKGRYKALSFEPTEKTSEG |
| | | FDKMYYDYFPDAAKMIPKCSTQLKAVTAHFQTHTTPILLSNNFIEPLEI |
| | | TKEIYDLNNPEKEPKKFQTAYAKKTGDQKGYREALCKWIDFTRDFLS |
| | | KYTKTTSIDLSSLRPSSQYKDLGEYYAELNPLLYHISFQRIAEKEIMDA |
| | | VETGKLYLFQIYNKDFAKGHHGKPNLHTLYWTGLFSPENLAKTSIKLN |
| | | GQAELFYRPKSRMKRMAHRLGEKMLNKKLKDQKTPIPDTLYQELYD |
| | | YVNHRLSHDLSDEARALLPNVITKEVSHEIIKDRRFTSDKFFFHVPITLN |
| | | YQAANSPSKFNQRVNAYLKEHPETPIIGIDRGERNLIYITVIDSTGKILE |
| | | QRSLNTIQQFDYQKKLDNREKERVAARQAWSVVGTIKDLKQGYLSQ |
| | | VIHEIVDLMIHYQAVVVLENLNFGFKSKRTGIAEKAVYQQFEKMLIDK |
| | | LNCLVLKDYPAEKVGGVLNPYQLTDQFTSFAKMGTQSGFLFYVPAPY |
| | | TSKIDPLTGFVDPFVWKTIKNHESRKHFLEGFDFLHYDVKTGDFILHFK |
| | | MNRNLSFQRGLPGFMPAWDIVFEKNETQFDAKGTPFIAGKRIVPVIEN |
| | | HRFTGRYRDLYPANELIALLEEKGIVFRDGSNILPKLLENDDSHAIDTM |
| | | VALIRSVLQMRNSNAATGEDYINSPVRDLNGVCFDSRFQNPEWPMDA |
| | | DANGAYHIALKGQLLLNHLKESKDLKLQNGISNQDWLAYIQELRN |
| | CRD domain sequences | |
| 53 | 7D12 | ATGGGTGGCGGTGGCAGCGGTGGCGGTGGCAGCCAGGTGAAACTG |
| | VHH | GAGGAAAGCGGTGGCGGTAGCGTTCAAACCGGCGGTAGCCTGCGT |
| | (nucleotide | CTGACCTGCGCGGCGAGCGGTCGTACCAGCCGTAGCTATGGTATGG |
| | sequence) | GTTGGTTTCGTCAGGCGCCGGGCAAGGAGCGTGAATTTGTGAGCGG |
| | | TATCAGCTGGCGTGGCGACAGCACCGGTTATGCGGATAGCGTGAA |
| | | GGGTCGTTTCACCATTAGCCGTGACAACGCGAAAAACACCGTTGAT |
| | | CTGCAAATGAACAGCCTGAAGCCGGAGGACACCGCGATCTACTAT |
| | | TGCGCGGCGGCGGCGGGTAGCGCGTGGTATGGTACCCTGTACGAA |
| | | TATGATTACTGGGGCCAGGGTACCCAAGTGACCGTTAGCAGCCTCG |
| | | AG |
| 54 | 7D12 | QVKLEESGGGSVQTGGSLRLTCAASGRTSRSYGMGWFRQAPGKEREF |
| | VHH | VSGISWRGDSTGYADSVKGRFTISRDNAKNTVDLQMNSLKPEDTAIY |
| | (protein | YCAAAAGSAWYGTLYEYDYWGQGTQVTVSSLE |
| | sequence) | |
| 55 | Triple | ATGGCATCACCATGGGTGGATAACAAATTTAACAAAGAATTTTCTT |
| | Helix 1 | ATGCGATTAATGAAATTGCCCTGCCGAACCTGAACGAAAAGCAGG |
| | (nucleotide | GCAGAGCGTTTATTAACAGCCTGCGTGATGATCCGAGCCAGAGCGC |
| | sequence) | GAACCTGCTGGCGGAAGCGAAAAAACTGAACGATGCGCAGGCGCC |
| | | GAAATGTTGTTGTTGT |
| 56 | Triple | MASPWVDNKFNKEFSYAINEIALPNLNEKQGRAFINSLRDDPSQSANL |
| | Helix 1 | LAEAKKLNDAQAPKCCCC |
| | (protein | |
| | sequence) | |
| 57 | Triple | ATGGCATCACCATGGGTGGATAACAAATTTAACAAAGAATGGTCC |
| | Helix2 | AAAGGCGGATGCCGAAATTGTTCTTCACCTGCCGAACCTGAACGAC |
| | (nucleotide | GCCCAGGGAGCGTTTATGGTGAGCCTGAGGATGCCTCCGAGCCAG |
| | sequence) | AGCGCGAACCTGCTGGCGGAAGCGAAAAAACTGAACGATGCGCAG |
| | | GCGCCGAAATGTTGTTGTGT |
| 58 | Triple | MASPWVDNKFNKEWSKGGCRNCSSPAEPERRPGSVYGEPEDASEPER |
| | Helix2 | EPAGGSEKTERCAGAEMLLC |
| | (protein | |
| | sequence) | |
| 59 | VHH3 | CTGCGTCTGACCTGCGCGGCGTCTGGTCGTACCTCTCGTTCTTACGG |
| | (CD1/2/3d | TATGGGTTGGTTCCGTCAGGCGCCGGGTAAAGAACGTGAATTCGTT |
| | omains, | TCTGGTATCTCTTGGCGTGGTGACTCTACCGGTTACGCGGACTCTGT |
| | nucleotide | TAAAGGTCGTTTCACCATCTCTCGTGACAACGCGAAAAACACCGTT |
| | sequence) | GACCTGCAGATGAACTCTCTGAAACCGGAAGACACCGCGATCTACT |
| | | ACTGCGCGGCGGCGGCGGGTTCTGCGTGGTACGGTACCCTGTACGA |
| | | ATACGACTACTGGGGTCAGGGTACCCAGGTTACC |
| 60 | VHH3 | LRLTCAASGRTSRSYGMGWFRQAPGKEREFVSGISWRGDSTGYADSV |
| | (CDl/2/3 | KGRFTISRDNAKNTVDLQMNSLKPEDTAIYYCAAAAGSAWYGTLYEY |
| | domains, | DYWGQGTQVT |
| | protein | |
| | sequence) | |
| | Linker sequences (wherein n is from 1 to 10) | |
| 61 | GPcPcPc | GlySer-polyPro(Glyc)-polyPro(Glyc)-polyPro(Glyc) repeated n times |
| 62 | GPPcP | GlySer-polyPro-polyPro(Glyc)-polyPro repeated n times |
| 63 | GS | Glycine-Serine repeated n times |
| 64 | GGGS | (Gly-Gly-Gly-GLY-Serine) repeated n times |
| 65 | G-CSF-Tf | A(EAAAK)₄ALEA(EAAAK)₄A |
| | Endosome Escape Sequences | |
| 16 | EE Motif | X₁X₂X₃X₄X₅X₆X₇X₈X₉; wherein |
| | 1 | X₁ is P or C; |
| | | X₂, X₃, X₄, and X₅ are independently selected from C, R, or K; and |
| | | X₆, X₇, X₈, and X₉ are independently selected from C, R, K, A, or W. |
| 17 | EE Motif | X₁X₂X₃X₄X₅X₆X₇X₈X₉; wherein |
| | 2 | X₁ is P or C; |
| | | X₂, X₃, X₄, and X₅ are independently selected from C, R, or K; and |
| | | X₆, X₇, X₈, and X₉ are independently selected from C, R, K, A, or W., |
| | | and wherein at least 3 of X₁-X₉ are C and no more than 8 of X₁-X₉ are |
| | | C. |
| 18 | EE1 | PCRKCACCA |
| 19 | EE2 | PRCCRWCCA |
| 20 | EE3 | PRRCKRCKC |
| 21 | EE4 | CKKCRKCCK |
| 22 | EE5 | CCRCKCWCC |
| 23 | EE6 | CCRKCCCCC |
| 24 | EE7 | PRKCCCCCC |
| 25 | EE8 | HHHHHHHHHH |
| 26 | EE9 | CCCCCC |

TABLE 6
Example PNME-CRD Fusion Proteins

| SEQ ID | | | Domain annotations (N-C terminus for |
|---|---|---|---|
| NO | Protein | Sequence | protein or 5′-3′ for nucleotide sequence) |
| 66 | 7d-md7- | ATGTACAGGATGCAACTCCTGTCTTGCATTGCACTAAGTCTT | Domains in order: |
| | L2 (7d12) | GCACTTGTCACGAACTCTCAGGTGAAACTGGAGGAGAGCGGGG | IL-2secretion sequence: bold |
| | (nucleotide | GCGGGAGCGTGCAGACTGGGGGGAGCCTGAGACTGACATGCGCA | Cell recognition domain: double underline |
| | sequence) | GCAAGCGGGCGGACAAGCCGGAGCTACGGAATGGGATGGTTCAG | Linker: italics |
| | | GCAGGCACCAGGCAAGGAGAGGGAGTTTGTGAGCGGCATCTCCT | Endonuclease: single underline |
| | | GGAGAGGCGATAGCACCGGCTATGCCGACTCCGTGAAGGGCAGG | NLS sequence: bold |
| | | TTCACCATCAGCCGCGATAATGCCAAGAACACAGTGGACCTGCA | TEV-cleavage sequence: underlined |
| | | GATGAACTCCCTGAAGCCCGAGGACACCGCAATCTACTATTGCG | Endosomal escape sequence: bold |
| | | CAGCAGCAGCAGGCTCCGCCTGGTACGGCACACTGTACGAGTAT | Residue numbering: |
| | | GATTACTGGGGCCAGGGCACCCAGGTGACAGTGAGCTCCGCCCT | IL-2 secretion sequence: 1-60 |
| | | GGAGGGAGGAGGAGGCTCTGGAGGAGGAGGCAGCATGAACAATG | Cell recognition domain 7dl2: 61-441 |
| | | GCACCAACAATTTCCAGAACTTCATCGGCATCTCTAGCCTGCAGA | Linker (n = 2): 442-471 |
| | | AGACCCTGAGGAACGCCCTGATCCCTACAGAGACAACACAGCAG | Endonuclease MAD7: 472-4260 |
| | | TTCATCGTGAAGAATGGCATCATCAAGGAGGATGAGCTGCGGGG | NLS: 4261-4308 |
| | | CGAGAACAGACAGATCCTGAAGGACATCATGGACGATTACTATC | Tev-cleavage sequence: 4309-4338 |
| | | GCGGCTTCATCTCTGAGACACTGTCCTCTATCGACGATATCGACT | Endosomal escape sequence: 4339-4371 |
| | | GGACAAGCCTGTTTGAGAAGATGGAGATCCAGCTGAAGAATGGC | |
| | | GATAACAAGGACACCCTGATCAAGGAGCAGACAGAGTACAGGA | |
| | | AGGCCATCCACAAGAAGTTCGCCAATGACGATCGCTTCAAGAAC | |
| | | ATGTTTTCCGCCAAGCTGATCTCTGATATCCTGCCAGAGTTTGTG | |
| | | ATCCACAACAATAACTACTCTGCCAGCGAGAAGGAGGAGAAGAC | |
| | | CCAGGTCATCAAGCTGTTCAGCCGGTTTGCCACATCCTTCAAGGA | |
| | | CTACTTCAAGAATAGAGCCAACTGCTTCTCCGCCGACGATATCAG | |
| | | CTCCTCTAGCTGTCACCGGATCGTGAATGATAACGCCGAGATCTT | |
| | | CTTTTCTAACGCCCTGGTGTACCGGAGAATCGTGAAGTCCCTGTC | |
| | | TAATGACGATATCAACAAGATCAGCGGCGATATGAAGGACTCTC | |
| | | TGAAGGAGATGAGCCTGGAGGAGATCTATTCCTACGAGAAGTAC | |
| | | GGCGAGTTCATCACCCAGGAGGGCATCTCCTTTTATAACGACATC | |
| | | TGCGGCAAGGTCAATTCTTTCATGAACCTGTACTGTCAGAAGAAT | |
| | | AAGGAGAATAAGAACCTGTATAAGCTGCAGAAGCTGCACAAGCA | |
| | | GATCCTGTGCATCGCCGATACAAGCTACGAGGTGCCCTATAAGTT | |
| | | CGAGTCCGACGAGGAGGTGTACCAGTCTGTGAATGGCTTTCTGG | |
| | | ATAACATCTCCTCTAAGCACATCGTGGAGCGGCTGAGAAAGATC | |
| | | GGCGATAATTACAACGGCTATAACCTGGACAAGATCTATATCGT | |
| | | GTCCAAGTTTTACGAGAGCGTGTCCCAGAAGACCTACAGAGACT | |
| | | GGGAGACAATCAACACAGCCCTGGAGATCCACTATAATAACATC | |
| | | CTGCCTGGCAACGGCAAGTCCAAGGCCGATAAGGTGAAGAAGGC | |
| | | CGTGAAGAATGACCTGCAGAAGTCTATCACCGAGATCAATGAGC | |
| | | TGGTGTCTAACTACAAGCTGTGCAGCGACGATAACATCAAGGCC | |
| | | GAGACATATATCCACGAGATCAGCCACATCCTGAATAACTTCGA | |
| | | GGCCCAGGAGCTGAAGTACAATCCTGAGATCCACCTGGTGGAGT | |
| | | CCGAGCTGAAGGCCTCTGAGCTGAAGAATGTGCTGGACGTGATC | |
| | | ATGAACGCCTTCCACTGGTGTTCCGTGTTTATGACCGAGGAGCTG | |
| | | GTGGACAAGGATAATAACTTTTATGCCGAGCTGGAGGAGATCTA | |
| | | CGATGAGATCTATCCAGTGATCTCTCTGTATAATCTGGTGCGGAA | |
| | | CTACGTGACCCAGAAGCCCTATAGCACAAAGAAGATCAAGCTGA | |
| | | ACTTCGGCATCCCTACCCTGGCAGACGGATGGTCTAAGAGCAAG | |
| | | GAGTACAGCAATAACGCCATCATCCTGATGAGAGATAATCTGTA | |
| | | CTATCTGGGCATCTTTAATGCCAAGAACAAGCCAGACAAGAAGA | |
| | | TCATCGAGGGCAATACATCCGAGAACAAGGGCGATTACAAGAAG | |
| | | ATGATCTATAATCTGCTGCCCGGCCCTAACAAGATGATCCCAAAG | |
| | | GTGTTCCTGAGCTCCAAGACCGGCGTGGAGACATACAAGCCCAG | |
| | | CGCCTATATCCTGGAGGGCTACAAGCAGAACAAGCACATCAAGT | |
| | | CTAGCAAGGACTTCGATATCACCTTTTGCCACGATCTGATCGACT | |
| | | ACTTCAAGAATTGTATCGCCATCCACCCCGAGTGGAAGAACTTCG | |
| | | GCTTTGATTTCTCTGACACCAGCACATACGAGGACATCTCTGGCT | |
| | | TTTATAGGGAGGTGGAGCTGCAGGGCTACAAGATCGATTGGACA | |
| | | TATATCAGCGAGAAGGACATCGATCTGCTGCAGGAGAAGGGCCA | |
| | | GCTGTATCTGTTCCAGATCTACAACAAGGATTTTTCCAAGAAGTC | |
| | | TACCGGCAATGACAACCTGCACACAATGTACCTGAAGAATCTGTT | |
| | | CAGCGAGGAGAACCTGAAGGACATCGTGCTGAAGCTGAATGGCG | |
| | | AGGCCGAGATCTTCTTTCGCAAGTCCTCTATCAAGAATCCCATCA | |
| | | TCCACAAGAAGGGCTCCATCCTGGTGAACAGGACCTACGAGGCC | |
| | | GAGGAGAAGGACCAGTTCGGCAACATCCAGATCGTGCGCAAGAA | |
| | | TATCCCTGAGAACATCTATCAGGAGCTGTATAAGTACTTTAATGA | |
| | | TAAGAGCGACAAGGAGCTGTCCGATGAGGCCGCCAAGCTGAAGA | |
| | | ATGTGGTGGGACACCACGAGGCAGCAACCAACATCGTGAAGGAT | |
| | | TATAGGTACACATATGACAAGTACTTCCTGCACATGCCCATCACC | |
| | | ATCAATTTCAAGGCCAACAAGACAGGCTTTATCAACGACCGCAT | |
| | | CCTGCAGTACATCGCCAAGGAGAAGGATCTGCACGTGATCGGCA | |
| | | TCGACAGGGGCGAGCGCAATCTGATCTACGTGAGCGTGATCGAC | |
| | | ACCTGCGGCAACATCGTGGAGCAGAAGTCTTTTAATATCGTGAAC | |
| | | GGCTACGATTATCAGATCAAGCTGAAGCAGCAGGAGGGAGCAAG | |
| | | GCAGATCGCAAGGAAGGAGTGGAAGGAGATCGGCAAGATCAAG | |
| | | GAGATCAAGGAGGGCTACCTGAGCCTGGTCATCCACGAGATCTC | |
| | | CAAGATGGTCATCAAGTACAACGCCATCATCGCCATGGAGGACC | |
| | | TGAGCTATGGCTTCAAGAAAGGCCGGTTTAAGGTGGAGAGACAG | |
| | | GTGTACCAGAAGTTCGAGACAATGCTGATCAATAAGCTGAACTA | |
| | | TCTGGTGTTTAAGGACATCTCCATCACCGAGAACGGCGGCCTGCT | |
| | | GAAGGGCTACCAGCTGACATATATCCCTGATAAGCTGAAGAATG | |
| | | TGGGCCACCAGTGCGGCTGTATCTTCTATGTGCCAGCCGCCTACA | |
| | | CCAGCAAGATCGACCCCACCACAGGCTTTGTGAACATCTTTAAGT | |
| | | TCAAGGATCTGACAGTGGACGCCAAGCGGGAGTTCATCAAGAAG | |
| | | TTTGATTCTATCAGATACGACAGCGAGAAGAACCTGTTTTGCTTC | |
| | | ACCTTTGATTACAACAACTTCATCACCCAGAACACAGTGATGTCC | |
| | | AAGAGCTCCTGGAGCGTGTACACATATGGCGTGAGGATCAAGAG | |
| | | GCGCTTCGTGAATGGCCGCTTTAGCAACGAGTCCGATACCATCGA | |
| | | CATCACAAAGGATATGGAGAAGACCCTGGAGATGACAGACATCA | |
| | | ACTGGAGGGATGGCCACGACCTGCGCCAGGATATCATCGACTAC | |
| | | GAGATCGTGCAGCACATCTTCGAGATCTTTCGGCTGACCGTGCAG | |
| | | ATGAGAAACTCCCTGTCTGAGCTGGAGGACCGGGATTACGACAG | |
| | | ACTGATCAGCCCTGTGCTGAATGAGAATAACATCTTCTATGATTC | |
| | | CGCCAAGGCAGGCGACGCACTGCCAAAGGATGCAGACGCCAACG | |
| | | GCGCCTACTGTATCGCCCTGAAGGGCCTGTATGAGATCAAGCAG | |
| | | ATCACAGAGAATTGGAAGGAGGATGGCAAGTTTTCTCGGGACAA | |
| | | GCTGAAGATCAGCAATAAGGATTGGTTCGACTTTATCCAGAACA | |
| | | AGCGGTACCTGCCCAAGAAGAAGCGGAAGGTGGAGGACCCCA | |
| | | AGAAGAAGCGGAAAGTGGAGAATCTGTATTTCCAGGGCGGGTC | |
| | | ATCTCATCACCACCACCATCACCATCATCATCACTAA | |
| 67 | 7d-md7- | MYRMQLLSCIALSLALVTNSQVKLEESGGGSVQTGGSLRLTCAAS | IL-2secretion sequence: bold |
| | L2 (7dl2) | GRTSRSYGMGWFRQAPGKEREFVSGISWRGDSTGYADSVKGRFTIS | Cell recognition domain: double underline |
| | (protein | RDNAKNTVDLQMNSLKPEDTAIYYCAAAAGSAWYGTLYEYDYWG | Linker: italics |
| | sequence) | QGTQVTVSSALEGGGGSGGGGSMNNGTNNFQNFIGISSLQKTLRNA | Endonuclease: single underline |
| | | LIPTETTQQFIVKNGIIKEDELRGENRQILKDIMDDYYRGFISETLSSID | NLS sequence: bold |
| | | DIDWTSLFEKMEIQLKNGDNKDTLIKEQTEYRKAIHKKFANDDRFK | TEV-cleavage sequence: underlined |
| | | NMFSAKLISDILPEFVIHNNNYSASEKEEKTQVIKLFSRFATSFKDYF | Endosomal release sequence: bold |
| | | KNRANCFSADDISSSSCHRFVNDNAEIFFSNALVYRRIVKSLSNDDIN | Residue numbering: |
| | | KISGDMKDSLKEMSLEEIYSYEKYGEFITQEGISFYNDICGKVNSFMN | IL-2 secretion sequence: 1-20 |
| | | LYCQKNKENKNLYKLQKLHKQILCIADTSYEVPYKFESDEEVYQSV | Cell recognition domain 7dl2: 21-147 |
| | | NGFLDNISSKHIVERLRKIGDNYNGYNLDKIYFVSKFYESVSQKTYRD | Linker (n = 2): 148-157 |
| | | WETINTALEIHYNNILPGNGKSKADKVKKAVKNDLQKSITEINELVS | Endonuclease MAD7: 158-1420 |
| | | NYKLCSDDNIKAETYIHEISHILNNFEAQELKYNPEIHLVESELKASEL | NLS: 1421-1436 |
| | | KNVLDVIMNAFHWCSVFMTEELVDKDNNFYAELEEIYDEIYPVISLY | Tev-cleavage sequence: 1437-1443 |
| | | NLVRNYVTQKPYSTKKIKLNFGIPTLADGWSKSKEYSNNAIILMRDN | Endosomal escape sequence: 1447-1456 |
| | | LYYLGIFNAKNKPDKKIIEGNTSENKGDYKKMIYNLLPGPNKMIPKV | |
| | | FLSSKTGVETYKPSAYILEGYKQNKHIKSSKDFDITFCHDLIDYFKNC | |
| | | IAIHPEWKNFGFDFSDTSTYEDISGFYREVELQGYKIDWTYISEKDID | |
| | | LLQEKGQLYLFQIYNKDFSKKSTGNDNLHTMYLKNLFSEENLKDIV | |
| | | LKLNGEAEIFFRKSSIKNPIIHKKGSILVNRTYEAEEKDQFGNIQIVRK | |
| | | NIPENIYQELYKYFNDKSDKELSDEAAKLKNVVGHHEAATNIVKDY | |
| | | RYTYDKYFLHMPITINFKANKTGFINDRILQYIAKEKDLHVIGIDRGE | |
| | | RNLIYVSVIDTCGNIVEQKSFNIVNGYDYQIKLKQQEGARQIARKEW | |
| | | KEIGKIKEIKEGYLSLVIHEISKMVIKYNAIIAMEDLSYGFKKGRFKVE | |
| | | RQVYQKFETMLINKLNYLVFKDISITENGGLLKGYQLTYIPDKLKNV | |
| | | GHQCGCIFYVPAAYTSKIDPTTGFVNIFKFKDLTVDAKREFIKKFDSI | |
| | | RYDSEKNLFCFTFDYNNFITQNTVMSKSSWSVYTYGVRIKRRFVNG | |
| | | RFSNESDTIDITKDMEKTLEMTDINWRDGHDLRQDIIDYEIVQHIFEIF | |
| | | RLTVQMRNSLSELEDRDYDRLISPVLNENNIFYDSAKAGDALPKDA | |
| | | DANGAYCIALKGLYEIKQITENWKEDGKFSRDKLKISNKDWFDFIQN | |
| | | KRYLPKKKRKVEDPKKKRKVENLYFQGGSSHHHHHHHHHH | |
| 68 | 7d-md7- | ATGTACAGGATGCAACTCCTGTCTTGCATTGCACTAAGTCTT | IL-2 secretion sequence: bold |
| | L3 | GCACTTGTCACGAACTCTCAGGTGAAGCTGGAGGAGAGCGGAG | Cell recognition domain: double underline |
| | (7dl3) | GAGGCTCCGTGCAGACCGGAGGCTCTCTGAGGCTGACATGCGCA | Linker: italics |
| | (nucleotide | GCAAGCGGAAGGACCTCCCGCTCTTACGGAATGGGATGGTTCAG | Endonuclease: single underline |
| | sequence) | GCAGGCACCAGGCAAGGAGAGAGAGTTCGTGAGCGGCATCTCTT | NLS sequence: bold |
| | | GGCGCGGCGATTCCACCGGCTATGCCGACTCTGTGAAGGGCCGG | TEV-cleavage sequence: underlined |
| | | TTTACAATCAGCAGAGATAATGCCAAGAACACCGTGGACCTGCA | Endosomal release sequence: bold |
| | | GATGAACTCCCTGAAGCCCGAGGACACAGCCATCTACTATTGTGC | Residue numbering: |
| | | AGCAGCAGCAGGCAGCGCCTGGTACGGCACCCTGTACGAGTATG | IL-2 secretion sequence: 1-60 |
| | | ATTACTGGGGCCAGGGCACCCAGGTGACAGTGAGCTCCGCCCTG | Cell recognition domain 7dl2: 61-441 |
| | | GAGGGCGGCGGCGGCTCTGGAGGAGGAGGCAGCGGCGGAGGAGG | Linker (n = 2): 442-486 |
| | | CTCCATGAACAATGGCACCAACAATTTCCAGAACTTCATCGGCAT | Endonuclease MAD7: 487-4275 |
| | | CTCTAGCCTGCAGAAGACACTGCGGAACGCCCTGATCCCTACCG | NLS: 4276-4323 |
| | | AGACCACACAGCAGTTCATCGTGAAGAATGGCATCATCAAGGAG | TEV-cleavage sequence: 4324-4347 |
| | | GATGAGCTGAGGGGCGAGAACCGCCAGATCCTGAAGGACATCAT | Endosomal escape sequence: 4348-4386 |
| | | GGACGATTACTATAGAGGCTTCATCTCTGAGACACTGTCCTCTAT | |
| | | CGACGATATCGACTGGACCAGCCTGTTTGAGAAGATGGAGATCC | |
| | | AGCTGAAGAATGGCGATAACAAGGACACCCTGATCAAGGAGCAG | |
| | | ACAGAGTACCGGAAGGCCATCCACAAGAAGTTCGCCAATGACGA | |
| | | TAGATTCAAGAACATGTTTTCTGCCAAGCTGATCAGCGATATCCT | |
| | | GCCAGAGTTTGTGATCCACAACAATAACTACAGCGCCTCCGAGA | |
| | | AGGAGGAGAAGACACAGGTCATCAAGCTGTTCAGCAGGTTTGCC | |
| | | ACCTCTTTCAAGGACTACTTCAAGAATCGCGCCAACTGCTTCTCC | |
| | | GCCGACGATATCAGCTCCTCTAGCTGTCACAGGATCGTGAATGAT | |
| | | AACGCCGAGATCTTCTTTTCTAACGCCCTGGTGTACCGGAGAATC | |
| | | GTGAAGTCTCTGAGCAATGACGATATCAACAAGATCAGCGGCGA | |
| | | TATGAAGGACAGCCTGAAGGAGATGTCCCTGGAGGAGATCTATT | |
| | | CCTACGAGAAGTACGGCGAGTTCATCACACAGGAGGGCATCTCC | |
| | | TTTTATAACGACATCTGCGGCAAGGTCAATTCTTTTATGAACCTG | |
| | | TACTGTCAGAAGAATAAGGAGAATAAGAACCTGTATAAGCTGCA | |
| | | GAAGCTGCACAAGCAGATCCTGTGCATCGCCGATACCTCCTACG | |
| | | AGGTGCCCTATAAGTTCGAGTCTGACGAGGAGGTGTACCAGAGC | |
| | | GTGAATGGCTTTCTGGATAACATCTCCTCTAAGCACATCGTGGAG | |
| | | CGGCTGAGAAAGATCGGCGATAATTACAACGGCTATAACCTGGA | |
| | | CAAGATCTATATCGTGAGCAAGTTCTACGAGTCCGTGTCTCAGAA | |
| | | GACCTACCGGGACTGGGAGACCATCAATACAGCCCTGGAGATCC | |
| | | ACTATAATAACATCCTGCCTGGCAACGGCAAGTCCAAGGCCGAT | |
| | | AAGGTGAAGAAGGCCGTGAAGAATGACCTGCAGAAGTCTATCAC | |
| | | AGAGATCAATGAGCTGGTGAGCAACTACAAGCTGTGCTCCGACG | |
| | | ATAACATCAAGGCCGAGACCTATATCCACGAGATCTCCCACATCC | |
| | | TGAATAACTTTGAGGCCCAGGAGCTGAAGTACAATCCTGAGATC | |
| | | CACCTGGTGGAGTCTGAGCTGAAGGCCAGCGAGCTGAAGAATGT | |
| | | GCTGGACGTGATCATGAACGCCTTCCACTGGTGTAGCGTGTTTAT | |
| | | GACCGAGGAGCTGGTGGACAAGGATAATAACTTCTATGCCGAGC | |
| | | TGGAGGAGATCTACGATGAGATCTATCCAGTGATCTCTCTGTATA | |
| | | ATCTGGTGAGGAACTACGTGACCCAGAAGCCCTATAGCACAAAG | |
| | | AAGATCAAGCTGAACTTCGGCATCCCTACACTGGCCGACGGCTG | |
| | | GAGCAAGTCCAAGGAGTACTCCAATAACGCCATCATCCTGATGC | |
| | | GCGATAATCTGTACTATCTGGGCATCTTTAATGCCAAGAACAAGC | |
| | | CAGACAAGAAGATCATCGAGGGCAATACCAGCGAGAACAAGGG | |
| | | CGATTACAAGAAGATGATCTATAATCTGCTGCCCGGCCCTAACAA | |
| | | GATGATCCCAAAGGTGTTCCTGAGCTCCAAGACCGGCGTGGAGA | |
| | | CATACAAGCCCAGCGCCTATATCCTGGAGGGCTACAAGCAGAAC | |
| | | AAGCACATCAAGTCTAGCAAGGACTTCGATATCACATTTTGCCAC | |
| | | GATCTGATCGACTACTTCAAGAATTGTATCGCCATCCACCCCGAG | |
| | | TGGAAAAACTTCGGCTTTGATTTCAGCGACACCTCCACATACGAG | |
| | | GACATCTCTGGCTTTTATCGGGAGGTGGAGCTGCAGGGCTACAA | |
| | | GATCGATTGGACCTATATCAGCGAGAAGGACATCGATCTGCTGC | |
| | | AGGAGAAGGGCCAGCTGTATCTGTTCCAGATCTACAACAAGGAT | |
| | | TTTTCTAAGAAGAGCACAGGCAATGACAACCTGCACACCATGTA | |
| | | CCTGAAGAATCTGTTCTCCGAGGAGAACCTGAAGGACATCGTGC | |
| | | TGAAGCTGAATGGCGAGGCCGAGATCTTCTTTAGAAAGTCCTCTA | |
| | | TCAAGAATCCCATCATCCACAAGAAGGGCAGCATCCTGGTGAAC | |
| | | CGGACCTACGAGGCCGAGGAGAAGGACCAGTTCGGCAACATCCA | |
| | | GATCGTGAGAAAGAATATCCCTGAGAACATCTATCAGGAGCTGT | |
| | | ATAAGTACTTTAATGATAAGTCCGACAAGGAGCTGTCTGATGAG | |
| | | GCCGCCAAGCTGAAGAATGTGGTGGGCCACCACGAGGCCGCCAC | |
| | | AAACATCGTGAAGGATTATAGGTACACCTATGACAAGTACTTTCT | |
| | | GCACATGCCCATCACAATCAATTTCAAGGCCAACAAGACCGGCT | |
| | | TTATCAACGACCGCATCCTGCAGTACATCGCCAAGGAGAAGGAT | |
| | | CTGCACGTGATCGGCATCGACCGGGGCGAGAGAAATCTGATCTA | |
| | | CGTGAGCGTGATCGACACCTGTGGCAACATCGTGGAGCAGAAGT | |
| | | CTTTCAATATCGTGAACGGCTACGATTATCAGATCAAGCTGAAGC | |
| | | AGCAGGAGGGAGCAAGGCAGATCGCAAGAAAGGAGTGGAAGGA | |
| | | GATCGGCAAGATCAAGGAGATCAAGGAGGGCTACCTGAGCCTGG | |
| | | TCATCCACGAGATCTCTAAGATGGTCATCAAGTACAACGCCATCA | |
| | | TCGCCATGGAGGACCTGTCCTATGGCTTCAAGAAGGGCAGGTTTA | |
| | | AGGTGGAGCGCCAGGTGTACCAGAAGTTCGAGACCATGCTGATC | |
| | | AATAAGCTGAACTATCTGGTGTTTAAGGACATCAGCATCACAGA | |
| | | GAACGGCGGCCTGCTGAAGGGCTACCAGCTGACCTATATCCCTG | |
| | | ATAAGCTGAAGAATGTGGGCCACCAGTGCGGCTGTATCTTCTATG | |
| | | TGCCAGCCGCCTACACAAGCAAGATCGACCCCACCACAGGCTTT | |
| | | GTGAATATCTTTAAGTTCAAGGATCTGACCGTGGACGCCAAGAG | |
| | | GGAGTTCATCAAGAAGTTTGATAGCATCCGCTACGACTCCGAGA | |
| | | AGAACCTGTTTTGCTTCACATTTGATTACAACAACTTCATCACCC | |
| | | AGAATACAGTGATGTCTAAGAGCTCCTGGAGCGTGTACACCTAT | |
| | | GGCGTGCGGATCAAGAGGCGCTTCGTGAATGGCAGATTTTCCAA | |
| | | CGAGTCTGATACCATCGACATCACAAAGGATATGGAGAAGACCC | |
| | | TGGAGATGACAGACATCAACTGGCGGGATGGCCACGACCTGAGA | |
| | | CAGGATATCATCGACTACGAGATCGTGCAGCACATCTTCGAGATC | |
| | | TTTAGGCTGACAGTGCAGATGCGCAACTCTCTGAGCGAGCTGGA | |
| | | GGACAGGGATTACGACCGCCTGATCAGCCCTGTGCTGAATGAGA | |
| | | ATAACATCTTCTATGATTCCGCCAAGGCAGGCGACGCACTGCCAA | |
| | | AGGATGCAGACGCCAACGGCGCCTACTGTATCGCCCTGAAGGGC | |
| | | CTGTATGAGATCAAGCAGATCACCGAGAATTGGAAGGAGGATGG | |
| | | CAAGTTTAGCCGGGACAAGCTGAAGATCTCCAATAAGGATTGGT | |
| | | TCGACTTTATCCAGAACAAGAGGTACCTGCCCAAGAAGAAGCG | |
| | | GAAGGTGGAGGACCCCAAGAAGAAGCGGAAAGTGGAGAACC | |
| | | TGTATTTCCAGGGCGGCTCTAGCCATCATCACCATCATCACCAC | |
| | | CACCACCACTGA | |
| 69 | 7d-md7- | MYRMQLLSCIALSLALVTNSQVKLEESGGGSVQTGGSLRLTCAAS | IL-2 secretion sequence: bold |
| | L3 | GRTSRSYGMGWFRQAPGKEREFVSGISWRGDSTGYADSVKGRFTIS | Cell recognition domain: double underline |
| | (7dl3) | RDNAKNTVDLQMNSLKPEDTAIYYCAAAAGSAWYGTLYEYDYWG | Linker: italics |
| | (protein | QGTQVTVSSALEGGGGSGGGGSGGGGSMNNGTNNFQNFIGISSLQK | Endonuclease: single underline |
| | sequence) | TLRNALIPTETTQQFIVKNGIIKEDELRGENRQILKDIMDDYYRGFISE | NLS sequence: bold |
| | | TLSSIDDIDWTSLFEKMEIQLKNGDNKDTLIKEQTEYRKAIHKKFAN | TEV-cleavage sequence: underlined |
| | | DDRFKNMFSAKLISDILPEFVIHNNNYSASEKEEKTQVIKLFSRFATS | Endosomal release sequence: bold |
| | | FKDYFKNRANCFSADDISSSSCHRIVNDNAEIFFSNALVYRRIVKSLS | Residue numbering: |
| | | NDDINKISGDMKDSLKEMSLEEIYSYEKYGEFITQEGISFYNDICGKV | IL-2 secretion sequence: 1-20 |
| | | NSFMNLYCQKNKENKNLYKLQKLHKQILCIADTSYEVPYKFESDEE | Cell recognition domain 7dl2: 21-147 |
| | | VYQSVNGFLDNISSKHIVERLRKIGDNYNGYNLDKIYIVSKFYESVS | Linker (n = 2): 148-162 |
| | | QKTYRDWETINTALEIHYNNILPGNGKSKADKVKKAVKNDLQKSIT | Endonuclease MAD7: 163-1425 |
| | | EINELVSNYKLCSDDNIKAETYIHEISHILNNFEAQELKYNPEIHLVES | NLS: 1426-1441 |
| | | ELKASELKNVLDVIMNAFHWCSVFMTEELVDKDNNFYAELEEIYDE | TEV-cleavage sequence: 1442-1448 |
| | | IYPVISLYNLVRNYVTQKPYSTKKIKLNFGIPTLADGWSKSKEYSNN | Endosomal escape sequence: 1452-1461 |
| | | AIILMRDNLYYLGIFNAKNKPDKKIIEGNTSENKGDYKKMIYNLLPG | |
| | | PNKMIPKVFLSSKTGVETYKPSAYILEGYKQNKHIKSSKDFDITFCH | |
| | | DLIDYFKNCIAIHPEWKNFGFDFSDTSTYEDISGFYREVELQGYKID | |
| | | WTYISEKDIDLLQEKGQLYLFQIYNKDFSKKSTGNDNLHTMYLKNL | |
| | | FSEENLKDIVLKLNGEAEIFFRKSSIKNPIIHKKGSILVNRTYEAEEKD | |
| | | QFGNIQIVRKNIPENIYQELYKYFNDKSDKELSDEAAKLKNVVGHH | |
| | | EAATNFVKDYRYTYDKYFLHMPITINFKANKTGFINDRILQYIAKEK | |
| | | DLHVIGIDRGERNLIYVSVIDTCGNIVEQKSFNIVNGYDYQIKLKQQE | |
| | | GARQIARKEWKEIGKIKEIKEGYLSLVIHEISKMVIKYNAIIAMEDLS | |
| | | YGFKKGRFKVERQVYQKFETMLINKLNYLVFKDISITENGGLLKGY | |
| | | QLTYIPDKLKNVGHQCGCIFYVPAAYTSKIDPTTGFVNIFKFKDLTV | |
| | | DAKREFIKKFDSIRYDSEKNLFCFTFDYNNFITQNTVMSKSSWSVYT | |
| | | YGVRIKRRFVNGRFSNESDTIDITKDMEKTLEMTDINWRDGHDLRQ | |
| | | DIIDYEIVQHIFEIFRLTVQMRNSLSELEDRDYDRLISPVLNENNIFYD | |
| | | SAKAGDALPKDADANGAYCIALKGLYEIKQITENWKEDGKFSRDK | |
| | | LKISNKDWFDFIQNKRYLPKKKRKVEDPKKKRKVENLYFQGGSS | |
| | | HHHHHHHHHH | |
| 70 | 7d-md7- | ATGTACAGGATGCAACTCCTGTCTTGCATTGCACTAAGTCTT | IL-2 secretion sequence: bold |
| | L4 (7dl4) | GCACTTGTCACGAACTCTCAGGTGAAGCTGGAGGAGAGCGGAG | Cell recognition domain: double underline |
| | (nucleotide | GAGGCTCCGTGCAGACCGGAGGCAGCCTGAGGCTGACATGCGCA | Linker: italics |
| | sequence) | GCATCCGGAAGGACCTCCCGCTCTTACGGAATGGGATGGTTCAG | Endonuclease: single underline |
| | | GCAGGCACCAGGCAAGGAGAGAGAGTTCGTGAGCGGCATCTCTT | NLS sequence: bold |
| | | GGCGCGGCGATTCTACCGGCTATGCCGACAGCGTGAAGGGCCGG | TEV-cleavage sequence: underlined |
| | | TTTACAATCTCCAGAGATAATGCCAAGAACACCGTGGACCTGCA | Endosomal release sequence: bold |
| | | GATGAACTCTCTGAAGCCCGAGGACACAGCCATCTACTATTGTGC | Residue numbering (translated amino |
| | | AGCAGCAGCAGGCAGCGCCTGGTACGGCACCCTGTACGAGTATG | acids): |
| | | ATTACTGGGGCCAGGGCACCCAGGTGACAGTGAGCTCCGCCCTG | IL-2 secretion sequence: 1-60 |
| | | GAGGGCGGCGGCGGCTCTGGAGGAGGAGGCAGCGGCGGAGGAGG | Cell recognition domain 7dl2: 61-441 |
| | | CTCCGGAGGCGGCGGCTCTATGAACAATGGCACCAACAATTTCCA | Linker (n = 2): 442-501 |
| | | GAACTTCATCGGCATCTCTAGCCTGCAGAAGACACTGCGGAACG | Endonuclease MAD7: 502-4290 |
| | | CCCTGATCCCTACCGAGACCACACAGCAGTTCATCGTGAAGAAT | NLS: 4291-4338 |
| | | GGCATCATCAAGGAGGATGAGCTGAGGGGCGAGAACCGCCAGAT | Tev-cleavage sequence: 4339-4368 |
| | | CCTGAAGGACATCATGGACGATTACTATAGAGGCTTCATCAGCG | Endosomal escape sequence: 4369-4401 |
| | | AGACACTGTCCTCTATCGACGATATCGACTGGACCTCCCTGTTTG | |
| | | AGAAGATGGAGATCCAGCTGAAGAATGGCGATAACAAGGACAC | |
| | | CCTGATCAAGGAGCAGACAGAGTACCGGAAGGCCATCCACAAGA | |
| | | AGTTCGCCAATGACGATAGATTCAAGAACATGTTTAGCGCCAAG | |
| | | CTGATCTCCGATATCCTGCCAGAGTTTGTGATCCACAACAATAAC | |
| | | TACAGCGCCTCCGAGAAGGAGGAGAAGACACAGGTCATCAAGCT | |
| | | GTTCAGCAGGTTTGCCACCAGCTTCAAGGACTACTTCAAGAATCG | |
| | | CGCCAACTGCTTCTCTGCCGACGATATCAGCTCCTCTAGCTGTCA | |
| | | CAGGATCGTGAATGATAACGCCGAGATCTTCTTTTCCAACGCCCT | |
| | | GGTGTACCGGAGAATCGTGAAGTCTCTGAGCAATGACGATATCA | |
| | | ACAAGATCTCCGGCGATATGAAGGACTCCCTGAAGGAGATGTCT | |
| | | CTGGAGGAGATCTATTCTTACGAGAAGTACGGCGAGTTCATCAC | |
| | | ACAGGAGGGCATCTCTTTTTATAACGACATCTGCGGCAAGGTCAA | |
| | | TAGCTTTATGAACCTGTACTGTCAGAAGAATAAGGAGAATAAGA | |
| | | ACCTGTATAAGCTGCAGAAGCTGCACAAGCAGATCCTGTGCATC | |
| | | GCCGATACCAGCTACGAGGTGCCCTATAAGTTCGAGAGCGACGA | |
| | | GGAGGTGTACCAGTCCGTGAATGGCTTTCTGGATAACATCTCCTC | |
| | | TAAGCACATCGTGGAGCGGCTGAGAAAGATCGGCGATAATTACA | |
| | | ACGGCTATAACCTGGACAAGATCTATATCGTGTCCAAGTTCTACG | |
| | | AGTCCGTGTCTCAGAAGACCTACCGGGACTGGGAGACCATCAAT | |
| | | ACAGCCCTGGAGATCCACTATAATAACATCCTGCCTGGCAACGG | |
| | | CAAGTCTAAGGCCGATAAGGTGAAGAAGGCCGTGAAGAATGACC | |
| | | TGCAGAAGAGCATCACAGAGATCAATGAGCTGGTGTCCAACTAC | |
| | | AAGCTGTGCTCTGACGATAACATCAAGGCCGAGACCTATATCCA | |
| | | CGAGATCAGCCACATCCTGAATAACTTTGAGGCCCAGGAGCTGA | |
| | | AGTACAATCCTGAGATCCACCTGGTGGAGAGCGAGCTGAAGGCC | |
| | | TCCGAGCTGAAGAATGTGCTGGACGTGATCATGAACGCCTTCCAC | |
| | | TGGTGTTCCGTGTTTATGACCGAGGAGCTGGTGGACAAGGATAAT | |
| | | AACTTCTATGCCGAGCTGGAGGAGATCTACGATGAGATCTATCCA | |
| | | GTGATCAGCCTGTATAATCTGGTGAGGAACTACGTGACCCAGAA | |
| | | GCCCTATTCCACAAAGAAGATCAAGCTGAACTTCGGCATCCCTAC | |
| | | ACTGGCCGACGGCTGGAGCAAGTCCAAGGAGTACAGCAATAACG | |
| | | CCATCATCCTGATGCGCGATAATCTGTACTATCTGGGCATCTTTA | |
| | | ATGCCAAGAACAAGCCAGACAAGAAGATCATCGAGGGCAATACC | |
| | | TCCGAGAACAAGGGCGATTACAAGAAGATGATCTATAATCTGCT | |
| | | GCCCGGCCCTAACAAGATGATCCCAAAGGTGTTCCTGAGCTCCA | |
| | | AGACCGGCGTGGAGACATACAAGCCCAGCGCCTATATCCTGGAG | |
| | | GGCTACAAGCAGAACAAGCACATCAAGTCTAGCAAGGACTTCGA | |
| | | TATCACATTTTGCCACGATCTGATCGACTACTTCAAGAATTGTAT | |
| | | CGCCATCCACCCCGAGTGGAAAAACTTCGGCTTTGATTTCAGCGA | |
| | | CACCTCCACATACGAGGACATCAGCGGCTTTTATCGGGAGGTGG | |
| | | AGCTGCAGGGCTACAAGATCGATTGGACCTATATCTCCGAGAAG | |
| | | GACATCGATCTGCTGCAGGAGAAGGGCCAGCTGTATCTGTTCCA | |
| | | GATCTACAACAAGGATTTTTCTAAGAAGAGCACAGGCAATGACA | |
| | | ACCTGCACACCATGTACCTGAAGAATCTGTTCAGCGAGGAGAAC | |
| | | CTGAAGGACATCGTGCTGAAGCTGAATGGCGAGGCCGAGATCTT | |
| | | CTTTAGAAAGTCCTCTATCAAGAATCCCATCATCCACAAGAAGGG | |
| | | CTCCATCCTGGTGAACCGGACCTACGAGGCCGAGGAGAAGGACC | |
| | | AGTTCGGCAACATCCAGATCGTGAGAAAGAATATCCCTGAGAAC | |
| | | ATCTATCAGGAGCTGTACAAGTACTTTAATGATAAGTCTGACAAG | |
| | | GAGCTGAGCGATGAGGCCGCCAAGCTGAAGAATGTGGTGGGCCA | |
| | | CCACGAGGCCGCCACAAACATCGTGAAGGATTATAGGTACACCT | |
| | | ATGACAAGTACTTTCTGCACATGCCCATCACAATCAATTTCAAGG | |
| | | CCAACAAGACCGGCTTTATCAACGACCGCATCCTGCAGTACATCG | |
| | | CCAAGGAGAAGGATCTGCACGTGATCGGCATCGACCGGGGCGAG | |
| | | AGAAATCTGATCTACGTGAGCGTGATCGACACCTGTGGCAACAT | |
| | | CGTGGAGCAGAAGAGCTTCAATATCGTGAACGGCTACGATTATC | |
| | | AGATCAAGCTGAAGCAGCAGGAGGGAGCAAGGCAGATCGCAAG | |
| | | AAAGGAGTGGAAGGAGATCGGCAAGATCAAGGAGATCAAGGAG | |
| | | GGCTACCTGAGCCTGGTCATCCACGAGATCAGCAAGATGGTCAT | |
| | | CAAGTACAACGCCATCATCGCCATGGAGGACCTGAGCTATGGCT | |
| | | TCAAGAAGGGCAGGTTTAAGGTGGAGCGCCAGGTGTACCAGAAG | |
| | | TTCGAGACCATGCTGATCAATAAGCTGAACTATCTGGTGTTTAAG | |
| | | GACATCTCCATCACAGAGAACGGCGGCCTGCTGAAGGGCTACCA | |
| | | GCTGACCTATATCCCTGATAAGCTGAAGAATGTGGGCCACCAGT | |
| | | GCGGCTGTATCTTCTATGTGCCAGCCGCCTACACAAGCAAGATCG | |
| | | ACCCCACCACAGGCTTTGTGAATATCTTTAAGTTCAAGGATCTGA | |
| | | CCGTGGACGCCAAGAGGGAGTTCATCAAGAAGTTTGATTCCATC | |
| | | CGCTACGACTCTGAGAAGAACCTGTTTTGCTTCACATTTGATTAC | |
| | | AACAACTTCATCACCCAGAATACAGTGATGAGCAAGAGCTCCTG | |
| | | GTCCGTGTACACCTATGGCGTGCGGATCAAGAGGCGCTTCGTGA | |
| | | ATGGCAGATTTTCCAACGAGTCTGATACCATCGACATCACAAAG | |
| | | GATATGGAGAAGACCCTGGAGATGACAGACATCAACTGGCGGGA | |
| | | TGGCCACGACCTGAGACAGGATATCATCGACTACGAGATCGTGC | |
| | | AGCACATCTTCGAGATCTTTAGGCTGACAGTGCAGATGCGCAACT | |
| | | CTCTGAGCGAGCTGGAGGACAGGGATTACGACCGCCTGATCTCC | |
| | | CCTGTGCTGAATGAGAATAACATCTTCTATGATTCTGCCAAGGCA | |
| | | GGCGACGCACTGCCAAAGGATGCAGACGCCAACGGCGCCTACTG | |
| | | TATCGCCCTGAAGGGCCTGTATGAGATCAAGCAGATCACCGAGA | |
| | | ATTGGAAGGAGGATGGCAAGTTTTCCCGGGACAAGCTGAAGATC | |
| | | TCTAATAAGGATTGGTTCGACTTTATCCAGAACAAGAGGTACCTG | |
| | | CCCAAGAAGAAGCGGAAGGTGGAGGACCCCAAGAAGAAGCG | |
| | | GAAAGTGGAGAACCTGTATTTCCAGGGCGGCTCTAGCCATCATC | |
| | | ACCATCATCACCACCACCACCACTGA | |
| 71 | 7d-md7- | MYRMQLLSCIALSLALVTNSQVKLEESGGGSVQTGGSLRLTCAAS | IL-2 secretion sequence: bold |
| | L4 (7dl4) | GRTSRSYGMGWFRQAPGKEREFVSGISWRGDSTGYADSVKGRFTIS | Cell recognition domain: double underline |
| | (protein | RDNAKNTVDLQMNSLKPEDTAIYYCAAAAGSAWYGTLYEYDYWG | Linker: italics |
| | sequence) | QGTQVTVSSALEGGGGSGGGGSGGGGSGGGGSMNNGTNNFQNFIGI | Endonuclease: single underline |
| | | SSLQKTLRNALIPTETTQQFIVKNGIIKEDELRGENRQILKDIMDDYY | NLS sequence: bold |
| | | RGFISETLSSIDDIDWTSLFEKMEIQLKNGDNKDTLIKEQTEYRKAIH | TEV-cleavage sequence: underlined |
| | | KKFANDDRFKNMFSAKLISDILPEFVIHNNNYSASEKEEKTQVIKLFS | Endosomal release sequence: bold |
| | | RFATSFKDYFKNRANCFSADDISSSSCHRIVNDNAEIFFSNALVYRRI | Residue numbering: |
| | | VKSLSNDDINKISGDMKDSLKEMSLEEIYSYEKYGEFITQEGISFYND | IL-2 secretion sequence: 1-20 |
| | | ICGKVNSFMNLYCQKNKENKNLYKLQKLHKQILCIADTSYEVPYKF | Cell recognition domain 7dl2: 21-147 |
| | | ESDEEVYQSVNGFLDNISSKHFVERLRKIGDNYNGYNLDKIYIVSKFY | Linker (n = 2): 148-167 |
| | | ESVSQKTYRDWETINTALEIHYNNILPGNGKSKADKVKKAVKNDLQ | Endonuclease MAD7: 168-1430 |
| | | KSITEINELVSNYKLCSDDNIKAETYIHEISHILNNFEAQELKYNPEIH | NLS: 1431-1446 |
| | | LVESELKASELKNVLDVIMNAFHWCSVFMTEELVDKDNNFYAELEE | Tev-cleavage sequence: 1447-1453 |
| | | IYDEIYPVISLYNLVRNYVTQKPYSTKKIKLNFGIPTLADGWSKSKEY | Endosomal escape sequence: 1457-1466 |
| | | SNNAIILMRDNLYYLGIFNAKNKPDKKIIEGNTSENKGDYKKMIYNL | |
| | | LPGPNKMIPKVFLSSKTGVETYKPSAYILEGYKQNKHIKSSKDFDITF | |
| | | CHDLIDYFKNCIAIHPEWKNFGFDFSDTSTYEDISGFYREVELQGYKI | |
| | | DWTYISEKDIDLLQEKGQLYLFQIYNKDFSKKSTGNDNLHTMYLKN | |
| | | LFSEENLKDIVLKLNGEAEIFFRKSSIKNPIIHKKGSILVNRTYEAEEK | |
| | | DQFGNIQIVRKNIPENIYQELYKYFNDKSDKELSDEAAKLKNVVGHH | |
| | | EAATNFVKDYRYTYDKYFLHMPITINFKANKTGFINDRILQYIAKEK | |
| | | DLHVIGIDRGERNLIYVSVIDTCGNIVEQKSFNIVNGYDYQIKLKQQE | |
| | | GARQIARKEWKEIGKIKEIKEGYLSLVIHEISKMVIKYNAIIAMEDLS | |
| | | YGFKKGRFKVERQVYQKFETMLINKLNYLVFKDISITENGGLLKGY | |
| | | QLTYIPDKLKNVGHQCGCIFYVPAAYTSKIDPTTGFVNIFKFKDLTV | |
| | | DAKREFIKKFDSIRYDSEKNLFCFTFDYNNFITQNTVMSKSSWSVYT | |
| | | YGVRIKRRFVNGRFSNESDTIDITKDMEKTLEMTDINWRDGHDLRQ | |
| | | DIIDYEFVQHIFEIFRLTVQMRNSLSELEDRDYDRLISPVLNENNIFYD | |
| | | SAKAGDALPKDADANGAYCLALKGLYEIKQITENWKEDGKFSRDKL | |
| | | KISNKDWFDFIQNKRYLPKKKRKVEDPKKKRKVENLYFQGGSSH | |
| | | HHHHHHHHH | |
| 72 | Md7-7d- | ATGTACAGGATGCAACTCCTGTCTTGCATTGCACTAAGTCTT | IL-2 secretion sequence: bold |
| | L2 (MDL2) | GCACTTGTCACGAACTCTATGAACAATGGCACCAACAATTTCCA | Endosomal release sequence: bold |
| | (nucleotide | GAACTTCATCGGCATCAGCTCCCTGCAGAAGACACTGCGGAACG | Linker: italics |
| | sequence) | CCCTGATCCCTACCGAGACCACACAGCAGTTCATCGTGAAGAAT | Cell recognition domain: double underline |
| | | GGCATCATCAAGGAGGATGAGCTGAGGGGCGAGAACCGCCAGAT | NLS sequence: bold |
| | | CCTGAAGGACATCATGGACGATTACTATAGAGGCTTCATCTCCGA | TEV-cleavage sequence: underlined |
| | | GACACTGTCTAGCATCGACGATATCGACTGGACCTCTCTGTTTGA | Endonuclease: single underline |
| | | GAAGATGGAGATCCAGCTGAAGAATGGCGATAACAAGGACACCC | Residue numbers: |
| | | TGATCAAGGAGCAGACAGAGTACCGGAAGGCCATCCACAAGAA | IL-2 secretion sequence: 1-60 |
| | | GTTCGCCAATGACGATAGATTCAAGAACATGTTTTCTGCCAAGCT | Endonuclease MAD7: 61-3849 |
| | | GATCAGCGATATCCTGCCAGAGTTTGTGATCCACAACAATAACTA | Linker: 3850-3879 |
| | | CTCCGCCTCTGAGAAGGAGGAGAAGACACAGGTCATCAAGCTGT | Cell recognition domain 7dl2: 3880-4260 |
| | | TCAGCAGGTTTGCCACCTCTTTCAAGGACTACTTCAAGAATCGCG | NLS: 4261-4308 |
| | | CCAACTGCTTCAGCGCCGACGATATCTCCTCTAGCTCCTGTCACA | Tev-cleavage sequence: 4309-4338 |
| | | GGATCGTGAATGATAACGCCGAGATCTTCTTTTCCAACGCCCTGG | Endosomal escape sequence: 4339-4371 |
| | | TGTACCGGAGAATCGTGAAGAGCCTGTCCAATGACGATATCAAC | |
| | | AAGATCTCTGGCGATATGAAGGACAGCCTGAAGGAGATGTCCCT | |
| | | GGAGGAGATCTACAGCTATGAGAAGTACGGCGAGTTCATCACAC | |
| | | AGGAGGGCATCAGCTTTTATAACGACATCTGCGGCAAGGTCAAT | |
| | | TCCTTCATGAACCTGTACTGTCAGAAGAATAAGGAGAATAAGAA | |
| | | CCTGTATAAGCTGCAGAAGCTGCACAAGCAGATCCTGTGCATCG | |
| | | CCGATACCAGCTACGAGGTGCCCTATAAGTTCGAGTCCGACGAG | |
| | | GAGGTGTACCAGTCTGTGAATGGCTTTCTGGATAACATCTCTAGC | |
| | | AAGCACATCGTGGAGCGGCTGAGAAAGATCGGCGATAATTACAA | |
| | | CGGCTATAACCTGGACAAGATCTATATCGTGTCCAAGTTTTACGA | |
| | | GTCTGTGAGCCAGAAGACCTACCGGGACTGGGAGACCATCAATA | |
| | | CAGCCCTGGAGATCCACTATAATAACATCCTGCCTGGCAACGGC | |
| | | AAGAGCAAGGCCGATAAGGTGAAGAAGGCCGTGAAGAATGACC | |
| | | TGCAGAAGTCCATCACAGAGATCAATGAGCTGGTGAGCAACTAC | |
| | | AAGCTGTGCTCCGACGATAACATCAAGGCCGAGACCTATATCCA | |
| | | CGAGATCAGCCACATCCTGAATAACTTCGAGGCCCAGGAGCTGA | |
| | | AGTACAATCCTGAGATCCACCTGGTGGAGTCTGAGCTGAAGGCC | |
| | | AGCGAGCTGAAGAATGTGCTGGACGTGATCATGAACGCCTTCCA | |
| | | CTGGTGTTCCGTGTTTATGACCGAGGAGCTGGTGGACAAGGATA | |
| | | ATAACTTTTATGCCGAGCTGGAGGAGATCTACGATGAGATCTATC | |
| | | CAGTGATCTCCCTGTATAATCTGGTGAGGAACTACGTGACCCAGA | |
| | | AGCCCTATTCTACAAAGAAGATCAAGCTGAACTTCGGCATCCCTA | |
| | | CACTGGCCGACGGCTGGTCCAAGTCTAAGGAGTACAGCAATAAC | |
| | | GCCATCATCCTGATGCGCGATAATCTGTACTATCTGGGCATCTTT | |
| | | AATGCCAAGAACAAGCCAGACAAGAAGATCATCGAGGGCAATA | |
| | | CCTCCGAGAACAAGGGCGATTACAAGAAGATGATCTATAATCTG | |
| | | CTGCCCGGCCCTAACAAGATGATCCCAAAGGTGTTCCTGTCCTCT | |
| | | AAGACCGGCGTGGAGACATACAAGCCCAGCGCCTATATCCTGGA | |
| | | GGGCTACAAGCAGAACAAGCACATCAAGAGCTCCAAGGACTTCG | |
| | | ATATCACATTTTGCCACGATCTGATCGACTACTTCAAGAATTGTA | |
| | | TCGCCATCCACCCCGAGTGGAAAAACTTCGGCTTTGATTTCTCCG | |
| | | ACACCTCTACATACGAGGACATCTCCGGCTTTTATCGGGAGGTGG | |
| | | AGCTGCAGGGCTACAAGATCGATTGGACCTATATCTCTGAGAAG | |
| | | GACATCGATCTGCTGCAGGAGAAGGGCCAGCTGTATCTGTTCCA | |
| | | GATCTACAACAAGGACTTCAGCAAGAAGAGCACCGGCAATGACA | |
| | | ACCTGCACACAATGTACCTGAAGAATCTGTTCAGCGAGGAGAAC | |
| | | CTGAAGGACATCGTGCTGAAGCTGAATGGCGAGGCCGAGATCTT | |
| | | CTTTAGAAAGTCTAGCATCAAGAATCCCATCATCCACAAGAAGG | |
| | | GCTCCATCCTGGTGAACCGGACCTACGAGGCCGAGGAGAAGGAC | |
| | | CAGTTCGGCAACATCCAGATCGTGAGAAAGAATATCCCTGAGAA | |
| | | CATCTATCAGGAGCTGTACAAGTACTTCAACGATAAATCCGACA | |
| | | AGGAGCTGTCTGATGAGGCCGCCAAGCTGAAGAATGTGGTGGGC | |
| | | CACCACGAGGCCGCCACAAACATCGTGAAGGATTACCGGTATAC | |
| | | CTACGATAAGTACTTCCTGCACATGCCCATCACAATCAATTTCAA | |
| | | GGCCAACAAGACCGGCTTTATCAACGACAGAATCCTGCAGTACA | |
| | | TCGCCAAGGAGAAGGATCTGCACGTGATCGGCATCGACAGGGGC | |
| | | GAGCGCAATCTGATCTATGTGAGCGTGATCGACACCTGTGGCAA | |
| | | CATCGTGGAGCAGAAGTCCTTTAATATCGTGAACGGCTATGATTA | |
| | | CCAGATCAAGCTGAAGCAGCAGGAGGGAGCAAGGCAGATCGCA | |
| | | AGAAAGGAGTGGAAGGAGATCGGCAAGATCAAGGAGATCAAGG | |
| | | AGGGCTACCTGAGCCTGGTCATCCACGAGATCTCCAAGATGGTC | |
| | | ATCAAGTACAACGCCATCATCGCCATGGAGGACCTGAGCTATGG | |
| | | CTTCAAGAAGGGCCGGTTTAAGGTGGAGAGACAGGTGTACCAGA | |
| | | AGTTCGAGACCATGCTGATCAATAAGCTGAACTATCTGGTGTTTA | |
| | | AGGACATCTCCATCACAGAGAACGGCGGCCTGCTGAAGGGCTAC | |
| | | CAGCTGACCTATATCCCTGATAAGCTGAAGAATGTGGGCCACCA | |
| | | GTGCGGCTGTATCTTCTATGTGCCAGCCGCCTACACAAGCAAGAT | |
| | | CGACCCCACCACAGGCTTTGTGAACATCTTTAAGTTCAAGGATCT | |
| | | GACCGTGGACGCCAAGAGGGAGTTCATCAAGAAGTTTGATAGCA | |
| | | TCCGCTACGACTCCGAGAAGAACCTGTTTTGCTTCACATTTGATT | |
| | | ACAACAACTTCATCACCCAGAATACAGTGATGTCTAAGTCCTCTT | |
| | | GGAGCGTGTATACCTACGGCGTGAGGATCAAGAGGCGCTTCGTG | |
| | | AATGGCCGCTTTTCTAACGAGAGCGATACCATCGACATCACAAA | |
| | | GGATATGGAGAAGACCCTGGAGATGACAGACATCAACTGGCGGG | |
| | | ATGGCCACGACCTGAGACAGGATATCATCGACTACGAGATCGTG | |
| | | CAGCACATCTTCGAGATCTTTAGGCTGACAGTGCAGATGCGCAAC | |
| | | AGCCTGTCCGAGCTGGAGGACAGGGATTACGACCGCCTGATCTC | |
| | | TCCTGTGCTGAATGAGAATAACATCTTCTATGATAGCGCCAAGGC | |
| | | AGGCGACGCACTGCCAAAGGATGCAGACGCCAACGGCGCCTACT | |
| | | GTATCGCCCTGAAGGGCCTGTATGAGATCAAGCAGATCACCGAG | |
| | | AATTGGAAGGAGGATGGCAAGTTTTCTAGGGACAAGCTGAAGAT | |
| | | CAGCAATAAGGATTGGTTCGACTTTATCCAGAACAAGCGGTACCT | |
| | | GGGAGGAGGAGGCTCCGGCGGAGGAGGCTCTCAGGAGAAGCAGG | |
| | | AGGAGAGCGGAGGAGGCTCCGTGCAGACCGGAGGCTCCCTGAGG | |
| | | CTGACATGCGCAGCATCTGGACGGACCTCTAGAAGCTACGGAAT | |
| | | GGGATGGTTCAGGCAGGCACCAGGCAAGGAGAGAGAGTTCGTGA | |
| | | GCGGCATCTCTTGGCGCGGCGATTCTACCGGCTATGCCGACAGCG | |
| | | TGAAGGGCAGGTTCACAATCTCTCGCGATAATGCCAAGAACACC | |
| | | GTGGACCTGCAGATGAACAGCCTGAAGCCCGAGGACACAGCCAT | |
| | | CTACTATTGTGCAGCAGCAGCAGGCAGCGCCTGGTACGGCACCC | |
| | | TGTATGAGTACGATTATTGGGGCCAGGGCACCCAGGTGACAGTG | |
| | | AGCTCCGCCCTGGAGCCCAAGAAGAAGCGGAAGGTGGAGGAC | |
| | | CCCAAGAAGAAGCGGAAAGTGGAGAATCTGTATTTTCAGGGCG | |
| | | GCTCTAGCCATCATCACCATCATCACCACCACCACCACTGA | |
| 73 | Md7-7d- | MYRMQLLSCIALSLALVTNSMNNGTNNFQNFIGISSLQKTLRNALI | IL-2 secretion sequence: bold |
| | L2 (MDl2) | PTETTQQFIVKNGIIKEDELRGENRQILKDIMDDYYRGFISETLSSIDDI | Endonuclease: single underline |
| | (protein | DWTSLFEKMEIQLKNGDNKDTLIKEQTEYRKAIHKKFANDDRFKNM | Linker: italics |
| | sequence) | FSAKLISDILPEFVIHNNNYSASEKEEKTQVIKLFSRFATSFKDYFKNR | Cell recognition domain: double underline |
| | | ANCFSADDISSSSCHRIVNDNAEIFFSNALVYRRIVKSLSNDDINKISG | NLS sequence: bold |
| | | DMKDSLKEMSLEEIYSYEKYGEFITQEGISFYNDICGKVNSFMNLYC | TEV-cleavage sequence: underlined |
| | | QKNKENKNLYKLQKLHKQILCIADTSYEVPYKFESDEEVYQSVNGF | Endosomal release sequence: bold |
| | | LDNISSKHFVERLRKIGDNYNGYNLDKIYIVSKFYESVSQKTYRDWE | Residue numbers: |
| | | TINTALEIHYNNILPGNGKSKADKVKKAVKNDLQKSITEINELVSNY | IL-2 secretion sequence: 1-20 |
| | | KLCSDDNIKAETYIHEISHILNNFEAQELKYNPEIHLVESELKASELKN | Endonuclease MAD7: 21-1283 |
| | | VLDVIMNAFHWCSVFMTEELVDKDNNFYAELEEIYDEIYPVISLYNL | Linker: 1284-1293 |
| | | VRNYVTQKPYSTKKIKLNFGIPTLADGWSKSKEYSNNAIILMRDNLY | Cell recognition domain 7dl2: 1294-1293 |
| | | YLGIFNAKNKPDKKIIEGNTSENKGDYKKMIYNLLPGPNKMIPKVFL | NLS: 1421-1436 |
| | | SSKTGVETYKPSAYILEGYKQNKHIKSSKDFDITFCHDLIDYFKNCIAI | Tev-cleavage sequence: 1437-1443 |
| | | HPEWKNFGFDFSDTSTYEDISGFYREVELQGYKIDWTYISEKDIDLL | Endosomal escape sequence: 1447-1456 |
| | | QEKGQLYLFQIYNKDFSKKSTGNDNLHTMYLKNLFSEENLKDIVLK | |
| | | LNGEAEIFFRKSSIKNPIIHKKGSILVNRTYEAEEKDQFGNIQIVRKNIP | |
| | | ENIYQELYKYFNDKSDKELSDEAAKLKNVVGHHEAATNIVKDYRY | |
| | | TYDKYFLHMPITINFKANKTGFINDRILQYIAKEKDLHVIGIDRGERN | |
| | | LIYVSVIDTCGNIVEQKSFNIVNGYDYQIKLKQQEGARQIARKEWKEI | |
| | | GKIKEIKEGYLSLVIHEISKMVIKYNAIIAMEDLSYGFKKGRFKVERQ | |
| | | VYQKFETMLINKLNYLVFKDISITENGGLLKGYQLTYIPDKLKNVGH | |
| | | QCGCIFYVPAAYTSKIDPTTGFVNIFKFKDLTVDAKREFIKKFDSIRY | |
| | | DSEKNLFCFTFDYNNFITQNTVMSKSSWSVYTYGVRIKRRFVNGRFS | |
| | | NESDTIDITKDMEKTLEMTDINWRDGHDLRQDIIDYEIVQHIFEIFRLT | |
| | | VQMRNSLSELEDRDYDRLISPVLNENNIFYDSAKAGDALPKDADAN | |
| | | GAYCIALKGLYEIKQITENWKEDGKFSRDKLKISNKDWFDFIQNKRY | |
| | | LGGGGSGGGGSQVKLEESGGGSVQTGGSLRLTCAASGRTSRSYGMG | |
| | | WFRQAPGKEREFVSGISWRGDSTGYADSVKGRFTISRDNAKNTVDL | |
| | | QMNSLKPEDTAIYYCAAAAGSAWYGTLYEYDYWGQGTQVTVSSA | |
| | | LEPKKKRKVEDPKKKRKVENLYFQGGSSHHHHHHHHHH | |
| 74 | md7-7d- | ATGTACAGGATGCAACTCCTGTCTTGCATTGCACTAAGTCTT | IL-2 secretion sequence: bold |
| | L3 (mdl3) | GCACTTGTCACGAACTCTATGAACAATGGCACCAACAATTTCCA | Endonuclease: single underline |
| | (nucleotide | GAACTTCATCGGCATCAGCTCCCTGCAGAAGACACTGCGGAACG | Linker: italics |
| | sequence) | CCCTGATCCCTACCGAGACCACACAGCAGTTCATCGTGAAGAAT | Cell recognition domain: double underline |
| | | GGCATCATCAAGGAGGATGAGCTGAGGGGCGAGAACCGCCAGAT | NLS sequence: bold |
| | | CCTGAAGGACATCATGGACGATTACTATAGAGGCTTCATCTCTGA | TEV-cleavage sequence: underlined |
| | | GACACTGTCTAGCATCGACGATATCGACTGGACCAGCCTGTTTGA | Endosomal release sequence: bold |
| | | GAAGATGGAGATCCAGCTGAAGAATGGCGATAACAAGGACACCC | Residue numbering (translated amino |
| | | TGATCAAGGAGCAGACAGAGTACCGGAAGGCCATCCACAAGAA | acids): |
| | | GTTCGCCAATGACGATAGATTCAAGAACATGTTTTCTGCCAAGCT | IL-2 secretion sequence: 1-60 |
| | | GATCAGCGATATCCTGCCAGAGTTTGTGATCCACAACAATAACTA | Endonuclease MAD7: 61-3849 |
| | | CTCCGCCTCTGAGAAGGAGGAGAAGACACAGGTCATCAAGCTGT | Linker: 3850-3894 |
| | | TCAGCAGGTTTGCCACCTCTTTCAAGGACTACTTCAAGAATCGCG | Cell recognition domain 7dl2: 3895-4275 |
| | | CCAACTGCTTCTCCGCCGACGATATCTCCTCTAGCTCCTGTCACA | NLS: 4276-4323 |
| | | GGATCGTGAATGATAACGCCGAGATCTTCTTTTCTAACGCCCTGG | Tev-cleavage sequence: 4324-4353 |
| | | TGTACCGGAGAATCGTGAAGAGCCTGTCCAATGACGATATCAAC | Endosomal escape sequence: 4354-4386 |
| | | AAGATCAGCGGCGATATGAAGGACAGCCTGAAGGAGATGTCCCT | |
| | | GGAGGAGATCTACTCCTATGAGAAGTACGGCGAGTTCATCACAC | |
| | | AGGAGGGCATCTCCTTTTATAACGACATCTGCGGCAAGGTCAATT | |
| | | CTTTCATGAACCTGTACTGTCAGAAGAATAAGGAGAATAAGAAC | |
| | | CTGTATAAGCTGCAGAAGCTGCACAAGCAGATCCTGTGCATCGC | |
| | | CGATACCTCCTACGAGGTGCCCTATAAGTTCGAGTCTGACGAGGA | |
| | | GGTGTACCAGAGCGTGAATGGCTTTCTGGATAACATCTCTAGCAA | |
| | | GCACATCGTGGAGCGGCTGAGAAAGATCGGCGATAATTACAACG | |
| | | GCTATAACCTGGACAAGATCTATATCGTGAGCAAGTTTTACGAGT | |
| | | CTGTGAGCCAGAAGACCTACCGGGACTGGGAGACCATCAATACA | |
| | | GCCCTGGAGATCCACTATAATAACATCCTGCCTGGCAACGGCAA | |
| | | GTCCAAGGCCGATAAGGTGAAGAAGGCCGTGAAGAATGACCTGC | |
| | | AGAAGTCTATCACAGAGATCAATGAGCTGGTGTCCAACTACAAG | |
| | | CTGTGCTCTGACGATAACATCAAGGCCGAGACCTATATCCACGA | |
| | | GATCTCCCACATCCTGAATAACTTCGAGGCCCAGGAGCTGAAGT | |
| | | ACAATCCTGAGATCCACCTGGTGGAGTCTGAGCTGAAGGCCAGC | |
| | | GAGCTGAAGAATGTGCTGGACGTGATCATGAACGCCTTCCACTG | |
| | | GTGTAGCGTGTTTATGACCGAGGAGCTGGTGGACAAGGATAATA | |
| | | ACTTTTATGCCGAGCTGGAGGAGATCTACGATGAGATCTATCCAG | |
| | | TGATCTCTCTGTATAATCTGGTGAGGAACTACGTGACCCAGAAGC | |
| | | CCTATAGCACAAAGAAGATCAAGCTGAACTTCGGCATCCCTACA | |
| | | CTGGCCGACGGCTGGTCCAAGTCTAAGGAGTACTCCAATAACGC | |
| | | CATCATCCTGATGCGCGATAATCTGTACTATCTGGGCATCTTTAA | |
| | | TGCCAAGAACAAGCCAGACAAGAAGATCATCGAGGGCAATACCA | |
| | | GCGAGAACAAGGGCGATTACAAGAAGATGATCTATAATCTGCTG | |
| | | CCCGGCCCTAACAAGATGATCCCAAAGGTGTTCCTGTCCTCTAAG | |
| | | ACCGGCGTGGAGACATACAAGCCCAGCGCCTATATCCTGGAGGG | |
| | | CTACAAGCAGAACAAGCACATCAAGAGCTCCAAGGACTTCGATA | |
| | | TCACATTTTGCCACGATCTGATCGACTACTTCAAGAATTGTATCG | |
| | | CCATCCACCCCGAGTGGAAGAACTTCGGCTTTGATTTCTCCGACA | |
| | | CCTCTACATACGAGGACATCTCTGGCTTTTATCGGGAGGTGGAGC | |
| | | TGCAGGGCTACAAGATCGATTGGACCTATATCAGCGAGAAGGAC | |
| | | ATCGATCTGCTGCAGGAGAAGGGCCAGCTGTATCTGTTCCAGATC | |
| | | TACAACAAGGACTTCAGCAAGAAGAGCACCGGCAATGACAACCT | |
| | | GCACACAATGTACCTGAAGAATCTGTTCTCCGAGGAGAACCTGA | |
| | | AGGACATCGTGCTGAAGCTGAATGGCGAGGCCGAGATCTTCTTT | |
| | | AGAAAGTCTAGCATCAAGAATCCCATCATCCACAAGAAGGGCAG | |
| | | CATCCTGGTGAACCGGACCTACGAGGCCGAGGAGAAGGACCAGT | |
| | | TCGGCAACATCCAGATCGTGAGAAAGAATATCCCTGAGAACATC | |
| | | TATCAGGAGCTGTACAAGTACTTCAACGATAAGTCCGACAAGGA | |
| | | GCTGTCTGATGAGGCCGCCAAGCTGAAGAATGTGGTGGGCCACC | |
| | | ACGAGGCCGCCACAAACATCGTGAAGGATTACCGGTATACCTAC | |
| | | GACAAGTACTTCCTGCACATGCCCATCACAATCAATTTCAAGGCC | |
| | | AACAAGACCGGCTTTATCAACGACAGAATCCTGCAGTACATCGC | |
| | | CAAGGAGAAGGATCTGCACGTGATCGGCATCGACAGGGGCGAGC | |
| | | GCAATCTGATCTACGTGAGCGTGATCGACACCTGTGGCAACATCG | |
| | | TGGAGCAGAAGTCTTTTAATATCGTGAACGGCTATGATTACCAGA | |
| | | TCAAGCTGAAGCAGCAGGAGGGAGCAAGGCAGATCGCAAGAAA | |
| | | GGAGTGGAAGGAGATCGGCAAGATCAAGGAGATCAAGGAGGGC | |
| | | TACCTGAGCCTGGTCATCCACGAGATCTCTAAGATGGTCATCAAG | |
| | | TACAACGCCATCATCGCCATGGAGGACCTGTCCTATGGCTTCAAG | |
| | | AAAGGCCGGTTTAAGGTGGAGAGACAGGTGTACCAGAAGTTCGA | |
| | | GACCATGCTGATCAATAAGCTGAACTATCTGGTGTTTAAGGACAT | |
| | | CAGCATCACAGAGAACGGCGGCCTGCTGAAGGGCTACCAGCTGA | |
| | | CCTATATCCCTGATAAGCTGAAGAATGTGGGCCACCAGTGCGGCT | |
| | | GTATCTTCTATGTGCCAGCCGCCTACACAAGCAAGATCGACCCCA | |
| | | CCACAGGCTTTGTGAACATCTTTAAGTTCAAGGATCTGACCGTGG | |
| | | ACGCCAAGAGGGAGTTCATCAAGAAGTTTGATAGCATCCGCTAC | |
| | | GACTCCGAGAAGAACCTGTTTTGCTTCACATTTGATTACAACAAC | |
| | | TTCATCACCCAGAATACAGTGATGTCTAAGTCCTCTTGGAGCGTG | |
| | | TATACCTACGGCGTGAGGATCAAGAGGCGCTTCGTGAATGGCCG | |
| | | CTTTTCTAACGAGAGCGATACCATCGACATCACAAAGGATATGG | |
| | | AGAAGACCCTGGAGATGACAGACATCAACTGGCGGGATGGCCAC | |
| | | GACCTGAGACAGGATATCATCGACTACGAGATCGTGCAGCACAT | |
| | | CTTCGAGATCTTTAGGCTGACAGTGCAGATGCGCAACAGCCTGTC | |
| | | CGAGCTGGAGGACAGGGATTACGACCGCCTGATCAGCCCTGTGC | |
| | | TGAATGAGAATAACATCTTCTATGATTCCGCCAAGGCAGGCGAC | |
| | | GCACTGCCAAAGGATGCAGACGCCAACGGCGCCTACTGTATCGC | |
| | | CCTGAAGGGCCTGTATGAGATCAAGCAGATCACCGAGAATTGGA | |
| | | AGGAGGATGGCAAGTTTAGCAGGGACAAGCTGAAGATCTCCAAT | |
| | | AAGGATTGGTTCGACTTTATCCAGAACAAGCGGTACCTGGGAGGA | |
| | | GGAGGCTCCGGCGGAGGAGGCTCTGGCGGCGGCGGCAGCCAGGT | |
| | | GAAGCTGGAGGAGAGCGGAGGAGGCTCCGTGCAGACCGGAGGC | |
| | | TCTCTGAGGCTGACATGCGCAGCAAGCGGACGGACCTCTAGAAG | |
| | | CTACGGAATGGGATGGTTCAGGCAGGCACCAGGCAAGGAGAGA | |
| | | GAGTTCGTGAGCGGCATCTCTTGGCGCGGCGATAGCACCGGCTAT | |
| | | GCCGACTCCGTGAAGGGCAGGTTCACAATCAGCCGCGATAATGC | |
| | | CAAGAACACCGTGGACCTGCAGATGAACTCCCTGAAGCCCGAGG | |
| | | ACACAGCCATCTACTATTGTGCAGCAGCAGCAGGCAGCGCCTGG | |
| | | TACGGCACCCTGTATGAGTACGATTATTGGGGCCAGGGCACCCA | |
| | | GGTGACAGTGAGCTCCGCCCTGGAGCCCAAGAAGAAGCGGAAG | |
| | | GTGGAGGACCCCAAGAAGAAGCGGAAAGTGGAGAATCTGTAT | |
| | | TTTCAGGGCGGCTCTAGCCATCATCACCATCATCACCACCACCA | |
| | | CCACTGA | |
| 75 | md7-7d- | MYRMQLLSCIALSLALVTNSMNNGTNNFQNFIGISSLQKTLRNALI | IL-2 secretion sequence: bold |
| | L3 (mdl3) | PTETTQQFIVKNGIIKEDELRGENRQILKDIMDDYYRGFISETLSSIDDI | Endonuclease: single underline |
| | (protein | DWTSLFEKMEIQLKNGDNKDTLIKEQTEYRKAIHKKFANDDRFKNM | Linker: italics |
| | sequence) | FSAKLISDILPEFVIHNNNYSASEKEEKTQVIKLFSRFATSFKDYFKNR | Cell recognition domain: double underline |
| | | ANCFSADDISSSSCHRIVNDNAEIFFSNALVYRRIVKSLSNDDINKISG | NLS sequence: bold |
| | | DMKDSLKEMSLEEIYSYEKYGEFITQEGISFYNDICGKVNSFMNLYC | TEV-cleavage sequence: underlined |
| | | QKNKENKNLYKLQKLHKQILCIADTSYEVPYKFESDEEVYQSVNGF | Endosomal release sequence: bold |
| | | LDNISSKHFVERLRKIGDNYNGYNLDKIYIVSKFYESVSQKTYRDWE | Residue numbering: |
| | | TINTALEIHYNNILPGNGKSKADKVKKAVKNDLQKSITEINELVSNY | IL-2 secretion sequence: 1-20 |
| | | KLCSDDNIKAETYIHEISHILNNFEAQELKYNPEIHLVESELKASELKN | Endonuclease MAD7: 21-1283 |
| | | VLDVIMNAFHWCSVFMTEELVDKDNNFYAELEEIYDEIYPVISLYNL | Linker: 1284- 1298 |
| | | VRNYVTQKPYSTKKIKLNFGIPTLADGWSKSKEYSNNAIILMRDNLY | Cell recognition domain 7dl2: 1299-1425 |
| | | YLGIFNAKNKPDKKIIEGNTSENKGDYKKMIYNLLPGPNKMIPKVFL | NLS: 1426- 1441 |
| | | SSKTGVETYKPSAYILEGYKQNKHIKSSKDFDITFCHDLIDYFKNCIAI | Tev-cleavage sequence: 1442-1448 |
| | | HPEWKNFGFDFSDTSTYEDISGFYREVELQGYKIDWTYISEKDIDLL | Endosomal escape sequence: 1452-1461 |
| | | QEKGQLYLFQIYNKDFSKKSTGNDNLHTMYLKNLFSEENLKDIVLK | |
| | | LNGEAEIFFRKSSIKNPIIHKKGSILVNRTYEAEEKDQFGNIQIVRKNIP | |
| | | ENIYQELYKYFNDKSDKELSDEAAKLKNVVGHHEAATNIVKDYRY | |
| | | TYDKYFLHMPITINFKANKTGFINDRILQYIAKEKDLHVIGIDRGERN | |
| | | LIYVSVIDTCGNIVEQKSFNIVNGYDYQIKLKQQEGARQIARKEWKEI | |
| | | GKIKEIKEGYLSLVIHEISKMVIKYNAIIAMEDLSYGFKKGRFKVERQ | |
| | | VYQKFETMLINKLNYLVFKDISITENGGLLKGYQLTYIPDKLKNVGH | |
| | | QCGCIFYVPAAYTSKIDPTTGFVNIFKFKDLTVDAKREFIKKFDSIRY | |
| | | DSEKNLFCFTFDYNNFITQNTVMSKSSWSVYTYGVRIKRRFVNGRFS | |
| | | NESDTIDITKDMEKTLEMTDINWRDGHDLRQDIIDYEIVQHIFEIFRLT | |
| | | VQMRNSLSELEDRDYDRLISPVLNENNIFYDSAKAGDALPKDADAN | |
| | | GAYCIALKGLYEIKQITENWKEDGKFSRDKLKISNKDWFDFIQNKRY | |
| | | LGGGGSGGGGSGGGGSQVKLEESGGGSVQTGGSLRLTCAASGRTSR | |
| | | SYGMGWFRQAPGKEREFVSGISWRGDSTGYADSVKGRFTISRDNAK | |
| | | NTVDLQMNSLKPEDTAIYYCAAAAGSAWYGTLYEYDYWGQGTQV | |
| | | TVSSALEPKKKRKVEDPKKKRKVENLYFQGGSSHHHHHHHHHH | |
| 76 | md7-7d- | ATGTACAGGATGCAACTCCTGTCTTGCATTGCACTAAGTCTT | IL-2 secretion sequence: bold |
| | L4 (Mdl4) | GCACTTGTCACGAACTCTATGAACAATGGCACCAACAATTTCCA | Endonuclease: single underline |
| | (nucleotide | GAACTTCATCGGCATCAGCTCCCTGCAGAAGACACTGCGGAACG | Linker: italics |
| | sequence) | CCCTGATCCCTACCGAGACCACACAGCAGTTCATCGTGAAGAAT | Cell recognition domain: double underline |
| | | GGCATCATCAAGGAGGATGAGCTGAGGGGCGAGAACCGCCAGAT | NLS sequence: bold |
| | | CCTGAAGGACATCATGGACGATTACTATAGAGGCTTCATCAGCG | TEV-cleavage sequence: underlined |
| | | AGACACTGTCTAGCATCGACGATATCGACTGGACCTCCCTGTTTG | Endosomal release sequence: bold |
| | | AGAAGATGGAGATCCAGCTGAAGAATGGCGATAACAAGGACAC | Residue numbering (translated amino |
| | | CCTGATCAAGGAGCAGACAGAGTACCGGAAGGCCATCCACAAGA | acids): |
| | | AGTTCGCCAATGACGATAGATTCAAGAACATGTTTTCTGCCAAGC | IL-2 secretion sequence: 1-60 |
| | | TGATCAGCGATATCCTGCCAGAGTTTGTGATCCACAACAATAACT | Endonuclease MAD7: 61-3849 |
| | | ACTCCGCCTCTGAGAAGGAGGAGAAGACACAGGTCATCAAGCTG | Linker: 3850:-3909 |
| | | TTCAGCAGGTTTGCCACCTCTTTCAAGGACTACTTCAAGAATCGC | Cell recognition domain 7dl2: 3910-4316 |
| | | GCCAACTGCTTCTCTGCCGACGATATCTCCTCTAGCTCCTGTCAC | NLS: 4317-4338 |
| | | AGGATCGTGAATGATAACGCCGAGATCTTCTTTTCCAACGCCCTG | Tev-cleavage sequence: 4339-4368 |
| | | GTGTACCGGAGAATCGTGAAGAGCCTGTCCAATGACGATATCAA | Endosomal escape sequence: 4369-4401 |
| | | CAAGATCTCCGGCGATATGAAGGACAGCCTGAAGGAGATGTCCC | |
| | | TGGAGGAGATCTACTCTTATGAGAAGTACGGCGAGTTCATCACA | |
| | | CAGGAGGGCATCTCTTTTTATAACGACATCTGCGGCAAGGTCAAT | |
| | | AGCTTCATGAACCTGTACTGTCAGAAGAATAAGGAGAATAAGAA | |
| | | CCTGTATAAGCTGCAGAAGCTGCACAAGCAGATCCTGTGCATCG | |
| | | CCGATACCAGCTACGAGGTGCCCTATAAGTTCGAGAGCGACGAG | |
| | | GAGGTGTACCAGTCCGTGAATGGCTTTCTGGATAACATCTCTAGC | |
| | | AAGCACATCGTGGAGCGGCTGAGAAAGATCGGCGATAATTACAA | |
| | | CGGCTATAACCTGGACAAGATCTATATCGTGTCCAAGTTTTACGA | |
| | | GTCTGTGAGCCAGAAGACCTACCGGGACTGGGAGACCATCAATA | |
| | | CAGCCCTGGAGATCCACTATAATAACATCCTGCCTGGCAACGGC | |
| | | AAGTCTAAGGCCGATAAGGTGAAGAAGGCCGTGAAGAATGACCT | |
| | | GCAGAAGAGCATCACAGAGATCAATGAGCTGGTGTCTAACTACA | |
| | | AGCTGTGCAGCGACGATAACATCAAGGCCGAGACCTATATCCAC | |
| | | GAGATCAGCCACATCCTGAATAACTTCGAGGCCCAGGAGCTGAA | |
| | | GTACAATCCTGAGATCCACCTGGTGGAGTCTGAGCTGAAGGCCA | |
| | | GCGAGCTGAAGAATGTGCTGGACGTGATCATGAACGCCTTCCAC | |
| | | TGGTGTTCCGTGTTTATGACCGAGGAGCTGGTGGACAAGGATAAT | |
| | | AACTTTTATGCCGAGCTGGAGGAGATCTACGATGAGATCTATCCA | |
| | | GTGATCAGCCTGTATAATCTGGTGAGGAACTACGTGACCCAGAA | |
| | | GCCCTATTCCACAAAGAAGATCAAGCTGAACTTCGGCATCCCTAC | |
| | | ACTGGCCGACGGCTGGTCCAAGTCTAAGGAGTACAGCAATAACG | |
| | | CCATCATCCTGATGCGCGATAATCTGTACTATCTGGGCATCTTTA | |
| | | ATGCCAAGAACAAGCCAGACAAGAAGATCATCGAGGGCAATACC | |
| | | TCCGAGAACAAGGGCGATTACAAGAAGATGATCTATAATCTGCT | |
| | | GCCCGGCCCTAACAAGATGATCCCAAAGGTGTTCCTGTCCTCTAA | |
| | | GACCGGCGTGGAGACATACAAGCCCAGCGCCTATATCCTGGAGG | |
| | | GCTACAAGCAGAACAAGCACATCAAGAGCTCCAAGGACTTCGAT | |
| | | ATCACATTTTGCCACGATCTGATCGACTACTTCAAGAATTGTATC | |
| | | GCCATCCACCCCGAGTGGAAAAACTTCGGCTTTGATTTCTCCGAC | |
| | | ACCTCTACATACGAGGACATCAGCGGCTTTTATCGGGAGGTGGA | |
| | | GCTGCAGGGCTACAAGATCGATTGGACCTATATCTCCGAGAAGG | |
| | | ACATCGATCTGCTGCAGGAGAAGGGCCAGCTGTATCTGTTCCAG | |
| | | ATCTACAACAAGGACTTCAGCAAGAAGAGCACCGGCAATGACAA | |
| | | CCTGCACACAATGTACCTGAAGAATCTGTTCAGCGAGGAGAACC | |
| | | TGAAGGACATCGTGCTGAAGCTGAATGGCGAGGCCGAGATCTTC | |
| | | TTTAGAAAGTCTAGCATCAAGAATCCCATCATCCACAAGAAGGG | |
| | | CTCCATCCTGGTGAACCGGACCTACGAGGCCGAGGAGAAGGACC | |
| | | AGTTCGGCAACATCCAGATCGTGAGAAAGAATATCCCTGAGAAC | |
| | | ATCTATCAGGAGCTGTACAAGTACTTCAACGATAAGTCCGACAA | |
| | | GGAGCTGTCTGATGAGGCCGCCAAGCTGAAGAATGTGGTGGGCC | |
| | | ACCACGAGGCCGCCACAAACATCGTGAAGGATTACCGGTATACC | |
| | | TACGACAAGTACTTCCTGCACATGCCCATCACAATCAATTTCAAG | |
| | | GCCAACAAGACCGGCTTTATCAACGACAGAATCCTGCAGTACAT | |
| | | CGCCAAGGAGAAGGATCTGCACGTGATCGGCATCGACAGGGGCG | |
| | | AGCGCAATCTGATCTACGTGAGCGTGATCGACACCTGTGGCAAC | |
| | | ATCGTGGAGCAGAAGAGCTTTAATATCGTGAACGGCTATGATTA | |
| | | CCAGATCAAGCTGAAGCAGCAGGAGGGAGCAAGGCAGATCGCA | |
| | | AGAAAGGAGTGGAAGGAGATCGGCAAGATCAAGGAGATCAAGG | |
| | | AGGGCTACCTGAGCCTGGTCATCCACGAGATCAGCAAGATGGTC | |
| | | ATCAAGTACAACGCCATCATCGCCATGGAGGACCTGAGCTATGG | |
| | | CTTCAAGAAGGGCCGGTTTAAGGTGGAGAGACAGGTGTACCAGA | |
| | | AGTTCGAGACCATGCTGATCAATAAGCTGAACTATCTGGTGTTTA | |
| | | AGGACATCTCCATCACAGAGAACGGCGGCCTGCTGAAGGGCTAC | |
| | | CAGCTGACCTATATCCCTGATAAGCTGAAGAATGTGGGCCACCA | |
| | | GTGCGGCTGTATCTTCTATGTGCCAGCCGCCTACACAAGCAAGAT | |
| | | CGACCCCACCACAGGCTTTGTGAACATCTTTAAGTTCAAGGATCT | |
| | | GACCGTGGACGCCAAGAGGGAGTTCATCAAGAAGTTTGATAGCA | |
| | | TCCGCTACGACTCCGAGAAGAACCTGTTTTGCTTCACATTTGATT | |
| | | ACAACAACTTCATCACCCAGAATACAGTGATGTCTAAGTCCTCTT | |
| | | GGAGCGTGTATACCTACGGCGTGAGGATCAAGAGGCGCTTCGTG | |
| | | AATGGCCGCTTTTCTAACGAGAGCGATACCATCGACATCACAAA | |
| | | GGATATGGAGAAGACCCTGGAGATGACAGACATCAACTGGCGGG | |
| | | ATGGCCACGACCTGAGACAGGATATCATCGACTACGAGATCGTG | |
| | | CAGCACATCTTCGAGATCTTTAGGCTGACAGTGCAGATGCGCAAC | |
| | | AGCCTGTCCGAGCTGGAGGACAGGGATTACGACCGCCTGATCTC | |
| | | CCCTGTGCTGAATGAGAATAACATCTTCTATGATTCTGCCAAGGC | |
| | | AGGCGACGCACTGCCAAAGGATGCAGACGCCAACGGCGCCTACT | |
| | | GTATCGCCCTGAAGGGCCTGTATGAGATCAAGCAGATCACCGAG | |
| | | AATTGGAAGGAGGATGGCAAGTTTTCCAGGGACAAGCTGAAGAT | |
| | | CTCTAATAAGGATTGGTTCGACTTTATCCAGAACAAGCGGTACCT | |
| | | GGGAGGAGGAGGCTCCGGCGGAGGAGGCTCTGGCGGCGGCGGCA | |
| | | GCGGAGGCGGCGGCTCCCAGGTGAAGCTGGAGGAGAGCGGAGG | |
| | | AGGCTCCGTGCAGACCGGAGGCAGCCTGAGGCTGACATGCGCAG | |
| | | CATCCGGACGGACCTCTAGAAGCTACGGAATGGGATGGTTCAGG | |
| | | CAGGCACCAGGCAAGGAGAGAGAGTTCGTGAGCGGCATCTCTTG | |
| | | GCGCGGCGATTCCACCGGCTATGCCGACTCTGTGAAGGGCAGGT | |
| | | TCACAATCTCCCGCGATAATGCCAAGAACACCGTGGACCTGCAG | |
| | | ATGAACTCTCTGAAGCCCGAGGACACAGCCATCTACTATTGTGCA | |
| | | GCAGCAGCAGGCAGCGCCTGGTACGGCACCCTGTATGAGTACGA | |
| | | TTATTGGGGCCAGGGCACCCAGGTGACAGTGAGCTCCGCCCTGG | |
| | | AGCCCAAGAAGAAGCGGAAGGTGGAGGACCCCAAGAAGAAGC | |
| | | GGAAAGTGGAGAATCTGTATTTTCAGGGCGGCTCTAGCCATCAT | |
| | | CACCATCATCACCACCACCACCACTGA | |
| 77 | md7-7d- | MYRMQLLSCIALSLALVTNSMNNGTNNFQNFIGISSLQKTLRNALI | IL-2 secretion sequence: bold |
| | L4 (Mdl4) | PTETTQQFIVKNGIIKEDELRGENRQILKDIMDDYYRGFISETLSSIDDI | Endonuclease: single underline |
| | (protein | DWTSLFEKMEIQLKNGDNKDTLIKEQTEYRKAIHKKFANDDRFKNM | Linker: italics |
| | sequence) | FSAKLISDILPEFVIHNNNYSASEKEEKTQVIKLFSRFATSFKDYFKNR | Cell recognition domain: double underline |
| | | ANCFSADDISSSSCHRIVNDNAEIFFSNALVYRRIVKSLSNDDINKISG | NLS sequence: bold |
| | | DMKDSLKEMSLEEIYSYEKYGEFITQEGISFYNDICGKVNSFMNLYC | TEV-cleavage sequence: underlined |
| | | QKNKENKNLYKLQKLHKQILCIADTSYEVPYKFESDEEVYQSVNGF | Endosomal release sequence: bold |
| | | LDNISSKHIVERLRKIGDNYNGYNLDKIYIVSKFYESVSQKTYRDWE | Residue numbering: |
| | | TINTALEIHYNNILPGNGKSKADKVKKAVKNDLQKSITEINELVSNY | IL-2 secretion sequence: 1-20 |
| | | KLCSDDNIKAETYIHEISHILNNFEAQELKYNPEIHLVESELKASELKN | Endonuclease MAD7: 21-1283 |
| | | VLDVIMNAFHWCSVFMTEELVDKDNNFYAELEEIYDEIYPVISLYNL | Linker: 1284- 1303 |
| | | VRNYVTQKPYSTKKIKLNFGIPTLADGWSKSKEYSNNAIILMRDNLY | Cell recognition domain 7dl2: 1304-1430 |
| | | YLGIFNAKNKPDKKIIEGNTSENKGDYKKMIYNLLPGPNKMIPKVFL | NLS: 1431-1446 |
| | | SSKTGVETYKPSAYILEGYKQNKHIKSSKDFDITFCHDLIDYFKNCIAI | Tev-cleavage sequence: 1447-1453 |
| | | HPEWKNFGFDFSDTSTYEDISGFYREVELQGYKIDWTYISEKDIDLL | Endosomal escape sequence: 1457-1466 |
| | | QEKGQLYLFQIYNKDFSKKSTGNDNLHTMYLKNLFSEENLKDIVLK | |
| | | LNGEAEIFFRKSSIKNPIIHKKGSILVNRTYEAEEKDQFGNIQFVRKNIP | |
| | | ENIYQELYKYFNDKSDKELSDEAAKLKNVVGHHEAATNIVKDYRY | |
| | | TYDKYFLHMPITINFKANKTGFINDRILQYIAKEKDLHVIGIDRGERN | |
| | | LIYVSVIDTCGNIVEQKSFNIVNGYDYQIKLKQQEGARQIARKEWKEI | |
| | | GKIKEIKEGYLSLVIHEISKMVIKYNAIIAMEDLSYGFKKGRFKVERQ | |
| | | VYQKFETMLINKLNYLVFKDISITENGGLLKGYQLTYIPDKLKNVGH | |
| | | QCGCIFYVPAAYTSKIDPTTGFVNIFKFKDLTVDAKREFIKKFDSIRY | |
| | | DSEKNLFCFTFDYNNFITQNTVMSKSSWSVYTYGVRIKRRFVNGRFS | |
| | | NESDTIDITKDMEKTLEMTDINWRDGHDLRQDIIDYEIVQHIFEIFRLT | |
| | | VQMRNSLSELEDRDYDRLISPVLNENNIFYDSAKAGDALPKDADAN | |
| | | GAYCIALKGLYEIKQITENWKEDGKFSRDKLKISNKDWFDFIQNKRY | |
| | | LGGGGSGGGGSGGGGSGGGGSQVKLEESGGGSVQTGGSLRLTCAAS | |
| | | GRTSRSYGMGWFRQAPGKEREFVSGISWRGDSTGYADSVKGRFTIS | |
| | | RDNAKNTVDLQMNSLKPEDTAIYYCAAAAGSAWYGTLYEYDYWG | |
| | | QGTQVTVSSALEPKKKRKVEDPKKKRKVENLYFQGGSSHHHHH | |
| | | HHHHH | |
| 78 | Md-MA- | ATGCATCATCATCATCATCACAGCAGCGGCAGAGAAAACTTG | His-TEV-cleavage sequence: bold |
| | 7d | TATTTCCAGGGCATGAACAACGGCACCAACAACTTTCAGAACTT | Endonuclease: single underline |
| | | TATTGGCATTAGCAGCCTGCAGAAAACCCTGCGCAACGCGCTGA | Linker: italics |
| | | TTCCGACCGAAACCACCCAGCAGTTTATTGTGAAAAACGGCATTA | NLS sequence: underlined bold |
| | | TTAAAGAAGATGAACTGCGCGGCGAAAACCGCCAGATTCTGAAA | Hapten binding domain: bold |
| | | GATATTATGGATGATTATTATCGCGGCTTTATTAGCGAAACCCTG | Linker 2: italics |
| | | AGCAGCATTGATGATATTGATTGGACCAGCCTGTTTGAAAAAATG | Cell recognition domain: double underline |
| | | GAAATTCAGCTGAAAAACGGCGATAACAAAGATACCCTGATTAA | Endosomal release sequence: bold |
| | | AGAACAGACCGAATATCGCAAAGCGATTCATAAAAAATTTGCGA | Residue numbering (translated amino |
| | | ACGATGATCGCTTTAAAAACATGTTTAGCGCGAAACTGATTAGCG | acids): |
| | | ATATTCTGCCGGAATTTGTGATTCATAACAACAACTATAGCGCGA | His-TEV sequence: 1-54 |
| | | GCGAAAAAGAAGAAAAAACCCAGGTGATTAAACTGTTTAGCCGC | Endonuclease MAD7: 55-3842 |
| | | TTTGCGACCAGCTTTAAAGATTATTTTAAAAACCGCGCGAACTGC | Linker: 3843-3939 |
| | | TTTAGCGCGGATGATATTAGCAGCAGCAGCTGCCATCGCATTGTG | NLS: 3940-3987 |
| | | AACGATAACGCGGAAATTTTTTTTAGCAACGCGCTGGTGTATCGC | 2nd His-tag: 3988-4005 |
| | | CGCATTGTGAAAAGCCTGAGCAACGATGATATTAACAAAATTAG | Hapten binding domain (monoavidin |
| | | CGGCGATATGAAAGATAGCCTGAAAGAAATGAGCCTGGAAGAA | binding domain): 4006-4476 |
| | | ATTTATAGCTATGAAAAATATGGCGAATTTATTACCCAGGAAGGC | Linker 2: 4477-4560 |
| | | ATTAGCTTTTATAACGATATTTGCGGCAAAGTGAACAGCTTTATG | Cell recognition domain 7dl2: 4561-4944 |
| | | AACCTGTATTGCCAGAAAAACAAAGAAAACAAAAACCTGTATAA | Endosomal escape sequence: 4945-4965 |
| | | ACTGCAGAAACTGCATAAACAGATTCTGTGCATTGCGGATACCA | |
| | | GCTATGAAGTGCCGTATAAATTTGAAAGCGATGAAGAAGTGTAT | |
| | | CAGAGCGTGAACGGCTTTCTGGATAACATTAGCAGCAAACATAT | |
| | | TGTGGAACGCCTGCGCAAAATTGGCGATAACTATAACGGCTATA | |
| | | ACCTGGATAAAATTTATATTGTGAGCAAATTTTATGAAAGCGTGA | |
| | | GCCAGAAAACCTATCGCGATTGGGAAACCATTAACACCGCGCTG | |
| | | GAAATTCATTATAACAACATTCTGCCGGGCAACGGCAAAAGCAA | |
| | | AGCGGATAAAGTGAAAAAAGCGGTGAAAAACGATCTGCAGAAA | |
| | | AGCATTACCGAAATTAACGAACTGGTGAGCAACTATAAACTGTG | |
| | | CAGCGATGATAACATTAAAGCGGAAACCTATATTCATGAAATTA | |
| | | GCCATATTCTGAACAACTTTGAAGCGCAGGAACTGAAATATAAC | |
| | | CCGGAAATTCATCTGGTGGAAAGCGAACTGAAAGCGAGCGAACT | |
| | | GAAAAACGTGCTGGATGTGATTATGAACGCGTTTCATTGGTGCAG | |
| | | CGTGTTTATGACCGAAGAACTGGTGGATAAAGATAACAACTTTTA | |
| | | TGCGGAACTGGAAGAAATTTATGATGAAATTTATCCGGTGATTAG | |
| | | CCTGTATAACCTGGTGCGCAACTATGTGACCCAGAAACCGTATAG | |
| | | CACCAAAAAAATTAAACTGAACTTTGGCATTCCGACCCTGGCGG | |
| | | ATGGCTGGAGCAAAAGCAAAGAATATAGCAACAACGCGATTATT | |
| | | CTGATGCGCGATAACCTGTATTATCTGGGCATTTTTAACGCGAAA | |
| | | AACAAACCGGATAAAAAAATTATTGAAGGCAACACCAGCGAAA | |
| | | ACAAAGGCGATTATAAAAAAATGATTTATAACCTGCTGCCGGGC | |
| | | CCGAACAAAATGATTCCGAAAGTGTTTCTGAGCAGCAAAACCGG | |
| | | CGTGGAAACCTATAAACCGAGCGCGTATATTCTGGAAGGCTATA | |
| | | AACAGAACAAACATATTAAAAGCAGCAAAGATTTTGATATTACC | |
| | | TTTTGCCATGATCTGATTGATTATTTTAAAAACTGCATTGCGATTC | |
| | | ATCCGGAATGGAAAAACTTTGGCTTTGATTTTAGCGATACCAGCA | |
| | | CCTATGAAGATATTAGCGGCTTTTATCGCGAAGTGGAACTGCAGG | |
| | | GCTATAAAATTGATTGGACCTATATTAGCGAAAAAGATATTGATC | |
| | | TGCTGCAGGAAAAAGGCCAGCTGTATCTGTTTCAGATTTATAACA | |
| | | AAGATTTTAGCAAAAAAAGCACCGGCAACGATAACCTGCATACC | |
| | | ATGTATCTGAAAAACCTGTTTAGCGAAGAAAACCTGAAAGATAT | |
| | | TGTGCTGAAACTGAACGGCGAAGCGGAAATTTTTTTTCGCAAAA | |
| | | GCAGCATTAAAAACCCGATTATTCATAAAAAAGGCAGCATTCTG | |
| | | GTGAACCGCACCTATGAAGCGGAAGAAAAAGATCAGTTTGGCAA | |
| | | CATTCAGATTGTGCGCAAAAACATTCCGGAAAACATTTATCAGG | |
| | | AACTGTATAAATATTTTAACGATAAAAGCGATAAAGAACTGAGC | |
| | | GATGAAGCGGCGAAACTGAAAAACGTGGTGGGCCATCATGAAGC | |
| | | GGCGACCAACATTGTGAAAGATTATCGCTATACCTATGATAAATA | |
| | | TTTTCTGCATATGCCGATTACCATTAACTTTAAAGCGAACAAAAC | |
| | | CGGCTTTATTAACGATCGCATTCTGCAGTATATTGCGAAAGAAAA | |
| | | AGATCTGCATGTGATTGGCATTGATCGCGGCGAACGCAACCTGAT | |
| | | TTATGTGAGCGTGATTGATACCTGCGGCAACATTGTGGAACAGA | |
| | | AAAGCTTTAACATTGTGAACGGCTATGATTATCAGATTAAACTGA | |
| | | AACAGCAGGAAGGCGCGCGCCAGATTGCGCGCAAAGAATGGAA | |
| | | AGAAATTGGCAAAATTAAAGAAATTAAAGAAGGCTATCTGAGCC | |
| | | TGGTGATTCATGAAATTAGCAAAATGGTGATTAAATATAACGCG | |
| | | ATTATTGCGATGGAAGATCTGAGCTATGGCTTTAAAAAAGGCCG | |
| | | CTTTAAAGTGGAACGCCAGGTGTATCAGAAATTTGAAACCATGCT | |
| | | GATTAACAAACTGAACTATCTGGTGTTTAAAGATATTAGCATTAC | |
| | | CGAAAACGGCGGCCTGCTGAAAGGCTATCAGCTGACCTATATTC | |
| | | CGGATAAACTGAAAAACGTGGGCCATCAGTGCGGCTGCATTTTTT | |
| | | ATGTGCCGGCGGCGTATACCAGCAAAATTGATCCGACCACCGGC | |
| | | TTTGTGAACATTTTTAAATTTAAAGATCTGACCGTGGATGCGAAA | |
| | | CGCGAATTTATTAAAAAATTTGATAGCATTCGCTATGATAGCGAA | |
| | | AAAAACCTGTTTTGCTTTACCTTTGATTATAACAACTTTATTACCC | |
| | | AGAACACCGTGATGAGCAAAAGCAGCTGGAGCGTGTATACCTAT | |
| | | GGCGTGCGCATTAAACGCCGCTTTGTGAACGGCCGCTTTAGCAAC | |
| | | GAAAGCGATACCATTGATATTACCAAAGATATGGAAAAAACCCT | |
| | | GGAAATGACCGATATTAACTGGCGCGATGGCCATGATCTGCGCC | |
| | | AGGATATTATTGATTATGAAATTGTGCAGCATATTTTTGAAATTT | |
| | | TTCGCCTGACCGTGCAGATGCGCAACAGCCTGAGCGAACTGGAA | |
| | | GATCGCGATTATGATCGCCTGATTAGCCCGGTGCTGAACGAAAA | |
| | | CAACATTTTTTATGATAGCGCGAAAGCGGGCGATGCGCTGCCGA | |
| | | AAGATGCGGATGCGAACGGCGCGTATTGCATTGCGCTGAAAGGC | |
| | | CTGTATGAAATTAAACAGATTACCGAAAACTGGAAAGAAGATGG | |
| | | CAAATTTAGCCGCGATAAACTGAAAATTAGCAACAAAGATTGGT | |
| | | TTGATTTTATTCAGAACAAACGCTATCTGGGCGGCGGCGGCAGCG | |
| | | GCGGCGGCGGCAGCGGCGGCGGCGGCAGCGGCGGCGGCGGCAGC | |
| | | GGCGGCGGCGGCAGCGGCGGCGGCGGCAGCACCAGCCCTAAGAA | |
| | | AAAACGAAAAGTTGAGGATCCTAAAAAGAAACGAAAAGTTCA | |
| | | TCATCATCATCATCATGAATTTGCGAGCGCGGAAGCGGGCATTA | |
| | | CCGGCACCTGGTATAACCAGCATGGCAGCACCTTTACCGTGA | |
| | | CCGCGGGCGCGGATGGCAACCTGACCGGCCAGTATGAAAAC | |
| | | CGCGCGCAGGGCACCGGCTGCCAGAACAGCCCGTATACCCT | |
| | | GACCGGCCGCTATAACGGCACCAAACTGGAATGGCGCGTGG | |
| | | AATGGAACAACAGCACCGAAAACTGCCATAGCCGCACCGAAT | |
| | | GGCGCGGCCAGTATCAGGGCGGCGCGGAAGCGCGCATTAAC | |
| | | ACCCAGTGGAACCTGACCTATGAAGGCGGCAGCGGCCCGGC | |
| | | GACCGAACAGGGCCAGGATACCTTTACCAAAGTGAAACCGAG | |
| | | CGCGGCGAGCGGCAGCGATTATAAAGATGATGATGATAAAAA | |
| | | ACGCAAAAGAAAATGCCGATATCCTATTGGCATTGACGTCAG | |
| | | GTGGCACTTTTCGAGGAGATCATGCACAGGCGGCGGCGGCAGC | |
| | | GGCGGCGGCGGCAGCGGCGGCGGCGGCAGCGGCGGCGGCGGCAG | |
| | | CGGCGGCGGCGGCAGCGGCGGCAGCCCATGGGCGGCGCAGGTTA | |
| | | AACTGGAAGAATCTGGTGGTGGTTCTGTTCAGACCGGTGGTTCTC | |
| | | TGCGTCTGACCTGCGCGGCGTCTGGTCGTACCTCTCGTTCTTACG | |
| | | GTATGGGTTGGTTCCGTCAGGCGCCGGGTAAAGAACGTGAATTC | |
| | | GTTTCTGGTATCTCTTGGCGTGGTGACTCTACCGGTTACGCGGAC | |
| | | TCTGTTAAAGGTCGTTTCACCATCTCTCGTGACAACGCGAAAAAC | |
| | | ACCGTTGACCTGCAGATGAACTCTCTGAAACCGGAAGACACCGC | |
| | | GATCTACTACTGCGCGGCGGCGGCGGGTTCTGCGTGGTACGGTAC | |
| | | CCTGTACGAATACGACTACTGGGGTCAGGGTACCCAGGTTACCGT | |
| | | TTCTTCTTGTTGTTGTTGTTGTTGTTAA | |
| 79 | Md-MA- | MHHHHHHSSGRENLYFQGMNNGTNNFQNFIGISSLQKTLRNALIP | His-TEV sequence: bold |
| | 7d | TETTQQFIVKNGIIKEDELRGENRQILKDIMDDYYRGFISETLSSIDDI | Endonuclease: single underline |
| | | DWTSLFEKMEIQLKNGDNKDTLIKEQTEYRKAIHKKFANDDRFKNM | Linker: italics |
| | | FSAKLISDILPEFVIHNNNYSASEKEEKTQVIKLFSRFATSFKDYFKNR | NLS sequence: underlined bold |
| | | ANCFSADDISSSSCHRIVNDNAEIFFSNALVYRRIVKSLSNDDINKISG | His-tag sequence: underlined italics |
| | | DMKDSLKEMSLEEIYSYEKYGEFITQEGISFYNDICGKVNSFMNLYC | Hapten binding domain: bold |
| | | QKNKENKNLYKLQKLHKQILCIADTSYEVPYKFESDEEVYQSVNGF | Linker 2: italics |
| | | LDNISSKHIVERLRKIGDNYNGYNLDKIYIVSKFYESVSQKTYRDWE | Cell recognition domain: double underline |
| | | TINTALEIHYNNILPGNGKSKADKVKKAVKNDLQKSITEINELVSNY | Endosomal release sequence: bold |
| | | KLCSDDNIKAETYIHEISHILNNFEAQELKYNPEIHLVESELKASELKN | Residue numbering: |
| | | VLDVIMNAFHWCSVFMTEELVDKDNNFYAELEEIYDEIYPVISLYNL | His-TEV-cleavage sequence 1: 1-18 |
| | | VRNYVTQKPYSTKKIKLNFGIPTLADGWSKSKEYSNNAIILMRDNLY | Endonuclease MAD7: 19-1281 |
| | | YLGIFNAKNKPDKKIIEGNTSENKGDYKKMIYNLLPGPNKMIPKVFL | Linker: 1282: to 1311 |
| | | SSKTGVETYKPSAYILEGYKQNKHIKSSKDFDITFCHDLIDYFKNCIAI | NLS: 1313-1329 |
| | | HPEWKNFGFDFSDTSTYEDISGFYREVELQGYKIDWTYISEKDIDLL | 2nd His-tag: 1330-1335 |
| | | QEKGQLYLFQIYNKDFSKKSTGNDNLHTMYLKNLFSEENLKDIVLK | Hapten binding domain (monoavidin |
| | | LNGEAEIFFRKSSIKNPIIHKKGSILVNRTYEAEEKDQFGNIQIVRKNIP | binding domain): 1336-1491 |
| | | ENIYQELYKYFNDKSDKELSDEAAKLKNVVGHHEAATNFVKDYRY | Linker 2: 1492- 1520 |
| | | TYDKYFLHMPITINFKANKTGFINDRILQYIAKEKDLHVIGIDRGERN | Cell recognition domain 7dl2: 1521-1648 |
| | | LIYVSVIDTCGNIVEQKSFNIVNGYDYQIKLKQQEGARQIARKEWKEI | Endosomal escape sequence: 1649-1654 |
| | | GKIKEIKEGYLSLVIHEISKMVIKYNAIIAMEDLSYGFKKGRFKVERQ | |
| | | VYQKFETMLINKLNYLVFKDISITENGGLLKGYQLTYIPDKLKNVGH | |
| | | QCGCIFYVPAAYTSKIDPTTGFVNIFKFKDLTVDAKREFIKKFDSIRY | |
| | | DSEKNLFCFTFDYNNFITQNTVMSKSSWSVYTYGVRIKRRFVNGRFS | |
| | | NESDTIDITKDMEKTLEMTDINWRDGHDLRQDIIDYEIVQHIFEIFRLT | |
| | | VQMRNSLSELEDRDYDRLISPVLNENNIFYDSAKAGDALPKDADAN | |
| | | GAYCIALKGLYEIKQITENWKEDGKFSRDKLKISNKDWFDFIQNKRY | |
| | | LGGGGSGGGGSGGGGSGGGGSGGGGSGGGGSTSPKKKRKVEDPK | |
| | | KKRKVHHHHHHEFASAEAGITGTWYNQHGSTFTVTAGADGNLT | |
| | | GQYENRAQGTGCQNSPYTLTGRYNGTKLEWRVEWNNSTENCH | |
| | | SRTEWRGQYQGGAEARINTQWNLTYEGGSGPATEQGQDTFTK | |
| | | VKPSAASGSDYKDDDDKKRKRKCRYPIGIDVRWHFSRRSCTGGG | |
| | | GSGGGGSGGGGSGGGGSGGGGSGGSPWAAQVKLEESGGGSVQEGG | |
| | | SLRLTCAASGRTSRSYGMGWFRQAPGKEREFVSGISWRGDSTGYAD | |
| | | SVKGRFTISRDNAKNTVDLQMNSLKPEDTAIYYCAAAAGSAWYGT | |
| | | LYEYDYWGQGTQVTVSSCCCCCC | |
| 80 | Md-MA- | ATGCATCATCATCATCATCACAGCAGCGGCAGAGAAAACTTG | His-TEV-cleavage sequence: bold |
| | 47 | TATTTCCAGGGCATGAACAACGGCACCAACAACTTTCAGAACTT | Endonuclease: single underline |
| | (nucleotide | TATTGGCATTAGCAGCCTGCAGAAAACCCTGCGCAACGCGCTGA | Linker: italics |
| | sequence) | TTCCGACCGAAACCACCCAGCAGTTTATTGTGAAAAACGGCATTA | NLS sequence: underlined bold |
| | | TTAAAGAAGATGAACTGCGCGGCGAAAACCGCCAGATTCTGAAA | His-tag sequence: underlined italics |
| | | GATATTATGGATGATTATTATCGCGGCTTTATTAGCGAAACCCTG | Hapten binding domain: bold |
| | | AGCAGCATTGATGATATTGATTGGACCAGCCTGTTTGAAAAAATG | Linker 2: italics |
| | | GAAATTCAGCTGAAAAACGGCGATAACAAAGATACCCTGATTAA | Cell recognition domain: double underline |
| | | AGAACAGACCGAATATCGCAAAGCGATTCATAAAAAATTTGCGA | Endosomal release sequence: bold |
| | | ACGATGATCGCTTTAAAAACATGTTTAGCGCGAAACTGATTAGCG | Residue numbering: |
| | | ATATTCTGCCGGAATTTGTGATTCATAACAACAACTATAGCGCGA | His-TEV cleavage sequence: 1-54 |
| | | GCGAAAAAGAAGAAAAAACCCAGGTGATTAAACTGTTTAGCCGC | Endonuclease MAD7: 55-3842 |
| | | TTTGCGACCAGCTTTAAAGATTATTTTAAAAACCGCGCGAACTGC | Linker: 3843-3939 |
| | | TTTAGCGCGGATGATATTAGCAGCAGCAGCTGCCATCGCATTGTG | NLS: 3940-3987 |
| | | AACGATAACGCGGAAATTTTTTTTAGCAACGCGCTGGTGTATCGC | 2nd His tag: 3988-4005 |
| | | CGCATTGTGAAAAGCCTGAGCAACGATGATATTAACAAAATTAG | Hapten binding domain (monoavidin |
| | | CGGCGATATGAAAGATAGCCTGAAAGAAATGAGCCTGGAAGAA | binding domain): 4006-4476 |
| | | ATTTATAGCTATGAAAAATATGGCGAATTTATTACCCAGGAAGGC | Linker2: 4477-4560 |
| | | ATTAGCTTTTATAACGATATTTGCGGCAAAGTGAACAGCTTTATG | Cell recognition domain 7dl2: 4560-4902 |
| | | AACCTGTATTGCCAGAAAAACAAAGAAAACAAAAACCTGTATAA | Endosomal escape sequence: 4903-4923 |
| | | ACTGCAGAAACTGCATAAACAGATTCTGTGCATTGCGGATACCA | |
| | | GCTATGAAGTGCCGTATAAATTTGAAAGCGATGAAGAAGTGTAT | |
| | | CAGAGCGTGAACGGCTTTCTGGATAACATTAGCAGCAAACATAT | |
| | | TGTGGAACGCCTGCGCAAAATTGGCGATAACTATAACGGCTATA | |
| | | ACCTGGATAAAATTTATATTGTGAGCAAATTTTATGAAAGCGTGA | |
| | | GCCAGAAAACCTATCGCGATTGGGAAACCATTAACACCGCGCTG | |
| | | GAAATTCATTATAACAACATTCTGCCGGGCAACGGCAAAAGCAA | |
| | | AGCGGATAAAGTGAAAAAAGCGGTGAAAAACGATCTGCAGAAA | |
| | | AGCATTACCGAAATTAACGAACTGGTGAGCAACTATAAACTGTG | |
| | | CAGCGATGATAACATTAAAGCGGAAACCTATATTCATGAAATTA | |
| | | GCCATATTCTGAACAACTTTGAAGCGCAGGAACTGAAATATAAC | |
| | | CCGGAAATTCATCTGGTGGAAAGCGAACTGAAAGCGAGCGAACT | |
| | | GAAAAACGTGCTGGATGTGATTATGAACGCGTTTCATTGGTGCAG | |
| | | CGTGTTTATGACCGAAGAACTGGTGGATAAAGATAACAACTTTTA | |
| | | TGCGGAACTGGAAGAAATTTATGATGAAATTTATCCGGTGATTAG | |
| | | CCTGTATAACCTGGTGCGCAACTATGTGACCCAGAAACCGTATAG | |
| | | CACCAAAAAAATTAAACTGAACTTTGGCATTCCGACCCTGGCGG | |
| | | ATGGCTGGAGCAAAAGCAAAGAATATAGCAACAACGCGATTATT | |
| | | CTGATGCGCGATAACCTGTATTATCTGGGCATTTTTAACGCGAAA | |
| | | AACAAACCGGATAAAAAAATTATTGAAGGCAACACCAGCGAAA | |
| | | ACAAAGGCGATTATAAAAAAATGATTTATAACCTGCTGCCGGGC | |
| | | CCGAACAAAATGATTCCGAAAGTGTTTCTGAGCAGCAAAACCGG | |
| | | CGTGGAAACCTATAAACCGAGCGCGTATATTCTGGAAGGCTATA | |
| | | AACAGAACAAACATATTAAAAGCAGCAAAGATTTTGATATTACC | |
| | | TTTTGCCATGATCTGATTGATTATTTTAAAAACTGCATTGCGATTC | |
| | | ATCCGGAATGGAAAAACTTTGGCTTTGATTTTAGCGATACCAGCA | |
| | | CCTATGAAGATATTAGCGGCTTTTATCGCGAAGTGGAACTGCAGG | |
| | | GCTATAAAATTGATTGGACCTATATTAGCGAAAAAGATATTGATC | |
| | | TGCTGCAGGAAAAAGGCCAGCTGTATCTGTTTCAGATTTATAACA | |
| | | AAGATTTTAGCAAAAAAAGCACCGGCAACGATAACCTGCATACC | |
| | | ATGTATCTGAAAAACCTGTTTAGCGAAGAAAACCTGAAAGATAT | |
| | | TGTGCTGAAACTGAACGGCGAAGCGGAAATTTTTTTTCGCAAAA | |
| | | GCAGCATTAAAAACCCGATTATTCATAAAAAAGGCAGCATTCTG | |
| | | GTGAACCGCACCTATGAAGCGGAAGAAAAAGATCAGTTTGGCAA | |
| | | CATTCAGATTGTGCGCAAAAACATTCCGGAAAACATTTATCAGG | |
| | | AACTGTATAAATATTTTAACGATAAAAGCGATAAAGAACTGAGC | |
| | | GATGAAGCGGCGAAACTGAAAAACGTGGTGGGCCATCATGAAGC | |
| | | GGCGACCAACATTGTGAAAGATTATCGCTATACCTATGATAAATA | |
| | | TTTTCTGCATATGCCGATTACCATTAACTTTAAAGCGAACAAAAC | |
| | | CGGCTTTATTAACGATCGCATTCTGCAGTATATTGCGAAAGAAAA | |
| | | AGATCTGCATGTGATTGGCATTGATCGCGGCGAACGCAACCTGAT | |
| | | TTATGTGAGCGTGATTGATACCTGCGGCAACATTGTGGAACAGA | |
| | | AAAGCTTTAACATTGTGAACGGCTATGATTATCAGATTAAACTGA | |
| | | AACAGCAGGAAGGCGCGCGCCAGATTGCGCGCAAAGAATGGAA | |
| | | AGAAATTGGCAAAATTAAAGAAATTAAAGAAGGCTATCTGAGCC | |
| | | TGGTGATTCATGAAATTAGCAAAATGGTGATTAAATATAACGCG | |
| | | ATTATTGCGATGGAAGATCTGAGCTATGGCTTTAAAAAAGGCCG | |
| | | CTTTAAAGTGGAACGCCAGGTGTATCAGAAATTTGAAACCATGCT | |
| | | GATTAACAAACTGAACTATCTGGTGTTTAAAGATATTAGCATTAC | |
| | | CGAAAACGGCGGCCTGCTGAAAGGCTATCAGCTGACCTATATTC | |
| | | CGGATAAACTGAAAAACGTGGGCCATCAGTGCGGCTGCATTTTTT | |
| | | ATGTGCCGGCGGCGTATACCAGCAAAATTGATCCGACCACCGGC | |
| | | TTTGTGAACATTTTTAAATTTAAAGATCTGACCGTGGATGCGAAA | |
| | | CGCGAATTTATTAAAAAATTTGATAGCATTCGCTATGATAGCGAA | |
| | | AAAAACCTGTTTTGCTTTACCTTTGATTATAACAACTTTATTACCC | |
| | | AGAACACCGTGATGAGCAAAAGCAGCTGGAGCGTGTATACCTAT | |
| | | GGCGTGCGCATTAAACGCCGCTTTGTGAACGGCCGCTTTAGCAAC | |
| | | GAAAGCGATACCATTGATATTACCAAAGATATGGAAAAAACCCT | |
| | | GGAAATGACCGATATTAACTGGCGCGATGGCCATGATCTGCGCC | |
| | | AGGATATTATTGATTATGAAATTGTGCAGCATATTTTTGAAATTT | |
| | | TTCGCCTGACCGTGCAGATGCGCAACAGCCTGAGCGAACTGGAA | |
| | | GATCGCGATTATGATCGCCTGATTAGCCCGGTGCTGAACGAAAA | |
| | | CAACATTTTTTATGATAGCGCGAAAGCGGGCGATGCGCTGCCGA | |
| | | AAGATGCGGATGCGAACGGCGCGTATTGCATTGCGCTGAAAGGC | |
| | | CTGTATGAAATTAAACAGATTACCGAAAACTGGAAAGAAGATGG | |
| | | CAAATTTAGCCGCGATAAACTGAAAATTAGCAACAAAGATTGGT | |
| | | TTGATTTTATTCAGAACAAACGCTATCTGGGCGGCGGCGGCAGCG | |
| | | GCGGCGGCGGCAGCGGCGGCGGCGGCAGCGGCGGCGGCGGCAGC | |
| | | GGCGGCGGCGGCAGCGGCGGCGGCGGCAGCACCAGCCCTAAGAA | |
| | | AAAACGAAAAGTTGAGGATCCTAAAAAGAAACGAAAAGTTCA | |
| | | TCATCATCATCATCATGAATTTGCGAGCGCGGAAGCGGGCATTA | |
| | | CCGGCACCTGGTATAACCAGCATGGCAGCACCTTTACCGTGA | |
| | | CCGCGGGCGCGGATGGCAACCTGACCGGCCAGTATGAAAAC | |
| | | CGCGCGCAGGGCACCGGCTGCCAGAACAGCCCGTATACCCT | |
| | | GACCGGCCGCTATAACGGCACCAAACTGGAATGGCGCGTGG | |
| | | AATGGAACAACAGCACCGAAAACTGCCATAGCCGCACCGAAT | |
| | | GGCGCGGCCAGTATCAGGGCGGCGCGGAAGCGCGCATTAAC | |
| | | ACCCAGTGGAACCTGACCTATGAAGGCGGCAGCGGCCCGGC | |
| | | GACCGAACAGGGCCAGGATACCTTTACCAAAGTGAAACCGAG | |
| | | CGCGGCGAGCGGCAGCGATTATAAAGATGATGATGATAAAAA | |
| | | ACGCAAAAGAAAATGCCGATATCCTATTGGCATTGACGTCAG | |
| | | GTGGCACTTTTCGAGGAGATCATGCACAGGCGGCGGCGGCAGC | |
| | | GGCGGCGGCGGCAGCGGCGGCGGCGGCAGCGGCGGCGGCGGCAG | |
| | | CGGCGGCGGCGGCAGCGGCGGCAGCCAGGAGCAGCAGCAGGAGA | |
| | | CTGGAGGAGGCTTGGTGCAGCCTGGGGGGTCTCTGAGACTCTCCT | |
| | | GTGCAGCCTCTGGATTCACATTCAGTAGCTACGACATGAGCTGGG | |
| | | TCCGCCAGGCTCCGGGGAAGGGGCTCGAGTGGGTCTCAGGTATG | |
| | | AATAGTGGTGGTGGTAGAACATACTATGAAGACTCCGTGAAGGG | |
| | | CCGATTCACCATCTCCAGGTCCAACGCCAAGAACACGCTGTATCT | |
| | | GCAACTGAACAGCCTGAAAACTGACGACACGGCCATGTATTACT | |
| | | GTGTCACATCCGACTTTGCTTACTGGGGCCAGGGGACCCAGGTCA | |
| | | CCGTCTCCTCATGTTGTTGTTGTTGTTGTTAA | |
| 81 | Md-MA- | MHHHHHHSSGRENLYFQGMNNGTNNFQNFIGISSLQKTLRNALIP | His-TEV sequence: bold |
| | 47 | TETTQQFIVKNGIIKEDELRGENRQILKDIMDDYYRGFISETLSSIDDI | Endonuclease: single underline |
| | (protein | DWTSLFEKMEIQLKNGDNKDTLIKEQTEYRKAIHKKFANDDRFKNM | Linker: italics |
| | sequence) | FSAKLISDILPEFVIHNNNYSASEKEEKTQVIKLFSRFATSFKDYFKNR | NLS sequence: underlined bold |
| | | ANCFSADDISSSSCHRIVNDNAEIFFSNALVYRRIVKSLSNDDINKISG | His-tag sequence: underlined italics |
| | | DMKDSLKEMSLEEIYSYEKYGEFITQEGISFYNDICGKVNSFMNLYC | Hapten binding domain: bold |
| | | QKNKENKNLYKLQKLHKQILCIADTSYEVPYKFESDEEVYQSVNGF | Linker 2: italics |
| | | LDNISSKHIVERLRKIGDNYNGYNLDKIYIVSKFYESVSQKTYRDWE | Cell recognition domain: double underline |
| | | TINTALEIHYNNILPGNGKSKADKVKKAVKNDLQKSITEINELVSNY | Endosomal release sequence: bold |
| | | KLCSDDNIKAETYIHEISHILNNFEAQELKYNPEIHLVESELKASELKN | Residue numbering: |
| | | VLDVIMNAFHWCSVFMTEELVDKDNNFYAELEEIYDEIYPVISLYNL | His-TEV cleavage sequence: 1-18 |
| | | VRNYVTQKPYSTKKIKLNFGIPTLADGWSKSKEYSNNAIILMRDNLY | Endonuclease MAD7: 19-1281 |
| | | YLGIFNAKNKPDKKIIEGNTSENKGDYKKMIYNLLPGPNKMIPKVFL | Linker: 1282: to 1311 |
| | | SSKTGVETYKPSAYILEGYKQNKHIKSSKDFDITFCHDLIDYFKNCIAI | NLS: 1313-1329 |
| | | HPEWKNFGFDFSDTSTYEDISGFYREVELQGYKIDWTYISEKDIDLL | 2nd His tag: 1330-1335 |
| | | QEKGQLYLFQIYNKDFSKKSTGNDNLHTMYLKNLFSEENLKDIVLK | Hapten binding domain (monoavidin |
| | | LNGEAEIFFRKSSIKNPIIHKKGSILVNRTYEAEEKDQFGNIQIVRKNIP | binding domain): 1336-1491 |
| | | ENIYQELYKYFNDKSDKELSDEAAKLKNVVGHHEAATNIVKDYRY | Linker 2: 1492- 1520 |
| | | TYDKYFLHMPITINFKANKTGFINDRILQYIAKEKDLHVIGIDRGERN | Cell recognition domain 7dl2: 1521-1648 |
| | | LIYVSVIDTCGNFVEQKSFNIVNGYDYQIKLKQQEGARQIARKEWKEI | Endosomal escape sequence: 1649-1654 |
| | | GKIKEIKEGYLSLVIHEISKMVIKYNAIIAMEDLSYGFKKGRFKVERQ | |
| | | VYQKFETMLINKLNYLVFKDISITENGGLLKGYQLTYIPDKLKNVGH | |
| | | QCGCIFYVPAAYTSKIDPTTGFVNIFKFKDLTVDAKREFIKKFDSIRY | |
| | | DSEKNLFCFTFDYNNFITQNTVMSKSSWSVYTYGVRIKRRFVNGRFS | |
| | | NESDTIDITKDMEKTLEMTDINWRDGHDLRQDIIDYEIVQHIFEIFRLT | |
| | | VQMRNSLSELEDRDYDRLISPVLNENNIFYDSAKAGDALPKDADAN | |
| | | GAYCIALKGLYEIKQITENWKEDGKFSRDKLKISNKDWFDFIQNKRY | |
| | | LGGGGSGGGGSGGGGSGGGGSGGGGSGGGGSTSPKKKRKVEDPK | |
| | | KKRKVHHHHHHEFASAEAGITGTWYNQHGSTFTVTAGADGNLT | |
| | | GQYENRAQGTGCQNSPYTLTGRYNGTKLEWRVEWNNSTENCH | |
| | | SRTEWRGQYQGGAEARINTQWNLTYEGGSGPATEQGQDTFTK | |
| | | VKPSAASGSDYKDDDDKKRKRKCRYPIGIDVRWHFSRRSCTGGG | |
| | | GSGGGGSGGGGSGGGGSGGGGSGGSQVQLQESGGGLVQPGGSLRLS | |
| | | CAASGFTFSSYDMSWVRQAPGKGLEWVSGMNSGGGRTYYEDSVK | |
| | | GRFTISRSNAKNTLYLQLNSLKTDDTAMYYCVTSDFAYWGQGTQV | |
| | | TVSSCCCCCC | |
| 82 | MA | GAATTTGCGAGCGCGGAAGCGGGCATTACCGGCACCTGGTATAACCAGC | Monoavidin Haptin binding domain used in |
| | (monoavidin) | ATGGCAGCACCTTTACCGTGACCGCGGGCGCGGATGGCAACCTGACCGG | fusion proteins herein |
| | Hapten | CCAGTATGAAAACCGCGCGCAGGGCACCGGCTGCCAGAACAGCCCGTA | |
| | binding | TACCCTGACCGGCCGCTATAACGGCACCAAACTGGAATGGCGCGTGGAA | |
| | domain | TGGAACAACAGCACCGAAAACTGCCATAGCCGCACCGAATGGCGCGGC | |
| | (nucleotide | CAGTATCAGGGCGGCGCGGAAGCGCGCATTAACACCCAGTGGAACCTG | |
| | sequence) | ACCTATGAAGGCGGCAGCGGCCCGGCGACCGAACAGGGCCAGGATACC | |
| | | TTTACCAAAGTGAAACCGAGCGCGGCGAGCGGCAGCGATTATAAAGAT | |
| | | GATGATGATAAAAAACGCAAAAGAAAATGCCGATATCCTATTGGCATTG | |
| | | ACGTCAGGTGGCACTTTTCGAGGAGATCATGCACA | |
| 83 | MA | FASAEAGITGTWYNQHGSTFTVTAGADGNLTGQYENRAQGTGCQNSPYTL | Monoavidin Haptin binding domain used in |
| | (monoavidin) | TGRYNGTKLEWRVEWNNSTENCHSRTEWRGQYQGGAEARINTQWNLTYE | fusion proteins herein |
| | Hapten | GGSGPATEQGQDTFTKVKPSAASGSDYKDDDDKKRKRKCRYPIGIDVRWH | |
| | binding | FSRRSCT | |
| | domain | | |
| | (protein | | |
| | sequence) | | |
| 84 | Cas9 7412 | ATGGATAAAAAATACAGCATTGGTCTGGACATTGGCACGAATAG | Residue annotation: |
| | fusion | CGTTGGTTGGGCAGTGATTACCGATGAATACAAAGTCCCGTCGA | Endonuclease (spCas9): 1-4104 |
| | (nucleotide | AAAAATTCAAAGTGCTGGGTAACACCGATCGCCATAGCATTAAG | Linker 1: 4105-4134 |
| | sequence) | AAAAACCTGATCGGTGCGCTGCTGTTTGATTCTGGCGAAACCGCG | NLS: 4135-4182 |
| | | GAAGCAACGCGTCTGAAACGTACCGCACGTCGCCGTTACACGCG | Linker2: 4183-4212 |
| | | CCGTAAAAATCGTATTTGCTATCTGCAGGAAATCTTTAGCAACGA | CRD/7D12: 4213-4593 |
| | | AATGGCGAAAGTCGATGACTCATTTTTCCACCGCCTGGAAGAATC | Endosomal escape sequence: 4594- |
| | | GTTTCTGGTGGAAGAAGATAAAAAACATGAACGTCACCCGATTT | 4614 |
| | | TCGGCAATATCGTTGATGAAGTCGCGTACCATGAAAAATATCCG | Endonuclease: single underline |
| | | ACGATTTACCACCTGCGTAAAAAACTGGTGGATTCTACCGACAA | Linker: italics |
| | | AGCCGATCTGCGCCTGATTTATCTGGCACTGGCTCATATGATCAA | NLS sequence: underlined bold |
| | | ATTTCGTGGTCACTTCCTGATTGAAGGCGACCTGAACCCGGATAA | Linker 2: italics |
| | | TAGTGACGTCGATAAACTGTTTATTCAGCTGGTGCAAACCTATAA | Cell recognition domain: double underline |
| | | TCAGCTGTTCGAAGAAAACCCGATCAATGCAAGTGGTGTTGATG | Endosomal release sequence: bold |
| | | CGAAAGCCATTCTGTCCGCTCGCCTGAGTAAATCCCGCCGTCTGG | |
| | | AAAACCTGATTGCACAGCTGCCGGGTGAAAAGAAAAACGGTCTG | |
| | | TTTGGCAATCTGATCGCTCTGTCACTGGGCCTGACGCCGAACTTT | |
| | | AAATCGAATTTCGACCTGGCAGAAGATGCTAAACTGCAGCTGAG | |
| | | CAAAGATACCTACGATGACGATCTGGACAACCTGCTGGCGCAAA | |
| | | TTGGCGACCAGTATGCCGACCTGTTTCTGGCGGCCAAAAATCTGT | |
| | | CAGATGCCATTCTGCTGTCGGACATCCTGCGCGTGAACACCGAAA | |
| | | TCACGAAAGCGCCGCTGTCAGCCTCGATGATTAAACGCTACGAT | |
| | | GAACATCACCAGGACCTGACCCTGCTGAAAGCACTGGTTCGTCA | |
| | | GCAACTGCCGGAAAAATACAAAGAAATTTTCTTTGACCAAAGTA | |
| | | AAAATGGTTATGCAGGCTACATCGATGGCGGTGCTTCCCAGGAA | |
| | | GAATTCTACAAATTCATCAAACCGATCCTGGAAAAAATGGATGG | |
| | | TACGGAAGAACTGCTGGTGAAACTGAATCGTGAAGATCTGCTGC | |
| | | GTAAACAACGCACCTTTGACAACGGTAGCATTCCGCATCAGATCC | |
| | | ACCTGGGCGAACTGCATGCGATTCTGCGCCGTCAGGAAGATTTTT | |
| | | ATCCGTTCCTGAAAGACAACCGTGAAAAAATCGAAAAAATCCTG | |
| | | ACGTTTCGCATCCCGTATTACGTTGGTCCGCTGGCACGTGGTAAT | |
| | | AGCCGCTTCGCATGGATGACCCGCAAATCTGAAGAAACCATTAC | |
| | | GCCGTGGAACTTTGAAGAAGTGGTTGATAAAGGCGCAAGCGCTC | |
| | | AGTCTTTTATCGAACGTATGACCAATTTCGATAAAAACCTGCCGA | |
| | | ATGAAAAAGTGCTGCCGAAACATTCTCTGCTGTATGAATACTTTA | |
| | | CCGTTTACAACGAACTGACGAAAGTGAAATATGTTACCGAGGGT | |
| | | ATGCGCAAACCGGCGTTTCTGAGTGGCGAACAGAAAAAAGCCAT | |
| | | TGTGGATCTGCTGTTCAAAACCAATCGTAAAGTTACGGTCAAACA | |
| | | GCTGAAAGAAGATTACTTCAAGAAAATTGAATGTTTCGACAGCG | |
| | | TGGAAATTTCTGGTGTTGAAGATCGTTTCAACGCCTCTCTGGGCA | |
| | | CCTATCATGACCTGCTGAAAATCATCAAAGACAAAGATTTTCTGG | |
| | | ATAACGAAGAAAACGAAGACATTCTGGAAGATATCGTGCTGACC | |
| | | CTGACGCTGTTCGAAGATCGTGAAATGATTGAAGAACGCCTGAA | |
| | | AACGTACGCACACCTGTTTGACGATAAAGTTATGAAACAGCTGA | |
| | | AACGCCGTCGCTATACCGGTTGGGGCCGTCTGAGCCGCAAACTG | |
| | | ATTAATGGTATCCGCGATAAACAATCAGGCAAAACGATTCTGGA | |
| | | TTTCCTGAAATCGGACGGCTTTGCCAACCGTAATTTCATGCAGCT | |
| | | GATCCATGACGATTCCCTGACCTTTAAAGAAGACATTCAGAAAG | |
| | | CACAAGTGTCAGGTCAAGGCGATTCGCTGCATGAACACATTGCG | |
| | | AACCTGGCCGGTTCACCGGCTATCAAAAAAGGCATCCTGCAGAC | |
| | | CGTGAAAGTCGTGGATGAACTGGTGAAAGTTATGGGTCGTCACA | |
| | | AACCGGAAAACATTGTTATCGAAATGGCGCGCGAAAATCAGACC | |
| | | ACGCAAAAAGGCCAGAAAAACTCGCGTGAACGCATGAAACGCAT | |
| | | TGAAGAAGGTATCAAAGAACTGGGCAGCCAGATTCTGAAAGAAC | |
| | | ATCCGGTCGAAAACACCCAGCTGCAAAATGAAAAACTGTACCTG | |
| | | TATTACCTGCAAAATGGTCGTGACATGTATGTGGATCAGGAACTG | |
| | | GACATCAACCGCCTGTCTGACTATGATGTCGACCACATTGTGCCG | |
| | | CAGAGCTTTCTGAAAGACGATTCTATCGATAACAAAGTTCTGACC | |
| | | CGTAGTGATAAAAACCGCGGCAAAAGCGACAATGTCCCGTCTGA | |
| | | AGAAGTTGTGAAGAAAATGAAAAACTACTGGCGTCAACTGCTGA | |
| | | ATGCGAAACTGATTACGCAGCGTAAATTCGATAACCTGACCAAA | |
| | | GCGGAACGCGGCGGTCTGTCCGAACTGGATAAAGCCGGTTTTAT | |
| | | CAAACGTCAACTGGTTGAAACCCGCCAGATTACGAAACATGTCG | |
| | | CCCAGATCCTGGATTCACGCATGAACACGAAATACGACGAAAAC | |
| | | GATAAACTGATCCGTGAAGTCAAAGTGATCACCCTGAAAAGTAA | |
| | | ACTGGTTTCCGATTTCCGTAAAGACTTTCAGTTCTACAAAGTCCG | |
| | | CGAAATTAACAATTACCATCACGCACACGATGCTTATCTGAATGC | |
| | | AGTGGTTGGTACCGCTCTGATCAAAAAATATCCGAAACTGGAAA | |
| | | GCGAATTTGTGTATGGCGATTACAAAGTCTATGACGTGCGCAAA | |
| | | ATGATTGCGAAATCCGAACAGGAAATCGGCAAAGCGACCGCCAA | |
| | | ATACTTTTTCTATTCAAACATCATGAACTTTTTCAAAACCGAAATT | |
| | | ACGCTGGCAAATGGTGAAATTCGTAAACGCCCGCTGATCGAAAC | |
| | | CAACGGTGAAACGGGCGAAATTGTGTGGGATAAAGGCCGTGACT | |
| | | TCGCGACCGTTCGCAAAGTCCTGTCGATGCCGCAAGTGAATATCG | |
| | | TGAAGAAAACCGAAGTGCAGACGGGCGGTTTTAGTAAAGAATCC | |
| | | ATCCTGCCGAAACGTAACAGCGATAAACTGATTGCGCGCAAAAA | |
| | | AGATTGGGACCCGAAAAAATACGGCGGTTTTGATAGTCCGACGG | |
| | | TTGCATATTCCGTCCTGGTCGTGGCTAAAGTCGAAAAAGGTAAAA | |
| | | GTAAAAAACTGAAATCCGTGAAAGAACTGCTGGGCATTACCATC | |
| | | ATGGAACGTAGCTCTTTTGAGAAAAACCCGATTGACTTCCTGGAA | |
| | | GCCAAAGGTTACAAAGAAGTGAAAAAAGATCTGATCATCAAACT | |
| | | GCCGAAATATAGCCTGTTCGAACTGGAAAACGGCCGTAAACGCA | |
| | | TGCTGGCATCTGCTGGTGAACTGCAGAAAGGCAATGAACTGGCA | |
| | | CTGCCGAGTAAATATGTTAACTTTCTGTACCTGGCTAGCCATTAT | |
| | | GAAAAACTGAAAGGTTCTCCGGAAGATAACGAACAGAAACAACT | |
| | | GTTCGTCGAACAACATAAACACTACCTGGATGAAATCATCGAAC | |
| | | AGATCTCAGAATTCTCGAAACGCGTGATTCTGGCGGATGCCAATC | |
| | | TGGACAAAGTTCTGAGCGCGTATAACAAACATCGTGATAAACCG | |
| | | ATTCGCGAACAGGCCGAAAATATTATCCACCTGTTTACCCTGACG | |
| | | AACCTGGGCGCACCGGCAGCTTTTAAATACTTCGATACCACGATC | |
| | | GACCGTAAACGCTATACCTCAACGAAAGAAGTTCTGGATGCTAC | |
| | | CCTGATTCATCAATCGATCACCGGTCTGTATGAAACGCGTATTGA | |
| | | TCTGAGTCAGCTGGGCGGTGACGGAGGAGGAGGCTCTGGAGGAGGAG | |
| | | GCAGCCCCAAGAAGAAGCGGAAGGTGGAGGACCCCAAGAAGAAGCG | |
| | | GAAAGTGGGAGGAGGAGGCTCTGGAGGAGGAGGCAGCCAGGTGAAACT | |
| | | GGAGGAGAGCGGGGGCGGGAGCGTGCAGACTGGGGGGAGCCTGAGACT | |
| | | GACATGCGCAGCAAGCGGGCGGACAAGCCGGAGCTACGGAATGGGATG | |
| | | GTTCAGGCAGGCACCAGGCAAGGAGAGGGAGTTTGTGAGCGGCATCTC | |
| | | CTGGAGAGGCGATAGCACCGGCTATGCCGACTCCGTGAAGGGCAGGTTC | |
| | | ACCATCAGCCGCGATAATGCCAAGAACACAGTGGACCTGCAGATGAAC | |
| | | TCCCTGAAGCCCGAGGACACCGCAATCTACTATTGCGCAGCAGCAGCAG | |
| | | GCTCCGCCTGGT | |
| | | ACGGCACACTGTACGAGTATGATTACTGGGGCCAGGGCACCCAGGTGAC | |
| | | AGTGAGCTCCGCCCTGGAGTGTTGTTGTTGTTGTTGTTAA | |
| 85 | Cas9 7d12 | MDKKYSIGLDIGTNSVGWAVITDEYKVPSKKFKVLGNTDRHSIKKNLIGAL | Residue annotation: |
| | fusion | LFDSGETAEATRLKRTARRRYTRRKNRICYLQEIFSNEMAKVDDSFFHRLEE | Endonuclease (spCas9): 1-1368 |
| | (protein | SFLVEEDKKHERHPIFGNIVDEVAYHEKYPTIYHLRKKLVDSTDKADLRLIY | Linker 1: 1369-1378 |
| | sequence) | LALAHMIKFRGHFLIEGDLNPDNSDVDKLFIQLVQTYNQLFEENPINASGVD | NLS: 1379-1394 |
| | | AKAILSARLSKSRRLENLIAQLPGEKKNGLFGNLIALSLGLTPNFKSNFDLAE | Linker2: 1395-1404 |
| | | DAKLQLSKDTYDDDLDNLLAQIGDQYADLFLAAKNLSDAILLSDILRVNTEI | CRD/7D12: 1405-1531 |
| | | TKAPLSASMIKRYDEHHQDLTLLKALVRQQLPEKYKEIFFDQSKNGYAGYI | Endosomal escape sequence: 1532- |
| | | DGGASQEEFYKFIKPILEKMDGTEELLVKLNREDLLRKQRTFDNGSIPHQIH | 1537 |
| | | LGELHAILRRQEDFYPFLKDNREKIEKILTFRIPYYVGPLARGNSRFAWMTR | Endonuclease: single underline |
| | | KSEETITPWNFEEVVDKGASAQSFIERMTNFDKNLPNEKVLPKHSLLYEYFT | Linker: italics |
| | | VYNELTKVKYVTEGMRKPAFLSGEQKKAIVDLLFKTNRKVTVKQLKEDYF | NLS sequence: underlined bold |
| | | KKIECFDSVEISGVEDRFNASLGTYHDLLKIIKDKDFLDNEENEDILEDIVLTL | Linker 2: italics |
| | | TLFEDREMIEERLKTYAHLFDDKVMKQLKRRRYTGWGRLSRKLINGIRDK | Cell recognition domain: double underline |
| | | QSGKTILDFLKSDGFANRNFMQLIHDDSLTFKEDIQKAQVSGQGDSLHEHIA | Endosomal release sequence: bold |
| | | NLAGSPAIKKGILQTVKVVDELVKVMGRHKPENIVIEMARENQTTQKGQK | |
| | | NSRERMKRIEEGIKELGSQILKEHPVENTQLQNEKLYLYYLQNGRDMYVDQ | |
| | | ELDINRLSDYDVDHIVPQSFLKDDSIDNKVLTRSDKNRGKSDNVPSEEVVK | |
| | | KMKNYWRQLLNAKLITQRKFDNLTKAERGGLSELDKAGFIKRQLVETRQIT | |
| | | KHVAQILDSRMNTKYDENDKLIREVKVITLKSKLVSDFRKDFQFYKVREIN | |
| | | NYHHAHDAYLNAVVGTALIKKYPKLESEFVYGDYKVYDVRKMIAKSEQEI | |
| | | GKATAKYFFYSNIMNFFKTEITLANGEIRKRPLIETNGETGEIVWDKGRDFA | |
| | | TVRKVLSMPQVNIVKKTEVQTGGFSKESILPKRNSDKLIARKKDWDPKKYG | |
| | | GFDSPTVAYSVLVVAKVEKGKSKKLKSVKELLGITIMERSSFEKNPIDFLEA | |
| | | KGYKEVKKDLIIKLPKYSLFELENGRKRMLASAGELQKGNELALPSKYVNF | |
| | | LYLASHYEKLKGSPEDNEQKQLFVEQHKHYLDEIIEQISEFSKRVILADANL | |
| | | DKVLSAYNKHRDKPIREQAENIIHLFTLTNLGAPAAFKYFDTTIDRKRYTST | |
| | | KEVLDATLIHQSITGLYETRIDLSQLGGDGGGGSGGGGSPKKKRKVEDPKK | |
| | | KRKVGGGGSGGGGSQVKLEESGGGSVQTGGSLRLTCAASGRTSRSYGMG | |
| | | WFRQAPGKEREFVSGISWRGDSTGYADSVKGRFTISRDNAKNTVDLQMNS | |
| | | LKPEDTAIYYCAAAAGSAWYGTLYEYDYWGQGTQVTVSSALECCCCCC | |
| 86 | Cas9(NLS)- | ATGGATAAAAAATACAGCATTGGTCTGGACATTGGCACGAATAG | Residue annotation (translated protein |
| | Monoavidin- | CGTTGGTTGGGCAGTGATTACCGATGAATACAAAGTCCCGTCGA | residues): |
| | GS | AAAAATTCAAAGTGCTGGGTAACACCGATCGCCATAGCATTAAG | Endonuclease (SpCas9): 1-4104 |
| | linker- | AAAAACCTGATCGGTGCGCTGCTGTTTGATTCTGGCGAAACCGCG | Linker1: 4105-4134 |
| | 7D12 | GAAGCAACGCGTCTGAAACGTACCGCACGTCGCCGTTACACGCG | Monoavidin haptin binding protein: |
| | (nucleotide | CCGTAAAAATCGTATTTGCTATCTGCAGGAAATCTTTAGCAACGA | 4135-4605 |
| | sequence) | AATGGCGAAAGTCGATGACTCATTTTTCCACCGCCTGGAAGAATC | NLS: 4606-4653 |
| | | GTTTCTGGTGGAAGAAGATAAAAAACATGAACGTCACCCGATTT | Linker2: 4654-4684 |
| | | TCGGCAATATCGTTGATGAAGTCGCGTACCATGAAAAATATCCG | CRD/7D12: 4685-5064 |
| | | ACGATTTACCACCTGCGTAAAAAACTGGTGGATTCTACCGACAA | Endosomal escape sequence: 5065-5085 |
| | | AGCCGATCTGCGCCTGATTTATCTGGCACTGGCTCATATGATCAA | Endonuclease: single underline |
| | | ATTTCGTGGTCACTTCCTGATTGAAGGCGACCTGAACCCGGATAA | Linker 1: italics |
| | | TAGTGACGTCGATAAACTGTTTATTCAGCTGGTGCAAACCTATAA | Hapten binding domain: bold |
| | | TCAGCTGTTCGAAGAAAACCCGATCAATGCAAGTGGTGTTGATG | NLS: underlined bold |
| | | CGAAAGCCATTCTGTCCGCTCGCCTGAGTAAATCCCGCCGTCTGG | Linker 2: italics |
| | | AAAACCTGATTGCACAGCTGCCGGGTGAAAAGAAAAACGGTCTG | Cell recognition domain: double underline |
| | | TTTGGCAATCTGATCGCTCTGTCACTGGGCCTGACGCCGAACTTT | Endosomal release sequence: bold |
| | | AAATCGAATTTCGACCTGGCAGAAGATGCTAAACTGCAGCTGAG | |
| | | CAAAGATACCTACGATGACGATCTGGACAACCTGCTGGCGCAAA | |
| | | TTGGCGACCAGTATGCCGACCTGTTTCTGGCGGCCAAAAATCTGT | |
| | | CAGATGCCATTCTGCTGTCGGACATCCTGCGCGTGAACACCGAAA | |
| | | TCACGAAAGCGCCGCTGTCAGCCTCGATGATTAAACGCTACGAT | |
| | | GAACATCACCAGGACCTGACCCTGCTGAAAGCACTGGTTCGTCA | |
| | | GCAACTGCCGGAAAAATACAAAGAAATTTTCTTTGACCAAAGTA | |
| | | AAAATGGTTATGCAGGCTACATCGATGGCGGTGCTTCCCAGGAA | |
| | | GAATTCTACAAATTCATCAAACCGATCCTGGAAAAAATGGATGG | |
| | | TACGGAAGAACTGCTGGTGAAACTGAATCGTGAAGATCTGCTGC | |
| | | GTAAACAACGCACCTTTGACAACGGTAGCATTCCGCATCAGATCC | |
| | | ACCTGGGCGAACTGCATGCGATTCTGCGCCGTCAGGAAGATTTTT | |
| | | ATCCGTTCCTGAAAGACAACCGTGAAAAAATCGAAAAAATCCTG | |
| | | ACGTTTCGCATCCCGTATTACGTTGGTCCGCTGGCACGTGGTAAT | |
| | | AGCCGCTTCGCATGGATGACCCGCAAATCTGAAGAAACCATTAC | |
| | | GCCGTGGAACTTTGAAGAAGTGGTTGATAAAGGCGCAAGCGCTC | |
| | | AGTCTTTTATCGAACGTATGACCAATTTCGATAAAAACCTGCCGA | |
| | | ATGAAAAAGTGCTGCCGAAACATTCTCTGCTGTATGAATACTTTA | |
| | | CCGTTTACAACGAACTGACGAAAGTGAAATATGTTACCGAGGGT | |
| | | ATGCGCAAACCGGCGTTTCTGAGTGGCGAACAGAAAAAAGCCAT | |
| | | TGTGGATCTGCTGTTCAAAACCAATCGTAAAGTTACGGTCAAACA | |
| | | GCTGAAAGAAGATTACTTCAAGAAAATTGAATGTTTCGACAGCG | |
| | | TGGAAATTTCTGGTGTTGAAGATCGTTTCAACGCCTCTCTGGGCA | |
| | | CCTATCATGACCTGCTGAAAATCATCAAAGACAAAGATTTTCTGG | |
| | | ATAACGAAGAAAACGAAGACATTCTGGAAGATATCGTGCTGACC | |
| | | CTGACGCTGTTCGAAGATCGTGAAATGATTGAAGAACGCCTGAA | |
| | | AACGTACGCACACCTGTTTGACGATAAAGTTATGAAACAGCTGA | |
| | | AACGCCGTCGCTATACCGGTTGGGGCCGTCTGAGCCGCAAACTG | |
| | | ATTAATGGTATCCGCGATAAACAATCAGGCAAAACGATTCTGGA | |
| | | TTTCCTGAAATCGGACGGCTTTGCCAACCGTAATTTCATGCAGCT | |
| | | GATCCATGACGATTCCCTGACCTTTAAAGAAGACATTCAGAAAG | |
| | | CACAAGTGTCAGGTCAAGGCGATTCGCTGCATGAACACATTGCG | |
| | | AACCTGGCCGGTTCACCGGCTATCAAAAAAGGCATCCTGCAGAC | |
| | | CGTGAAAGTCGTGGATGAACTGGTGAAAGTTATGGGTCGTCACA | |
| | | AACCGGAAAACATTGTTATCGAAATGGCGCGCGAAAATCAGACC | |
| | | ACGCAAAAAGGCCAGAAAAACTCGCGTGAACGCATGAAACGCAT | |
| | | TGAAGAAGGTATCAAAGAACTGGGCAGCCAGATTCTGAAAGAAC | |
| | | ATCCGGTCGAAAACACCCAGCTGCAAAATGAAAAACTGTACCTG | |
| | | TATTACCTGCAAAATGGTCGTGACATGTATGTGGATCAGGAACTG | |
| | | GACATCAACCGCCTGTCTGACTATGATGTCGACCACATTGTGCCG | |
| | | CAGAGCTTTCTGAAAGACGATTCTATCGATAACAAAGTTCTGACC | |
| | | CGTAGTGATAAAAACCGCGGCAAAAGCGACAATGTCCCGTCTGA | |
| | | AGAAGTTGTGAAGAAAATGAAAAACTACTGGCGTCAACTGCTGA | |
| | | ATGCGAAACTGATTACGCAGCGTAAATTCGATAACCTGACCAAA | |
| | | GCGGAACGCGGCGGTCTGTCCGAACTGGATAAAGCCGGTTTTAT | |
| | | CAAACGTCAACTGGTTGAAACCCGCCAGATTACGAAACATGTCG | |
| | | CCCAGATCCTGGATTCACGCATGAACACGAAATACGACGAAAAC | |
| | | GATAAACTGATCCGTGAAGTCAAAGTGATCACCCTGAAAAGTAA | |
| | | ACTGGTTTCCGATTTCCGTAAAGACTTTCAGTTCTACAAAGTCCG | |
| | | CGAAATTAACAATTACCATCACGCACACGATGCTTATCTGAATGC | |
| | | AGTGGTTGGTACCGCTCTGATCAAAAAATATCCGAAACTGGAAA | |
| | | GCGAATTTGTGTATGGCGATTACAAAGTCTATGACGTGCGCAAA | |
| | | ATGATTGCGAAATCCGAACAGGAAATCGGCAAAGCGACCGCCAA | |
| | | ATACTTTTTCTATTCAAACATCATGAACTTTTTCAAAACCGAAATT | |
| | | ACGCTGGCAAATGGTGAAATTCGTAAACGCCCGCTGATCGAAAC | |
| | | CAACGGTGAAACGGGCGAAATTGTGTGGGATAAAGGCCGTGACT | |
| | | TCGCGACCGTTCGCAAAGTCCTGTCGATGCCGCAAGTGAATATCG | |
| | | TGAAGAAAACCGAAGTGCAGACGGGCGGTTTTAGTAAAGAATCC | |
| | | ATCCTGCCGAAACGTAACAGCGATAAACTGATTGCGCGCAAAAA | |
| | | AGATTGGGACCCGAAAAAATACGGCGGTTTTGATAGTCCGACGG | |
| | | TTGCATATTCCGTCCTGGTCGTGGCTAAAGTCGAAAAAGGTAAAA | |
| | | GTAAAAAACTGAAATCCGTGAAAGAACTGCTGGGCATTACCATC | |
| | | ATGGAACGTAGCTCTTTTGAGAAAAACCCGATTGACTTCCTGGAA | |
| | | GCCAAAGGTTACAAAGAAGTGAAAAAAGATCTGATCATCAAACT | |
| | | GCCGAAATATAGCCTGTTCGAACTGGAAAACGGCCGTAAACGCA | |
| | | TGCTGGCATCTGCTGGTGAACTGCAGAAAGGCAATGAACTGGCA | |
| | | CTGCCGAGTAAATATGTTAACTTTCTGTACCTGGCTAGCCATTAT | |
| | | GAAAAACTGAAAGGTTCTCCGGAAGATAACGAACAGAAACAACT | |
| | | GTTCGTCGAACAACATAAACACTACCTGGATGAAATCATCGAAC | |
| | | AGATCTCAGAATTCTCGAAACGCGTGATTCTGGCGGATGCCAATC | |
| | | TGGACAAAGTTCTGAGCGCGTATAACAAACATCGTGATAAACCG | |
| | | ATTCGCGAACAGGCCGAAAATATTATCCACCTGTTTACCCTGACG | |
| | | AACCTGGGCGCACCGGCAGCTTTTAAATACTTCGATACCACGATC | |
| | | GACCGTAAACGCTATACCTCAACGAAAGAAGTTCTGGATGCTAC | |
| | | CCTGATTCATCAATCGATCACCGGTCTGTATGAAACGCGTATTGA | |
| | | TCTGAGTCAGCTGGGCGGTGACGGAGGAGGAGGCTCTGGAGGAGG | |
| | | AGGCAGCGAATTTGCGAGCGCGGAAGCGGGCATTACCGGCAC | |
| | | CTGGTATAACCAGCATGGCAGCACCTTTACCGTGACCGCGGG | |
| | | CGCGGATGGCAACCTGACCGGCCAGTATGAAAACCGCGCGC | |
| | | AGGGCACCGGCTGCCAGAACAGCCCGTATACCCTGACCGGC | |
| | | CGCTATAACGGCACCAAACTGGAATGGCGCGTGGAATGGAAC | |
| | | AACAGCACCGAAAACTGCCATAGCCGCACCGAATGGCGCGG | |
| | | CCAGTATCAGGGCGGCGCGGAAGCGCGCATTAACACCCAGT | |
| | | GGAACCTGACCTATGAAGGCGGCAGCGGCCCGGCGACCGAA | |
| | | CAGGGCCAGGATACCTTTACCAAAGTGAAACCGAGCGCGGC | |
| | | GAGCGGCAGCGATTATAAAGATGATGATGATAAAAAACGCAA | |
| | | AAGAAAATGCCGATATCCTATTGGCATTGACGTCAGGTGGCA | |
| | | CTTTTCGAGGAGATCATGCACACCCAAGAAGAAGCGGAAGGT | |
| | | GGAGGACCCCAAGAAGAAGCGGAAAGTGGGAGGAGGAGGCTC | |
| | | TGGAGGAGGAGGCAGCCAGGTGAAACTGGAGGAGAGCGGGGGCG | |
| | | GGAGCGTGCAGACTGGGGGGAGCCTGAGACTGACATGCGCAGCA | |
| | | AGCGGGCGGACAAGCCGGAGCTACGGAATGGGATGGTTCAGGCA | |
| | | GGCACCAGGCAAGGAGAGGGAGTTTGTGAGCGGCATCTCCTGGA | |
| | | GAGGCGATAGCACCGGCTATGCCGACTCCGTGAAGGGCAGGTTC | |
| | | ACCATCAGCCGCGATAATGCCAAGAACACAGTGGACCTGCAGAT | |
| | | GAACTCCCTGAAGCCCGAGGACACCGCAATCTACTATTGCGCAG | |
| | | CAGCAGCAGGCTCCGCCTGGTACGGCACACTGTACGAGTATGAT | |
| | | TACTGGGGCCAGGGCACCCAGGTGACAGTGAGCTCCGCCCTGGA | |
| | | GTGTTGTTGTTGTTGTTGTTAA | |
| 87 | Cas9(NLS)- | MDKKYSIGLDIGTNSVGWAVITDEYKVPSKKFKVLGNTDRHSIKKNLIGAL | Residue annotation: |
| | Monoavidin- | LFDSGETAEATRLKRTARRRYTRRKNRICYLQEIFSNEMAKVDDSFFHRLEE | Endonuclease (SpCas9): 1-1368 |
| | GS | SFLVEEDKKHERHPIFGNIVDEVAYHEKYPTIYHLRKKLVDSTDKADLRLIY | Linker1: 1369-13708 |
| | linker- | LALAHMIKFRGHFLIEGDLNPDNSDVDKLFIQLVQTYNQLFEENPINASGVD | Monoavidin haptin binding protein: |
| | 7D12 | AKAILSARLSKSRRLENLIAQLPGEKKNGLFGNLIALSLGLTPNFKSNFDLAE | 1379-1535 |
| | | DAKLQLSKDTYDDDLDNLLAQIGDQYADLFLAAKNLSDAILLSDILRVNTEI | NLS: 1536-1551 |
| | | TKAPLSASMIKRYDEHHQDLTLLKALVRQQLPEKYKEIFFDQSKNGYAGYI | Linker2: 1552-1561 |
| | | DGGASQEEFYKFIKPILEKMDGTEELLVKLNREDLLRKQRTFDNGSIPHQIH | CRD/7D12: 1562-1688 |
| | | LGELHAILRRQEDFYPFLKDNREKIEKILTFRIPYYVGPLARGNSRFAWMTR | Endosomal escape sequence: 1689-1694 |
| | | KSEETITPWNFEEVVDKGASAQSFIERMTNFDKNLPNEKVLPKHSLLYEYFT | Endonuclease: underlined |
| | | VYNELTKVKYVTEGMRKPAFLSGEQKKAIVDLLFKTNRKVTVKQLKEDYF | Linkers: italics |
| | | KKIECFDSVEISGVEDRFNASLGTYHDLLKIIKDKDFLDNEENEDILEDIVLTL | Hapten: plain text |
| | | TLFEDREMIEERLKTYAHLFDDKVMKQLKRRRYTGWGRLSRKLINGIRDK | NLS: bold, italics underlined |
| | | QSGKTILDFLKSDGFANRNFMQLIHDDSLTFKEDIQKAQVSGQGDSLHEHIA | CRD: Bold and underlined |
| | | NLAGSPAIKKGILQTVKVVDELVKVMGRHKPENIVIEMARENQTTQKGQK | EES: Bold |
| | | NSRERMKRIEEGIKELGSQILKEHPVENTQLQNEKLYLYYLQNGRDMYVDQ | |
| | | ELDINRLSDYDVDHIVPQSFLKDDSIDNKVLTRSDKNRGKSDNVPSEEVVK | |
| | | KMKNYWRQLLNAKLITQRKFDNLTKAERGGLSELDKAGFIKRQLVETRQIT | |
| | | KHVAQILDSRMNTKYDENDKLIREVKVITLKSKLVSDFRKDFQFYKVREIN | |
| | | NYHHAHDAYLNAVVGTALIKKYPKLESEFVYGDYKVYDVRKMIAKSEQEI | |
| | | GKATAKYFFYSNIMNFFKTEITLANGEIRKRPLIETNGETGEIVWDKGRDFA | |
| | | TVRKVLSMPQVNIVKKTEVQTGGFSKESILPKRNSDKLIARKKDWDPKKYG | |
| | | GFDSPTVAYSVLVVAKVEKGKSKKLKSVKELLGITIMERSSFEKNPIDFLEA | |
| | | KGYKEVKKDLIIKLPKYSLFELENGRKRMLASAGELQKGNELALPSKYVNF | |
| | | LYLASHYEKLKGSPEDNEQKQLFVEQHKHYLDEIIEQISEFSKRVILADANL | |
| | | DKVLSAYNKHRDKPIREQAENIIHLFTLTNLGAPAAFKYFDTTIDRKRYTST | |
| | | KEVLDATLIHQSITGLYETRIDLSQLGGDGGGGSGGGGSEFASAEAGITGTW | |
| | | YNQHGSTFTVTAGADGNLTGQYENRAQGTGCQNSPYTLTGRYNGTKLEW | |
| | | RVEWNNSTENCHSRTEWRGQYQGGAEARINTQWNLTYEGGSGPATEQGQ | |
| | | DTFTKVKPSAASGSDYKDDDDKKRKRKCRYPIGIDVRWHFSRRSCT [symbol] | |
| | | [symbol] GGGGSGGGGSQVKLEESGGGSVQTGGSLRLTCAASGRT | |
| | | SRSYGMGWFRQAPGKEREFVSGISWRGDSTGYADSVKGRFTISRDNAK | |
| | | NTVDLQMNSLKPEDTAIYYCAAAAGSAWYGTLYEYDYWGQGTQVTVS | |
| | | SALECCCCCC | |

TABLE 7
Example Targeting sequences and gRNAs used to target EML4-ALK gene

| | | | | | | SEQ |
|---|---|---|---|---|---|---|
| | | | | | | ID |
| | | | | SEQ | | NO: |
| | | Sequence | | ID | | Full |
| Target | Guide | (5′-3′) target | RNA | NO: | Full length | length |
| Name | Name | sequence | conversion | RNA | guide (56mer) | guide |
| EML4- | Variant | | | | | |
| ALK | dependent | | | | | |
| EML4- | Variant 1 | CGGCGGTAC | CGGCGGU | 88 | GUCAAAAGACCUUUU | 89 |
| ALK | | ACTTTAGGT | ACACUUU | | UAAUUUCUACUCUUG | |
| | | CCT | AGGUCCU | | UAGAUCGGCGGUACA | |
| | | | | | CUUUAGGUCCU | |
| | Variant 3a | CGGCGGTAC | CGGCGGU | 90 | GUCAAAAGACCUUUU | 91 |
| | | ACTTGGTTG | ACACUUG | | UAAUUUCUACUCUUG | |
| | | ATG | GUUGAUG | | UAGAUCGGCGGUACA | |
| | | | | | CUUGGUUGAUG | |
| EML4- | Variant 3b | CGGCGGTAC | CGGCGGU | 92 | GUCAAAAGACCUUUU | 93 |
| ALK | | ACTTGGCTG | ACACUUG | | UAAUUUCUACUCUUG | |
| | | TTT | GCUGUUU | | UAGAUCGGCGGUACA | |
| | | | | | CUUGGCUGUUU | |
| EML4- | Variant | | | | | |
| ALK | Independent | | | | | |
| EML4- | I1 | CAGCTCCTG | CAGCUCCU | 94 | GUCAAAAGACCUUUU | 95 |
| ALK | | GTGCTTCCG | GGUGCUU | | UAAUUUCUACUCUUG | |
| | | GCG | CCGGCG | | UAGAUCAGCUCCUGG | |
| | | | | | UGCUUCCGGCG | |
| EML4- | I2 | TACTCAGGG | UACUCAG | 96 | GUCAAAAGACCUUUU | 97 |
| ALK | | CTCTGCAGC | GGCUCUGC | | UAAUUUCUACUCUUG | |
| | | TCC | AGCUCC | | UAGAUUACUCAGGGC | |
| | | | | | UCUGCAGCUCC | |
| EML4- | I3 | CTCAGCTTG | CUCAGCUU | 98 | GUCAAAAGACCUUUU | 99 |
| ALK | | TACTCAGGG | GUACUCA | | UAAUUUCUACUCUUG | |
| | | CTC | GGGCUC | | UAGAUCUCAGCUUGU | |
| | | | | | ACUCAGGGCUC | |
| EML4- | I4 | CTGGCAAGA | CUGGCAA | 100 | GUCAAAAGACCUUUU | 101 |
| ALK | | CCTCCTCCA | GACCUCCU | | UAAUUUCUACUCUUG | |
| | | TCA | CCAUCA | | UAGAUCUGGCAAGAC | |
| | | | | | CUCCUCCAUCA | |
| EML4- | I5 | AGGTCACTG | AGGUCAC | 102 | GUCAAAAGACCUUUU | 103 |
| ALK | | ATGGAGGA | UGAUGGA | | UAAUUUCUACUCUUG | |
| | | GGTC | GGAGGUC | | UAGAUAGGUCACUGA | |
| | | | | | UGGAGGAGGUC | |
| EML4- | I6 | CGCGGCACC | CGCGGCAC | 104 | GUCAAAAGACCUUUU | 105 |
| ALK | | TCCTTCAGG | CUCCUUCA | | UAAUUUCUACUCUUG | |
| | | TCA | GGUCA | | UAGAUCGCGGCACCUC | |
| | | | | | CUUCAGGUCA | |
| BRCA | | GCAGGTTCA | GCAGGUU | 106 | GCAGGTTCAGAATTAT | 107 |
| | | GAATTATAG | CAGAAUU | | AGGGGUUUUAGAGCU | |
| | | GG | AUAGGG | | AGAAA | |
| | | | | | UAGCAAGUUAAAAUA | |
| | | | | | AGGCUAGUCCGUUAU | |
| | | | | | CAACUUGAAAAAGUG | |
| | | | | | GCACCGAGUCGGUGC | |
| | | | | | UUU | |
| CXCR4 | | GGGCAAUG | GGGCAAU | 108 | GGGCAATGGATTGGTC | 109 |
| | | GATTGGTCA | GGAUUGG | | ATCC | |
| | | TCC | UCAUCC | | GUUUUAGAGCUAGAA | |
| | | | | | A | |
| | | | | | UAGCAAGUUAAAAUA | |
| | | | | | AGGCUAGUCCGUUAU | |
| | | | | | CAACUUGAAAAAGUG | |
| | | | | | GCACCGAGUCGGUGC | |
| | | | | | UUU | |

In some embodiments, compositions according to the disclosure comprise a gRNA having at least 75% identity, at least 78% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity to any one of SEQ ID NOs: 88-109, or any of the sequences in Table 7.

In some embodiments, the domains within a PNME composition are directly linked by peptide bonds, e.g. expressed as a single fusion polypeptide. In some embodiments, the domains within a PNME composition are linked by bivalent reactive chemical crosslinking agents (e.g. Disuccinimidyl suberate, Sulfosuccinimidyl 4-(N-maleimidomethyl) cyclohexane-1-carboxylate). In some cases, the domains within a PNME composition are linked by expressed protein ligation; example protocols for expressed protein ligation, which typically involves expression of a domain with a C-terminal cysteine followed by an intein sequence, followed by transthioesterification using an N-terminally thiol-linked peptide, can be found in e.g. Berrade et al. Cell Mol Life Sci. 2009 December; 66(24): 3909-3922. In some embodiments, the domains within a PNME composition are linked by any of the linkers described herein. In some embodiments, the PNME domain is located at the N- or C-terminal position of the PSME composition. In some embodiments, the endosome escape domain is located at the N- or C-terminal position of the PSME composition. In some embodiments, the cell recognition domain is located at the N- or C-terminal position of the PSME composition. In some embodiments, the domain structure of the PSME composition is configured such that the total molecular weight of the PSME composition is between 100 kDa and 240 kDa. In some embodiments the PSME composition is between 100 kDa and 200 kDa. In some embodiments, the domain structure of the PSME composition is configured such that the average hydrodynamic radius of the PSME composition in solution is less than 100 nm, less than 90 nm, less than 80 nm, less than 70 nm, or less than 60 nm.

In some embodiments, PSME-CRD conjugates according to the present disclosure comprise particular protein sequences. In some embodiments, PSME-CRD conjugates comprise a protein sequence having at least 75% identity, at least 78% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity to any one of SEQ ID NOs: 16-26, 44, 46, 48, 50, 52, 54, 56, 58, 60, 61-65, 67, 69, 71, 73, 75, 77, 79, 81, 83, 85, 87, or a variant thereof. In some embodiments, PSME-CRD conjugates comprise a protein sequence substantially identical to any one of SEQ ID NOs: 16-26, 44, 46, 48, 50, 52, 54, 56, 58, 60, 61-65, 67, 69, 71, 73, 75, 77, 79, 81, 83, 85, 87, or a variant thereof. In some embodiments, PSME-CRD conjugates comprise a protein sequence having at least 75% identity, at least 78% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity to any one of SEQ ID NOs 67, 69, 71, 73, 75, 77, 79, 81, 83, 85, 87, or a variant thereof. In some embodiments, PSME-CRD conjugates comprise a protein sequence substantially identical to any one of SEQ ID NOs: 67, 69, 71, 73, 75, 77, 79, 81, 83, 85, 87, or a variant thereof. In some embodiments, PSME-CRD conjugates comprise a PSME protein sequence having at least 75% identity, at least 78% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity to any one of SEQ ID NOs: 44, 46, 48, 50, or 52, or a variant thereof. In some embodiments, PSME-CRD conjugates comprise a PSME protein sequence substantially identical to any one of SEQ ID NOs: 44, 46, 48, 50, or 52.

Included in the current disclosure are variants of any of the enzymes or proteins described herein with one or more conservative amino acid substitutions. Such conservative substitutions can be made in the amino acid sequence of a polypeptide without disrupting the three-dimensional structure or function of the polypeptide. Conservative substitutions can be accomplished by substituting amino acids with similar hydrophobicity, polarity, and R chain length for one another. Additionally or alternatively, by comparing aligned sequences of homologous proteins from different species, conservative substitutions can be identified by locating amino acid residues that have been mutated between species (e.g. non-conserved residues without altering the basic functions of the encoded proteins. Such conservatively substituted variants may include variants with at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity any one of the systems described herein. In some embodiments, such conservatively substituted variants are functional variants. Such functional variants can encompass sequences with substitutions such that the activity of critical active site residues of the endonuclease are not disrupted. In some embodiments, a functional variant of any of the systems described herein lack substitution of at least one of the conserved or functional residues described herein. In some embodiments, a functional variant of any of the systems described herein lacks substitution of all of the conserved or functional residues described herein.

Conservative substitution tables providing functionally similar amino acids are available from a variety of references (see, for example, Creighton, Proteins: Structures and Molecular Properties (W H Freeman & Co.; 2nd Edition (December 1993)). The following eight groups each contain amino acids that are conservative substitutions for one another:
a. Alanine (A), Glycine (G);
b. Aspartic acid (D), Glutamic acid (E);
c. Asparagine (N), Glutamine (Q);
d. Arginine (R), Lysine (K);
e. Isoleucine (I), Leucine (L), Methionine (M), Valine (V);
f. Phenylalanine (F), Tyrosine (Y), Tryptophan (W);
g. Serine (S), Threonine (T); and
h. Cysteine (C), Methionine (M).

In some cases, PSME-CRD conjugates according to the present disclosure further comprise a specific guide polynucleotide. In some embodiments, the guide polynucleotide comprises a sequence having at least 75% identity, at least 78% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity to any one of SEQ ID NOs: 43-60, or a variant thereof.

In some cases, PSME compositions described herein are expressed using recombinant expression systems.

Accordingly, in some aspects the present disclosure provides for a vector comprising a nucleotide sequence encoding a cell recognition domain, an endosome escape domain, and a polynucleotide-modifying enzyme domain. In some cases, the vector further comprises a hapten-binding domain within the same ORF as the cell recognition domain, endosome escape domain, and polynucleotide-modifying enzyme domain. A “vector” is a nucleic acid sequence capable of transferring other operably-linked heterologous or recombinant nucleic acid sequences to target cells. In some examples, a vector is a minicircle, plasmid, yeast artificial chromosome (YAC), bacterial artificial chromosome (BAC), cosmid, phagemid, bacteriophage genome, or baculovirus genome. Suitable vectors also include vectors derived from bacteriophages or plant, invertebrate, or animal (including human) viruses such as CELiD vectors, adeno-associated viral vectors (e.g. AAV1, AAV2, AAV4, AAVS, AAV6, AAV7, AAV8, AAV9, or pseudotyped combinations thereof such as AAV2/5, AAV2/2, AAV-DJ, or AAV-DJ8), retroviral vectors (e.g. MLV or self-inactivating or SIN versions thereof, or pseudotyped versions thereof), herpesviral (e.g. HSV- or EBV-based), lentiviral vectors (e.g. HIV-, FIV-, or EIAV-based, or pseudotyped versions thereof), adenoviral vectors (e.g. Ad5-based, including replication-deficient, replication-competent, or helper-dependent versions thereof), or baculoviral vectors (which are suitable to transfect insect cells as described herein). In some embodiments, a vector is a replication competent viral-derived vector.

Accordingly, in some aspects the present disclosure also provides for host cells comprising any of the vectors described herein.

In some embodiments, the host cells are animal cells. The term “animal cells” encompasses any animal cell, including but not limiting to, invertebrate, non-mammalian vertebrate (e.g., avian, reptile, and amphibian), and mammalian cells. A number of mammalian cell lines are suitable host cells for recombinant expression of polypeptides of interest. Mammalian host cell lines include, for example, COS, PER.C6, TM4, VERO076, MDCK, BRL-3A, W138, Hep G2, MMT, MRC 5, FS4, CHO, 293T, A431, 3T3, CV-1, C3H10T1/2, Colo205, 293, HeLa, L cells, BHK, HL-60, FRhL-2, U937, HaK, Jurkat cells, Rat2, BaF3, 32D, FDCP-1, PC12, M1x, murine myelomas (e.g., SP2/0 and NSO) and C2C12 cells, as well as transformed primate cell lines, hybridomas, normal diploid cells, and cell strains derived from in vitro culture of primary tissue and primary explants. Any eukaryotic cell that is capable of expressing recombinant and/or transgenic proteins may be used in the disclosed cell culture methods. Numerous cell lines are available from commercial sources such as the American Type Culture Collection (ATCC). The host cells can be CHO cells. In some embodiments, the host cells are bacterial cells suitable for protein expression such as derivatives of E. coli K12 strain. In some embodiments, the host cells comprise plant cells into which genes have been introduced by a vector single-stranded RNA virus tobacco mosaic virus. “Host cells” can be insect cells which are utilized for the production of large quantities of the polypeptides according to the disclosure. In some embodiments, the baculovirus system (which provides all the advantages of higher eukaryotic organisms) is utilized. The host cells for the baculovirus system include, but are not limited to Spodoptera frugiperda ovarian cell lines SF9 and SF21 and the Trichoplusia ni egg-derived cell line High Five.

In some embodiments, PNME compositions described herein are delivered to cells (e.g. in vitro or in a patient) via a liquid composition or dose form of particular design. The liquid composition may comprise sterile water alongside a biologically compatible buffering agent and electrolytes to ensure the composition is isotonic. Because compositions as described herein do not require chemical transfection agents to enter cells, in some cases, a liquid formulation for delivery does not comprise a PEI, PEG, PAMAN, or sugar (dextran) derivative polymer comprising more than three subunits.

In some aspects, the present disclosure provides for kits for editing a gene in a cell. Kits can comprise instructions for performing gene editing. In some embodiments, kits as described herein comprise any of the vectors described herein alongside a donor DNA polynucleotide. In some cases, the kits further comprise a suitable guide RNA (when the PNME is a CRISPR enzyme).

### EXAMPLES

### Example 1. Microscopic Examination of PNME-CRD Uptake by Cultured Cells

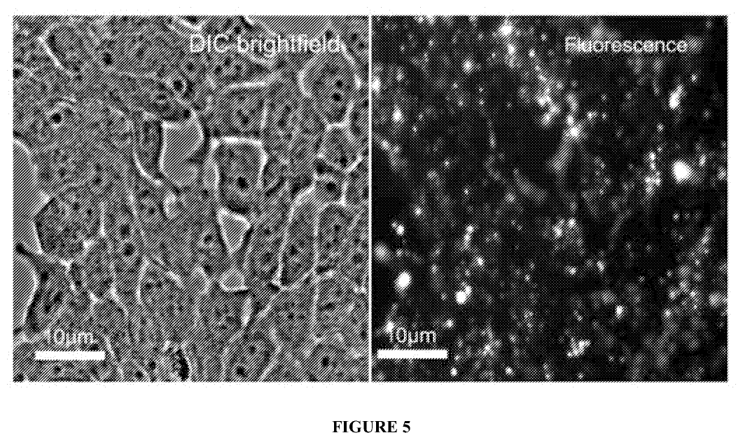

A PNME-CRD fusion construct was generated by fusing DNA encoding Cas9(NLS) to DNA encoding 7D12, an EGFR-binding heavy chain variable domain only antibody (see e.g. Roovers RC et al. Int J Cancer. 2011; 129:2013-2024). The Cas9(NLS)-7D12 fusion protein (comprising SEQ ID NO: 44 endonuclease, SEQ ID No: 64 linker, SEQ ID NO: 54 cell recognition domain, and SEQ ID NO: 24 endosomal escape sequence, whole sequence of SEQ ID NO: 84 for nucleotide and SEQ ID NO: 85 for protein) was recombinantly expressed and then conjugated to tetramethylrhodamine (TAMRA) to form a TAMRA-labeled PNME-CRD complex. Cultured A549 cells were incubated in cell culture medium for 48 hr with the TAMRA-labeled PNME-CRD complex followed by washing with cell culture medium. FIG. 5 shows 20× DIC-brightfield (left) and 20× epifluorescence (right) photomicrographs of the A549 cells after treatment and washing. Residual fluorescence is localized to punctate spots within cells, demonstrating cellular uptake of the PNME-CRD composition.

### Example 2. Efficiency of Indel Formation by a PNME-CRD Composition

The Cas9(NLS)-7D12 PNME-CRD fusion protein from Example 1 was mixed with a gRNA (targeting sequence 5′-GCAGGUUCAGAAUUAUAGGG-3′, in SpyCas9 sgRNA backbone; targeting sequence SEQ ID NO: 106 and full-length gRNA SEQ ID NO: 107) directed against Exon 6 of the BRCA1 locus (chr17: 43, 104, 149-43, 104, 207) and then administered to cultured A549 cells. The cells were incubated for 48 hours and then washed three times with PBS. Exon 6 of the BRCA1 gene was amplified by PCR on genomic DNA extracted from the cells. Indel formation was assessed by annealing PCR products from control cells and edited cells followed by cleavage of mismatched DNA by T7 endonuclease. Vouillot L et al G3 (Bethesda). 2015; 5(3):407-415.

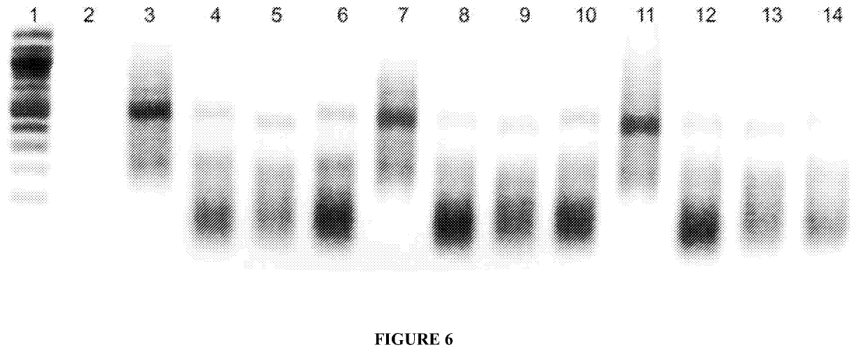

FIG. 6 demonstrates that the Cas9(NLS)-7D12 PNME-CRD composition can cleave genomic DNA. Mismatches due to internal deletions (indels) generated by successful editing allow cleavage by T7 endonuclease to generate products of a smaller size (100-300 bp) than the original PCR amplicon (500 bp). The percentage of Cas9(NLS)-7D12 treatments resulting in indel formation was 30%±5%.

### Example 3. Gene Editing via Homologous Recombination by a PNME-Hapten BD-CRD Composition

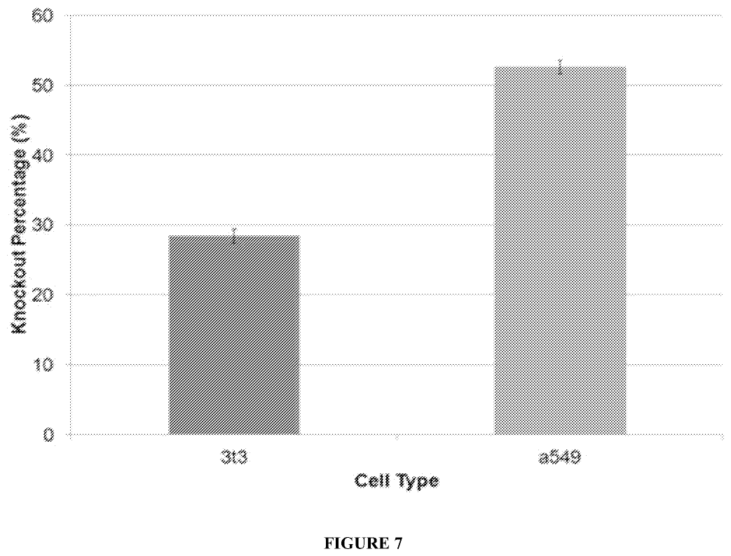

A Cas9(NLS)-Monoavidin-GS linker-7D12 fusion protein (SEQ ID NO: 86 for nucleotide and SEQ ID NO: 87 for protein) was recombinantly expressed and mixed with a gRNA (5′-GGGCAAUGGAUUGGUCAUCC-3′, in an SpyCas9 sgRNA backbone, SEQ ID NO: 108 for targeting sequence, SEQ ID NO: 109 for full gRNA)directed against the CXCR4 locus (chr2:136115548-136115966) and a biotin-labeled donor oligonucleotide. The donor nucleotide (SEQ ID NO: 110 with a 5′ biotin modification) had a TAGTGATAG insert sequence flanked by a 91 nucleotide 5′ homology arm and a 36 nucleotide 3′ homology arm. The two homology arms were designed to hybridize to sequences flanking the expected CXCR4 cut site and result in a TAGTGATAG (repeat stop codon) insertion which truncates mRNA translation, in addition to separating PAM and seed sequence of the target to preventing re-cutting. CXCR4 expression by cultured A549 or NIH 3T3 cells treated with the PNME-Hapten BD-CRD composition was measured by an ELISA assay performed directly on the cells using a primary mouse CXCR4 monoclonal antibody, an HRP-conjugated anti-mouse mAb secondary antibody, and chromophoric detection with DAB, as described by Kohl and Ascoli, Cold Spring Harbor Protocols, 2017 (doi:10.1101/pdb.prot093732, available at https://cshprotocols.cshlp.org/content/2017/5/pdb.prot093732.abstract). FIG. 7 depicts remaining cell surface CXCR4 expression in 3T3 or A549 cells treated with the PNME composition. A substantial decrease in CXCR4 expression indicating successful gene editing was observed in both cell lines.

SEQ ID NO: 110 used for the donor nucleotide is provided below:

| SEQ ID | |
|---|---|
| NO: | Nucleotide sequence (5′ to 3′) |
| 110 | GTGATGACAAAGAGGAGGTCGGCCACTGACAGGTGCAGCCT |
| | GTACTTGTCCGTCATGCTTCTCAGTTTCTTCTGGTAACCCA |
| | TGACCAGGATAGTGATAGTGACCAATCCATTGCCCACAATG |
| | CCAGTTAAGAAGA |

### Example 4. Eukaryotic Expression of PNME-CRD Molecules

The MDL4 (md7-7d-L4, SEQ ID NO: 76 for nucleotide and SEQ ID NO: 77 for protein) PNME-CRD was expressed using an Sf9 insect cell-based (e.g. baculovirus) eukaryotic expression system. MDL4 has an N-terminal IL-2 signal sequence followed by a Mad7 endonuclease domain, a (GGGGS)₄ linker, a 7D12 cell recognition domain for EGFR binding, an NLS, a TEV-cleavage site, and a C-terminal polyhistidine endosomal escape sequence. The nucleotide sequence encoding MDL4 with an N-terminal IL-2 secretion tag (to facilitate secretion of the protein into medium) was codon-optimized for insect cell expression and inserted into a pFastbac vector for the baculovirus expression system. Subsequently, this vector was transformed into DH10Bac E.coli MAX Efficiency (Thermofisher) E.coli, which contained a baculovirus shuttle vector (bMON14272) and a helper plasmid (pMON7142), allowing site-specific recombination of pFastBac and bMON14272 leading to bacmid formation containing MDL4. The bacmid containing MDL4 was then transfected into SF9 cells using Epifect (Thermofisher) for P0 baculovirus generation. Subsequent passage baculovirus generation was performed by re-infecting untransfected SF9 to create a scaled viral P1 stock and initiate protein production in the cells. P1 was used to infect non transfected SF9 cells at a multiplicity of infection of 0.1 and cultured at 28° C. for 6 days in SF900+10% fetal bovine serum rotating at 180 rpm. After infection, medium was harvested and cells removed by centrifugation at 6 days, and protease inhibitor cocktail minus EDTA was added to the medium.

The protease-inhibitor stabilized medium was then passed through a Nickel capture column (IMAC-Ni NTA. volume 1-4 ml depending on volume of media). Media was re-circulated through the NiNTA column overnight at 4° C. Medium was then removed and the column washed with 10 column volumes of PBS+5 mM imidazole to remove non-specifically bound proteins. Elution of protein was performed with 500 mM Imidazole. Fractions were evaluated by SDS page gel & coomassie protein staining. Addition of TAMRA dye was accomplished by incubation with protein of a N-succinimide ester modified TAMRA dye, at pH8 at 4° C. overnight. Size exclusion chromatography was used to remove unreacted dye and purify fluorescently labelled protein conjugate.

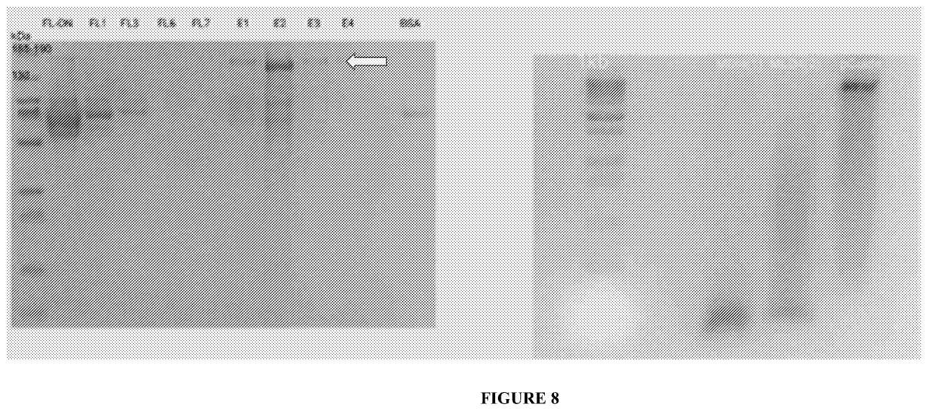

Purification and activity validation of MDL4 secreted into the medium by Sf9 cells is illustrated in FIG. 8. The left panel of FIG. 8 illustrates the isolation of secreted MDL4 from Sf9 media by IMAC affinity chromatography, as detected on a Coomassie (total protein) stained SDS-page gel. The isolated MDL4 for further purified by size-exclusion chromatography (SEC) and then tested in an in vitro cleavage assay as illustrated in the right panel of FIG. 8. MDL4 complexed with a guide RNA targeting a GFP sequence was able to cleave the pGuide plasmid. A no-gRNA control established the specificity of cleavage.

### Example 5. The EGFR-Binding Domain of the MDL4 PNME-CRD Fusion Protein Mediates Specific Uptake by Cells EGFR-Positive Cells.

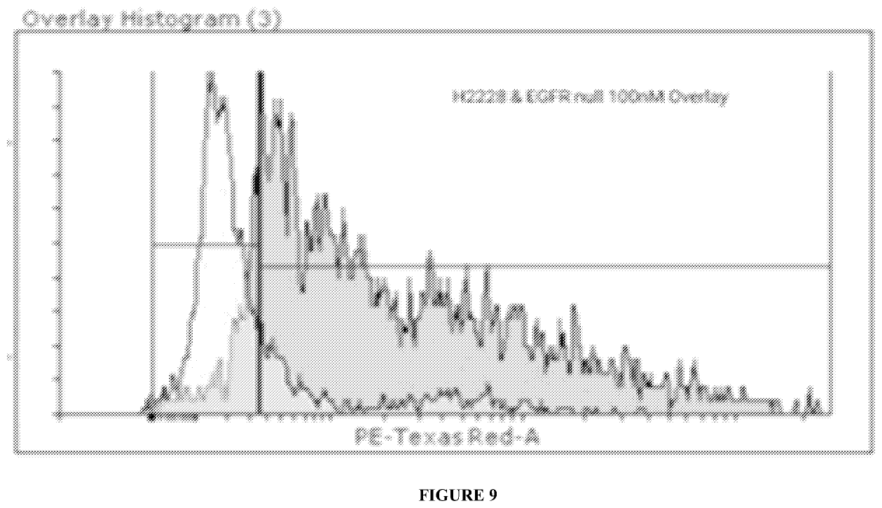

The specificity of MDL4 uptake was demonstrated in two flow cytometry experiments using TAMRA-labelled MDL4. The first experiment compared uptake into EGFR-positive H2228 cells versus EGFR-null A549 cells. 50000 cells of each cell line were incubated with 100nM of MDL4-TAMRA for 45 mins at room temperature, washed with PBS, fixed with 70% ethanol, and then suspended in 10%FBS/PBS for analysis by flow cytometry. The results are shown in FIG. 9, which illustrates an overlay of FACS traces of EGFR-positive cells (grey trace) and EGFR-negative cells (white trace). To quantify the differences between specific and non-specific uptake, Table 8 shows the mean MDL4-TAMRA intensity in the two cell populations and the percentage of cells with fluorescence above the threshold indicated by the vertical bar in FIG. 9. The ˜10-fold increase in MDL4-TAMRA uptake by the EGFR-positive H2228 cells indicates specific uptake mediated by the EGFR targeted CRD. The low level of uptake into the EGFR-null A549 cells may represent non-specific uptake by pinocytosis.

TABLE 8
Quantitation of Distinct Endocytic populations in EGFR-
positive (H2228) and EGFR-negative (A549) cells.

| | EGFR-null A549 cells | H2228 cells |
|---|---|---|
| Mean intensity | 1,139 | 11,415 |
| MDL4-TAMRA high cells | 24.9% | 89.4% |

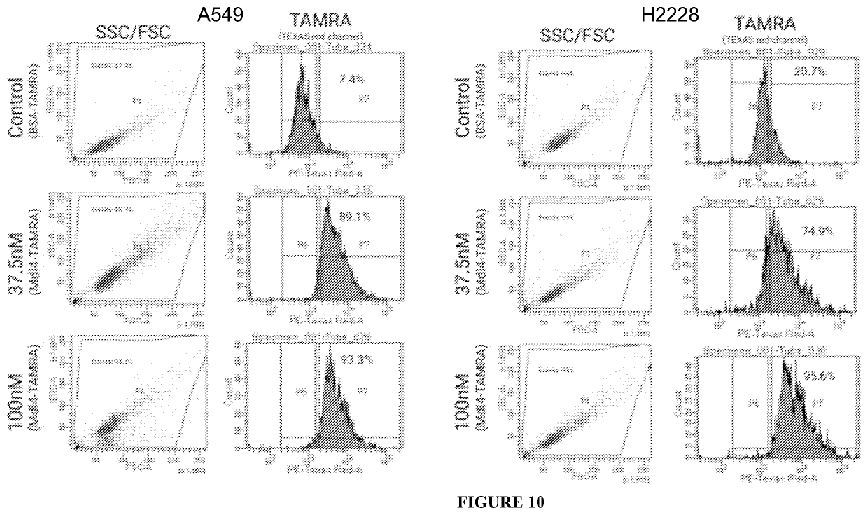

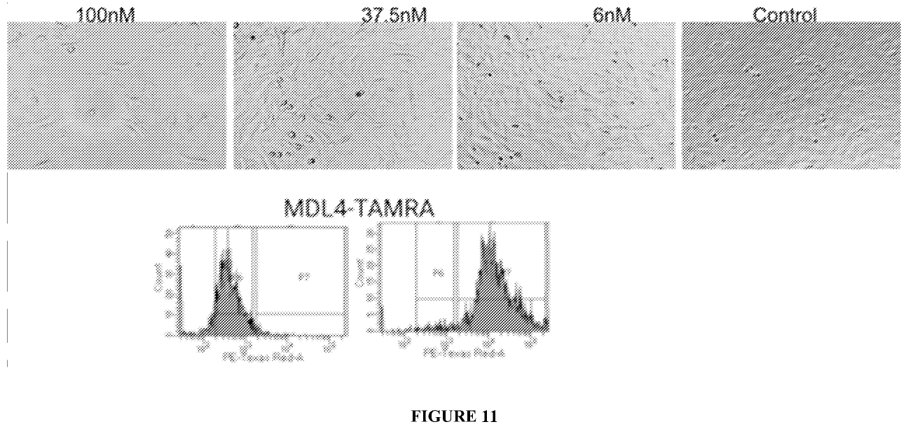

The second experiment compared the uptake of MDL4-TAMRA versus BSA-TAMRA by H2228 cells and EGFR-positive A549 cells. 100 nM BSA-TAMRA and 37.5 nM or 100 nM MDL4-TAMRA were incubated with 50,000 A549 or H2228 cells (both EGFR-positive) for 45 mins at room temperature. The cells were washed with PBS, fixed in 70% ethanol, suspended in 10% FBS/PBS, and then analyzed by flow cytometry, as shown in FIG. 10. The results show low, non-specific uptake of BSA-TAMRA and higher, dose-dependent uptake of MDL4-TAMRA. In summary, the specificity of MDL4 uptake by EGFR-positive H2228 cells was demonstrated by reduced uptake in the absence of EGFR expression (FIG. 9) or in the absence of the 7D12 EGFR binding domain (FIG. 10).

### Example 6. MDL4 Inhibits Cell Proliferation When Complexed With a gRNA Targeting the EML4-ALK Oncogenic Fusion

The EML4-ALK oncogenic fusion is an established therapeutic target for lung cancer, and is formed by fusion between EML4 (echinoderm microtubule associated protein-like 4), a microtubule-associated protein, and ALK (anaplastic lymphoma kinase), a tyrosine kinase receptor belonging to the insulin receptor superfamily. Fusion of EML4 to the kinase domain of ALK results in abnormal signaling and consequently increased cell growth, proliferation, and cell survival. Sabir et al, Cancers (Basel) 2017, 9(9):118. The H2228 cell line is a human lung (non small cell) carcinoma cell line carrying the ELM4-ALK translocation.

To investigate the effects of EML4-ALK editing in vivo, MDL4-TAMRA was complexed with I2 gRNA (SEQ ID NO: 96 for targeting sequence and SEQ ID NO: 97 for full-length gRNA), a gRNA targeting a sequence in the kinase domain of ALK. Application of MDL4-TAMRA/I2 to H2228 cells caused a dose-dependent growth inhibition, as illustrated in the upper panel of FIG. 11. At the highest dose of MDL4-TAMRA/I2 (100 nM), there was an 80% reduction in cell confluence after 72 hours. No growth inhibition was observed when H2228 cells were treated with 100 nM MDL4-TAMRA without a gRNA, demonstrating specificity. Dose dependent uptake of MDL4-TAMRA/I2 in this experiment was confirmed by flow cytometry, as illustrated in the lower panel of FIG. 11, which demonstrates MDL4-TAMRA/I2 uptake into over 90% of the H2228 cells treated with the 100 mM dose. The 100 nM dose was therefore selected for further studies.

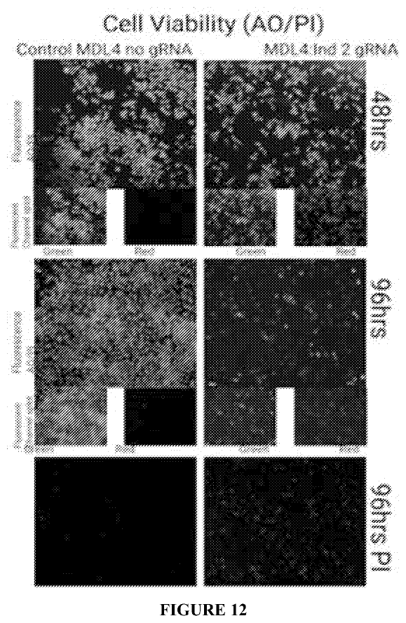

The viability of H2228 cells after MDL4/I2 treatment was investigated by staining with Acridine Orange and Propidium iodide. Acridine Orange is a cell-permeant nucleic acid binding dye that emits green fluorescence when bound to dsDNA and red fluorescence when bound to ssDNA or RNA. Propidium iodide is a red fluorescent dye that stains dead cells. In this AO/PI staining scheme, live cells are stained bright green, where apoptotic cells are orange and fully necrotic cells are stained red as membrane integrity is broken allowing propidium iodide to freely enter the cells. MDL4/I2 is toxic to H2228 cells, as shown in FIG. 12. After 48 hours of treatment, there was a reduction in the number of viable cells stained with Acridine Orange compared to control H2228 cells treated with MDL4 without a gRNA, and an increase in dead cells stained with Propidium iodide. Full progression to apoptosis and necrosis was observed 96 hours after MDL4/I2 treatment, with over 90% of cells having been killed, whereas the control H2228 cells continued growing to confluence.

### Example 7. Specific Toxicity of MDL4 Complexed with gRNAs Targeting Various EML4-ALK Sequences

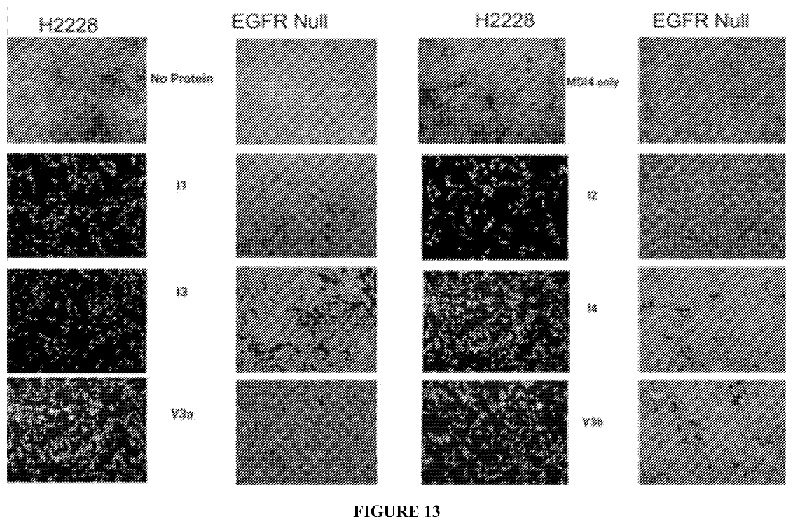

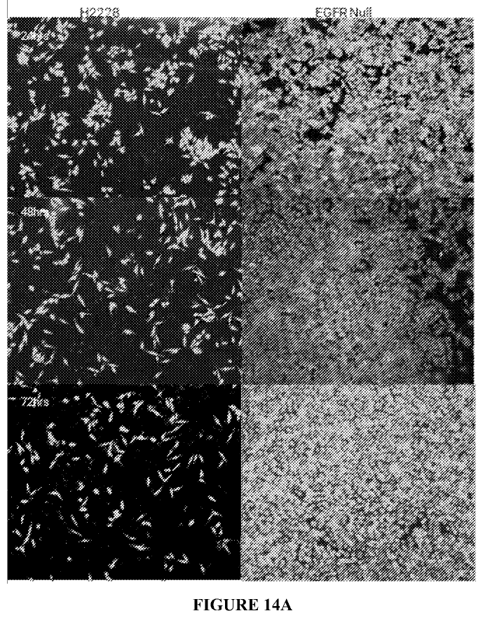

To determine whether gene editing at different sites within the EML5-ALK target gene could also be toxic, 100 nM MDL4 was complexed in a 1:1 ratio with various gRNAs and then applied to H2228 cells. The tested gRNAs included I1, I2, I3, and I4 (SEQ ID NOs: 94/95, 96/97, 98/99, and 100/101 from Table 7), which target different sequences within the kinase domain of ALK, and V3a and V3b (SEQ ID NOs: 90/91 and 92/93), which target EML5-ALK gene fusion variants expressed in H2228 cells. All of these EML5-ALK-specific gRNAs elicited more than a 50% reduction in the viability of H2228 cells, as shown in FIGS. 13. I2 and I3 were the most effective at early time points and caused the highest levels of necrosis. EGRF-null A549 cells were insensitive to all tested MDL4/gRNA complexes because they lack the EGFR receptor for MDL4 uptake and their growth is not dependent on ALK kinase. Additionally, H2228 cells grew to confluence when treated without MDL4 or without RNAs targeting the ALK kinase domain/fusion site.

### Example 8. Cellular Toxicity by MDL4/I2 is Correlated With Efficient In Vivo Genome Editing

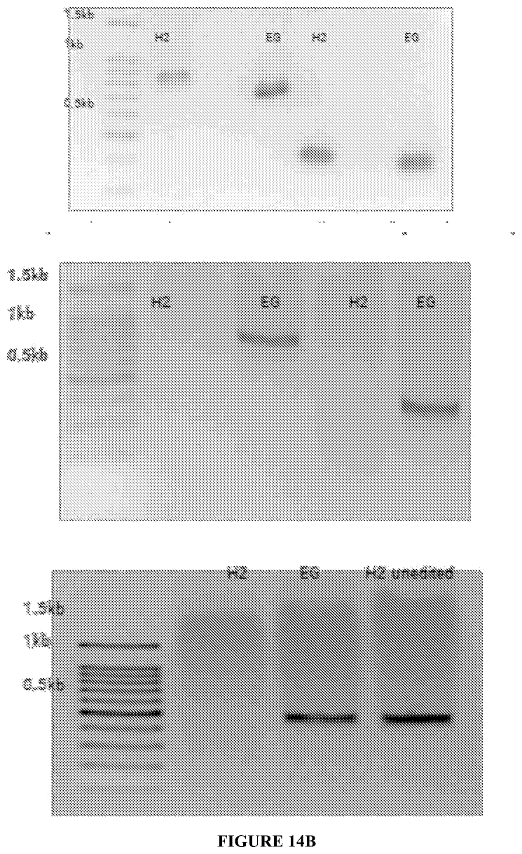

To investigate whether the toxicity caused by MDL4/I2 in H2228 cells is caused by editing the EML5-ALK oncogenic fusion, MDL4/I2 treated H2228 cells were stained with AO/PI to measure toxicity and tested for EML5-ALK edits using a T7 endonuclease assay. MDL4/I2 was applied to H2228 and EGFR null A549 cells. Toxicity and a clear reduction in proliferation were observed in H228 cells as early as 24 hours after treatment, whereas the EGRR null A549 cells were unaffected, as previously described. FIG. 14A. Two regions of the ALK gene were amplified by PCR at the 24-hour timepoint using two different sets of primers two generate two differently sized amplicons (Primer set 1: F-ind 5′-tgatggaaaggttcagagctcag-3′ and R-ind 5′-ggtagacttggagagagcacatc-3′, generating a 750 bp amplicon; Primer set 2: F-IndX 5′-CTGTAGGAAGTGGCCTGTGT-3′ and R-IndX 5′-GCTGTGATAACATTCAGCCCC-3′, generating a 450 bp amplicon). The amplicons from both regions were larger when amplified from H2228 cells, suggesting the presence of a 30-80 bp insertion. FIG. 14B, top panel. T7 endonuclease assays were performed to detect heteroduplexes. Large heteroduplexes were detected in the PCR products from H2228 cells, consistent with the observed size increase. FIG. 14B, middle panel. Heteroduplex formation was also detected in a T7 endonuclease assay on an ALK amplicon from H2228 cells after 48 hours of MDL4/I2 treatment, but not on ALK from MDL4/I2-treated EGFR null A549 cells or H2228 cells treated with MDL4 without a gRNA, as illustrate in FIG. 14B, lower panel. These results confirm that the specific toxicity observed in MDL4/I2-treated H2228 cells is likely caused by indels introduced into the EML5-ALK oncogenic fusion gene.

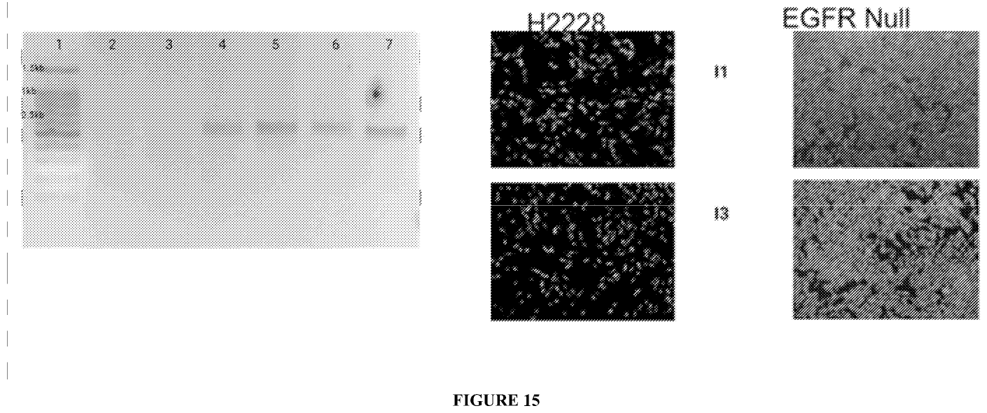

The same experiment above (looking simultaneously at cell viability in H228 vs EGFR-null A549 cells and editing using T7 endonuclease assays) using I2 gRNA was repeated for I1 and I3 gRNAs (see FIG. 15). The degradation of product in lanes 2 and 3 (representing I1/I3 gRNA respectively in H2228 cells) versus lanes 4 and 5 (representing I1/I3 gRNA respectively in EGFR-null A549 cells) or 6 and 7 (representing respectively no gRNA in H2228 cells and no gRNA in EGFR-null A549 cells) indicates that the I1 and I3 gRNAs have similarly selective activity to I2.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A composition for modifying a gene comprising
a cell recognition domain (CRD);
an endosome escape domain (EE); and
a polynucleotide-modifying enzyme domain (PMNE);
wherein the endosome escape domain is covalently coupled to the cell recognition domain.

2. The composition of claim 1, further comprising a hapten binding-domain.

3. The composition of claim 2, wherein the cell recognition domain, endosome escape domain, polynucleotide-modify enzyme domain, and the hapten-binding domain are physically linked.

4. The composition of claim 1, further comprising a bispecific scaffold, wherein the bispecific scaffold binds non-covalently to the cell recognition domain and the polynucleotide-modifying enzyme domain.

5. The composition of claim 4, wherein the bispecific scaffold comprises a hapten and the hapten-binding domain binds to the hapten.

6. The composition of claim 1, wherein one or more of the domains are physically linked by protein ligation.

7. The composition of claim 1, wherein one or more of the domains are linked in the order of any one of the following:
a. PNME-CRD-EE;
b. CRD-PNME-EE; or
c. EE-CRD-PNME.

8. The composition of claim 2, wherein one or more of the domains are linked in the order of any one of the following:
a. PNME-CRD-EE;
b. CRD-PNME-EE;
c. EE-CRD-PNME;
d. PNME-Hapten binding domain-EE;
e. PNME-Hapten binding domain-CRD-EE;
f. EE-CRD-PNME-Hapten binding domain; or
g. EE-Hapten binding domain-PNME-CRD.

9. The composition of claim 8, wherein one or more of the domains are linked in the order of any one of the following:
a. PNME-CRD-EE; or
b. PNME-Hapten binding domain-CRD-EE.

10. The composition of claim 1, wherein one or more of the domains are physically linked by one or more peptide linkers, or one or more chemical cross-linkers.

11. The composition of claim 3, wherein one or more of the cell recognition domain, the endosome escape domain, and the polynucleotide-modifying enzyme domain are physically linked in the form of a fusion polypeptide.

12. The composition of claim 11, wherein the fusion polypeptide further comprises a non-structural linker domain (L).

13-14. (canceled)

15. The composition of claim 11, wherein the fusion polypeptide further comprises the hapten-binding domain.

16. The composition of claim 11, wherein the polynucleotide-modifying enzyme domain is located at the N-terminus of the fusion polypeptide.

17. The composition of claim 11, wherein the cell recognition domain is located at the N-terminus of the fusion polypeptide.

18. The composition of claim 11, wherein the endosome escape domain is located at the N-terminus of the fusion polypeptide.

19. The composition of claim 11, wherein the endosome escape domain is located at the C-terminus of the fusion polypeptide.

20. The composition of claim 11, wherein the cell recognition domain is located at the C-terminus of the fusion polypeptide.

21. The composition of claim 11, wherein the polynucleotide-modifying enzyme domain is located at the C-terminus of the fusion polypeptide.

22. The composition of claims 11, wherein the hapten-binging domain is located at the C-terminus of the fusion polypeptide.

23. The composition of claim 1, wherein the total molecular weight of the composition is between 100 kDa and 240 kDa.

24. The composition of claim 23, wherein the total molecular weight of the composition is between 100 kDa and 200 kDa.

25. The composition of claim 1, wherein the hydrodynamic radius of the composition is less than 100 nm.

26. The composition of claim 25, wherein the hydrodynamic radius of the composition is less than 90 nm, 80 nm, 70 nm or 60 nm.

27. The composition of claim 1, wherein the cell recognition domain binds to an epitopes on a cell-surface antigen.

28. The composition of claim 27, wherein the epitope is an epitope of a receptor displayed on the surface of a cell.

29. The composition of claim 27, wherein the epitope is a protein ligand and the ligand binds to a receptor displayed on the surface of a cell.

30. The composition of claim 28, wherein the cell internalizes the receptor by clathrin-mediated endocytosis, calveolin-mediated endocytosis, or micropinocytosis.

31. The composition of claim 30, wherein binding of the cell recognition domain to the receptor induces the cell to internalize the receptor.

32. The composition of claim 27, wherein the receptor is selectively expressed on a target cell or class of target cells, and the receptor is not expressed, or poorly expressed on a cell that is not the target cell.

33. The composition of claim 32, wherein the target cell is a diseased cell or a cancer cell.

34. The composition of claim 27, wherein the epitope is an epitope of a G-protein coupled receptor.

35. The composition of claim 27, wherein the epitope is an epitope of a protein selected from the group consisting of L-SIGN (liver/lymph node-specific intracellular adhesion molecules-3 grabbing non-integrin), ASGPR (Asialoglycoprotein receptor), AT1 (Angiotensin II Receptor Type 1), B2/B1 receptor (Bradykinin Receptor B1 or B2), and Muscarinic receptors.

36. The composition of claim 27, wherein the epitope is selected from the group consisting of L-SIGN, ASGPR, AT1, B2/B1 receptor, Muscarinic receptors, FGFR4 (Fibroblast Growth Factor Receptor 4), FGFR3 (Fibroblast Growth Factor Receptor 3), FGFR1 (Fibroblast Growth Factor Receptor 1), Frizzled 4, S1PR1 (Sphingosine-1-Phosphate Receptor 1), TSHR (Thyroid Stimulating Hormone Receptor), GPR41 (G Protein-Coupled Receptor 41), GPR43 (G Protein-Coupled Receptor 43), GPR109A (G Protein-Coupled Receptor 109A), TFRC (Transferrin Receptor), Insulin receptor (INSR), Insulin-like growth factor 2 receptor (IGF2R), LRP1 (LDL Receptor Related Protein 1), IGF1R (Insulin Like Growth Factor 1 Receptor), Prolactin receptor (PRLR), and Follicle stimulating hormone receptor (FSHR).

37. The composition of claim 27, wherein the epitope is selected from the group consisting of cd44v6, CAIX (Carbonic Anhydrase 9), CEA (Cell Adhesion Molecule), CD133, cMet hepatocyte growth factor receptor, EGFR (Epidermal Growth Factor Receptor), EGFR vIII, EPCAM (Epithelial Cell Adhesion Molecule), EphA2 (EPH Receptor A2), Fetal acetylcholine receptor, FRalpha folate receptor (FOLR1), GD2 (Ganglioside G2), GPC3 (Glypican 3), GUCY2C (Guanylate Cyclase 2C), HER2 (human epidermal growth factor receptor 2), ICAM1 (Intercellular Adhesion Molecule 1), IL13Ralpha2, IL11 receptor alpha, Kras (Kirsten rat sarcoma viral oncogene homolog), Kras G12D, L1cam (L1 Cell Adhesion Molecule), MAGE (melanoma-associated antigen), Mesothelin MSLN), MUC1 (Mucin 1), MUC16 (Mucin 16), NKG2D (Killer Cell Lectin Like Receptor K1), NY-ESO1 (New York Esophageal Squamous Cell Carcinoma 1), PSCA (Prostate Stem Cell Antigen), WT1 (Wilms Tumor 1 Transcription Factor), PSMA (prostate-specific membrane antigen), TPBG (Trophoblast Glycoprotein), Transferrin receptor (TFRC), GPNMB Breast cancer, melanoma, LeY (Lewis y antigen), CA6 (Carbonic anhydrase 6), Av integrin (Integrin Subunit Alpha V), SLC44A4 (Solute Carrier Family 44 Member 4), Nectin-4 (solid tumors, Ectonucleotide Pyrophosphatase/Phosphodiesterase 3 (ENPP3), Cripto, TENB2 (Transmembrane Protein With EGF Like And Two Follistatin Like Domains 2), EPCAM (epithelial cell adhesion molecule), and CD166.

38. The composition of claim 27, wherein the cell recognition domain comprises two or more binding components, wherein the first binding component binds to a first epitope and the second binding component binds to a second epitope.

39. The composition of claim 38, wherein the cell recognition domain comprises at least three binding components, and the third binding component binds to a third epitope.

40. The composition of claim 39, wherein the cell recognition domain comprises at least four binding components, and the fourth binding component binds to a fourth epitope.

41. The composition of claim 38, wherein the first epitope and the second epitope, and, optionally, the third epitope and the fourth epitope are located on the same cell surface antigen or receptor.

42. The composition of claim 38, wherein the first epitope is located on a first cell surface antigen or receptor and the second epitope is located on a second cell surface antigen or receptor and, optionally, the third epitope is located on a third cell surface antigen or receptor and, optionally, the fourth epitope is located on a fourth cell surface antigen or receptor.

43. The composition of claim 42, wherein the first cell surface receptor is a driver receptor that is internalized by a target cell and the second cell surface receptor is a passenger receptor that is internalized by the target cell, wherein the passenger receptor is internalized less rapidly than the driver receptor.

44. The composition of claim 43, wherein the first cell surface receptor is EPCAM and the second cell surface receptor is ALCAM (activated leukocyte cell adhesion molecule).

45. The composition of claim 1, wherein the cell recognition domain is a protein ligand.

46. The composition of claim 45, wherein the protein ligand comprises 5 to 15 amino acids in length.

47. The composition of claim 45, wherein the protein ligand has a globular or cyclical structure.

48. The composition of claim 45, wherein the protein ligand is an antibody or antigen-binding domain thereof.

49. The composition of claim 48, wherein the antigen-binding domain is a Fab, scFv, single-domain antibody (sdAb), V_{HH}, or camelid antibody domain.

50. The composition of claim 45, wherein the protein ligand is an antibody mimetic.

51. The composition of claim 50, wherein the antibody mimetic is selected from the group consisting of affibody, an affilin, an affimer, an affitin, an alphabody, an anticalin, an atrimer, an avimer, a DARPin, a fynomer, a knottin, a Kunitz domain peptide, a monobody, a nanoCLAMP, and a linear peptide comprising 6-20 amino acids in length.

52. The composition of claim 27, wherein the cell recognition domain is an oligonucleotide.

53. The composition of claim 52, wherein the oligonucleotide is a ribonucleotide or deoxyribonucleotide.

54. The composition of claim 52, wherein the oligonucleotide comprises a non-canonical nucleotide.

55. The composition of claim 54, wherein the non-canonical nucleotide is selected from the group consisting of 2′-OMe nucleotide, 2′-F nucleotide, ′-S nucleotides, 2′-F ANAs, HNAs, and locked nucleic acid residues.

56. The composition of claim 27, wherein the cell recognition domain comprises a chemical ligand with a molecular weight of less than about 800 Da.

57. The composition of claim 56, wherein the endosome escape domain comprises between 3 and 9 amino acids.

58. The composition of claim 57, wherein
the amino acid residue at position 1 of the endosome escape domain is a proline or cysteine;
the amino acid residues at positions 2-5 of the endosome escape domain are cysteines, arginines, or lysines; and
the amino acid residues at positions 6-9 of the endosome escape domain are cysteines, arginines, lysines, alanines or tryptophans.

59. The composition of claim 57, wherein the endosome escape domain comprises at least 3 cysteines and no more than 8 cysteines.

60. The composition of claim 1, wherein the polynucleotide-modifying enzyme domain comprises a nuclear localization sequence (NLS).

61. The composition of claim 59, wherein the NLS sequence is located in a linker domain fused to the N-terminus of the polynucleotide-modifying enzyme domain.

62. The composition of claim 59, wherein the NLS sequence is located in a linker domain fused to the C-terminus of the polynucleotide-modifying enzyme domain.

63. The composition of claim 59, wherein the NLS sequence comprises 7-25 amino acid residues.

64. The composition of claim 59, wherein the NLS is a bipartite NLS wherein amino acids within an N-terminal portion of the NLS involved in the recognition of an importin and amino acids within an a C-terminal portion of the NLS involved in the recognition of an importin are split by an amino acid sequence not involved in the recognition of an importin.

65. The composition of claim 59, wherein the polynucleotide-modifying enzyme domain further comprises a linker sequence separating the NLS from the polynucleotide-modifying enzyme.

66. The composition of claim 59, wherein the linker sequence comprises between 6 and 20 amino acid residues.

67. The composition of claim 66, wherein the NLS comprises a sequence having at least 90% or 95% identity to a sequence selected from the group consisting of SEQ ID NOs: 27-42.

68. The composition of claim 60, wherein the polynucleotide-modifying enzyme domain comprises two or more NLSs.

69. The composition of claim 68, wherein the two or more NLSs comprise a first NLS and a second NLS, wherein the first NLS has the same sequence as the second NLS, and wherein the first NLS is separated from the second NLS by a linker sequence comprising 1-7 amino acid residues.

70. The composition of claim 69, further comprising a third NLS with the same sequence as the first NLS and the second NLS.

71. The composition of claim 68, wherein the two or more NLSs comprise a first NLS and a second NLS, and the first NLS has a different sequence than the second NLS.

72. The composition of claim 2, wherein the hapten binding domain can bind to a hapten that is covalently attached to a peptide, a protein, an oligonucleotide, or a polynucleotide.

73. The composition of claim 72, wherein the protein is selected from the group consisting of an adenosine deaminase, a cytosine deaminase, a transcriptional activator, and a transcriptional suppressor.

74. The composition of claim 72, wherein the oligonucleotide is a deoxyoligoribonucleotide or ribooligonucleotide.

75. The composition of claim 72, wherein the oligonucleotide is a single-stranded oligonucleotide or a double-stranded oligonucleotide.

76. The composition of claim 72, wherein the hapten is selected form the group consisting of fluorescein, biotin, and digoxin.

77. The composition of claim 1, wherein the polynucleotide-modifying enzyme domain is a nuclease, a recombinase, or an RNA editing enzyme.

78. The composition of claim 73, wherein the nuclease comprises a programmable component that directs the nuclease against either DNA or RNA in response to target nucleotide sequence.

79. The composition of claim 77, wherein the nuclease cleaves a ribonucleic acid target or a deoxyribonucleic acid target.

80. The composition of claim 77, wherein the nuclease cleaves a single-stranded polynucleotide target.

81. The composition of claim 77, wherein the nuclease cleaves a double-stranded polynucleotide target.

82. The composition of claim 81, wherein the cleaved double-stranded polynucleotide target has a blunt end, two staggered ends, or a nick in one strand and an intact second strand.

83. The composition of claim 77, wherein the polynucleotide target is a double stranded polynucleotide target and the nuclease cleaves one strand of the double-stranded polynucleotide target.

84. The composition of claim 77, wherein the polynucleotide-modifying enzyme domain comprises a programmable endonuclease.

85. The composition of claim 84, wherein the site-specific endonuclease comprises a Class II Cas enzyme, a TALEN, a meganuclease, a Zn-finger nuclease derivative, or nuclease-deficient variants thereof.

86. The composition of claim 85, wherein the class II Cas enzyme comprises a type II, type V, or type VI Cas enzyme.

87. The composition of claim 86, wherein the class II Cas enzyme comprises a type V Cas enzyme.

88. The composition of claim 87, wherein the type V Cas enzyme comprises asCpfI or MAD7.

89. The composition of claim 77, further comprising a guide oligonucleotide complementary to a target gene, wherein the guide oligonucleotide is non-covalently bound to the polynucleotide-modifying enzyme domain.

90. The composition of claim 89, wherein said guide oligonucleotide comprises a non-complementary region derived from a naturally occurring type II, type V, or type VI crRNA or tracrRNA.

91. The composition of claim 86, wherein the guide oligonucleotide comprises a ribonucleotide or a ribonucleotide and a deoxyribonucleotide.

92. The composition of claim 86, wherein the guide oligonucleotide comprises a non-canonical nucleotide.

93. The composition for claim 92, wherein the non-canonical nucleotide comprises a modification at the 2′ position of a sugar moiety.

94. The composition for claim 92, wherein the non-canonical nucleotide is selected from the group consisting of 2′-OMe nucleotide, 2′-F nucleotide, 4′-S nucleotides, 2′-FANAs, HNA, and locked nucleic acid residues.

95. The composition of claim 92, wherein the guide oligonucleotide comprises one or more bridged nucleotides in a seed region of the guide oligonucleotide.

96. The composition of claim 92, wherein the guide oligonucleotide comprises a sequence of n nucleotides counting from a 1^{st} nucleotide at a 5′ end to an n^{th} nucleotide at a 3′ end, wherein one or more of the nucleotides at positions 1, 2, n-1 and n are phosphorothioate modified nucleotides.

97. The composition of claim 85, wherein the nuclease-deficient polynucleotide-modifying domain can bind DNA and is fused to second enzyme that is capable of epigenetic modifications or base chemical conversion.

98. The composition of claim 97, wherein the epigenetic modification is selected from the group consisting of methylation, RNA cleavage, cytosine deamination, and adenosine deamination.

99. The composition of claim 97, wherein the base chemical conversion is selected from adenosine deamidation and cytosine deamidation.

100. The composition of claim 77, wherein the recombinase is a mammalian recombinase or a eukaryotic recombinase.

101. The composition of claim 77, wherein the recombinase is a Rad52/51 recombinase or a CRE recombinase.

102. The composition of claim 1, further comprising a donor DNA polynucleotide comprising a 5′ homology region and a 3′ homology region, wherein the 5′ homology region comprises a nucleotide sequence with sequence identity to a nucleotide sequence on the 5′ side of the target nucleotide sequence and the 3′ homology region comprises a nucleotide sequence with sequence identity to a nucleotide sequence on the 3′ side of the target nucleotide sequence.

103. The composition of claim 102, wherein the donor DNA polynucleotide further comprises an insert region, and the insert region lies between the 5′ homology region and the 3′ homology region.

104. The composition of claim 103, wherein the insert region comprises an exon, an intron, a transgene, a selectable marker, or a stop codon.

105. The composition of claim 104, wherein the target nucleotide sequence comprises a mutation and the insert region does not comprise a mutation.

106. The composition of claim 102, wherein the 5′ homology region and the 3′ homology region have the same length.

107. The composition of claim 102, wherein the 5′ homology region and the 3′ homology region have different lengths.

108. The composition of claim 102, wherein the donor DNA polynucleotide is a single stranded polynucleotide and the 5′ homology region comprises 50-100 nucleotides and the 3′ homology region comprises 20-60 nucleotides.

109. The composition of claim 102, wherein the 3′ end of the 5′ homology region is homologous to a sequence within 5 nucleotides of the double-stranded break and the 5′ end of the 3′ homology region is homologous to a sequence within 5 nucleotides of the double strand break.

110. The composition of claim 109, wherein the nuclease is a type II or a type V nuclease.

111. The composition of claim 110, wherein the nuclease is a type V nuclease, the target polynucleotide sequence comprises a protospacer adjacent motif (PAM) located within 30 nucleotides of the cleavage site, the cleaved double-stranded polynucleotide target has two staggered ends, and the staggered ends have 4 nucleotide 5′ or 3′ overhangs.

112. The composition of claim 102, wherein a hapten is conjugated to the donor DNA polynucleotide and the hapten binds to the hapten-binding domain.

113. The composition of claim 102, wherein a peptide of less than 20 amino acids in length is conjugated to the donor DNA polynucleotide and the peptide binds to the cell recognition domain.

114. The composition of claim 1, wherein the composition is free of PEI, PEG, PAMAN, or sugar (dextran) derivative polymer comprising more than three subunits.

115. The composition of claim 1, comprising a protein sequence having at least 80% identity to any one of SEQ ID NOs: 16-26, 44, 46, 48, 50, 52, 54, 56, 58, 60, 61-65, 67, 69, 71, 73, 75, 77, 79, 81, 83, 85, 87, or a variant thereof.

116. The composition of claim 1, comprising a protein sequence having at least 80% identity to any one of SEQ ID NOs 67, 69, 71, 73, 75, 77, 79, 81, 83, 85, 87, or a variant thereof.

117. The composition of claim 1, comprising a protein sequence having at least 80% identity to SEQ ID NO 77, 85, 87, or a variant thereof.

118. The composition of claim 89, comprising a guide oligonucleotide complementary to a target gene, wherein the guide oligonucleotide comprises a nucleotide sequence having at least 80% identity to any one of SEQ ID NOs: 88-109, or a variant thereof.

119. The composition of claim 89, comprising a guide oligonucleotide complementary to a target gene, wherein the guide oligonucleotide comprises a nucleotide sequence having at least 80% identity to any one of SEQ ID NOs: 94, 95, 96, 97, 98 99, 100, 101, or a variant thereof.

120. A vector comprising a nucleotide sequence encoding a cell recognition domain, an endosome escape domain, and a polynucleotide-modifying enzyme domain.

121. The vector of claim 120, further comprising a nucleotide sequence encoding a hapten-binding domain.

122. A vector comprising a nucleotide sequence encoding the composition of claim 11.

123. The vector of claim 120, wherein the vector is a plasmid.

124. A host cell comprising the vector of claim 120.

125. The host cell of claim 124, wherein the fusion polypeptide of claim 11 is secreted from the cell.

126. The host cell of claim 124, wherein the host cell is a prokaryotic cell, a eukaryotic cell, an E. coli cell, an insect cell, or an Sf9 cell.

127. A kit for editing a gene in a cell comprising the composition of claim 1, a guide oligonucleotide and a donor DNA polynucleotide.

128. A kit for editing a gene in a cell comprising the vector of claim 120, a guide oligonucleotide and a donor DNA polynucleotide.

129. A kit for editing a gene in a cell comprising the host cell of claim 124, a guide oligonucleotide and a donor DNA polynucleotide.

130. A method of editing a gene by random insertion or deletion comprising contacting the composition of claim 1 with a cell.

131. A method of editing a gene by homology directed repair comprising contacting the composition of claim 1 to with a cell.

132. The method of claim 131, wherein the gene is modified by insertion of a label.

133. The method of claim 132, wherein the label is selected from the list consisting of epitope tag and a fluorescent protein tag.

134. The method of claim 131, wherein a mutation in the gene is repaired.

135. A method of inserting a transgene into the genome of a cell by homologous recombination comprising contacting the composition of claim 1 with the cell.

136. A method of generating a cell amenable to gene editing comprising expressing a receptor in the cell, wherein the cell recognition domain of the composition of claim 1 binds to the receptor.

137. A method of editing a gene in a cell comprising, expressing a receptor on the surface of the cell, and contacting the cell with the composition of claim 1.

138. A method of targeting the composition of claim 1 to the nucleus of a cell comprising contacting the cell with the composition of claim 1, wherein the composition is detected in the nucleus.

139. A method of generating the cell recognition domain of the composition of claim 1 comprising displaying a receptor on a solid surface.

140. The method of claim 139, wherein the solid surface is a well of a multi-well plate or a bead.

141. The method of claim 139, further comprising screening a library of polypeptides displayed on a mammalian cell, a yeast cell, a bacterial cell, or a bacteriophage by ribosomal display, DNA/RNA systematic evolution of ligands by exponential enrichment (SELEX™), or DNA-encoded library approaches.

142. A method for inducing death of cells bearing an EML4-ALK fusion gene, comprising contacting to said cell a composition comprising:
a protein having at least 80% identity to SEQ ID NO 77, or a variant thereof, and
a guide RNA targeting ALK4.

143. The method of claim 142, wherein said guide RNA has at least 80% identity to any one of SEQ ID NOs: 88-105, or a variant thereof.

144. A method for increasing cell resistance to HIV infection, comprising contacting to said cell a composition comprising:
a protein having at least 80% identity to SEQ ID NO: 87, or a variant thereof, and
a guide RNA targeting the CXCR4 locus.

145. The method of claim 144, wherein said guide RNA targeting the CXCR4 locus has at least 80% identity to any one of SEQ ID NOs:108-109, or a variant thereof.


SEQUENCE LISTING (127 sequences)

SEQ ID NO: 1-15 - skipped (15 entries)

SEQ ID NO: 16 - protein, 9 aa, Artificial Sequence
  Description of Artificial Sequence: Synthetic peptide
  MOD_RES (1)..(1): P or C
  MOD_RES (2)..(2): C, R or K
  MOD_RES (3)..(3): C, R or K
  MOD_RES (4)..(4): C, R or K
  MOD_RES (5)..(5): C, R or K
  MOD_RES (6)..(6): C, R, K, A or W
  MOD_RES (7)..(7): C, R, K, A or W
  MOD_RES (8)..(8): C, R, K, A or W
  MOD_RES (9)..(9): C, R, K, A or W
XXXXXXXXX

SEQ ID NO: 17 - protein, 9 aa, Artificial Sequence
  Description of Artificial Sequence: Synthetic peptide
  MOD_RES (1)..(1): P or C
  MOD_RES (2)..(2): C, R or K
  MOD_RES (3)..(3): C, R or K
  MOD_RES (4)..(4): C, R or K
  MOD_RES (5)..(5): C, R or K
  MOD_RES (6)..(6): C, R, K, A or W
  MOD_RES (7)..(7): C, R, K, A or W
  MOD_RES (8)..(8): C, R, K, A or W
  MOD_RES (9)..(9): C, R, K, A or W
  See specification as filed for detailed description of substitutions and preferred embodiments
XXXXXXXXX

SEQ ID NO: 18 - protein, 9 aa, Artificial Sequence
  Description of Artificial Sequence: Synthetic peptide
PCRKCACCA

SEQ ID NO: 19 - protein, 9 aa, Artificial Sequence
  Description of Artificial Sequence: Synthetic peptide
PRCCRWCCA

SEQ ID NO: 20 - protein, 9 aa, Artificial Sequence
  Description of Artificial Sequence: Synthetic peptide
PRRCKRCKC

SEQ ID NO: 21 - protein, 9 aa, Artificial Sequence
  Description of Artificial Sequence: Synthetic peptide
CKKCRKCCK

SEQ ID NO: 22 - protein, 9 aa, Artificial Sequence
  Description of Artificial Sequence: Synthetic peptide
CCRCKCWCC

SEQ ID NO: 23 - protein, 9 aa, Artificial Sequence
  Description of Artificial Sequence: Synthetic peptide
CCRKCCCCC

SEQ ID NO: 24 - protein, 9 aa, Artificial Sequence
  Description of Artificial Sequence: Synthetic peptide
PRKCCCCCC

SEQ ID NO: 25 - protein, 10 aa, Artificial Sequence
  Description of Artificial Sequence: Synthetic peptide
HHHHHHHHHH

SEQ ID NO: 26 - protein, 6 aa, Artificial Sequence
  Description of Artificial Sequence: Synthetic peptide
CCCCCC

SEQ ID NO: 27 - protein, 16 aa, Artificial Sequence
  Description of Artificial Sequence: Synthetic peptide
KRRRRQERAKEREKRR

SEQ ID NO: 28 - protein, 11 aa, Artificial Sequence
  Description of Artificial Sequence: Synthetic peptide
MRKTKALAPTA

SEQ ID NO: 29 - protein, 6 aa, Artificial Sequence
  Description of Artificial Sequence: Synthetic peptide
KKKRRP

SEQ ID NO: 30 - protein, 4 aa, Artificial Sequence
  Description of Artificial Sequence: Synthetic peptide
KKFK

SEQ ID NO: 31 - protein, 6 aa, Artificial Sequence
  Description of Artificial Sequence: Synthetic peptide
KKKKYN

SEQ ID NO: 32 - protein, 9 aa, Artificial Sequence
  Description of Artificial Sequence: Synthetic peptide
PPAKRERLD

SEQ ID NO: 33 - protein, 12 aa, Artificial Sequence
  Description of Artificial Sequence: Synthetic peptide
RGRGRRRRRRRR

SEQ ID NO: 34 - protein, 10 aa, Artificial Sequence
  Description of Artificial Sequence: Synthetic peptide
PKKNKLKKKS

SEQ ID NO: 35 - protein, 7 aa, Artificial Sequence
  Description of Artificial Sequence: Synthetic peptide
PKKKRKV

SEQ ID NO: 36 - protein, 20 aa, Artificial Sequence
  Description of Artificial Sequence: Synthetic peptide
NYKRPMDGTYGPPAKRHEGE

SEQ ID NO: 37 - protein, 8 aa, Artificial Sequence
  Description of Artificial Sequence: Synthetic peptide
KRSGSKAF

SEQ ID NO: 38 - protein, 9 aa, Artificial Sequence
  Description of Artificial Sequence: Synthetic peptide
PPAKRERLD

SEQ ID NO: 39 - protein, 18 aa, Artificial Sequence
  Description of Artificial Sequence: Synthetic peptide
RKKSGMQIALNDHLKQRR

SEQ ID NO: 40 - protein, 16 aa, Artificial Sequence
  Description of Artificial Sequence: Synthetic peptide
KKAFQNVLRIQCLCRK

SEQ ID NO: 41 - protein, 35 aa, Artificial Sequence
  Description of Artificial Sequence: Synthetic polypeptide
RRLLCRCGRRLPPEPCAAARPALFPSGVPAARSSP

SEQ ID NO: 42 - protein, 9 aa, Artificial Sequence
  Description of Artificial Sequence: Synthetic peptide
SVLGKRKFA

SEQ ID NO: 43 - DNA, 4104 bp, Artificial Sequence
  Description of Artificial Sequence: Synthetic polynucleotide
atggataaaaaatacagcattggtctggacattggcacgaatagcgttggttgggcagtgattaccgatgaatacaaagtcccgtcgaaaaaattcaaagtgctgggtaacaccgatcgccatagcattaagaaaaacctgatcggtgcgctgctgtttgattctggcgaaaccgcggaagcaacgcgtctgaaacgtaccgcacgtcgccgttacacgcgccgtaaaaatcgtatttgctatctgcaggaaatctttagcaacgaaatggcgaaagtcgatgactcatttttccaccgcctggaagaatcgtttctggtggaagaagataaaaaacatgaacgtcacccgattttcggcaatatcgttgatgaagtcgcgtaccatgaaaaatatccgacgatttaccacctgcgtaaaaaactggtggattctaccgacaaagccgatctgcgcctgatttatctggcactggctcatatgatcaaatttcgtggtcacttcctgattgaaggcgacctgaacccggataatagtgacgtcgataaactgtttattcagctggtgcaaacctataatcagctgttcgaagaaaacccgatcaatgcaagtggtgttgatgcgaaagccattctgtccgctcgcctgagtaaatcccgccgtctggaaaacctgattgcacagctgccgggtgaaaagaaaaacggtctgtttggcaatctgatcgctctgtcactgggcctgacgccgaactttaaatcgaatttcgacctggcagaagatgctaaactgcagctgagcaaagatacctacgatgacgatctggacaacctgctggcgcaaattggcgaccagtatgccgacctgtttctggcggccaaaaatctgtcagatgccattctgctgtcggacatcctgcgcgtgaacaccgaaatcacgaaagcgccgctgtcagcctcgatgattaaacgctacgatgaacatcaccaggacctgaccctgctgaaagcactggttcgtcagcaactgccggaaaaatacaaagaaattttctttgaccaaagtaaaaatggttatgcaggctacatcgatggcggtgcttcccaggaagaattctacaaattcatcaaaccgatcctggaaaaaatggatggtacggaagaactgctggtgaaactgaatcgtgaagatctgctgcgtaaacaacgcacctttgacaacggtagcattccgcatcagatccacctgggcgaactgcatgcgattctgcgccgtcaggaagatttttatccgttcctgaaagacaaccgtgaaaaaatcgaaaaaatcctgacgtttcgcatcccgtattacgttggtccgctggcacgtggtaatagccgcttcgcatggatgacccgcaaatctgaagaaaccattacgccgtggaactttgaagaagtggttgataaaggcgcaagcgctcagtcttttatcgaacgtatgaccaatttcgataaaaacctgccgaatgaaaaagtgctgccgaaacattctctgctgtatgaatactttaccgtttacaacgaactgacgaaagtgaaatatgttaccgagggtatgcgcaaaccggcgtttctgagtggcgaacagaaaaaagccattgtggatctgctgttcaaaaccaatcgtaaagttacggtcaaacagctgaaagaagattacttcaagaaaattgaatgtttcgacagcgtggaaatttctggtgttgaagatcgtttcaacgcctctctgggcacctatcatgacctgctgaaaatcatcaaagacaaagattttctggataacgaagaaaacgaagacattctggaagatatcgtgctgaccctgacgctgttcgaagatcgtgaaatgattgaagaacgcctgaaaacgtacgcacacctgtttgacgataaagttatgaaacagctgaaacgccgtcgctataccggttggggccgtctgagccgcaaactgattaatggtatccgcgataaacaatcaggcaaaacgattctggatttcctgaaatcggacggctttgccaaccgtaatttcatgcagctgatccatgacgattccctgacctttaaagaagacattcagaaagcacaagtgtcaggtcaaggcgattcgctgcatgaacacattgcgaacctggccggttcaccggctatcaaaaaaggcatcctgcagaccgtgaaagtcgtggatgaactggtgaaagttatgggtcgtcacaaaccggaaaacattgttatcgaaatggcgcgcgaaaatcagaccacgcaaaaaggccagaaaaactcgcgtgaacgcatgaaacgcattgaagaaggtatcaaagaactgggcagccagattctgaaagaacatccggtcgaaaacacccagctgcaaaatgaaaaactgtacctgtattacctgcaaaatggtcgtgacatgtatgtggatcaggaactggacatcaaccgcctgtctgactatgatgtcgaccacattgtgccgcagagctttctgaaagacgattctatcgataacaaagttctgacccgtagtgataaaaaccgcggcaaaagcgacaatgtcccgtctgaagaagttgtgaagaaaatgaaaaactactggcgtcaactgctgaatgcgaaactgattacgcagcgtaaattcgataacctgaccaaagcggaacgcggcggtctgtccgaactggataaagccggttttatcaaacgtcaactggttgaaacccgccagattacgaaacatgtcgcccagatcctggattcacgcatgaacacgaaatacgacgaaaacgataaactgatccgtgaagtcaaagtgatcaccctgaaaagtaaactggtttccgatttccgtaaagactttcagttctacaaagtccgcgaaattaacaattaccatcacgcacacgatgcttatctgaatgcagtggttggtaccgctctgatcaaaaaatatccgaaactggaaagcgaatttgtgtatggcgattacaaagtctatgacgtgcgcaaaatgattgcgaaatccgaacaggaaatcggcaaagcgaccgccaaatactttttctattcaaacatcatgaactttttcaaaaccgaaattacgctggcaaatggtgaaattcgtaaacgcccgctgatcgaaaccaacggtgaaacgggcgaaattgtgtgggataaaggccgtgacttcgcgaccgttcgcaaagtcctgtcgatgccgcaagtgaatatcgtgaagaaaaccgaagtgcagacgggcggttttagtaaagaatccatcctgccgaaacgtaacagcgataaactgattgcgcgcaaaaaagattgggacccgaaaaaatacggcggttttgatagtccgacggttgcatattccgtcctggtcgtggctaaagtcgaaaaaggtaaaagtaaaaaactgaaatccgtgaaagaactgctgggcattaccatcatggaacgtagctcttttgagaaaaacccgattgacttcctggaagccaaaggttacaaagaagtgaaaaaagatctgatcatcaaactgccgaaatatagcctgttcgaactggaaaacggccgtaaacgcatgctggcatctgctggtgaactgcagaaaggcaatgaactggcactgccgagtaaatatgttaactttctgtacctggctagccattatgaaaaactgaaaggttctccggaagataacgaacagaaacaactgttcgtcgaacaacataaacactacctggatgaaatcatcgaacagatctcagaattctcgaaacgcgtgattctggcggatgccaatctggacaaagttctgagcgcgtataacaaacatcgtgataaaccgattcgcgaacaggccgaaaatattatccacctgtttaccctgacgaacctgggcgcaccggcagcttttaaatacttcgataccacgatcgaccgtaaacgctatacctcaacgaaagaagttctggatgctaccctgattcatcaatcgatcaccggtctgtatgaaacgcgtattgatctgagtcagctgggcggtgac

SEQ ID NO: 44 - protein, 1367 aa, Artificial Sequence
  Description of Artificial Sequence: Synthetic polypeptide
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGG

SEQ ID NO: 45 - DNA, 3684 bp, Artificial Sequence
  Description of Artificial Sequence: Synthetic polynucleotide
atgtcaaagctggagaaattcaccaactgttatagcctgtctaagaccctgcgcttcaaggcaatcccagtgggcaagacacaagagaacattgacaacaaacggctcctggtggaggatgagaagagggctgaagattacaagggcgttaagaagctgctggataggtactatctgtcattcatcaacgatgtcctccacagtatcaagctgaagaatctgaacaattacatttctctgttccggaagaagacacggaccgagaaggagaacaaagagctggagaatctggagatcaacctgaggaaagaaatagctaaggctttcaaagggaacgagggttacaagtccctgttcaagaaagacattatcgagactattctgcctgagttcctggacgataaagatgagatcgccctcgtcaattccttcaatgggtttaccacagcctttaccggcttcttcgacaatagagagaatatgttctctgaagaggccaaatccactagcatcgcctttcgctgcataaacgagaacctgactaggtacatcagcaatatggacatctttgagaaagtcgatgccatattcgacaaacatgaggtgcaggagattaaggagaagatcctgaactcagattacgatgtcgaagatttcttcgagggagagttcttcaacttcgtgctcacacaagagggcattgatgtgtacaatgcaatcattggagggttcgtgacagagagtggcgagaagataaagggcctgaacgagtatatcaacctctacaaccagaaaaccaagcagaaactgcctaagttcaagccactgtacaaacaagtgctctcagatagggaaagcctgagcttctacggtgaagggtatacatcagatgaagaagtgctcgaagtgttccgcaacaccctcaataagaacagtgaaatcttctcttcaatcaagaagctggagaaactgttcaagaatttcgatgagtactcctctgccggaatctttgtgaagaatggccctgcaatatccactattagcaaagacatctttggcgagtggaacgttatcagggataagtggaatgccgagtacgatgatattcatctcaagaagaaagccgtggttacagagaaatacgaggatgatagacgcaagagctttaagaagattggtagcttctctctcgaacagctgcaggagtacgccgacgctgacctgtcagtcgtggagaaactcaaggagatcataatccagaaggtggatgaaatctacaaagtgtatggaagctctgagaaactcttcgatgcagactttgttctggagaagagtctgaagaagaacgacgcagtggttgctatcatgaaggacctgctggattctgttaagtctttcgagaattacattaaggcattctttggtgaagggaaggagacaaatagggacgagagcttctatggcgactttgttctggcctacgacatcctcctcaaggttgaccacatctatgacgctatacggaattacgttacccagaagccctatagcaaagacaagttcaagctgtatttccagaatccacagtttatgggtgggtgggataaagacaaagaaacagattacagggccactatcctgcggtacggcagcaaatactatctggctatcatggataagaagtacgccaaatgcctccagaagatcgacaaggacgacgtgaacggtaactacgagaagatcaattacaagctcctgccaggacctaacaagatgctgcccaaggtgttcttctccaagaaatggatggcctactataacccaagcgaggacattcagaagatatacaagaatgggacattcaagaagggcgatatgttcaacctcaacgactgccacaagctgattgatttcttcaaggatagcatttctcgctatcccaagtggtctaatgcatacgatttcaacttcagcgagactgagaagtacaaagacatcgctggcttctaccgggaggtggaagagcaaggctataaggtgtcattcgaatccgcttctaagaaggaagtggataagctcgtggaagagggtaagctgtacatgttccagatatacaacaaagacttcagcgataagagccacggcactccaaacctccatactatgtatttcaagctgctgtttgacgagaacaaccacggacagattaggctgtcaggaggcgcagaactcttcatgcgcagagcttcactgaagaaggaggaactcgttgtccacccagccaatagccctatagccaataagaatccagacaatcctaagaaaaccactactctgtcttacgatgtgtataaggataagagattctctgaagatcagtacgaactgcacatacccattgccattaacaagtgccctaagaacatcttcaagattaacacagaggttagagtgctcctgaaacacgacgataacccttatgttataggcattgatcgcggagagagaaacctgctgtacatcgtcgtggtggacggcaaaggcaacatcgtggaacagtacagtctcaatgaaatcattaacaatttcaacggaatccgcattaagaccgactaccattctctcctcgacaagaaggagaaagaaaggttcgaagcaagacagaattggacaagtatagagaatatcaaagaactgaaggctgggtacatctctcaggttgtgcacaagatatgtgagctggtggagaagtacgacgctgttatcgccctcgaggacctgaatagcggcttcaagaactccagggtgaaggtggagaagcaggtgtatcagaagttcgagaagatgctgatcgacaagctcaactatatggtggacaagaaatccaatccttgcgctactggtggagccctgaagggctatcaaatcaccaataagttcgaatctttcaagtctatgagcacccagaatggcttcatcttctacatacccgcatggctgacatccaagattgatccctctaccggatttgttaatctgctcaagactaagtacacctctattgctgactcaaagaagttcatatcatcatttgaccgcatcatgtacgtgccagaagaggacctgttcgagtttgccctggattacaagaatttctctcggactgacgccgactacatcaagaagtggaagctctactcttatggtaatcggattcgcatattccgcaatcccaagaagaataacgtgttcgattgggaggaagtttgcctcaccagcgcttacaaggagctgttcaataagtatgggattaactaccagcagggcgacataagagccctgctgtgcgaacaatctgataaggcattctattcctctttcatggcactgatgtcactgatgctgcaaatgcgcaattccatcaccggaagaacagacgtggactttctgatctctcctgtcaagaactcagatggcatcttctacgattcccgcaactatgaagcacaggagaatgctatcctgcctaagaatgccgatgcaaatggagcctataacatcgccagaaaggtcctctgggccataggacaattcaagaaagctgaagatgagaagctggacaaggtgaagatcgccatttcaaacaaagagtggctcgaatatgctcagacctcagtgaagcat

SEQ ID NO: 46 - protein, 1228 aa, Artificial Sequence
  Description of Artificial Sequence: Synthetic polypeptide
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 47 - DNA, 3792 bp, Artificial Sequence
  Description of Artificial Sequence: Synthetic polynucleotide
atgaacaacggcacaaataattttcagaacttcatcgggatctcaagtttgcagaaaacgctgcgcaatgctctgatccccacggaaaccacgcaacagttcatcgtcaagaacggaataattaaagaagatgagttacgtggcgagaaccgccagattctgaaagatatcatggatgactactaccgcggattcatctctgagactctgagttctattgatgacatagattggactagcctgttcgaaaaaatggaaattcagctgaaaaatggtgataataaagataccttaattaaggaacagacagagtatcggaaagcaatccataaaaaatttgcgaacgacgatcggtttaagaacatgtttagcgccaaactgattagtgacatattacctgaatttgtcatccacaacaataattattcggcatcagagaaagaggaaaaaacccaggtgataaaattgttttcgcgctttgcgactagctttaaagattacttcaagaaccgtgcaaattgcttttcagcggacgatatttcatcaagcagctgccatcgcatcgtcaacgacaatgcagagatattcttttcaaatgcgctggtctaccgccggatcgtaaaatcgctgagcaatgacgatatcaacaaaatttcgggcgatatgaaagattcattaaaagaaatgagtctggaagaaatatattcttacgagaagtatggggaatttattacccaggaaggcattagcttctataatgatatctgtgggaaagtgaattcttttatgaacctgtattgtcagaaaaataaagaaaacaaaaatttatacaaacttcagaaacttcacaaacagattctatgcattgcggacactagctatgaggtcccgtataaatttgaaagtgacgaggaagtgtaccaatcagttaacggcttccttgataacattagcagcaaacatatagtcgaaagattacgcaaaatcggcgataactataacggctacaacctggataaaatttatatcgtgtccaaattttacgagagcgttagccaaaaaacctaccgcgactgggaaacaattaataccgccctcgaaattcattacaataatatcttgccgggtaacggtaaaagtaaagccgacaaagtaaaaaaagcggttaagaatgatttacagaaatccatcaccgaaataaatgaactagtgtcaaactataagctgtgcagtgacgacaacatcaaagcggagacttatatacatgagattagccatatcttgaataactttgaagcacaggaattgaaatacaatccggaaattcacctagttgaatccgagctcaaagcgagtgagcttaaaaacgtgctggacgtgatcatgaatgcgtttcattggtgttcggtttttatgactgaggaacttgttgataaagacaacaatttttatgcggaactggaggagatttacgatgaaatttatccagtaattagtctgtacaacctggttcgtaactacgttacccagaaaccgtacagcacgaaaaagattaaattgaactttggaataccgacgttagcagacggttggtcaaagtccaaagagtattctaataacgctatcatactgatgcgcgacaatctgtattatctgggcatctttaatgcgaagaataaaccggacaagaagattatcgagggtaatacgtcagaaaataagggtgactacaaaaagatgatttataatttgctcccgggtcccaacaaaatgatcccgaaagttttcttgagcagcaagacgggggtggaaacgtataaaccgagcgcctatatcctagaggggtataaacagaataaacatatcaagtcttcaaaagactttgatatcactttctgtcatgatctgatcgactacttcaaaaactgtattgcaattcatcccgagtggaaaaacttcggttttgattttagcgacaccagtacttatgaagacatttccgggttttatcgtgaggtagagttacaaggttacaagattgattggacatacattagcgaaaaagacattgatctgctgcaggaaaaaggtcaactgtatctgttccagatatataacaaagatttttcgaaaaaatcaaccgggaatgacaaccttcacaccatgtacctgaaaaatcttttctcagaagaaaatcttaaggatatcgtcctgaaacttaacggcgaagcggaaatcttcttcaggaagagcagcataaagaacccaatcattcataaaaaaggctcgattttagtcaaccgtacctacgaagcagaagaaaaagaccagtttggcaacattcaaattgtgcgtaaaaatattccggaaaacatttatcaggagctgtacaaatacttcaacgataaaagcgacaaagagctgtctgatgaagcagccaaactgaagaatgtagtgggacaccacgaggcagcgacgaatatagtcaaggactatcgctacacgtatgataaatacttccttcatatgcctattacgatcaatttcaaagccaataaaacgggttttattaatgataggatcttacagtatatcgctaaagaaaaagacttacatgtgatcggcattgatcggggcgagcgtaacctgatctacgtgtccgtgattgatacttgtggtaatatagttgaacagaaaagctttaacattgtaaacggctacgactatcagataaaactgaaacaacaggagggcgctagacagattgcgcggaaagaatggaaagaaattggtaaaattaaagagatcaaagagggctacctgagcttagtaatccacgagatctctaaaatggtaatcaaatacaatgcaattatagcgatggaggatttgtcttatggttttaaaaaagggcgctttaaggtcgaacggcaagtttaccagaaatttgaaaccatgctcatcaataaactcaactatctggtatttaaagatatttcgattaccgagaatggcggtctcctgaaaggttatcagctgacatacattcctgataaacttaaaaacgtgggtcatcagtgcggctgcattttttatgtgcctgctgcatacacgagcaaaattgatccgaccaccggctttgtgaatatctttaaatttaaagacctgacagtggacgcaaaacgtgaattcattaaaaaatttgactcaattcgttatgacagtgaaaaaaatctgttctgctttacatttgactacaataactttattacgcaaaacacggtcatgagcaaatcatcgtggagtgtgtatacatacggcgtgcgcatcaaacgtcgctttgtgaacggccgcttctcaaacgaaagtgataccattgacataaccaaagatatggagaaaacgttggaaatgacggacattaactggcgcgatggccacgatcttcgtcaagacattatagattatgaaattgttcagcacatattcgaaattttccgtttaacagtgcaaatgcgtaactccttgtctgaactggaggaccgtgattacgatcgtctcatttcacctgtactgaacgaaaataacattttttatgacagcgcgaaagcgggggatgcacttcctaaggatgccgatgcaaatggtgcgtattgtattgcattaaaagggttatatgaaattaaacaaattaccgaaaattggaaagaagatggtaaattttcgcgcgataaactcaaaatcagcaataaagattggttcgactttatccagaataagcgctatctctaa

SEQ ID NO: 48 - protein, 1263 aa, Artificial Sequence
  Description of Artificial Sequence: Synthetic polypeptide
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 49 - DNA, 3162 bp, Artificial Sequence
  Description of Artificial Sequence: Synthetic polynucleotide
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctacagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagttccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtaccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaatcgcctctaagactcagagtatcaaaaagtactcaaccgacattctgggaaacctgtatgaggtgaagagcaaaaagcaccctcagattatcaaaaagggctaa

SEQ ID NO: 50 - protein, 1053 aa, Artificial Sequence
  Description of Artificial Sequence: Synthetic polypeptide
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 51 - DNA, 3921 bp, Artificial Sequence
  Description of Artificial Sequence: Synthetic polynucleotide
atgacccagttcgaggggtttaccaatctgtatcaagtgagcaagacgctgcgctttgaactgatcccacagggaaaaaccttaaaacatattcaagagcagggctttatcgaagaagataaggcccgtaatgaccattacaaagagttaaagccgattattgatcgtatctacaagacctatgcggaccagtgcttacaattggtacagcttgattgggagaacctctctgccgccatcgattcctatcgtaaagaaaaaactgaagaaacgcgcaacgccctgattgaagagcaggccacctatcgtaacgcgattcatgactattttattggccgtacggacaatctgacggacgcgatcaacaagcgccatgcggagatttacaaaggactgtttaaggctgaactgttcaatggtaaggtccttaaacagcttgggaccgtcacaacgacggaacatgaaaacgcgttattacgtagcttcgacaagtttaccacgtatttctccggcttttacgaaaatcgcaaaaacgttttcagtgccgaggatatttccactgctatccctcatcgcattgtgcaagacaacttcccaaaattcaaagaaaattgtcatatcttcacccgcttaatcaccgctgtaccgtccctgcgtgagcatttcgaaaacgtgaaaaaggccattggtatcttcgtgtctacttcgattgaggaggtattttcctttccattctataatcagctgctgacccagacccaaattgatctgtacaaccagctgcttggcggtatttctcgtgaagcaggaaccgaaaaaatcaaagggttgaacgaggtgcttaatctggcaatccagaaaaatgatgaaaccgcccacatcattgcttcgttacctcatcgttttatcccgttgttcaagcaaattttaagtgatcgcaatacgctgtcgtttattctggaagaattcaaaagtgatgaagaggtaattcagtcgttttgcaaatataaaaccctgttacgtaacgaaaatgtcctggaaacagccgaggctttgtttaacgaactgaatagcattgacctgacgcatatctttattagccacaaaaaattagagaccatctcatcagctctgtgcgatcattgggatacactgcgcaatgcgctgtatgaacgtcgtatttcggaattgactggcaaaatcactaaaagcgcgaaagagaaagtacagcgctcgcttaaacatgaagatatcaacctgcaggagatcatcagcgccgcgggtaaagaactgtcggaggcatttaaacagaagacgagcgagattctgtcccacgcacatgccgccttagaccagccgctcccgaccactctgaagaaacaggaagagaaagaaatccttaaaagtcaactggacagtttactgggtctctatcatctgctggattggtttgcggtagacgaaagcaatgaagtggatccggagtttagtgcccgtctgacaggaatcaagctggaaatggagccttcgcttagcttctacaacaaagcccgcaattatgccacgaaaaaaccctatagtgtcgaaaaatttaaactcaactttcaaatgccgacccttgcgtcgggctgggatgtcaacaaagaaaaaaacaacggagctattctgttcgttaaaaatggtctgtactacctgggcatcatgccgaaacagaaaggtcgctacaaagccctttcgttcgagcccacggaaaaaacaagcgaaggcttcgacaaaatgtactacgattactttccggatgcagcaaaaatgatcccgaaatgttccacacagctgaaagccgttacagcacattttcagacgcacaccacccccatcttactgtccaacaattttattgaaccgctggagattactaaagaaatttatgatttgaacaatccggaaaaagagccaaaaaagtttcaaaccgcctacgctaaaaaaaccggggatcagaaagggtaccgcgaagcgttgtgcaagtggattgatttcacccgcgattttctcagtaaatataccaagactacctcgattgacctgagctcactgcgcccgagctctcaatataaggatttgggtgagtactatgctgaattaaaccctttattgtaccacatttcttttcagcgcatcgccgaaaaggaaattatggacgcagtcgaaaccgggaaactgtacctgttccagatctataataaggacttcgccaaaggacatcatggcaaaccgaacctgcacaccctttactggaccgggcttttctctccggaaaatttggcgaaaacctcgatcaagcttaacggtcaagctgagctgttttaccgtccaaaatcccgcatgaagcgcatggcgcatcgtttaggtgaaaaaatgctgaataagaaactgaaagatcagaaaacccctatcccggataccctctaccaggaactgtatgattacgtgaaccatcgtctctcgcatgacctgtcagacgaagcgcgtgcgttactgcccaatgtaatcacaaaagaagtttcgcatgaaattattaaagatcgtcgttttacatctgataaattcttttttcatgttccgatcaccctcaactatcaggccgcaaacagtccaagtaagtttaaccagcgcgttaatgcttacctgaaggaacatccggagactccgattattggaattgatcgcggtgaacgtaatttgatctatatcactgtgatcgatagtaccggtaagattctggagcagcgcagcttgaacacaattcaacagtttgattatcagaaaaaattagacaaccgcgaaaaagagcgcgtggctgcccgtcaggcgtggtctgttgtcggtaccattaaagatctgaagcagggctatctttctcaggttattcacgaaattgtagatctgatgatccattatcaggcggttgttgtgttggagaatctcaatttcggttttaagagtaagcgcacaggcatcgctgaaaaagcagtttatcagcagtttgaaaaaatgctgatcgacaaattgaactgtttagttctcaaagattacccagcggaaaaggtgggcggagtgctgaatccgtaccaattaacggatcaattcacttccttcgcaaagatgggtacccaaagcggctttctgttctatgtgccggccccgtatacctcgaaaatcgatccactgacgggcttcgtagatccgttcgtgtggaaaaccattaaaaatcatgaaagtcgtaaacattttctcgaaggcttcgacttcctgcactacgacgtgaaaactggcgatttcattctgcattttaaaatgaaccgcaacctttcgtttcagcgcggtctgccgggctttatgccggcttgggacattgtttttgagaaaaatgaaacccagtttgatgctaaaggcactcctttcatcgccggtaaacgcatcgtacctgtgattgaaaaccatcgttttacagggcgttaccgtgatttatacccggcgaacgaattgatcgcgctgctggaggaaaagggcatcgttttccgtgacggctccaatattctgccgaaattactggaaaacgacgattcacacgcaattgataccatggtcgcactgattcgctcagtcttacagatgcgtaactctaatgcagccacaggagaagattatattaattcgccagtccgcgatttgaacggtgtttgcttcgacagccgttttcagaatcctgaatggccgatggacgctgatgccaacggagcttatcatatcgccctgaaaggccagctcctgctgaaccacctgaaggaaagcaaagatctgaaattgcagaacggcattagcaaccaggactggttagcatacatccaggaactgcgtaac

SEQ ID NO: 52 - protein, 1307 aa, Artificial Sequence
  Description of Artificial Sequence: Synthetic polypeptide
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 53 - DNA, 411 bp, Artificial Sequence
  Description of Artificial Sequence: Synthetic polynucleotide
atgggtggcggtggcagcggtggcggtggcagccaggtgaaactggaggaaagcggtggcggtagcgttcaaaccggcggtagcctgcgtctgacctgcgcggcgagcggtcgtaccagccgtagctatggtatgggttggtttcgtcaggcgccgggcaaggagcgtgaatttgtgagcggtatcagctggcgtggcgacagcaccggttatgcggatagcgtgaagggtcgtttcaccattagccgtgacaacgcgaaaaacaccgttgatctgcaaatgaacagcctgaagccggaggacaccgcgatctactattgcgcggcggcggcgggtagcgcgtggtatggtaccctgtacgaatatgattactggggccagggtacccaagtgaccgttagcagcctcgag

SEQ ID NO: 54 - protein, 126 aa, Artificial Sequence
  Description of Artificial Sequence: Synthetic polypeptide
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSLE

SEQ ID NO: 55 - DNA, 198 bp, Artificial Sequence
  Description of Artificial Sequence: Synthetic polynucleotide
atggcatcaccatgggtggataacaaatttaacaaagaattttcttatgcgattaatgaaattgccctgccgaacctgaacgaaaagcagggcagagcgtttattaacagcctgcgtgatgatccgagccagagcgcgaacctgctggcggaagcgaaaaaactgaacgatgcgcaggcgccgaaatgttgttgttgt

SEQ ID NO: 56 - protein, 66 aa, Artificial Sequence
  Description of Artificial Sequence: Synthetic polypeptide
MASPWVDNKFNKEFSYAINEIALPNLNEKQGRAFINSLRDDPSQSANLLAEAKKLNDAQAPKCCCC

SEQ ID NO: 57 - DNA, 201 bp, Artificial Sequence
  Description of Artificial Sequence: Synthetic polynucleotide
atggcatcaccatgggtggataacaaatttaacaaagaatggtccaaaggcggatgccgaaattgttcttcacctgccgaacctgaacgacgcccagggagcgtttatggtgagcctgaggatgcctccgagccagagcgcgaacctgctggcggaagcgaaaaaactgaacgatgcgcaggcgccgaaatgttgttgtgt

SEQ ID NO: 58 - protein, 67 aa, Artificial Sequence
  Description of Artificial Sequence: Synthetic polypeptide
MASPWVDNKFNKEWSKGGCRNCSSPAEPERRPGSVYGEPEDASEPEREPAGGSEKTERCAGAEMLLC

SEQ ID NO: 59 - DNA, 312 bp, Artificial Sequence
  Description of Artificial Sequence: Synthetic polynucleotide
ctgcgtctgacctgcgcggcgtctggtcgtacctctcgttcttacggtatgggttggttccgtcaggcgccgggtaaagaacgtgaattcgtttctggtatctcttggcgtggtgactctaccggttacgcggactctgttaaaggtcgtttcaccatctctcgtgacaacgcgaaaaacaccgttgacctgcagatgaactctctgaaaccggaagacaccgcgatctactactgcgcggcggcggcgggttctgcgtggtacggtaccctgtacgaatacgactactggggtcagggtacccaggttacc

SEQ ID NO: 60 - protein, 104 aa, Artificial Sequence
  Description of Artificial Sequence: Synthetic polypeptide
LRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVT

SEQ ID NO: 61 - protein, 80 aa, Artificial Sequence
  Description of Artificial Sequence: Synthetic polypeptide
  SITE (9)..(80): This region may encompass 1-9 “Gly Ser Pro Gly Pro Gly Pro Gly” repeating units wherein some positions may be absent
  See specification as filed for detailed description of substitutions and preferred embodiments
GSPGPGPGXXXXXXXXXXXXXXXXXXXXXXXXXXXXXXXXXXXXXXXXXXXXXXXXXXXXXXXXXXXXXXXXXXXXXXXX

SEQ ID NO: 62 - protein, 60 aa, Artificial Sequence
  Description of Artificial Sequence: Synthetic polypeptide
  SITE (7)..(60): This region may encompass 1-9 “Gly Ser Pro Pro Gly Pro” repeating units wherein some positions may be absent
  See specification as filed for detailed description of substitutions and preferred embodiments
GSPPGPXXXXXXXXXXXXXXXXXXXXXXXXXXXXXXXXXXXXXXXXXXXXXXXXXXXXXX

SEQ ID NO: 63 - protein, 20 aa, Artificial Sequence
  Description of Artificial Sequence: Synthetic peptide
  SITE (3)..(20): This region may encompass 1-9 “Gly Ser” repeating units wherein some positions may be absent
GSXXXXXXXXXXXXXXXXXX

SEQ ID NO: 64 - protein, 50 aa, Artificial Sequence
  Description of Artificial Sequence: Synthetic polypeptide
  SITE (6)..(50): This region may encompass 1-9 “Gly Gly Gly Gly Ser” repeating units wherein some positions may be absent
GGGGSXXXXXXXXXXXXXXXXXXXXXXXXXXXXXXXXXXXXXXXXXXXXX

SEQ ID NO: 65 - protein, 46 aa, Artificial Sequence
  Description of Artificial Sequence: Synthetic polypeptide
AEAAAKEAAAKEAAAKEAAAKALEAEAAAKEAAAKEAAAKEAAAKA

SEQ ID NO: 66 - DNA, 4371 bp, Artificial Sequence
  Description of Artificial Sequence: Synthetic polynucleotide
atgtacaggatgcaactcctgtcttgcattgcactaagtcttgcacttgtcacgaactctcaggtgaaactggaggagagcgggggcgggagcgtgcagactggggggagcctgagactgacatgcgcagcaagcgggcggacaagccggagctacggaatgggatggttcaggcaggcaccaggcaaggagagggagtttgtgagcggcatctcctggagaggcgatagcaccggctatgccgactccgtgaagggcaggttcaccatcagccgcgataatgccaagaacacagtggacctgcagatgaactccctgaagcccgaggacaccgcaatctactattgcgcagcagcagcaggctccgcctggtacggcacactgtacgagtatgattactggggccagggcacccaggtgacagtgagctccgccctggagggaggaggaggctctggaggaggaggcagcatgaacaatggcaccaacaatttccagaacttcatcggcatctctagcctgcagaagaccctgaggaacgccctgatccctacagagacaacacagcagttcatcgtgaagaatggcatcatcaaggaggatgagctgcggggcgagaacagacagatcctgaaggacatcatggacgattactatcgcggcttcatctctgagacactgtcctctatcgacgatatcgactggacaagcctgtttgagaagatggagatccagctgaagaatggcgataacaaggacaccctgatcaaggagcagacagagtacaggaaggccatccacaagaagttcgccaatgacgatcgcttcaagaacatgttttccgccaagctgatctctgatatcctgccagagtttgtgatccacaacaataactactctgccagcgagaaggaggagaagacccaggtcatcaagctgttcagccggtttgccacatccttcaaggactacttcaagaatagagccaactgcttctccgccgacgatatcagctcctctagctgtcaccggatcgtgaatgataacgccgagatcttcttttctaacgccctggtgtaccggagaatcgtgaagtccctgtctaatgacgatatcaacaagatcagcggcgatatgaaggactctctgaaggagatgagcctggaggagatctattcctacgagaagtacggcgagttcatcacccaggagggcatctccttttataacgacatctgcggcaaggtcaattctttcatgaacctgtactgtcagaagaataaggagaataagaacctgtataagctgcagaagctgcacaagcagatcctgtgcatcgccgatacaagctacgaggtgccctataagttcgagtccgacgaggaggtgtaccagtctgtgaatggctttctggataacatctcctctaagcacatcgtggagcggctgagaaagatcggcgataattacaacggctataacctggacaagatctatatcgtgtccaagttttacgagagcgtgtcccagaagacctacagagactgggagacaatcaacacagccctggagatccactataataacatcctgcctggcaacggcaagtccaaggccgataaggtgaagaaggccgtgaagaatgacctgcagaagtctatcaccgagatcaatgagctggtgtctaactacaagctgtgcagcgacgataacatcaaggccgagacatatatccacgagatcagccacatcctgaataacttcgaggcccaggagctgaagtacaatcctgagatccacctggtggagtccgagctgaaggcctctgagctgaagaatgtgctggacgtgatcatgaacgccttccactggtgttccgtgtttatgaccgaggagctggtggacaaggataataacttttatgccgagctggaggagatctacgatgagatctatccagtgatctctctgtataatctggtgcggaactacgtgacccagaagccctatagcacaaagaagatcaagctgaacttcggcatccctaccctggcagacggatggtctaagagcaaggagtacagcaataacgccatcatcctgatgagagataatctgtactatctgggcatctttaatgccaagaacaagccagacaagaagatcatcgagggcaatacatccgagaacaagggcgattacaagaagatgatctataatctgctgcccggccctaacaagatgatcccaaaggtgttcctgagctccaagaccggcgtggagacatacaagcccagcgcctatatcctggagggctacaagcagaacaagcacatcaagtctagcaaggacttcgatatcaccttttgccacgatctgatcgactacttcaagaattgtatcgccatccaccccgagtggaagaacttcggctttgatttctctgacaccagcacatacgaggacatctctggcttttatagggaggtggagctgcagggctacaagatcgattggacatatatcagcgagaaggacatcgatctgctgcaggagaagggccagctgtatctgttccagatctacaacaaggatttttccaagaagtctaccggcaatgacaacctgcacacaatgtacctgaagaatctgttcagcgaggagaacctgaaggacatcgtgctgaagctgaatggcgaggccgagatcttctttcgcaagtcctctatcaagaatcccatcatccacaagaagggctccatcctggtgaacaggacctacgaggccgaggagaaggaccagttcggcaacatccagatcgtgcgcaagaatatccctgagaacatctatcaggagctgtataagtactttaatgataagagcgacaaggagctgtccgatgaggccgccaagctgaagaatgtggtgggacaccacgaggcagcaaccaacatcgtgaaggattataggtacacatatgacaagtacttcctgcacatgcccatcaccatcaatttcaaggccaacaagacaggctttatcaacgaccgcatcctgcagtacatcgccaaggagaaggatctgcacgtgatcggcatcgacaggggcgagcgcaatctgatctacgtgagcgtgatcgacacctgcggcaacatcgtggagcagaagtcttttaatatcgtgaacggctacgattatcagatcaagctgaagcagcaggagggagcaaggcagatcgcaaggaaggagtggaaggagatcggcaagatcaaggagatcaaggagggctacctgagcctggtcatccacgagatctccaagatggtcatcaagtacaacgccatcatcgccatggaggacctgagctatggcttcaagaaaggccggtttaaggtggagagacaggtgtaccagaagttcgagacaatgctgatcaataagctgaactatctggtgtttaaggacatctccatcaccgagaacggcggcctgctgaagggctaccagctgacatatatccctgataagctgaagaatgtgggccaccagtgcggctgtatcttctatgtgccagccgcctacaccagcaagatcgaccccaccacaggctttgtgaacatctttaagttcaaggatctgacagtggacgccaagcgggagttcatcaagaagtttgattctatcagatacgacagcgagaagaacctgttttgcttcacctttgattacaacaacttcatcacccagaacacagtgatgtccaagagctcctggagcgtgtacacatatggcgtgaggatcaagaggcgcttcgtgaatggccgctttagcaacgagtccgataccatcgacatcacaaaggatatggagaagaccctggagatgacagacatcaactggagggatggccacgacctgcgccaggatatcatcgactacgagatcgtgcagcacatcttcgagatctttcggctgaccgtgcagatgagaaactccctgtctgagctggaggaccgggattacgacagactgatcagccctgtgctgaatgagaataacatcttctatgattccgccaaggcaggcgacgcactgccaaaggatgcagacgccaacggcgcctactgtatcgccctgaagggcctgtatgagatcaagcagatcacagagaattggaaggaggatggcaagttttctcgggacaagctgaagatcagcaataaggattggttcgactttatccagaacaagcggtacctgcccaagaagaagcggaaggtggaggaccccaagaagaagcggaaagtggagaatctgtatttccagggcgggtcatctcatcaccaccaccatcaccatcatcatcactaa

SEQ ID NO: 67 - protein, 1456 aa, Artificial Sequence
  Description of Artificial Sequence: Synthetic polypeptide
MYRMQLLSCIALSLALVTNSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSALEGGGGSGGGGSMNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYLPKKKRKVEDPKKKRKVENLYFQGGSSHHHHHHHHHH

SEQ ID NO: 68 - DNA, 4386 bp, Artificial Sequence
  Description of Artificial Sequence: Synthetic polynucleotide
atgtacaggatgcaactcctgtcttgcattgcactaagtcttgcacttgtcacgaactctcaggtgaagctggaggagagcggaggaggctccgtgcagaccggaggctctctgaggctgacatgcgcagcaagcggaaggacctcccgctcttacggaatgggatggttcaggcaggcaccaggcaaggagagagagttcgtgagcggcatctcttggcgcggcgattccaccggctatgccgactctgtgaagggccggtttacaatcagcagagataatgccaagaacaccgtggacctgcagatgaactccctgaagcccgaggacacagccatctactattgtgcagcagcagcaggcagcgcctggtacggcaccctgtacgagtatgattactggggccagggcacccaggtgacagtgagctccgccctggagggcggcggcggctctggaggaggaggcagcggcggaggaggctccatgaacaatggcaccaacaatttccagaacttcatcggcatctctagcctgcagaagacactgcggaacgccctgatccctaccgagaccacacagcagttcatcgtgaagaatggcatcatcaaggaggatgagctgaggggcgagaaccgccagatcctgaaggacatcatggacgattactatagaggcttcatctctgagacactgtcctctatcgacgatatcgactggaccagcctgtttgagaagatggagatccagctgaagaatggcgataacaaggacaccctgatcaaggagcagacagagtaccggaaggccatccacaagaagttcgccaatgacgatagattcaagaacatgttttctgccaagctgatcagcgatatcctgccagagtttgtgatccacaacaataactacagcgcctccgagaaggaggagaagacacaggtcatcaagctgttcagcaggtttgccacctctttcaaggactacttcaagaatcgcgccaactgcttctccgccgacgatatcagctcctctagctgtcacaggatcgtgaatgataacgccgagatcttcttttctaacgccctggtgtaccggagaatcgtgaagtctctgagcaatgacgatatcaacaagatcagcggcgatatgaaggacagcctgaaggagatgtccctggaggagatctattcctacgagaagtacggcgagttcatcacacaggagggcatctccttttataacgacatctgcggcaaggtcaattcttttatgaacctgtactgtcagaagaataaggagaataagaacctgtataagctgcagaagctgcacaagcagatcctgtgcatcgccgatacctcctacgaggtgccctataagttcgagtctgacgaggaggtgtaccagagcgtgaatggctttctggataacatctcctctaagcacatcgtggagcggctgagaaagatcggcgataattacaacggctataacctggacaagatctatatcgtgagcaagttctacgagtccgtgtctcagaagacctaccgggactgggagaccatcaatacagccctggagatccactataataacatcctgcctggcaacggcaagtccaaggccgataaggtgaagaaggccgtgaagaatgacctgcagaagtctatcacagagatcaatgagctggtgagcaactacaagctgtgctccgacgataacatcaaggccgagacctatatccacgagatctcccacatcctgaataactttgaggcccaggagctgaagtacaatcctgagatccacctggtggagtctgagctgaaggccagcgagctgaagaatgtgctggacgtgatcatgaacgccttccactggtgtagcgtgtttatgaccgaggagctggtggacaaggataataacttctatgccgagctggaggagatctacgatgagatctatccagtgatctctctgtataatctggtgaggaactacgtgacccagaagccctatagcacaaagaagatcaagctgaacttcggcatccctacactggccgacggctggagcaagtccaaggagtactccaataacgccatcatcctgatgcgcgataatctgtactatctgggcatctttaatgccaagaacaagccagacaagaagatcatcgagggcaataccagcgagaacaagggcgattacaagaagatgatctataatctgctgcccggccctaacaagatgatcccaaaggtgttcctgagctccaagaccggcgtggagacatacaagcccagcgcctatatcctggagggctacaagcagaacaagcacatcaagtctagcaaggacttcgatatcacattttgccacgatctgatcgactacttcaagaattgtatcgccatccaccccgagtggaaaaacttcggctttgatttcagcgacacctccacatacgaggacatctctggcttttatcgggaggtggagctgcagggctacaagatcgattggacctatatcagcgagaaggacatcgatctgctgcaggagaagggccagctgtatctgttccagatctacaacaaggatttttctaagaagagcacaggcaatgacaacctgcacaccatgtacctgaagaatctgttctccgaggagaacctgaaggacatcgtgctgaagctgaatggcgaggccgagatcttctttagaaagtcctctatcaagaatcccatcatccacaagaagggcagcatcctggtgaaccggacctacgaggccgaggagaaggaccagttcggcaacatccagatcgtgagaaagaatatccctgagaacatctatcaggagctgtataagtactttaatgataagtccgacaaggagctgtctgatgaggccgccaagctgaagaatgtggtgggccaccacgaggccgccacaaacatcgtgaaggattataggtacacctatgacaagtactttctgcacatgcccatcacaatcaatttcaaggccaacaagaccggctttatcaacgaccgcatcctgcagtacatcgccaaggagaaggatctgcacgtgatcggcatcgaccggggcgagagaaatctgatctacgtgagcgtgatcgacacctgtggcaacatcgtggagcagaagtctttcaatatcgtgaacggctacgattatcagatcaagctgaagcagcaggagggagcaaggcagatcgcaagaaaggagtggaaggagatcggcaagatcaaggagatcaaggagggctacctgagcctggtcatccacgagatctctaagatggtcatcaagtacaacgccatcatcgccatggaggacctgtcctatggcttcaagaagggcaggtttaaggtggagcgccaggtgtaccagaagttcgagaccatgctgatcaataagctgaactatctggtgtttaaggacatcagcatcacagagaacggcggcctgctgaagggctaccagctgacctatatccctgataagctgaagaatgtgggccaccagtgcggctgtatcttctatgtgccagccgcctacacaagcaagatcgaccccaccacaggctttgtgaatatctttaagttcaaggatctgaccgtggacgccaagagggagttcatcaagaagtttgatagcatccgctacgactccgagaagaacctgttttgcttcacatttgattacaacaacttcatcacccagaatacagtgatgtctaagagctcctggagcgtgtacacctatggcgtgcggatcaagaggcgcttcgtgaatggcagattttccaacgagtctgataccatcgacatcacaaaggatatggagaagaccctggagatgacagacatcaactggcgggatggccacgacctgagacaggatatcatcgactacgagatcgtgcagcacatcttcgagatctttaggctgacagtgcagatgcgcaactctctgagcgagctggaggacagggattacgaccgcctgatcagccctgtgctgaatgagaataacatcttctatgattccgccaaggcaggcgacgcactgccaaaggatgcagacgccaacggcgcctactgtatcgccctgaagggcctgtatgagatcaagcagatcaccgagaattggaaggaggatggcaagtttagccgggacaagctgaagatctccaataaggattggttcgactttatccagaacaagaggtacctgcccaagaagaagcggaaggtggaggaccccaagaagaagcggaaagtggagaacctgtatttccagggcggctctagccatcatcaccatcatcaccaccaccaccactga

SEQ ID NO: 69 - protein, 1461 aa, Artificial Sequence
  Description of Artificial Sequence: Synthetic polypeptide
MYRMQLLSCIALSLALVTNSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSALEGGGGSGGGGSGGGGSMNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYLPKKKRKVEDPKKKRKVENLYFQGGSSHHHHHHHHHH

SEQ ID NO: 70 - DNA, 4401 bp, Artificial Sequence
  Description of Artificial Sequence: Synthetic polynucleotide
atgtacaggatgcaactcctgtcttgcattgcactaagtcttgcacttgtcacgaactctcaggtgaagctggaggagagcggaggaggctccgtgcagaccggaggcagcctgaggctgacatgcgcagcatccggaaggacctcccgctcttacggaatgggatggttcaggcaggcaccaggcaaggagagagagttcgtgagcggcatctcttggcgcggcgattctaccggctatgccgacagcgtgaagggccggtttacaatctccagagataatgccaagaacaccgtggacctgcagatgaactctctgaagcccgaggacacagccatctactattgtgcagcagcagcaggcagcgcctggtacggcaccctgtacgagtatgattactggggccagggcacccaggtgacagtgagctccgccctggagggcggcggcggctctggaggaggaggcagcggcggaggaggctccggaggcggcggctctatgaacaatggcaccaacaatttccagaacttcatcggcatctctagcctgcagaagacactgcggaacgccctgatccctaccgagaccacacagcagttcatcgtgaagaatggcatcatcaaggaggatgagctgaggggcgagaaccgccagatcctgaaggacatcatggacgattactatagaggcttcatcagcgagacactgtcctctatcgacgatatcgactggacctccctgtttgagaagatggagatccagctgaagaatggcgataacaaggacaccctgatcaaggagcagacagagtaccggaaggccatccacaagaagttcgccaatgacgatagattcaagaacatgtttagcgccaagctgatctccgatatcctgccagagtttgtgatccacaacaataactacagcgcctccgagaaggaggagaagacacaggtcatcaagctgttcagcaggtttgccaccagcttcaaggactacttcaagaatcgcgccaactgcttctctgccgacgatatcagctcctctagctgtcacaggatcgtgaatgataacgccgagatcttcttttccaacgccctggtgtaccggagaatcgtgaagtctctgagcaatgacgatatcaacaagatctccggcgatatgaaggactccctgaaggagatgtctctggaggagatctattcttacgagaagtacggcgagttcatcacacaggagggcatctctttttataacgacatctgcggcaaggtcaatagctttatgaacctgtactgtcagaagaataaggagaataagaacctgtataagctgcagaagctgcacaagcagatcctgtgcatcgccgataccagctacgaggtgccctataagttcgagagcgacgaggaggtgtaccagtccgtgaatggctttctggataacatctcctctaagcacatcgtggagcggctgagaaagatcggcgataattacaacggctataacctggacaagatctatatcgtgtccaagttctacgagtccgtgtctcagaagacctaccgggactgggagaccatcaatacagccctggagatccactataataacatcctgcctggcaacggcaagtctaaggccgataaggtgaagaaggccgtgaagaatgacctgcagaagagcatcacagagatcaatgagctggtgtccaactacaagctgtgctctgacgataacatcaaggccgagacctatatccacgagatcagccacatcctgaataactttgaggcccaggagctgaagtacaatcctgagatccacctggtggagagcgagctgaaggcctccgagctgaagaatgtgctggacgtgatcatgaacgccttccactggtgttccgtgtttatgaccgaggagctggtggacaaggataataacttctatgccgagctggaggagatctacgatgagatctatccagtgatcagcctgtataatctggtgaggaactacgtgacccagaagccctattccacaaagaagatcaagctgaacttcggcatccctacactggccgacggctggagcaagtccaaggagtacagcaataacgccatcatcctgatgcgcgataatctgtactatctgggcatctttaatgccaagaacaagccagacaagaagatcatcgagggcaatacctccgagaacaagggcgattacaagaagatgatctataatctgctgcccggccctaacaagatgatcccaaaggtgttcctgagctccaagaccggcgtggagacatacaagcccagcgcctatatcctggagggctacaagcagaacaagcacatcaagtctagcaaggacttcgatatcacattttgccacgatctgatcgactacttcaagaattgtatcgccatccaccccgagtggaaaaacttcggctttgatttcagcgacacctccacatacgaggacatcagcggcttttatcgggaggtggagctgcagggctacaagatcgattggacctatatctccgagaaggacatcgatctgctgcaggagaagggccagctgtatctgttccagatctacaacaaggatttttctaagaagagcacaggcaatgacaacctgcacaccatgtacctgaagaatctgttcagcgaggagaacctgaaggacatcgtgctgaagctgaatggcgaggccgagatcttctttagaaagtcctctatcaagaatcccatcatccacaagaagggctccatcctggtgaaccggacctacgaggccgaggagaaggaccagttcggcaacatccagatcgtgagaaagaatatccctgagaacatctatcaggagctgtacaagtactttaatgataagtctgacaaggagctgagcgatgaggccgccaagctgaagaatgtggtgggccaccacgaggccgccacaaacatcgtgaaggattataggtacacctatgacaagtactttctgcacatgcccatcacaatcaatttcaaggccaacaagaccggctttatcaacgaccgcatcctgcagtacatcgccaaggagaaggatctgcacgtgatcggcatcgaccggggcgagagaaatctgatctacgtgagcgtgatcgacacctgtggcaacatcgtggagcagaagagcttcaatatcgtgaacggctacgattatcagatcaagctgaagcagcaggagggagcaaggcagatcgcaagaaaggagtggaaggagatcggcaagatcaaggagatcaaggagggctacctgagcctggtcatccacgagatcagcaagatggtcatcaagtacaacgccatcatcgccatggaggacctgagctatggcttcaagaagggcaggtttaaggtggagcgccaggtgtaccagaagttcgagaccatgctgatcaataagctgaactatctggtgtttaaggacatctccatcacagagaacggcggcctgctgaagggctaccagctgacctatatccctgataagctgaagaatgtgggccaccagtgcggctgtatcttctatgtgccagccgcctacacaagcaagatcgaccccaccacaggctttgtgaatatctttaagttcaaggatctgaccgtggacgccaagagggagttcatcaagaagtttgattccatccgctacgactctgagaagaacctgttttgcttcacatttgattacaacaacttcatcacccagaatacagtgatgagcaagagctcctggtccgtgtacacctatggcgtgcggatcaagaggcgcttcgtgaatggcagattttccaacgagtctgataccatcgacatcacaaaggatatggagaagaccctggagatgacagacatcaactggcgggatggccacgacctgagacaggatatcatcgactacgagatcgtgcagcacatcttcgagatctttaggctgacagtgcagatgcgcaactctctgagcgagctggaggacagggattacgaccgcctgatctcccctgtgctgaatgagaataacatcttctatgattctgccaaggcaggcgacgcactgccaaaggatgcagacgccaacggcgcctactgtatcgccctgaagggcctgtatgagatcaagcagatcaccgagaattggaaggaggatggcaagttttcccgggacaagctgaagatctctaataaggattggttcgactttatccagaacaagaggtacctgcccaagaagaagcggaaggtggaggaccccaagaagaagcggaaagtggagaacctgtatttccagggcggctctagccatcatcaccatcatcaccaccaccaccactga

SEQ ID NO: 71 - protein, 1466 aa, Artificial Sequence
  Description of Artificial Sequence: Synthetic polypeptide
MYRMQLLSCIALSLALVTNSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSALEGGGGSGGGGSGGGGSGGGGSMNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYLPKKKRKVEDPKKKRKVENLYFQGGSSHHHHHHHHHH

SEQ ID NO: 72 - DNA, 4371 bp, Artificial Sequence
  Description of Artificial Sequence: Synthetic polynucleotide
atgtacaggatgcaactcctgtcttgcattgcactaagtcttgcacttgtcacgaactctatgaacaatggcaccaacaatttccagaacttcatcggcatcagctccctgcagaagacactgcggaacgccctgatccctaccgagaccacacagcagttcatcgtgaagaatggcatcatcaaggaggatgagctgaggggcgagaaccgccagatcctgaaggacatcatggacgattactatagaggcttcatctccgagacactgtctagcatcgacgatatcgactggacctctctgtttgagaagatggagatccagctgaagaatggcgataacaaggacaccctgatcaaggagcagacagagtaccggaaggccatccacaagaagttcgccaatgacgatagattcaagaacatgttttctgccaagctgatcagcgatatcctgccagagtttgtgatccacaacaataactactccgcctctgagaaggaggagaagacacaggtcatcaagctgttcagcaggtttgccacctctttcaaggactacttcaagaatcgcgccaactgcttcagcgccgacgatatctcctctagctcctgtcacaggatcgtgaatgataacgccgagatcttcttttccaacgccctggtgtaccggagaatcgtgaagagcctgtccaatgacgatatcaacaagatctctggcgatatgaaggacagcctgaaggagatgtccctggaggagatctacagctatgagaagtacggcgagttcatcacacaggagggcatcagcttttataacgacatctgcggcaaggtcaattccttcatgaacctgtactgtcagaagaataaggagaataagaacctgtataagctgcagaagctgcacaagcagatcctgtgcatcgccgataccagctacgaggtgccctataagttcgagtccgacgaggaggtgtaccagtctgtgaatggctttctggataacatctctagcaagcacatcgtggagcggctgagaaagatcggcgataattacaacggctataacctggacaagatctatatcgtgtccaagttttacgagtctgtgagccagaagacctaccgggactgggagaccatcaatacagccctggagatccactataataacatcctgcctggcaacggcaagagcaaggccgataaggtgaagaaggccgtgaagaatgacctgcagaagtccatcacagagatcaatgagctggtgagcaactacaagctgtgctccgacgataacatcaaggccgagacctatatccacgagatcagccacatcctgaataacttcgaggcccaggagctgaagtacaatcctgagatccacctggtggagtctgagctgaaggccagcgagctgaagaatgtgctggacgtgatcatgaacgccttccactggtgttccgtgtttatgaccgaggagctggtggacaaggataataacttttatgccgagctggaggagatctacgatgagatctatccagtgatctccctgtataatctggtgaggaactacgtgacccagaagccctattctacaaagaagatcaagctgaacttcggcatccctacactggccgacggctggtccaagtctaaggagtacagcaataacgccatcatcctgatgcgcgataatctgtactatctgggcatctttaatgccaagaacaagccagacaagaagatcatcgagggcaatacctccgagaacaagggcgattacaagaagatgatctataatctgctgcccggccctaacaagatgatcccaaaggtgttcctgtcctctaagaccggcgtggagacatacaagcccagcgcctatatcctggagggctacaagcagaacaagcacatcaagagctccaaggacttcgatatcacattttgccacgatctgatcgactacttcaagaattgtatcgccatccaccccgagtggaaaaacttcggctttgatttctccgacacctctacatacgaggacatctccggcttttatcgggaggtggagctgcagggctacaagatcgattggacctatatctctgagaaggacatcgatctgctgcaggagaagggccagctgtatctgttccagatctacaacaaggacttcagcaagaagagcaccggcaatgacaacctgcacacaatgtacctgaagaatctgttcagcgaggagaacctgaaggacatcgtgctgaagctgaatggcgaggccgagatcttctttagaaagtctagcatcaagaatcccatcatccacaagaagggctccatcctggtgaaccggacctacgaggccgaggagaaggaccagttcggcaacatccagatcgtgagaaagaatatccctgagaacatctatcaggagctgtacaagtacttcaacgataaatccgacaaggagctgtctgatgaggccgccaagctgaagaatgtggtgggccaccacgaggccgccacaaacatcgtgaaggattaccggtatacctacgataagtacttcctgcacatgcccatcacaatcaatttcaaggccaacaagaccggctttatcaacgacagaatcctgcagtacatcgccaaggagaaggatctgcacgtgatcggcatcgacaggggcgagcgcaatctgatctatgtgagcgtgatcgacacctgtggcaacatcgtggagcagaagtcctttaatatcgtgaacggctatgattaccagatcaagctgaagcagcaggagggagcaaggcagatcgcaagaaaggagtggaaggagatcggcaagatcaaggagatcaaggagggctacctgagcctggtcatccacgagatctccaagatggtcatcaagtacaacgccatcatcgccatggaggacctgagctatggcttcaagaagggccggtttaaggtggagagacaggtgtaccagaagttcgagaccatgctgatcaataagctgaactatctggtgtttaaggacatctccatcacagagaacggcggcctgctgaagggctaccagctgacctatatccctgataagctgaagaatgtgggccaccagtgcggctgtatcttctatgtgccagccgcctacacaagcaagatcgaccccaccacaggctttgtgaacatctttaagttcaaggatctgaccgtggacgccaagagggagttcatcaagaagtttgatagcatccgctacgactccgagaagaacctgttttgcttcacatttgattacaacaacttcatcacccagaatacagtgatgtctaagtcctcttggagcgtgtatacctacggcgtgaggatcaagaggcgcttcgtgaatggccgcttttctaacgagagcgataccatcgacatcacaaaggatatggagaagaccctggagatgacagacatcaactggcgggatggccacgacctgagacaggatatcatcgactacgagatcgtgcagcacatcttcgagatctttaggctgacagtgcagatgcgcaacagcctgtccgagctggaggacagggattacgaccgcctgatctctcctgtgctgaatgagaataacatcttctatgatagcgccaaggcaggcgacgcactgccaaaggatgcagacgccaacggcgcctactgtatcgccctgaagggcctgtatgagatcaagcagatcaccgagaattggaaggaggatggcaagttttctagggacaagctgaagatcagcaataaggattggttcgactttatccagaacaagcggtacctgggaggaggaggctccggcggaggaggctctcaggtgaagctggaggagagcggaggaggctccgtgcagaccggaggctccctgaggctgacatgcgcagcatctggacggacctctagaagctacggaatgggatggttcaggcaggcaccaggcaaggagagagagttcgtgagcggcatctcttggcgcggcgattctaccggctatgccgacagcgtgaagggcaggttcacaatctctcgcgataatgccaagaacaccgtggacctgcagatgaacagcctgaagcccgaggacacagccatctactattgtgcagcagcagcaggcagcgcctggtacggcaccctgtatgagtacgattattggggccagggcacccaggtgacagtgagctccgccctggagcccaagaagaagcggaaggtggaggaccccaagaagaagcggaaagtggagaatctgtattttcagggcggctctagccatcatcaccatcatcaccaccaccaccactga

SEQ ID NO: 73 - protein, 1456 aa, Artificial Sequence
  Description of Artificial Sequence: Synthetic polypeptide
MYRMQLLSCIALSLALVTNSMNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYLGGGGSGGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSALEPKKKRKVEDPKKKRKVENLYFQGGSSHHHHHHHHHH

SEQ ID NO: 74 - DNA, 4386 bp, Artificial Sequence
  Description of Artificial Sequence: Synthetic polynucleotide
atgtacaggatgcaactcctgtcttgcattgcactaagtcttgcacttgtcacgaactctatgaacaatggcaccaacaatttccagaacttcatcggcatcagctccctgcagaagacactgcggaacgccctgatccctaccgagaccacacagcagttcatcgtgaagaatggcatcatcaaggaggatgagctgaggggcgagaaccgccagatcctgaaggacatcatggacgattactatagaggcttcatctctgagacactgtctagcatcgacgatatcgactggaccagcctgtttgagaagatggagatccagctgaagaatggcgataacaaggacaccctgatcaaggagcagacagagtaccggaaggccatccacaagaagttcgccaatgacgatagattcaagaacatgttttctgccaagctgatcagcgatatcctgccagagtttgtgatccacaacaataactactccgcctctgagaaggaggagaagacacaggtcatcaagctgttcagcaggtttgccacctctttcaaggactacttcaagaatcgcgccaactgcttctccgccgacgatatctcctctagctcctgtcacaggatcgtgaatgataacgccgagatcttcttttctaacgccctggtgtaccggagaatcgtgaagagcctgtccaatgacgatatcaacaagatcagcggcgatatgaaggacagcctgaaggagatgtccctggaggagatctactcctatgagaagtacggcgagttcatcacacaggagggcatctccttttataacgacatctgcggcaaggtcaattctttcatgaacctgtactgtcagaagaataaggagaataagaacctgtataagctgcagaagctgcacaagcagatcctgtgcatcgccgatacctcctacgaggtgccctataagttcgagtctgacgaggaggtgtaccagagcgtgaatggctttctggataacatctctagcaagcacatcgtggagcggctgagaaagatcggcgataattacaacggctataacctggacaagatctatatcgtgagcaagttttacgagtctgtgagccagaagacctaccgggactgggagaccatcaatacagccctggagatccactataataacatcctgcctggcaacggcaagtccaaggccgataaggtgaagaaggccgtgaagaatgacctgcagaagtctatcacagagatcaatgagctggtgtccaactacaagctgtgctctgacgataacatcaaggccgagacctatatccacgagatctcccacatcctgaataacttcgaggcccaggagctgaagtacaatcctgagatccacctggtggagtctgagctgaaggccagcgagctgaagaatgtgctggacgtgatcatgaacgccttccactggtgtagcgtgtttatgaccgaggagctggtggacaaggataataacttttatgccgagctggaggagatctacgatgagatctatccagtgatctctctgtataatctggtgaggaactacgtgacccagaagccctatagcacaaagaagatcaagctgaacttcggcatccctacactggccgacggctggtccaagtctaaggagtactccaataacgccatcatcctgatgcgcgataatctgtactatctgggcatctttaatgccaagaacaagccagacaagaagatcatcgagggcaataccagcgagaacaagggcgattacaagaagatgatctataatctgctgcccggccctaacaagatgatcccaaaggtgttcctgtcctctaagaccggcgtggagacatacaagcccagcgcctatatcctggagggctacaagcagaacaagcacatcaagagctccaaggacttcgatatcacattttgccacgatctgatcgactacttcaagaattgtatcgccatccaccccgagtggaagaacttcggctttgatttctccgacacctctacatacgaggacatctctggcttttatcgggaggtggagctgcagggctacaagatcgattggacctatatcagcgagaaggacatcgatctgctgcaggagaagggccagctgtatctgttccagatctacaacaaggacttcagcaagaagagcaccggcaatgacaacctgcacacaatgtacctgaagaatctgttctccgaggagaacctgaaggacatcgtgctgaagctgaatggcgaggccgagatcttctttagaaagtctagcatcaagaatcccatcatccacaagaagggcagcatcctggtgaaccggacctacgaggccgaggagaaggaccagttcggcaacatccagatcgtgagaaagaatatccctgagaacatctatcaggagctgtacaagtacttcaacgataagtccgacaaggagctgtctgatgaggccgccaagctgaagaatgtggtgggccaccacgaggccgccacaaacatcgtgaaggattaccggtatacctacgacaagtacttcctgcacatgcccatcacaatcaatttcaaggccaacaagaccggctttatcaacgacagaatcctgcagtacatcgccaaggagaaggatctgcacgtgatcggcatcgacaggggcgagcgcaatctgatctacgtgagcgtgatcgacacctgtggcaacatcgtggagcagaagtcttttaatatcgtgaacggctatgattaccagatcaagctgaagcagcaggagggagcaaggcagatcgcaagaaaggagtggaaggagatcggcaagatcaaggagatcaaggagggctacctgagcctggtcatccacgagatctctaagatggtcatcaagtacaacgccatcatcgccatggaggacctgtcctatggcttcaagaaaggccggtttaaggtggagagacaggtgtaccagaagttcgagaccatgctgatcaataagctgaactatctggtgtttaaggacatcagcatcacagagaacggcggcctgctgaagggctaccagctgacctatatccctgataagctgaagaatgtgggccaccagtgcggctgtatcttctatgtgccagccgcctacacaagcaagatcgaccccaccacaggctttgtgaacatctttaagttcaaggatctgaccgtggacgccaagagggagttcatcaagaagtttgatagcatccgctacgactccgagaagaacctgttttgcttcacatttgattacaacaacttcatcacccagaatacagtgatgtctaagtcctcttggagcgtgtatacctacggcgtgaggatcaagaggcgcttcgtgaatggccgcttttctaacgagagcgataccatcgacatcacaaaggatatggagaagaccctggagatgacagacatcaactggcgggatggccacgacctgagacaggatatcatcgactacgagatcgtgcagcacatcttcgagatctttaggctgacagtgcagatgcgcaacagcctgtccgagctggaggacagggattacgaccgcctgatcagccctgtgctgaatgagaataacatcttctatgattccgccaaggcaggcgacgcactgccaaaggatgcagacgccaacggcgcctactgtatcgccctgaagggcctgtatgagatcaagcagatcaccgagaattggaaggaggatggcaagtttagcagggacaagctgaagatctccaataaggattggttcgactttatccagaacaagcggtacctgggaggaggaggctccggcggaggaggctctggcggcggcggcagccaggtgaagctggaggagagcggaggaggctccgtgcagaccggaggctctctgaggctgacatgcgcagcaagcggacggacctctagaagctacggaatgggatggttcaggcaggcaccaggcaaggagagagagttcgtgagcggcatctcttggcgcggcgatagcaccggctatgccgactccgtgaagggcaggttcacaatcagccgcgataatgccaagaacaccgtggacctgcagatgaactccctgaagcccgaggacacagccatctactattgtgcagcagcagcaggcagcgcctggtacggcaccctgtatgagtacgattattggggccagggcacccaggtgacagtgagctccgccctggagcccaagaagaagcggaaggtggaggaccccaagaagaagcggaaagtggagaatctgtattttcagggcggctctagccatcatcaccatcatcaccaccaccaccactga

SEQ ID NO: 75 - protein, 1461 aa, Artificial Sequence
  Description of Artificial Sequence: Synthetic polypeptide
MYRMQLLSCIALSLALVTNSMNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYLGGGGSGGGGSGGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSALEPKKKRKVEDPKKKRKVENLYFQGGSSHHHHHHHHHH

SEQ ID NO: 76 - DNA, 4401 bp, Artificial Sequence
  Description of Artificial Sequence: Synthetic polynucleotide
atgtacaggatgcaactcctgtcttgcattgcactaagtcttgcacttgtcacgaactctatgaacaatggcaccaacaatttccagaacttcatcggcatcagctccctgcagaagacactgcggaacgccctgatccctaccgagaccacacagcagttcatcgtgaagaatggcatcatcaaggaggatgagctgaggggcgagaaccgccagatcctgaaggacatcatggacgattactatagaggcttcatcagcgagacactgtctagcatcgacgatatcgactggacctccctgtttgagaagatggagatccagctgaagaatggcgataacaaggacaccctgatcaaggagcagacagagtaccggaaggccatccacaagaagttcgccaatgacgatagattcaagaacatgttttctgccaagctgatcagcgatatcctgccagagtttgtgatccacaacaataactactccgcctctgagaaggaggagaagacacaggtcatcaagctgttcagcaggtttgccacctctttcaaggactacttcaagaatcgcgccaactgcttctctgccgacgatatctcctctagctcctgtcacaggatcgtgaatgataacgccgagatcttcttttccaacgccctggtgtaccggagaatcgtgaagagcctgtccaatgacgatatcaacaagatctccggcgatatgaaggacagcctgaaggagatgtccctggaggagatctactcttatgagaagtacggcgagttcatcacacaggagggcatctctttttataacgacatctgcggcaaggtcaatagcttcatgaacctgtactgtcagaagaataaggagaataagaacctgtataagctgcagaagctgcacaagcagatcctgtgcatcgccgataccagctacgaggtgccctataagttcgagagcgacgaggaggtgtaccagtccgtgaatggctttctggataacatctctagcaagcacatcgtggagcggctgagaaagatcggcgataattacaacggctataacctggacaagatctatatcgtgtccaagttttacgagtctgtgagccagaagacctaccgggactgggagaccatcaatacagccctggagatccactataataacatcctgcctggcaacggcaagtctaaggccgataaggtgaagaaggccgtgaagaatgacctgcagaagagcatcacagagatcaatgagctggtgtctaactacaagctgtgcagcgacgataacatcaaggccgagacctatatccacgagatcagccacatcctgaataacttcgaggcccaggagctgaagtacaatcctgagatccacctggtggagtctgagctgaaggccagcgagctgaagaatgtgctggacgtgatcatgaacgccttccactggtgttccgtgtttatgaccgaggagctggtggacaaggataataacttttatgccgagctggaggagatctacgatgagatctatccagtgatcagcctgtataatctggtgaggaactacgtgacccagaagccctattccacaaagaagatcaagctgaacttcggcatccctacactggccgacggctggtccaagtctaaggagtacagcaataacgccatcatcctgatgcgcgataatctgtactatctgggcatctttaatgccaagaacaagccagacaagaagatcatcgagggcaatacctccgagaacaagggcgattacaagaagatgatctataatctgctgcccggccctaacaagatgatcccaaaggtgttcctgtcctctaagaccggcgtggagacatacaagcccagcgcctatatcctggagggctacaagcagaacaagcacatcaagagctccaaggacttcgatatcacattttgccacgatctgatcgactacttcaagaattgtatcgccatccaccccgagtggaaaaacttcggctttgatttctccgacacctctacatacgaggacatcagcggcttttatcgggaggtggagctgcagggctacaagatcgattggacctatatctccgagaaggacatcgatctgctgcaggagaagggccagctgtatctgttccagatctacaacaaggacttcagcaagaagagcaccggcaatgacaacctgcacacaatgtacctgaagaatctgttcagcgaggagaacctgaaggacatcgtgctgaagctgaatggcgaggccgagatcttctttagaaagtctagcatcaagaatcccatcatccacaagaagggctccatcctggtgaaccggacctacgaggccgaggagaaggaccagttcggcaacatccagatcgtgagaaagaatatccctgagaacatctatcaggagctgtacaagtacttcaacgataagtccgacaaggagctgtctgatgaggccgccaagctgaagaatgtggtgggccaccacgaggccgccacaaacatcgtgaaggattaccggtatacctacgacaagtacttcctgcacatgcccatcacaatcaatttcaaggccaacaagaccggctttatcaacgacagaatcctgcagtacatcgccaaggagaaggatctgcacgtgatcggcatcgacaggggcgagcgcaatctgatctacgtgagcgtgatcgacacctgtggcaacatcgtggagcagaagagctttaatatcgtgaacggctatgattaccagatcaagctgaagcagcaggagggagcaaggcagatcgcaagaaaggagtggaaggagatcggcaagatcaaggagatcaaggagggctacctgagcctggtcatccacgagatcagcaagatggtcatcaagtacaacgccatcatcgccatggaggacctgagctatggcttcaagaagggccggtttaaggtggagagacaggtgtaccagaagttcgagaccatgctgatcaataagctgaactatctggtgtttaaggacatctccatcacagagaacggcggcctgctgaagggctaccagctgacctatatccctgataagctgaagaatgtgggccaccagtgcggctgtatcttctatgtgccagccgcctacacaagcaagatcgaccccaccacaggctttgtgaacatctttaagttcaaggatctgaccgtggacgccaagagggagttcatcaagaagtttgatagcatccgctacgactccgagaagaacctgttttgcttcacatttgattacaacaacttcatcacccagaatacagtgatgtctaagtcctcttggagcgtgtatacctacggcgtgaggatcaagaggcgcttcgtgaatggccgcttttctaacgagagcgataccatcgacatcacaaaggatatggagaagaccctggagatgacagacatcaactggcgggatggccacgacctgagacaggatatcatcgactacgagatcgtgcagcacatcttcgagatctttaggctgacagtgcagatgcgcaacagcctgtccgagctggaggacagggattacgaccgcctgatctcccctgtgctgaatgagaataacatcttctatgattctgccaaggcaggcgacgcactgccaaaggatgcagacgccaacggcgcctactgtatcgccctgaagggcctgtatgagatcaagcagatcaccgagaattggaaggaggatggcaagttttccagggacaagctgaagatctctaataaggattggttcgactttatccagaacaagcggtacctgggaggaggaggctccggcggaggaggctctggcggcggcggcagcggaggcggcggctcccaggtgaagctggaggagagcggaggaggctccgtgcagaccggaggcagcctgaggctgacatgcgcagcatccggacggacctctagaagctacggaatgggatggttcaggcaggcaccaggcaaggagagagagttcgtgagcggcatctcttggcgcggcgattccaccggctatgccgactctgtgaagggcaggttcacaatctcccgcgataatgccaagaacaccgtggacctgcagatgaactctctgaagcccgaggacacagccatctactattgtgcagcagcagcaggcagcgcctggtacggcaccctgtatgagtacgattattggggccagggcacccaggtgacagtgagctccgccctggagcccaagaagaagcggaaggtggaggaccccaagaagaagcggaaagtggagaatctgtattttcagggcggctctagccatcatcaccatcatcaccaccaccaccactga

SEQ ID NO: 77 - protein, 1466 aa, Artificial Sequence
  Description of Artificial Sequence: Synthetic polypeptide
MYRMQLLSCIALSLALVTNSMNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYLGGGGSGGGGSGGGGSGGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSALEPKKKRKVEDPKKKRKVENLYFQGGSSHHHHHHHHHH

SEQ ID NO: 78 - DNA, 4965 bp, Artificial Sequence
  Description of Artificial Sequence: Synthetic polynucleotide
atgcatcatcatcatcatcacagcagcggcagagaaaacttgtatttccagggcatgaacaacggcaccaacaactttcagaactttattggcattagcagcctgcagaaaaccctgcgcaacgcgctgattccgaccgaaaccacccagcagtttattgtgaaaaacggcattattaaagaagatgaactgcgcggcgaaaaccgccagattctgaaagatattatggatgattattatcgcggctttattagcgaaaccctgagcagcattgatgatattgattggaccagcctgtttgaaaaaatggaaattcagctgaaaaacggcgataacaaagataccctgattaaagaacagaccgaatatcgcaaagcgattcataaaaaatttgcgaacgatgatcgctttaaaaacatgtttagcgcgaaactgattagcgatattctgccggaatttgtgattcataacaacaactatagcgcgagcgaaaaagaagaaaaaacccaggtgattaaactgtttagccgctttgcgaccagctttaaagattattttaaaaaccgcgcgaactgctttagcgcggatgatattagcagcagcagctgccatcgcattgtgaacgataacgcggaaattttttttagcaacgcgctggtgtatcgccgcattgtgaaaagcctgagcaacgatgatattaacaaaattagcggcgatatgaaagatagcctgaaagaaatgagcctggaagaaatttatagctatgaaaaatatggcgaatttattacccaggaaggcattagcttttataacgatatttgcggcaaagtgaacagctttatgaacctgtattgccagaaaaacaaagaaaacaaaaacctgtataaactgcagaaactgcataaacagattctgtgcattgcggataccagctatgaagtgccgtataaatttgaaagcgatgaagaagtgtatcagagcgtgaacggctttctggataacattagcagcaaacatattgtggaacgcctgcgcaaaattggcgataactataacggctataacctggataaaatttatattgtgagcaaattttatgaaagcgtgagccagaaaacctatcgcgattgggaaaccattaacaccgcgctggaaattcattataacaacattctgccgggcaacggcaaaagcaaagcggataaagtgaaaaaagcggtgaaaaacgatctgcagaaaagcattaccgaaattaacgaactggtgagcaactataaactgtgcagcgatgataacattaaagcggaaacctatattcatgaaattagccatattctgaacaactttgaagcgcaggaactgaaatataacccggaaattcatctggtggaaagcgaactgaaagcgagcgaactgaaaaacgtgctggatgtgattatgaacgcgtttcattggtgcagcgtgtttatgaccgaagaactggtggataaagataacaacttttatgcggaactggaagaaatttatgatgaaatttatccggtgattagcctgtataacctggtgcgcaactatgtgacccagaaaccgtatagcaccaaaaaaattaaactgaactttggcattccgaccctggcggatggctggagcaaaagcaaagaatatagcaacaacgcgattattctgatgcgcgataacctgtattatctgggcatttttaacgcgaaaaacaaaccggataaaaaaattattgaaggcaacaccagcgaaaacaaaggcgattataaaaaaatgatttataacctgctgccgggcccgaacaaaatgattccgaaagtgtttctgagcagcaaaaccggcgtggaaacctataaaccgagcgcgtatattctggaaggctataaacagaacaaacatattaaaagcagcaaagattttgatattaccttttgccatgatctgattgattattttaaaaactgcattgcgattcatccggaatggaaaaactttggctttgattttagcgataccagcacctatgaagatattagcggcttttatcgcgaagtggaactgcagggctataaaattgattggacctatattagcgaaaaagatattgatctgctgcaggaaaaaggccagctgtatctgtttcagatttataacaaagattttagcaaaaaaagcaccggcaacgataacctgcataccatgtatctgaaaaacctgtttagcgaagaaaacctgaaagatattgtgctgaaactgaacggcgaagcggaaattttttttcgcaaaagcagcattaaaaacccgattattcataaaaaaggcagcattctggtgaaccgcacctatgaagcggaagaaaaagatcagtttggcaacattcagattgtgcgcaaaaacattccggaaaacatttatcaggaactgtataaatattttaacgataaaagcgataaagaactgagcgatgaagcggcgaaactgaaaaacgtggtgggccatcatgaagcggcgaccaacattgtgaaagattatcgctatacctatgataaatattttctgcatatgccgattaccattaactttaaagcgaacaaaaccggctttattaacgatcgcattctgcagtatattgcgaaagaaaaagatctgcatgtgattggcattgatcgcggcgaacgcaacctgatttatgtgagcgtgattgatacctgcggcaacattgtggaacagaaaagctttaacattgtgaacggctatgattatcagattaaactgaaacagcaggaaggcgcgcgccagattgcgcgcaaagaatggaaagaaattggcaaaattaaagaaattaaagaaggctatctgagcctggtgattcatgaaattagcaaaatggtgattaaatataacgcgattattgcgatggaagatctgagctatggctttaaaaaaggccgctttaaagtggaacgccaggtgtatcagaaatttgaaaccatgctgattaacaaactgaactatctggtgtttaaagatattagcattaccgaaaacggcggcctgctgaaaggctatcagctgacctatattccggataaactgaaaaacgtgggccatcagtgcggctgcattttttatgtgccggcggcgtataccagcaaaattgatccgaccaccggctttgtgaacatttttaaatttaaagatctgaccgtggatgcgaaacgcgaatttattaaaaaatttgatagcattcgctatgatagcgaaaaaaacctgttttgctttacctttgattataacaactttattacccagaacaccgtgatgagcaaaagcagctggagcgtgtatacctatggcgtgcgcattaaacgccgctttgtgaacggccgctttagcaacgaaagcgataccattgatattaccaaagatatggaaaaaaccctggaaatgaccgatattaactggcgcgatggccatgatctgcgccaggatattattgattatgaaattgtgcagcatatttttgaaatttttcgcctgaccgtgcagatgcgcaacagcctgagcgaactggaagatcgcgattatgatcgcctgattagcccggtgctgaacgaaaacaacattttttatgatagcgcgaaagcgggcgatgcgctgccgaaagatgcggatgcgaacggcgcgtattgcattgcgctgaaaggcctgtatgaaattaaacagattaccgaaaactggaaagaagatggcaaatttagccgcgataaactgaaaattagcaacaaagattggtttgattttattcagaacaaacgctatctgggcggcggcggcagcggcggcggcggcagcggcggcggcggcagcggcggcggcggcagcggcggcggcggcagcggcggcggcggcagcaccagccctaagaaaaaacgaaaagttgaggatcctaaaaagaaacgaaaagttcatcatcatcatcatcatgaatttgcgagcgcggaagcgggcattaccggcacctggtataaccagcatggcagcacctttaccgtgaccgcgggcgcggatggcaacctgaccggccagtatgaaaaccgcgcgcagggcaccggctgccagaacagcccgtataccctgaccggccgctataacggcaccaaactggaatggcgcgtggaatggaacaacagcaccgaaaactgccatagccgcaccgaatggcgcggccagtatcagggcggcgcggaagcgcgcattaacacccagtggaacctgacctatgaaggcggcagcggcccggcgaccgaacagggccaggatacctttaccaaagtgaaaccgagcgcggcgagcggcagcgattataaagatgatgatgataaaaaacgcaaaagaaaatgccgatatcctattggcattgacgtcaggtggcacttttcgaggagatcatgcacaggcggcggcggcagcggcggcggcggcagcggcggcggcggcagcggcggcggcggcagcggcggcggcggcagcggcggcagcccatgggcggcgcaggttaaactggaagaatctggtggtggttctgttcagaccggtggttctctgcgtctgacctgcgcggcgtctggtcgtacctctcgttcttacggtatgggttggttccgtcaggcgccgggtaaagaacgtgaattcgtttctggtatctcttggcgtggtgactctaccggttacgcggactctgttaaaggtcgtttcaccatctctcgtgacaacgcgaaaaacaccgttgacctgcagatgaactctctgaaaccggaagacaccgcgatctactactgcgcggcggcggcgggttctgcgtggtacggtaccctgtacgaatacgactactggggtcagggtacccaggttaccgtttcttcttgttgttgttgttgttgttaa

SEQ ID NO: 79 - protein, 1654 aa, Artificial Sequence
  Description of Artificial Sequence: Synthetic polypeptide
MHHHHHHSSGRENLYFQGMNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYLGGGGSGGGGSGGGGSGGGGSGGGGSGGGGSTSPKKKRKVEDPKKKRKVHHHHHHEFASAEAGITGTWYNQHGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKPSAASGSDYKDDDDKKRKRKCRYPIGIDVRWHFSRRSCTGGGGSGGGGSGGGGSGGGGSGGGGSGGSPWAAQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSCCCCCC

SEQ ID NO: 80 - DNA, 4923 bp, Artificial Sequence
  Description of Artificial Sequence: Synthetic polynucleotide
atgcatcatcatcatcatcacagcagcggcagagaaaacttgtatttccagggcatgaacaacggcaccaacaactttcagaactttattggcattagcagcctgcagaaaaccctgcgcaacgcgctgattccgaccgaaaccacccagcagtttattgtgaaaaacggcattattaaagaagatgaactgcgcggcgaaaaccgccagattctgaaagatattatggatgattattatcgcggctttattagcgaaaccctgagcagcattgatgatattgattggaccagcctgtttgaaaaaatggaaattcagctgaaaaacggcgataacaaagataccctgattaaagaacagaccgaatatcgcaaagcgattcataaaaaatttgcgaacgatgatcgctttaaaaacatgtttagcgcgaaactgattagcgatattctgccggaatttgtgattcataacaacaactatagcgcgagcgaaaaagaagaaaaaacccaggtgattaaactgtttagccgctttgcgaccagctttaaagattattttaaaaaccgcgcgaactgctttagcgcggatgatattagcagcagcagctgccatcgcattgtgaacgataacgcggaaattttttttagcaacgcgctggtgtatcgccgcattgtgaaaagcctgagcaacgatgatattaacaaaattagcggcgatatgaaagatagcctgaaagaaatgagcctggaagaaatttatagctatgaaaaatatggcgaatttattacccaggaaggcattagcttttataacgatatttgcggcaaagtgaacagctttatgaacctgtattgccagaaaaacaaagaaaacaaaaacctgtataaactgcagaaactgcataaacagattctgtgcattgcggataccagctatgaagtgccgtataaatttgaaagcgatgaagaagtgtatcagagcgtgaacggctttctggataacattagcagcaaacatattgtggaacgcctgcgcaaaattggcgataactataacggctataacctggataaaatttatattgtgagcaaattttatgaaagcgtgagccagaaaacctatcgcgattgggaaaccattaacaccgcgctggaaattcattataacaacattctgccgggcaacggcaaaagcaaagcggataaagtgaaaaaagcggtgaaaaacgatctgcagaaaagcattaccgaaattaacgaactggtgagcaactataaactgtgcagcgatgataacattaaagcggaaacctatattcatgaaattagccatattctgaacaactttgaagcgcaggaactgaaatataacccggaaattcatctggtggaaagcgaactgaaagcgagcgaactgaaaaacgtgctggatgtgattatgaacgcgtttcattggtgcagcgtgtttatgaccgaagaactggtggataaagataacaacttttatgcggaactggaagaaatttatgatgaaatttatccggtgattagcctgtataacctggtgcgcaactatgtgacccagaaaccgtatagcaccaaaaaaattaaactgaactttggcattccgaccctggcggatggctggagcaaaagcaaagaatatagcaacaacgcgattattctgatgcgcgataacctgtattatctgggcatttttaacgcgaaaaacaaaccggataaaaaaattattgaaggcaacaccagcgaaaacaaaggcgattataaaaaaatgatttataacctgctgccgggcccgaacaaaatgattccgaaagtgtttctgagcagcaaaaccggcgtggaaacctataaaccgagcgcgtatattctggaaggctataaacagaacaaacatattaaaagcagcaaagattttgatattaccttttgccatgatctgattgattattttaaaaactgcattgcgattcatccggaatggaaaaactttggctttgattttagcgataccagcacctatgaagatattagcggcttttatcgcgaagtggaactgcagggctataaaattgattggacctatattagcgaaaaagatattgatctgctgcaggaaaaaggccagctgtatctgtttcagatttataacaaagattttagcaaaaaaagcaccggcaacgataacctgcataccatgtatctgaaaaacctgtttagcgaagaaaacctgaaagatattgtgctgaaactgaacggcgaagcggaaattttttttcgcaaaagcagcattaaaaacccgattattcataaaaaaggcagcattctggtgaaccgcacctatgaagcggaagaaaaagatcagtttggcaacattcagattgtgcgcaaaaacattccggaaaacatttatcaggaactgtataaatattttaacgataaaagcgataaagaactgagcgatgaagcggcgaaactgaaaaacgtggtgggccatcatgaagcggcgaccaacattgtgaaagattatcgctatacctatgataaatattttctgcatatgccgattaccattaactttaaagcgaacaaaaccggctttattaacgatcgcattctgcagtatattgcgaaagaaaaagatctgcatgtgattggcattgatcgcggcgaacgcaacctgatttatgtgagcgtgattgatacctgcggcaacattgtggaacagaaaagctttaacattgtgaacggctatgattatcagattaaactgaaacagcaggaaggcgcgcgccagattgcgcgcaaagaatggaaagaaattggcaaaattaaagaaattaaagaaggctatctgagcctggtgattcatgaaattagcaaaatggtgattaaatataacgcgattattgcgatggaagatctgagctatggctttaaaaaaggccgctttaaagtggaacgccaggtgtatcagaaatttgaaaccatgctgattaacaaactgaactatctggtgtttaaagatattagcattaccgaaaacggcggcctgctgaaaggctatcagctgacctatattccggataaactgaaaaacgtgggccatcagtgcggctgcattttttatgtgccggcggcgtataccagcaaaattgatccgaccaccggctttgtgaacatttttaaatttaaagatctgaccgtggatgcgaaacgcgaatttattaaaaaatttgatagcattcgctatgatagcgaaaaaaacctgttttgctttacctttgattataacaactttattacccagaacaccgtgatgagcaaaagcagctggagcgtgtatacctatggcgtgcgcattaaacgccgctttgtgaacggccgctttagcaacgaaagcgataccattgatattaccaaagatatggaaaaaaccctggaaatgaccgatattaactggcgcgatggccatgatctgcgccaggatattattgattatgaaattgtgcagcatatttttgaaatttttcgcctgaccgtgcagatgcgcaacagcctgagcgaactggaagatcgcgattatgatcgcctgattagcccggtgctgaacgaaaacaacattttttatgatagcgcgaaagcgggcgatgcgctgccgaaagatgcggatgcgaacggcgcgtattgcattgcgctgaaaggcctgtatgaaattaaacagattaccgaaaactggaaagaagatggcaaatttagccgcgataaactgaaaattagcaacaaagattggtttgattttattcagaacaaacgctatctgggcggcggcggcagcggcggcggcggcagcggcggcggcggcagcggcggcggcggcagcggcggcggcggcagcggcggcggcggcagcaccagccctaagaaaaaacgaaaagttgaggatcctaaaaagaaacgaaaagttcatcatcatcatcatcatgaatttgcgagcgcggaagcgggcattaccggcacctggtataaccagcatggcagcacctttaccgtgaccgcgggcgcggatggcaacctgaccggccagtatgaaaaccgcgcgcagggcaccggctgccagaacagcccgtataccctgaccggccgctataacggcaccaaactggaatggcgcgtggaatggaacaacagcaccgaaaactgccatagccgcaccgaatggcgcggccagtatcagggcggcgcggaagcgcgcattaacacccagtggaacctgacctatgaaggcggcagcggcccggcgaccgaacagggccaggatacctttaccaaagtgaaaccgagcgcggcgagcggcagcgattataaagatgatgatgataaaaaacgcaaaagaaaatgccgatatcctattggcattgacgtcaggtggcacttttcgaggagatcatgcacaggcggcggcggcagcggcggcggcggcagcggcggcggcggcagcggcggcggcggcagcggcggcggcggcagcggcggcagccaggtgcagctgcaggagtctggaggaggcttggtgcagcctggggggtctctgagactctcctgtgcagcctctggattcacattcagtagctacgacatgagctgggtccgccaggctccggggaaggggctcgagtgggtctcaggtatgaatagtggtggtggtagaacatactatgaagactccgtgaagggccgattcaccatctccaggtccaacgccaagaacacgctgtatctgcaactgaacagcctgaaaactgacgacacggccatgtattactgtgtcacatccgactttgcttactggggccaggggacccaggtcaccgtctcctcatgttgttgttgttgttgttaa

SEQ ID NO: 81 - protein, 1640 aa, Artificial Sequence
  Description of Artificial Sequence: Synthetic polypeptide
MHHHHHHSSGRENLYFQGMNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYLGGGGSGGGGSGGGGSGGGGSGGGGSGGGGSTSPKKKRKVEDPKKKRKVHHHHHHEFASAEAGITGTWYNQHGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKPSAASGSDYKDDDDKKRKRKCRYPIGIDVRWHFSRRSCTGGGGSGGGGSGGGGSGGGGSGGGGSGGSQVQLQESGGGLVQPGGSLRLSCAASGFTFSSYDMSWVRQAPGKGLEWVSGMNSGGGRTYYEDSVKGRFTISRSNAKNTLYLQLNSLKTDDTAMYYCVTSDFAYWGQGTQVTVSSCCCCCC

SEQ ID NO: 82 - DNA, 471 bp, Artificial Sequence
  Description of Artificial Sequence: Synthetic polynucleotide
gaatttgcgagcgcggaagcgggcattaccggcacctggtataaccagcatggcagcacctttaccgtgaccgcgggcgcggatggcaacctgaccggccagtatgaaaaccgcgcgcagggcaccggctgccagaacagcccgtataccctgaccggccgctataacggcaccaaactggaatggcgcgtggaatggaacaacagcaccgaaaactgccatagccgcaccgaatggcgcggccagtatcagggcggcgcggaagcgcgcattaacacccagtggaacctgacctatgaaggcggcagcggcccggcgaccgaacagggccaggatacctttaccaaagtgaaaccgagcgcggcgagcggcagcgattataaagatgatgatgataaaaaacgcaaaagaaaatgccgatatcctattggcattgacgtcaggtggcacttttcgaggagatcatgcaca

SEQ ID NO: 83 - protein, 156 aa, Artificial Sequence
  Description of Artificial Sequence: Synthetic polypeptide
FASAEAGITGTWYNQHGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKPSAASGSDYKDDDDKKRKRKCRYPIGIDVRWHFSRRSCT

SEQ ID NO: 84 - DNA, 4614 bp, Artificial Sequence
  Description of Artificial Sequence: Synthetic polynucleotide
atggataaaaaatacagcattggtctggacattggcacgaatagcgttggttgggcagtgattaccgatgaatacaaagtcccgtcgaaaaaattcaaagtgctgggtaacaccgatcgccatagcattaagaaaaacctgatcggtgcgctgctgtttgattctggcgaaaccgcggaagcaacgcgtctgaaacgtaccgcacgtcgccgttacacgcgccgtaaaaatcgtatttgctatctgcaggaaatctttagcaacgaaatggcgaaagtcgatgactcatttttccaccgcctggaagaatcgtttctggtggaagaagataaaaaacatgaacgtcacccgattttcggcaatatcgttgatgaagtcgcgtaccatgaaaaatatccgacgatttaccacctgcgtaaaaaactggtggattctaccgacaaagccgatctgcgcctgatttatctggcactggctcatatgatcaaatttcgtggtcacttcctgattgaaggcgacctgaacccggataatagtgacgtcgataaactgtttattcagctggtgcaaacctataatcagctgttcgaagaaaacccgatcaatgcaagtggtgttgatgcgaaagccattctgtccgctcgcctgagtaaatcccgccgtctggaaaacctgattgcacagctgccgggtgaaaagaaaaacggtctgtttggcaatctgatcgctctgtcactgggcctgacgccgaactttaaatcgaatttcgacctggcagaagatgctaaactgcagctgagcaaagatacctacgatgacgatctggacaacctgctggcgcaaattggcgaccagtatgccgacctgtttctggcggccaaaaatctgtcagatgccattctgctgtcggacatcctgcgcgtgaacaccgaaatcacgaaagcgccgctgtcagcctcgatgattaaacgctacgatgaacatcaccaggacctgaccctgctgaaagcactggttcgtcagcaactgccggaaaaatacaaagaaattttctttgaccaaagtaaaaatggttatgcaggctacatcgatggcggtgcttcccaggaagaattctacaaattcatcaaaccgatcctggaaaaaatggatggtacggaagaactgctggtgaaactgaatcgtgaagatctgctgcgtaaacaacgcacctttgacaacggtagcattccgcatcagatccacctgggcgaactgcatgcgattctgcgccgtcaggaagatttttatccgttcctgaaagacaaccgtgaaaaaatcgaaaaaatcctgacgtttcgcatcccgtattacgttggtccgctggcacgtggtaatagccgcttcgcatggatgacccgcaaatctgaagaaaccattacgccgtggaactttgaagaagtggttgataaaggcgcaagcgctcagtcttttatcgaacgtatgaccaatttcgataaaaacctgccgaatgaaaaagtgctgccgaaacattctctgctgtatgaatactttaccgtttacaacgaactgacgaaagtgaaatatgttaccgagggtatgcgcaaaccggcgtttctgagtggcgaacagaaaaaagccattgtggatctgctgttcaaaaccaatcgtaaagttacggtcaaacagctgaaagaagattacttcaagaaaattgaatgtttcgacagcgtggaaatttctggtgttgaagatcgtttcaacgcctctctgggcacctatcatgacctgctgaaaatcatcaaagacaaagattttctggataacgaagaaaacgaagacattctggaagatatcgtgctgaccctgacgctgttcgaagatcgtgaaatgattgaagaacgcctgaaaacgtacgcacacctgtttgacgataaagttatgaaacagctgaaacgccgtcgctataccggttggggccgtctgagccgcaaactgattaatggtatccgcgataaacaatcaggcaaaacgattctggatttcctgaaatcggacggctttgccaaccgtaatttcatgcagctgatccatgacgattccctgacctttaaagaagacattcagaaagcacaagtgtcaggtcaaggcgattcgctgcatgaacacattgcgaacctggccggttcaccggctatcaaaaaaggcatcctgcagaccgtgaaagtcgtggatgaactggtgaaagttatgggtcgtcacaaaccggaaaacattgttatcgaaatggcgcgcgaaaatcagaccacgcaaaaaggccagaaaaactcgcgtgaacgcatgaaacgcattgaagaaggtatcaaagaactgggcagccagattctgaaagaacatccggtcgaaaacacccagctgcaaaatgaaaaactgtacctgtattacctgcaaaatggtcgtgacatgtatgtggatcaggaactggacatcaaccgcctgtctgactatgatgtcgaccacattgtgccgcagagctttctgaaagacgattctatcgataacaaagttctgacccgtagtgataaaaaccgcggcaaaagcgacaatgtcccgtctgaagaagttgtgaagaaaatgaaaaactactggcgtcaactgctgaatgcgaaactgattacgcagcgtaaattcgataacctgaccaaagcggaacgcggcggtctgtccgaactggataaagccggttttatcaaacgtcaactggttgaaacccgccagattacgaaacatgtcgcccagatcctggattcacgcatgaacacgaaatacgacgaaaacgataaactgatccgtgaagtcaaagtgatcaccctgaaaagtaaactggtttccgatttccgtaaagactttcagttctacaaagtccgcgaaattaacaattaccatcacgcacacgatgcttatctgaatgcagtggttggtaccgctctgatcaaaaaatatccgaaactggaaagcgaatttgtgtatggcgattacaaagtctatgacgtgcgcaaaatgattgcgaaatccgaacaggaaatcggcaaagcgaccgccaaatactttttctattcaaacatcatgaactttttcaaaaccgaaattacgctggcaaatggtgaaattcgtaaacgcccgctgatcgaaaccaacggtgaaacgggcgaaattgtgtgggataaaggccgtgacttcgcgaccgttcgcaaagtcctgtcgatgccgcaagtgaatatcgtgaagaaaaccgaagtgcagacgggcggttttagtaaagaatccatcctgccgaaacgtaacagcgataaactgattgcgcgcaaaaaagattgggacccgaaaaaatacggcggttttgatagtccgacggttgcatattccgtcctggtcgtggctaaagtcgaaaaaggtaaaagtaaaaaactgaaatccgtgaaagaactgctgggcattaccatcatggaacgtagctcttttgagaaaaacccgattgacttcctggaagccaaaggttacaaagaagtgaaaaaagatctgatcatcaaactgccgaaatatagcctgttcgaactggaaaacggccgtaaacgcatgctggcatctgctggtgaactgcagaaaggcaatgaactggcactgccgagtaaatatgttaactttctgtacctggctagccattatgaaaaactgaaaggttctccggaagataacgaacagaaacaactgttcgtcgaacaacataaacactacctggatgaaatcatcgaacagatctcagaattctcgaaacgcgtgattctggcggatgccaatctggacaaagttctgagcgcgtataacaaacatcgtgataaaccgattcgcgaacaggccgaaaatattatccacctgtttaccctgacgaacctgggcgcaccggcagcttttaaatacttcgataccacgatcgaccgtaaacgctatacctcaacgaaagaagttctggatgctaccctgattcatcaatcgatcaccggtctgtatgaaacgcgtattgatctgagtcagctgggcggtgacggaggaggaggctctggaggaggaggcagccccaagaagaagcggaaggtggaggaccccaagaagaagcggaaagtgggaggaggaggctctggaggaggaggcagccaggtgaaactggaggagagcgggggcgggagcgtgcagactggggggagcctgagactgacatgcgcagcaagcgggcggacaagccggagctacggaatgggatggttcaggcaggcaccaggcaaggagagggagtttgtgagcggcatctcctggagaggcgatagcaccggctatgccgactccgtgaagggcaggttcaccatcagccgcgataatgccaagaacacagtggacctgcagatgaactccctgaagcccgaggacaccgcaatctactattgcgcagcagcagcaggctccgcctggtacggcacactgtacgagtatgattactggggccagggcacccaggtgacagtgagctccgccctggagtgttgttgttgttgttgttaa

SEQ ID NO: 85 - protein, 1537 aa, Artificial Sequence
  Description of Artificial Sequence: Synthetic polypeptide
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGGSGGGGSPKKKRKVEDPKKKRKVGGGGSGGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSALECCCCCC

SEQ ID NO: 86 - DNA, 5085 bp, Artificial Sequence
  Description of Artificial Sequence: Synthetic polynucleotide
atggataaaaaatacagcattggtctggacattggcacgaatagcgttggttgggcagtgattaccgatgaatacaaagtcccgtcgaaaaaattcaaagtgctgggtaacaccgatcgccatagcattaagaaaaacctgatcggtgcgctgctgtttgattctggcgaaaccgcggaagcaacgcgtctgaaacgtaccgcacgtcgccgttacacgcgccgtaaaaatcgtatttgctatctgcaggaaatctttagcaacgaaatggcgaaagtcgatgactcatttttccaccgcctggaagaatcgtttctggtggaagaagataaaaaacatgaacgtcacccgattttcggcaatatcgttgatgaagtcgcgtaccatgaaaaatatccgacgatttaccacctgcgtaaaaaactggtggattctaccgacaaagccgatctgcgcctgatttatctggcactggctcatatgatcaaatttcgtggtcacttcctgattgaaggcgacctgaacccggataatagtgacgtcgataaactgtttattcagctggtgcaaacctataatcagctgttcgaagaaaacccgatcaatgcaagtggtgttgatgcgaaagccattctgtccgctcgcctgagtaaatcccgccgtctggaaaacctgattgcacagctgccgggtgaaaagaaaaacggtctgtttggcaatctgatcgctctgtcactgggcctgacgccgaactttaaatcgaatttcgacctggcagaagatgctaaactgcagctgagcaaagatacctacgatgacgatctggacaacctgctggcgcaaattggcgaccagtatgccgacctgtttctggcggccaaaaatctgtcagatgccattctgctgtcggacatcctgcgcgtgaacaccgaaatcacgaaagcgccgctgtcagcctcgatgattaaacgctacgatgaacatcaccaggacctgaccctgctgaaagcactggttcgtcagcaactgccggaaaaatacaaagaaattttctttgaccaaagtaaaaatggttatgcaggctacatcgatggcggtgcttcccaggaagaattctacaaattcatcaaaccgatcctggaaaaaatggatggtacggaagaactgctggtgaaactgaatcgtgaagatctgctgcgtaaacaacgcacctttgacaacggtagcattccgcatcagatccacctgggcgaactgcatgcgattctgcgccgtcaggaagatttttatccgttcctgaaagacaaccgtgaaaaaatcgaaaaaatcctgacgtttcgcatcccgtattacgttggtccgctggcacgtggtaatagccgcttcgcatggatgacccgcaaatctgaagaaaccattacgccgtggaactttgaagaagtggttgataaaggcgcaagcgctcagtcttttatcgaacgtatgaccaatttcgataaaaacctgccgaatgaaaaagtgctgccgaaacattctctgctgtatgaatactttaccgtttacaacgaactgacgaaagtgaaatatgttaccgagggtatgcgcaaaccggcgtttctgagtggcgaacagaaaaaagccattgtggatctgctgttcaaaaccaatcgtaaagttacggtcaaacagctgaaagaagattacttcaagaaaattgaatgtttcgacagcgtggaaatttctggtgttgaagatcgtttcaacgcctctctgggcacctatcatgacctgctgaaaatcatcaaagacaaagattttctggataacgaagaaaacgaagacattctggaagatatcgtgctgaccctgacgctgttcgaagatcgtgaaatgattgaagaacgcctgaaaacgtacgcacacctgtttgacgataaagttatgaaacagctgaaacgccgtcgctataccggttggggccgtctgagccgcaaactgattaatggtatccgcgataaacaatcaggcaaaacgattctggatttcctgaaatcggacggctttgccaaccgtaatttcatgcagctgatccatgacgattccctgacctttaaagaagacattcagaaagcacaagtgtcaggtcaaggcgattcgctgcatgaacacattgcgaacctggccggttcaccggctatcaaaaaaggcatcctgcagaccgtgaaagtcgtggatgaactggtgaaagttatgggtcgtcacaaaccggaaaacattgttatcgaaatggcgcgcgaaaatcagaccacgcaaaaaggccagaaaaactcgcgtgaacgcatgaaacgcattgaagaaggtatcaaagaactgggcagccagattctgaaagaacatccggtcgaaaacacccagctgcaaaatgaaaaactgtacctgtattacctgcaaaatggtcgtgacatgtatgtggatcaggaactggacatcaaccgcctgtctgactatgatgtcgaccacattgtgccgcagagctttctgaaagacgattctatcgataacaaagttctgacccgtagtgataaaaaccgcggcaaaagcgacaatgtcccgtctgaagaagttgtgaagaaaatgaaaaactactggcgtcaactgctgaatgcgaaactgattacgcagcgtaaattcgataacctgaccaaagcggaacgcggcggtctgtccgaactggataaagccggttttatcaaacgtcaactggttgaaacccgccagattacgaaacatgtcgcccagatcctggattcacgcatgaacacgaaatacgacgaaaacgataaactgatccgtgaagtcaaagtgatcaccctgaaaagtaaactggtttccgatttccgtaaagactttcagttctacaaagtccgcgaaattaacaattaccatcacgcacacgatgcttatctgaatgcagtggttggtaccgctctgatcaaaaaatatccgaaactggaaagcgaatttgtgtatggcgattacaaagtctatgacgtgcgcaaaatgattgcgaaatccgaacaggaaatcggcaaagcgaccgccaaatactttttctattcaaacatcatgaactttttcaaaaccgaaattacgctggcaaatggtgaaattcgtaaacgcccgctgatcgaaaccaacggtgaaacgggcgaaattgtgtgggataaaggccgtgacttcgcgaccgttcgcaaagtcctgtcgatgccgcaagtgaatatcgtgaagaaaaccgaagtgcagacgggcggttttagtaaagaatccatcctgccgaaacgtaacagcgataaactgattgcgcgcaaaaaagattgggacccgaaaaaatacggcggttttgatagtccgacggttgcatattccgtcctggtcgtggctaaagtcgaaaaaggtaaaagtaaaaaactgaaatccgtgaaagaactgctgggcattaccatcatggaacgtagctcttttgagaaaaacccgattgacttcctggaagccaaaggttacaaagaagtgaaaaaagatctgatcatcaaactgccgaaatatagcctgttcgaactggaaaacggccgtaaacgcatgctggcatctgctggtgaactgcagaaaggcaatgaactggcactgccgagtaaatatgttaactttctgtacctggctagccattatgaaaaactgaaaggttctccggaagataacgaacagaaacaactgttcgtcgaacaacataaacactacctggatgaaatcatcgaacagatctcagaattctcgaaacgcgtgattctggcggatgccaatctggacaaagttctgagcgcgtataacaaacatcgtgataaaccgattcgcgaacaggccgaaaatattatccacctgtttaccctgacgaacctgggcgcaccggcagcttttaaatacttcgataccacgatcgaccgtaaacgctatacctcaacgaaagaagttctggatgctaccctgattcatcaatcgatcaccggtctgtatgaaacgcgtattgatctgagtcagctgggcggtgacggaggaggaggctctggaggaggaggcagcgaatttgcgagcgcggaagcgggcattaccggcacctggtataaccagcatggcagcacctttaccgtgaccgcgggcgcggatggcaacctgaccggccagtatgaaaaccgcgcgcagggcaccggctgccagaacagcccgtataccctgaccggccgctataacggcaccaaactggaatggcgcgtggaatggaacaacagcaccgaaaactgccatagccgcaccgaatggcgcggccagtatcagggcggcgcggaagcgcgcattaacacccagtggaacctgacctatgaaggcggcagcggcccggcgaccgaacagggccaggatacctttaccaaagtgaaaccgagcgcggcgagcggcagcgattataaagatgatgatgataaaaaacgcaaaagaaaatgccgatatcctattggcattgacgtcaggtggcacttttcgaggagatcatgcacacccaagaagaagcggaaggtggaggaccccaagaagaagcggaaagtgggaggaggaggctctggaggaggaggcagccaggtgaaactggaggagagcgggggcgggagcgtgcagactggggggagcctgagactgacatgcgcagcaagcgggcggacaagccggagctacggaatgggatggttcaggcaggcaccaggcaaggagagggagtttgtgagcggcatctcctggagaggcgatagcaccggctatgccgactccgtgaagggcaggttcaccatcagccgcgataatgccaagaacacagtggacctgcagatgaactccctgaagcccgaggacaccgcaatctactattgcgcagcagcagcaggctccgcctggtacggcacactgtacgagtatgattactggggccagggcacccaggtgacagtgagctccgccctggagtgttgttgttgttgttgttaa

SEQ ID NO: 87 - protein, 1694 aa, Artificial Sequence
  Description of Artificial Sequence: Synthetic polypeptide
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGGSGGGGSEFASAEAGITGTWYNQHGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKPSAASGSDYKDDDDKKRKRKCRYPIGIDVRWHFSRRSCTPKKKRKVEDPKKKRKVGGGGSGGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSALECCCCCC

SEQ ID NO: 88 - RNA, 21 nt, Artificial Sequence
  Description of Artificial Sequence: Synthetic oligonucleotide
cggcgguacacuuuagguccu

SEQ ID NO: 89 - RNA, 56 nt, Artificial Sequence
  Description of Artificial Sequence: Synthetic oligonucleotide
gucaaaagaccuuuuuaauuucuacucuuguagaucggcgguacacuuuagguccu

SEQ ID NO: 90 - RNA, 21 nt, Artificial Sequence
  Description of Artificial Sequence: Synthetic oligonucleotide
cggcgguacacuugguugaug

SEQ ID NO: 91 - RNA, 56 nt, Artificial Sequence
  Description of Artificial Sequence: Synthetic oligonucleotide
gucaaaagaccuuuuuaauuucuacucuuguagaucggcgguacacuugguugaug

SEQ ID NO: 92 - RNA, 21 nt, Artificial Sequence
  Description of Artificial Sequence: Synthetic oligonucleotide
cggcgguacacuuggcuguuu

SEQ ID NO: 93 - RNA, 56 nt, Artificial Sequence
  Description of Artificial Sequence: Synthetic oligonucleotide
gucaaaagaccuuuuuaauuucuacucuuguagaucggcgguacacuuggcuguuu

SEQ ID NO: 94 - RNA, 21 nt, Artificial Sequence
  Description of Artificial Sequence: Synthetic oligonucleotide
cagcuccuggugcuuccggcg

SEQ ID NO: 95 - RNA, 56 nt, Artificial Sequence
  Description of Artificial Sequence: Synthetic oligonucleotide
gucaaaagaccuuuuuaauuucuacucuuguagaucagcuccuggugcuuccggcg

SEQ ID NO: 96 - RNA, 21 nt, Artificial Sequence
  Description of Artificial Sequence: Synthetic oligonucleotide
uacucagggcucugcagcucc

SEQ ID NO: 97 - RNA, 56 nt, Artificial Sequence
  Description of Artificial Sequence: Synthetic oligonucleotide
gucaaaagaccuuuuuaauuucuacucuuguagauuacucagggcucugcagcucc

SEQ ID NO: 98 - RNA, 21 nt, Artificial Sequence
  Description of Artificial Sequence: Synthetic oligonucleotide
cucagcuuguacucagggcuc

SEQ ID NO: 99 - RNA, 56 nt, Artificial Sequence
  Description of Artificial Sequence: Synthetic oligonucleotide
gucaaaagaccuuuuuaauuucuacucuuguagaucucagcuuguacucagggcuc

SEQ ID NO: 100 - RNA, 21 nt, Artificial Sequence
  Description of Artificial Sequence: Synthetic oligonucleotide
cuggcaagaccuccuccauca

SEQ ID NO: 101 - RNA, 56 nt, Artificial Sequence
  Description of Artificial Sequence: Synthetic oligonucleotide
gucaaaagaccuuuuuaauuucuacucuuguagaucuggcaagaccuccuccauca

SEQ ID NO: 102 - RNA, 21 nt, Artificial Sequence
  Description of Artificial Sequence: Synthetic oligonucleotide
aggucacugauggaggagguc

SEQ ID NO: 103 - RNA, 56 nt, Artificial Sequence
  Description of Artificial Sequence: Synthetic oligonucleotide
gucaaaagaccuuuuuaauuucuacucuuguagauaggucacugauggaggagguc

SEQ ID NO: 104 - RNA, 21 nt, Artificial Sequence
  Description of Artificial Sequence: Synthetic oligonucleotide
cgcggcaccuccuucagguca

SEQ ID NO: 105 - RNA, 56 nt, Artificial Sequence
  Description of Artificial Sequence: Synthetic oligonucleotide
gucaaaagaccuuuuuaauuucuacucuuguagaucgcggcaccuccuucagguca

SEQ ID NO: 106 - RNA, 20 nt, Artificial Sequence
  Description of Artificial Sequence: Synthetic oligonucleotide
gcagguucagaauuauaggg

SEQ ID NO: 107 - DNA, 99 bp, Artificial Sequence
  Description of Artificial Sequence: Synthetic oligonucleotide
  Description of Combined DNA/RNA Molecule: Synthetic oligonucleotide
gcaggttcagaattatagggguuuuagagcuagaaauagcaaguuaaaauaaggcuaguccguuaucaacuugaaaaaguggcaccgagucggugcuuu

SEQ ID NO: 108 - RNA, 20 nt, Artificial Sequence
  Description of Artificial Sequence: Synthetic oligonucleotide
gggcaauggauuggucaucc

SEQ ID NO: 109 - DNA, 99 bp, Artificial Sequence
  Description of Artificial Sequence: Synthetic oligonucleotide
  Description of Combined DNA/RNA Molecule: Synthetic oligonucleotide
gggcaatggattggtcatccguuuuagagcuagaaauagcaaguuaaaauaaggcuaguccguuaucaacuugaaaaaguggcaccgagucggugcuuu

SEQ ID NO: 110 - DNA, 136 bp, Artificial Sequence
  Description of Artificial Sequence: Synthetic polynucleotide
gtgatgacaaagaggaggtcggccactgacaggtgcagcctgtacttgtccgtcatgcttctcagtttcttctggtaacccatgaccaggatagtgatagtgaccaatccattgcccacaatgccagttaagaaga

SEQ ID NO: 111 - DNA, 21 bp, Unknown
  Description of Unknown: target sequence
cggcggtacactttaggtcct

SEQ ID NO: 112 - DNA, 21 bp, Unknown
  Description of Unknown: target sequence
cggcggtacacttggttgatg

SEQ ID NO: 113 - DNA, 21 bp, Unknown
  Description of Unknown: target sequence
cggcggtacacttggctgttt

SEQ ID NO: 114 - DNA, 21 bp, Unknown
  Description of Unknown: target sequence
cagctcctggtgcttccggcg

SEQ ID NO: 115 - DNA, 21 bp, Unknown
  Description of Unknown: target sequence
tactcagggctctgcagctcc

SEQ ID NO: 116 - DNA, 21 bp, Unknown
  Description of Unknown: target sequence
ctcagcttgtactcagggctc

SEQ ID NO: 117 - DNA, 21 bp, Unknown
  Description of Unknown: target sequence
ctggcaagacctcctccatca

SEQ ID NO: 118 - DNA, 21 bp, Unknown
  Description of Unknown: target sequence
aggtcactgatggaggaggtc

SEQ ID NO: 119 - DNA, 21 bp, Unknown
  Description of Unknown: target sequence
cgcggcacctccttcaggtca

SEQ ID NO: 120 - DNA, 20 bp, Unknown
  Description of Unknown: target sequence
gcaggttcagaattataggg

SEQ ID NO: 121 - DNA, 20 bp, Artificial Sequence
  Description of Artificial Sequence: Synthetic oligonucleotide
  Description of Combined DNA/RNA Molecule: Synthetic oligonucleotide
gggcaauggattggtcatcc

SEQ ID NO: 122 - protein, 30 aa, Artificial Sequence
  Description of Artificial Sequence: Synthetic polypeptide
  SITE (4)..(30): This region may encompass 1-9 “Gly Gly Ser” repeating units wherein some positions may be absent
  See specification as filed for detailed description of substitutions and preferred embodiments
GGSXXXXXXXXXXXXXXXXXXXXXXXXXXX

SEQ ID NO: 123 - protein, 20 aa, Artificial Sequence
  Description of Artificial Sequence: Synthetic peptide
GGGGSGGGGSGGGGSGGGGS

SEQ ID NO: 124 - DNA, 23 bp, Artificial Sequence
  Description of Artificial Sequence: Synthetic primer
tgatggaaaggttcagagctcag

SEQ ID NO: 125 - DNA, 23 bp, Artificial Sequence
  Description of Artificial Sequence: Synthetic primer
ggtagacttggagagagcacatc

SEQ ID NO: 126 - DNA, 20 bp, Artificial Sequence
  Description of Artificial Sequence: Synthetic primer
ctgtaggaagtggcctgtgt

SEQ ID NO: 127 - DNA, 21 bp, Artificial Sequence
  Description of Artificial Sequence: Synthetic primer
gctgtgataacattcagcccc